(19) United States
(12) Patent Application Publication
(10) Pub. No.: US 2023/0218573 A1
(43) Pub. Date: Jul. 13, 2023

(54) INDOLE COMPOUNDS FOR THE TREATMENT OF NEURODEGENERATIVE DISEASES

(71) Applicant: GALYAN BIO, INC., Berkeley, CA (US)

(72) Inventors: Simon Marius GALYAN, Krailling (DE); Duncan Bruce JUDD, Hertfordshire (GB)

(21) Appl. No.: 17/904,829

(22) PCT Filed: Feb. 23, 2021
(86) PCT No.: PCT/US2021/019296

§ 371 Date: Aug. 23, 2022

Related U.S. Application Data
(60) Provisional application No. 62/980,632, filed on Feb. 24, 2020.
(60) Provisional application No. 63/119,402, filed on Nov. 30, 2020.

(51) Int. Cl. A61K 31/404; A61K 31/5377; A61K 31/496; A61P 25/28; A61K 31/454; A61K 31/519; A61K 31/437

(52) U.S. Cl. CPC A61K 31/404; A61K 31/437; A61K 31/454; A61K 31/496; A61K 31/519; A61K 31/5377; A61P 25/28

(57) ABSTRACT

Methods of treating polyQ diseases or disorders such as Huntington’s Disease are presented. Compounds of the disclosure are capable of binding directly to the polyQ segment of mutated huntingtin. This binding results in at least partial reversal of the conformation of mutated huntingtin to wild type huntingtin. The binding also facilitates autophagic removal of misfolded and/or mutated proteins such as mutated huntingtin.

### RELATED APPLICATIONS

This application claims priority to and the benefit of U.S. Provisional Pat. Application Number 62/980,632 filed Feb. 24, 2020 and U.S. Provisional Pat. Application Number 63/119,402 filed Nov. 30, 2020, each of which is incorporated by reference.

### BACKGROUND OF THE INVENTION

Huntington’s Disease (HD) is an autosomal dominant inherited neurodegenerative disorder caused by the expansion of the polyQ segment of huntingtin above a threshold of 36 CAG trinucleotide repeats. As the disease progresses, patients show movement disorder, speech impairment, cognitive impairment, anxiety, and difficulty in swallowing. Patients typically die within ten to twenty years of diagnosis. Currently there are no therapies available to mitigate the effects of Huntington’s Disease. Accordingly, there is a need for effective therapies for Huntington’s Disease.

### SUMMARY OF THE INVENTION

The present disclosure relates to compounds capable of reducing misfolded or mutated proteins (e.g., huntingtin), and the use of such compounds to treat disease (e.g., Huntington’s disease). As discussed below, the compounds disclosed can disrupt the pathological interaction of mutated and/or misfolded proteins (e.g., mutated huntingtin; “mHTT”) with other proteins.

Accordingly, in one aspect the present disclosure provides a method of treating Huntington’s Disease, comprising administering to a subject in need thereof an effective amount of a compound of Formula I:

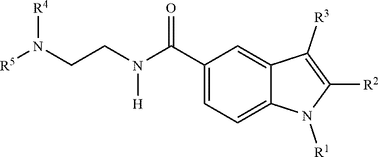

or a pharmaceutically acceptable salt, prodrug, solvate, hydrate, tautomer or isomer thereof, wherein:
R¹ is independently —H, —C1—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
R² is independently —H, —C1—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
or R¹ and R² can optionally combine, together with the atoms to which they are attached, to form a C₄-C₈heterocycle, wherein the heterocycle is optionally substituted with one or more substituents independently selected from the group consisting of —Br, —F, —Ci—C₆alkyl, —C₂—C₆alkenyl, and —C₂—C₆alkynyl, and wherein each alkyl, alkenyl, and alkynyl is optionally substituted with one or more —Br or —F;
R³ is independently —H, —C1—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
or R² and R³ can optionally combine, together with the atoms to which they are attached, to form a C₄-C₈carbocycle or a C₄-C₈heterocycle, wherein the carbocycle or heterocycle is optionally substituted with one or more substituents independently selected from the group consisting of —Br, —F, —C1—C₆alkyl, —C₂—C₆alkenyl, and —C₂—C₆alkynyl, and wherein each alkyl, alkenyl, and alkynyl is optionally substituted with one or more —Br or —F;
R⁴ is independently —H, —C1—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
R⁵ is independently —H, —C1—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
or R⁴ and R⁵ can optionally combine, together with the nitrogen atom to which they are attached, to form a C₃—C₈heterocycle, wherein the heterocycle is optionally substituted with one or more substituents independently selected from the group consisting of —Br, —F, —Ci—C₆alkyl, —C₂—C₆alkenyl, and —C₂—C₆alkynyl, and wherein each alkyl, alkenyl and alkynyl is optionally substituted with one or more —Br or —F.

In another aspect, the present disclosure provides a method of reversing a conformational change of mutated huntingtin, comprising administering to a subject in need thereof an effective amount of a compound of Formula I:

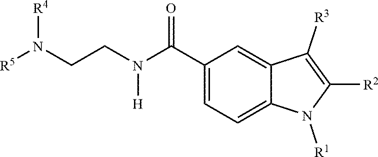

or a pharmaceutically acceptable salt, prodrug, solvate, hydrate, tautomer or isomer thereof, wherein:
R¹ is independently —H, —C1—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
R² is independently —H, —C1—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
or R¹ and R² can optionally combine, together with the atoms to which they are attached, to form a C₄-C₈heterocycle, wherein the heterocycle is optionally substituted with one or more substituents independently selected from the group consisting of —Br, —F, —Ci—C₆alkyl, —C₂—C₆alkenyl, and —C₂—C₆alkynyl, and wherein each alkyl, alkenyl, and alkynyl is optionally substituted with one or more —Br or —F;
R³ is independently —H, —C1—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
or R² and R³ can optionally combine, together with the atoms to which they are attached, to form a C₄-C₈carbocycle or a C₄-C₈heterocycle, wherein the carbocycle or heterocycle is optionally substituted with one or more substituents independently selected from the group consisting of —Br, —F, —C1—C₆alkyl, —C₂—C₆alkenyl, and —C₂—C₆alkynyl, and wherein each alkyl, alkenyl, and alkynyl is optionally substituted with one or more —Br or —F;
R⁴ is independently —H, —C1—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
R⁵ is independently —H, —C1—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
or R⁴ and R⁵ can optionally combine, together with the nitrogen atom to which they are attached, to form a C₃-C₈heterocycle, wherein the heterocycle is optionally substituted with one or more substituents independently selected from the group consisting of —Br, —F, —Ci—C₆alkyl, —C₂—C₆alkenyl, and —C₂—C₆alkynyl, and wherein each alkyl, alkenyl and alkynyl is optionally substituted with one or more —Br or —F

In another aspect, the present disclosure provides a method of treating a polyQ disease, comprising administering to a subject in need thereof an effective amount of a compound of Formula I:

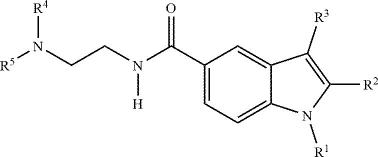

or a pharmaceutically acceptable salt, prodrug, solvate, hydrate, tautomer or isomer thereof, wherein:
R¹ is independently —H, —C1—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
R² is independently —H, —C1—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
or R¹ and R² can optionally combine, together with the atoms to which they are attached, to form a C₄-C₈heterocycle, wherein the heterocycle is optionally substituted with one or more substituents independently selected from the group consisting of —Br, —F, —Ci—C₆alkyl, —C₂—C₆alkenyl, and —C₂—C₆alkynyl, and wherein each alkyl, alkenyl, and alkynyl is optionally substituted with one or more —Br or —F;
R³ is independently —H, —C1—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
or R² and R³ can optionally combine, together with the atoms to which they are attached, to form a C₄-C₈carbocycle or a C₄-C₈heterocycle, wherein the carbocycle or heterocycle is optionally substituted with one or more substituents independently selected from the group consisting of —Br, —F, —C1—C₆alkyl, —C₂—C₆alkenyl, and —C₂—C₆alkynyl, and wherein each alkyl, alkenyl, and alkynyl is optionally substituted with one or more —Br or —F;
R⁴ is independently —H, —C1—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
R⁵ is independently —H, —C1—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
or R⁴ and R⁵ can optionally combine, together with the nitrogen atom to which they are attached, to form a C₃-C₈heterocycle, wherein the heterocycle is optionally substituted with one or more substituents independently selected from the group consisting of —Br, —F, —Ci—C₆alkyl, —C₂—C₆alkenyl, and —C₂—C₆alkynyl, and wherein each alkyl, alkenyl and alkynyl is optionally substituted with one or more —Br or —F.

In another aspect, the present disclosure provides a method of reducing mutated or misfolded proteins, comprising administering to a subject in need thereof an effective amount of a compound of Formula I:

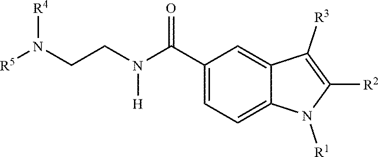

or a pharmaceutically acceptable salt, prodrug, solvate, hydrate, tautomer or isomer thereof, wherein:
R¹ is independently —H, —C1—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
R² is independently —H, —C1—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
or R¹ and R² can optionally combine, together with the atoms to which they are attached, to form a C₄-C₈heterocycle, wherein the heterocycle is optionally substituted with one or more substituents independently selected from the group consisting of —Br, —F, —Ci—C₆alkyl, —C₂—C₆alkenyl, and —C₂—C₆alkynyl, and wherein each alkyl, alkenyl, and alkynyl is optionally substituted with one or more —Br or —F;
R³ is independently —H, —C₁—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
or R² and R³ can optionally combine, together with the atoms to which they are attached, to form a C₄-C₈carbocycle or a C₄-C₈heterocycle, wherein the carbocycle or heterocycle is optionally substituted with one or more substituents independently selected from the group consisting of —Br, —F, —C1—C₆alkyl, —C₂—C₆alkenyl, and —C₂—C₆alkynyl, and wherein each alkyl, alkenyl, and alkynyl is optionally substituted with one or more —Br or —F;
R⁴ is independently —H, —C1—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
R⁵ is independently —H, —C1—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
or R⁴ and R⁵ can optionally combine, together with the nitrogen atom to which they are attached, to form a C₃-C₈heterocycle, wherein the heterocycle is optionally substituted with one or more substituents independently selected from the group consisting of —Br, —F, —Ci—C₆alkyl, —C₂—C₆alkenyl, and —C₂—C₆alkynyl, and wherein each alkyl, alkenyl and alkynyl is optionally substituted with one or more —Br or —F.

In another aspect, the present disclosure provides the use of a compound of Formula I in the manufacture of a medicament for treating Huntington’s Disease; reversing a conformational change of mutated huntingtin; treating a polyQ disease; or reducing mutated or misfolded proteins.

In another aspect, the present disclosure provides the use of a compound of Formula I for treating Huntington’s Disease; reversing a conformational change of mutated huntingtin; treating a polyQ disease; or reducing mutated or misfolded proteins.

In another aspect, the present disclosure provides a compound of Formula I or a pharmaceutically acceptable salt, prodrug, solvate, hydrate, tautomer or isomer thereof, for use in treating Huntington’s Disease; reversing a conformational change of mutated huntingtin; treating a polyQ disease; or reducing mutated or misfolded proteins.

In some embodiments of any of the above aspects, R¹ is —H. In some embodiments, R¹ is —Ci—C₆alkyl. In some embodiments, R² is —Ci—C₆alkyl. In some embodiments, R³ is —Ci—C₆alkyl . In some embodiments, R¹ and R² are —Ci—C₆alkyl. In some embodiments, R² and R³ are —Ci—C₆alkyl. In some embodiments, R² and R³ combine to form a carbocycle. In some embodiments, R² and R³ combine to form a heterocycle. In some embodiments, R⁴ is —Ci—C₆alkyl. In some embodiments, R⁵ is —Ci—C₆alkyl. In some embodiments, R⁴ and R⁵ combine to form a heterocycle. In some embodiments, the heterocycle is substituted with one or more —C₁—C₆alkyl.

In some embodiments, the compound has a structure of Formula I-A:

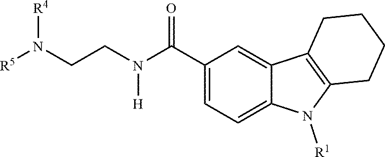

In some embodiments, the compound has a structure of Formula I-B:

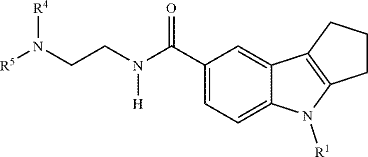

In some embodiments, the compound has a structure of Formula I-C:

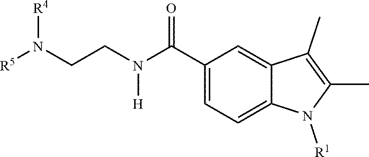

In some embodiments, the compound has a structure of Formula I-D:

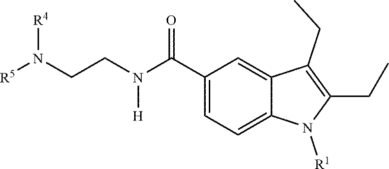

In some embodiments, the compound has a structure of Formula I-E:

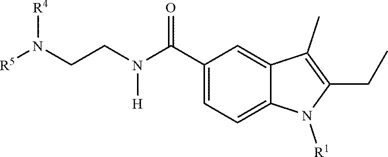

In some embodiments, the compound has a structure of Formula I-F:

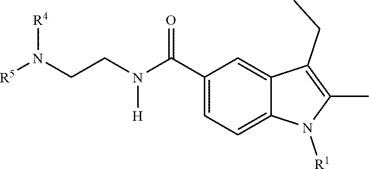

In some embodiments, the compound has a structure of Formula I-G:

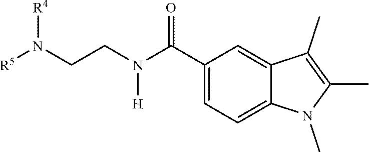

In some embodiments, the compound has a structure of Formula I-H:

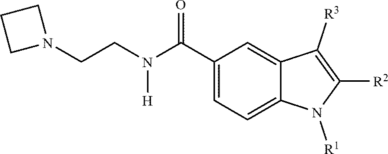

In some embodiments, the compound has a structure of Formula I-I:

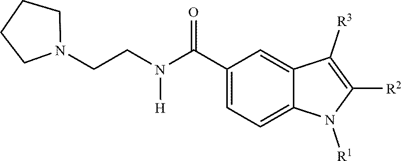

In some embodiments, the compound has a structure of Formula I-J:

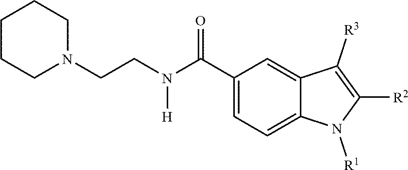

In some embodiments, the compound has a structure of Formula I-K:

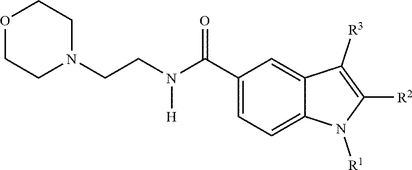

In some embodiments, the compound has a structure of Formula I-L:

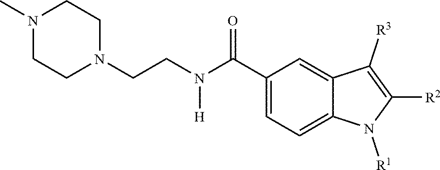

In some embodiments, the compound has a structure of Formula I-M:

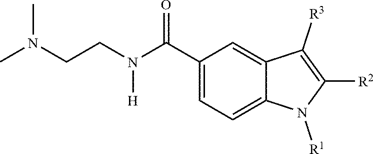

In some embodiments, the compound has a structure of Formula I-N:

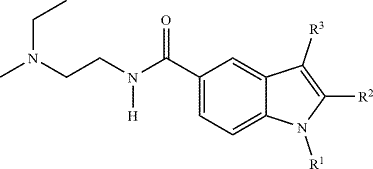

In some embodiments, the compound has a structure of Formula I-O:

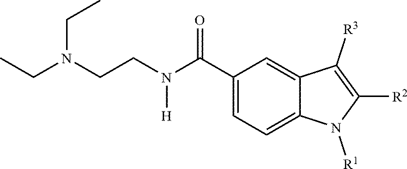

In another aspect, the present disclosure provides a compound selected from the group consisting of:

| Compound No. | Structure |
|---|---|
| 1 | 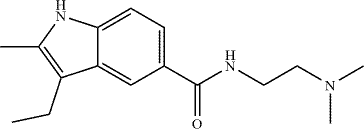 |
| 2 | 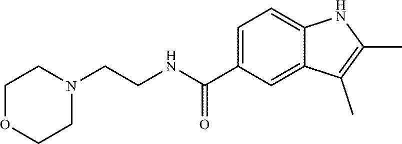 [SMILES: Cc1[nH]c2ccc(C(=O)NCCN3CCOCC3)cc2c1C] |
| 3 | 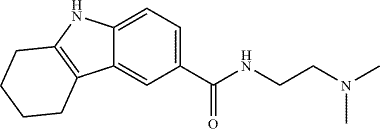 [SMILES: CN(C)CCNC(=O)c1ccc2[nH]c3c(c2c1)CCCC3] |
| 4 | 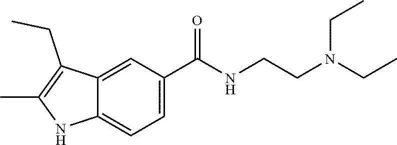 [SMILES: CCc1c(C)[nH]c2ccc(C(=O)NCCN(CC)CC)cc12] |
| 5 | 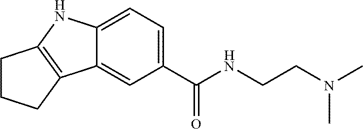 [SMILES: CN(C)CCNC(=O)c1ccc2[nH]c3c(c2c1)CCC3] |
| 6 | 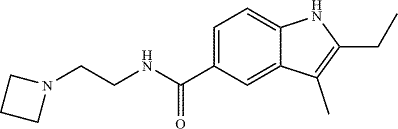 [SMILES: CCc1[nH]c2ccc(C(=O)NCCN3CCC3)cc2c1C] |
| 7 | 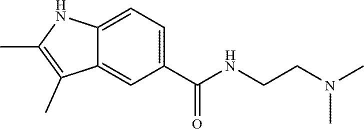 |
| 8 | 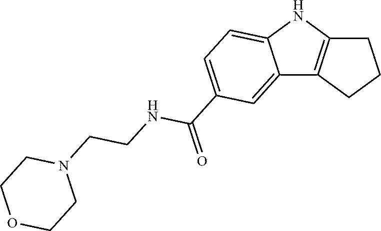 [SMILES: O=C(NCCN1CCOCC1)c1ccc2[nH]c3c(c2c1)CCC3] |
| 9 | 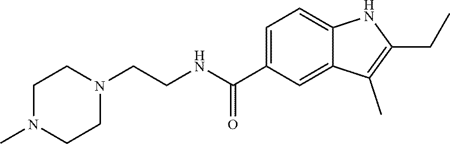 [SMILES: CCc1[nH]c2ccc(C(=O)NCCN3CCN(C)CC3)cc2c1C] |
| 10 | 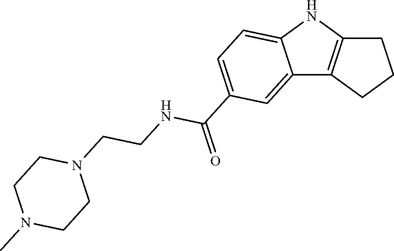 [SMILES: CN1CCN(CCNC(=O)c2ccc3[nH]c4c(c3c2)CCC4)CC1] |
| 11 | 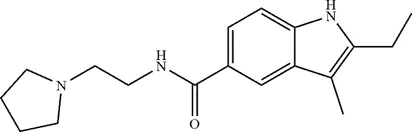 [SMILES: CCc1[nH]c2ccc(C(=O)NCCN3CCCC3)cc2c1C] |
| 12 | 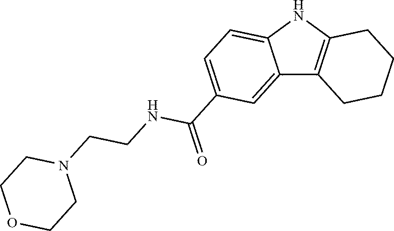 [SMILES: O=C(NCCN1CCOCC1)c1ccc2[nH]c3c(c2c1)CCCC3] |
| 13 | 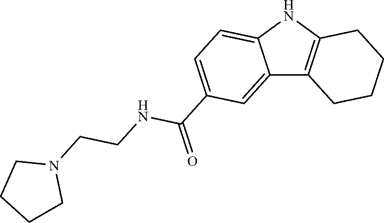 [SMILES: O=C(NCCN1CCCC1)c1ccc2[nH]c3c(c2c1)CCCC3] |
| 14 | 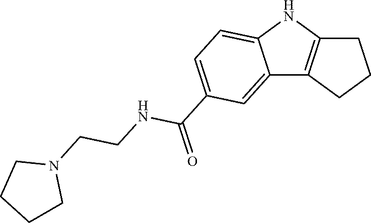 [SMILES: O=C(NCCN1CCCC1)c1ccc2[nH]c3c(c2c1)CCC3] |
| 15 | 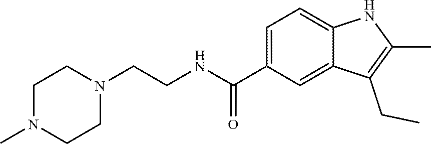 |
| 16 | 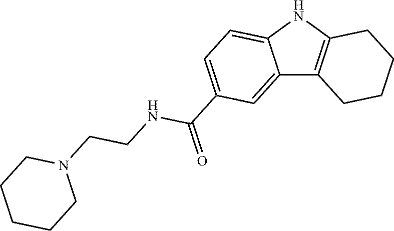 [SMILES: O=C(NCCN1CCCCC1)c1ccc2[nH]c3c(c2c1)CCCC3] |
| 17 | 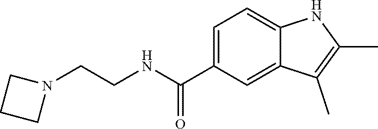 [SMILES: Cc1[nH]c2ccc(C(=O)NCCN3CCC3)cc2c1C] |
| 18 | 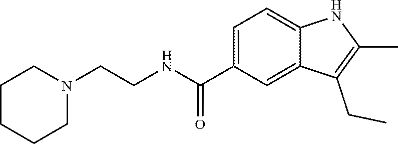 [SMILES: CCc1c(C)[nH]c2ccc(C(=O)NCCN3CCCCC3)cc12] |
| 19 | 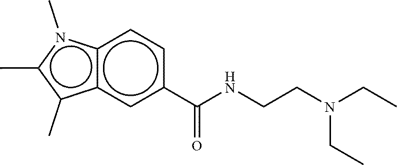 |
| 20 | 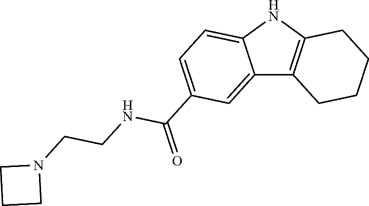 [SMILES: O=C(NCCN1CCC1)c1ccc2[nH]c3c(c2c1)CCCC3] |
| 21 | 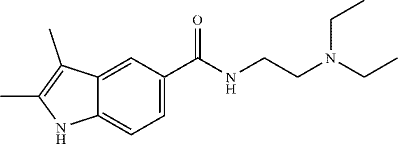 [SMILES: CCN(CC)CCNC(=O)c1ccc2[nH]c(C)c(C)c2c1] |
| 22 | 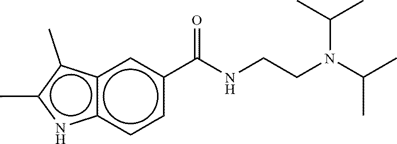 |
| 23 | 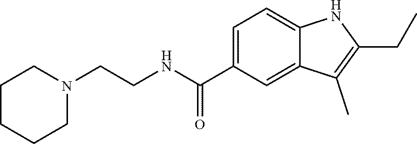 [SMILES: CCc1[nH]c2ccc(C(=O)NCCN3CCCCC3)cc2c1C] |
| 24 | |
| 25 | 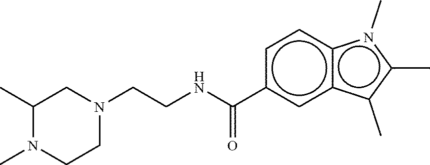 |
| 26 | 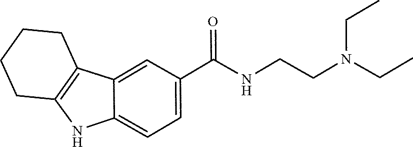 [SMILES: CCN(CC)CCNC(=O)c1ccc2[nH]c3c(c2c1)CCCC3] |
| 27 | 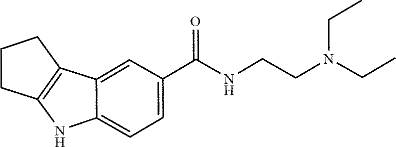 [SMILES: CCN(CC)CCNC(=O)c1ccc2[nH]c3c(c2c1)CCC3] |
| 28 | 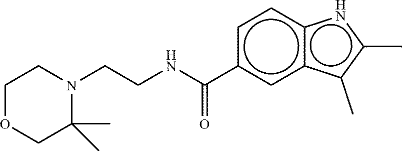 |
| 29 | 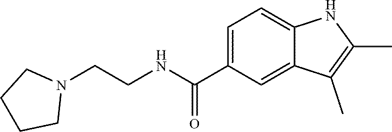 [SMILES: Cc1[nH]c2ccc(C(=O)NCCN3CCCC3)cc2c1C] |
| 30 | 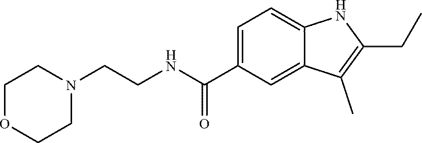 [SMILES: CCc1[nH]c2ccc(C(=O)NCCN3CCOCC3)cc2c1C] |
| 31 | |
| 32 | 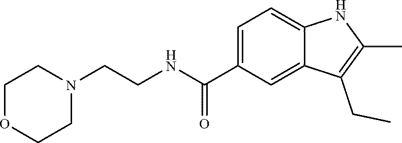 [SMILES: CCc1c(C)[nH]c2ccc(C(=O)NCCN3CCOCC3)cc12] |
| 33 | 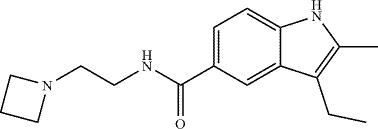 [SMILES: CCc1c(C)[nH]c2ccc(C(=O)NCCN3CCC3)cc12] |
| 34 | 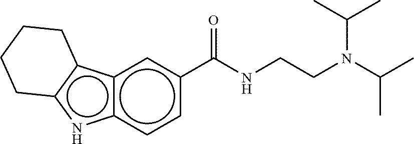 |
| 35 | 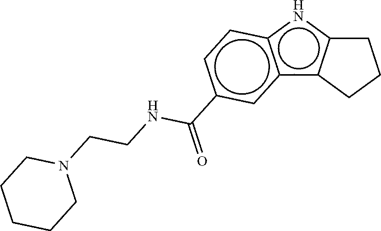 |
| 36 | 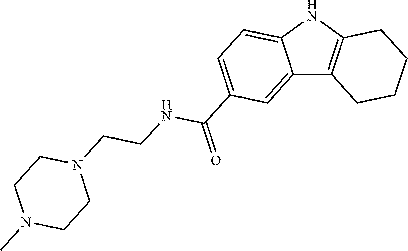 [SMILES: CN1CCN(CCNC(=O)c2ccc3[nH]c4c(c3c2)CCCC4)CC1] |
| 37 | 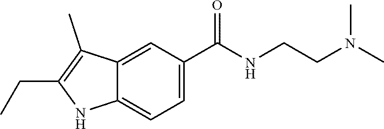 [SMILES: CCc1[nH]c2ccc(C(=O)NCCN(C)C)cc2c1C] |
| 38 | 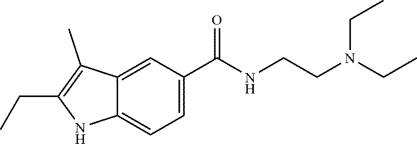 [SMILES: CCc1[nH]c2ccc(C(=O)NCCN(CC)CC)cc2c1C] |
| 39 | 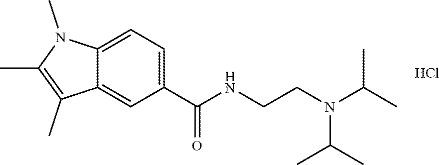 [SMILES: Cc1c(C)n(C)c2ccc(C(=O)NCCN(C(C)C)C(C)C)cc12 ; Cl] |
| 40 | 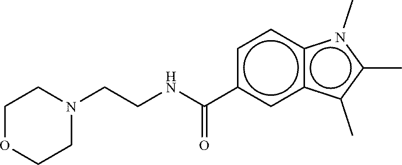 |
| 41 | 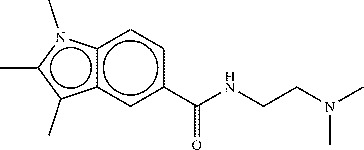 |
| 42 | 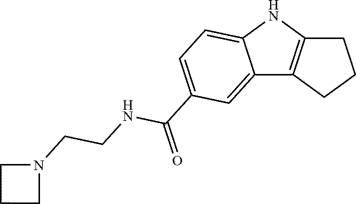 [SMILES: O=C(NCCN1CCC1)c1ccc2[nH]c3c(c2c1)CCC3] |
| 43 | 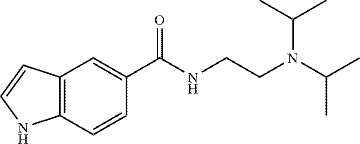 [SMILES: CC(C)N(CCNC(=O)c1ccc2[nH]ccc2c1)C(C)C] |
| 44 | 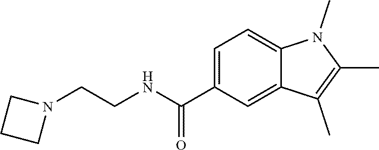 [SMILES: Cc1c(C)n(C)c2ccc(C(=O)NCCN3CCC3)cc12] |
| 45 | 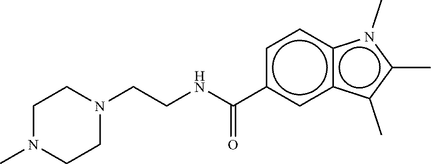 |
| 46 | 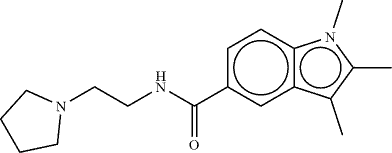 |
| 47 | 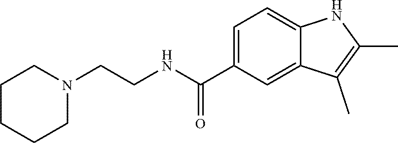 [SMILES: Cc1[nH]c2ccc(C(=O)NCCN3CCCCC3)cc2c1C] |
| 48 | 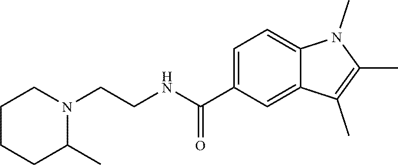 [SMILES: Cc1c(C)n(C)c2ccc(C(=O)NCCN3CCCCC3C)cc12] |
| 50 | 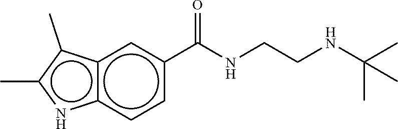 |
| 52 | 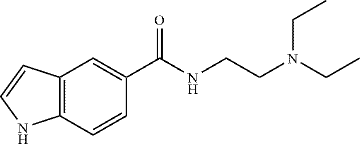 [SMILES: CCN(CC)CCNC(=O)c1ccc2[nH]ccc2c1] |
| 53 | 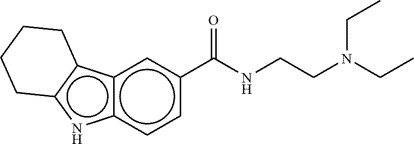 |
| 54 | 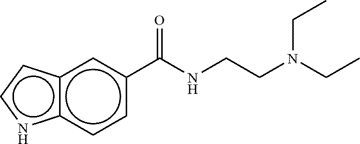 |
| 55 | |
| 56 | 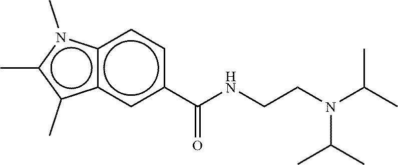 |
| 57 | 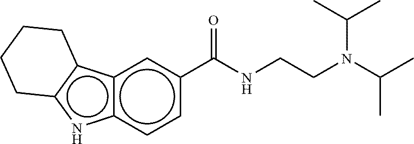 |
| 58 | |
| 59 | 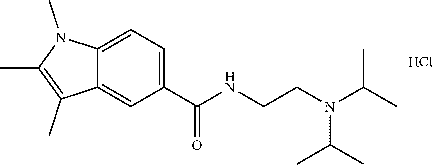 [SMILES: Cc1c(C)n(C)c2ccc(C(=O)NCCN(C(C)C)C(C)C)cc12 ; Cl] |
| 72 | 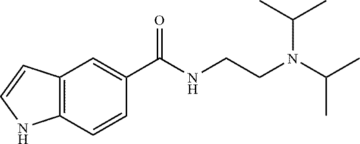 [SMILES: CC(C)N(CCNC(=O)c1ccc2[nH]ccc2c1)C(C)C] |
| 105 | 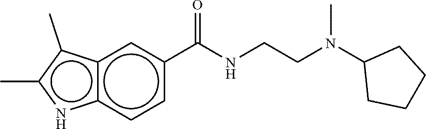 |
| 106 | 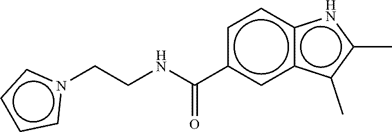 |
| 114 | 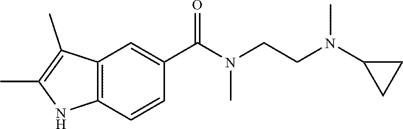 [SMILES: Cc1[nH]c2ccc(C(=O)N(C)CCN(C)C3CC3)cc2c1C] |

or a pharmaceutically acceptable salt thereof.

In some embodiments, the compound has a formula selected from the group consisting of:

Structure

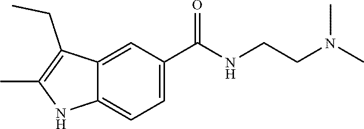

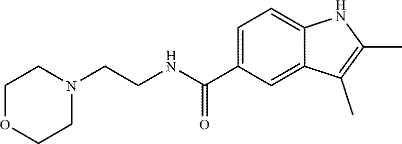 [SMILES: Cc1[nH]c2ccc(C(=O)NCCN3CCOCC3)cc2c1C]

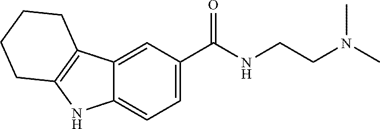 [SMILES: CN(C)CCNC(=O)c1ccc2[nH]c3c(c2c1)CCCC3]

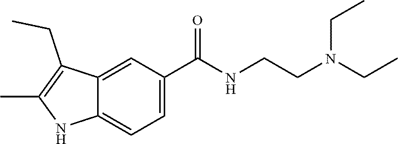 [SMILES: CCc1c(C)[nH]c2ccc(C(=O)NCCN(CC)CC)cc12]

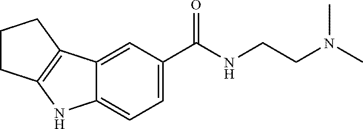 [SMILES: CN(C)CCNC(=O)c1ccc2[nH]c3c(c2c1)CCC3]

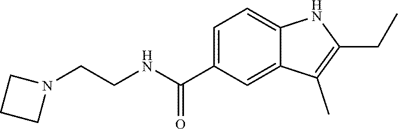 [SMILES: CCc1[nH]c2ccc(C(=O)NCCN3CCC3)cc2c1C]

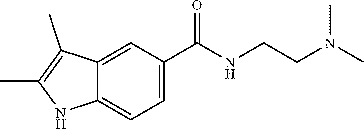

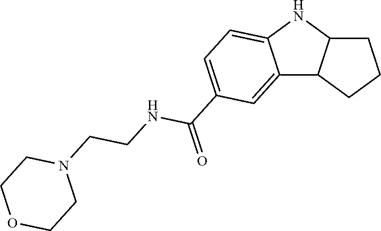 [SMILES: O=C(NCCN1CCOCC1)c1ccc2c(c1)C1CCCC1N2]

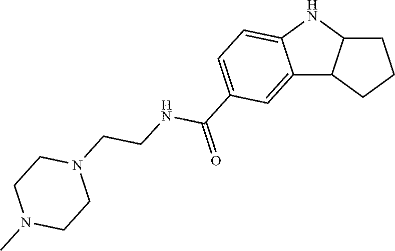 [SMILES: CN1CCN(CCNC(=O)c2ccc3c(c2)C2CCCC2N3)CC1]

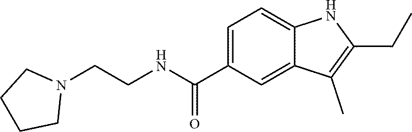 [SMILES: CCc1[nH]c2ccc(C(=O)NCCN3CCCC3)cc2c1C]

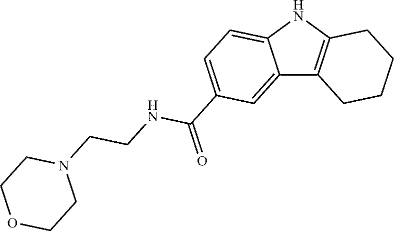 [SMILES: O=C(NCCN1CCOCC1)c1ccc2[nH]c3c(c2c1)CCCC3]

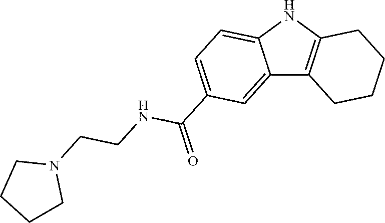 [SMILES: O=C(NCCN1CCCC1)c1ccc2[nH]c3c(c2c1)CCCC3]

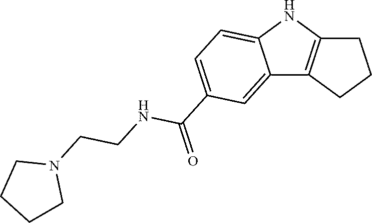 [SMILES: O=C(NCCN1CCCC1)c1ccc2[nH]c3c(c2c1)CCC3]

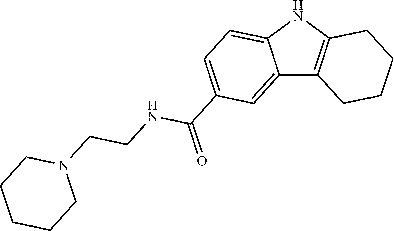 [SMILES: O=C(NCCN1CCCCC1)c1ccc2[nH]c3c(c2c1)CCCC3]

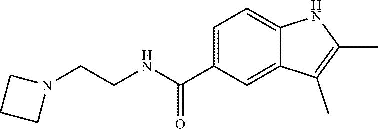 [SMILES: Cc1[nH]c2ccc(C(=O)NCCN3CCC3)cc2c1C]

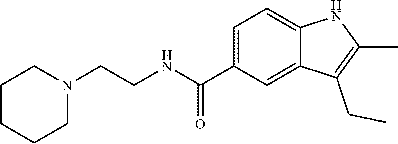 [SMILES: CCc1c(C)[nH]c2ccc(C(=O)NCCN3CCCCC3)cc12]

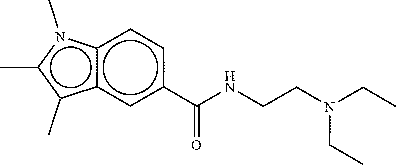

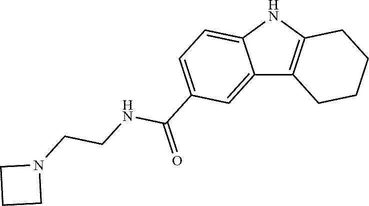 [SMILES: O=C(NCCN1CCC1)c1ccc2[nH]c3c(c2c1)CCCC3]

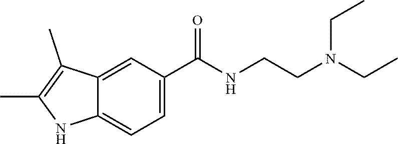 [SMILES: CCN(CC)CCNC(=O)c1ccc2[nH]c(C)c(C)c2c1]

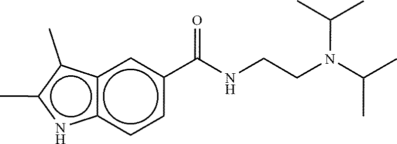

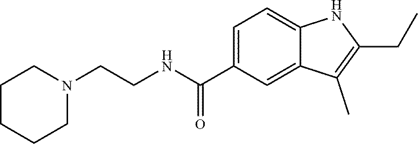 [SMILES: CCc1[nH]c2ccc(C(=O)NCCN3CCCCC3)cc2c1C]

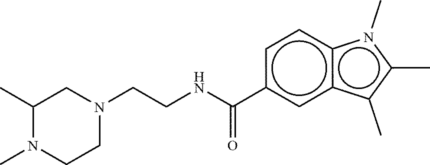

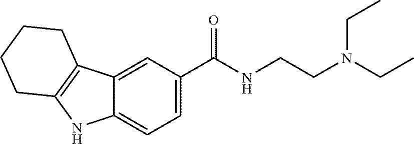 [SMILES: CCN(CC)CCNC(=O)c1ccc2[nH]c3c(c2c1)CCCC3]

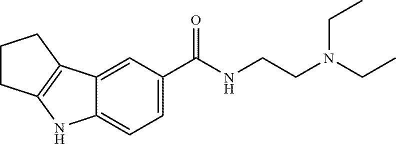 [SMILES: CCN(CC)CCNC(=O)c1ccc2[nH]c3c(c2c1)CCC3]

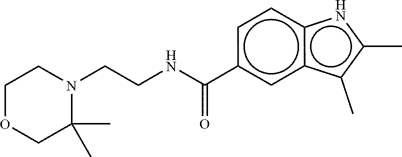

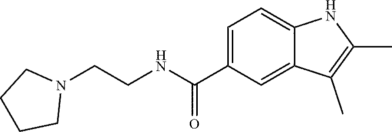 [SMILES: Cc1[nH]c2ccc(C(=O)NCCN3CCCC3)cc2c1C]

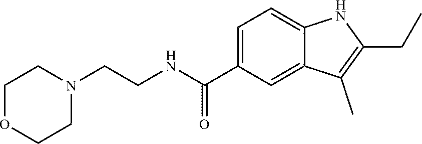 [SMILES: CCc1[nH]c2ccc(C(=O)NCCN3CCOCC3)cc2c1C]

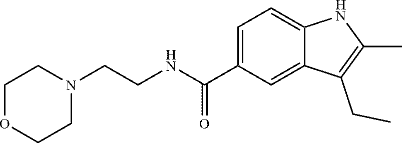 [SMILES: CCc1c(C)[nH]c2ccc(C(=O)NCCN3CCOCC3)cc12]

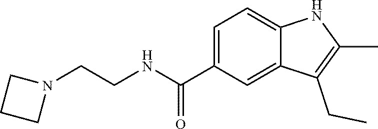 [SMILES: CCc1c(C)[nH]c2ccc(C(=O)NCCN3CCC3)cc12]

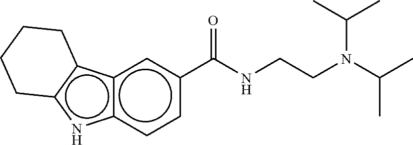

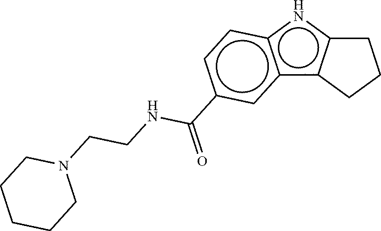

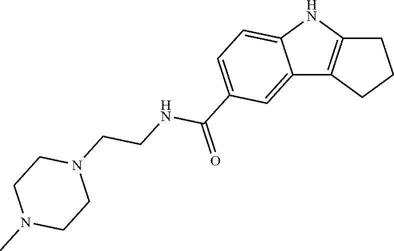 [SMILES: CN1CCN(CCNC(=O)c2ccc3[nH]c4c(c3c2)CCC4)CC1]

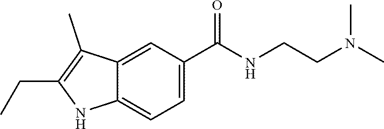 [SMILES: CCc1[nH]c2ccc(C(=O)NCCN(C)C)cc2c1C]

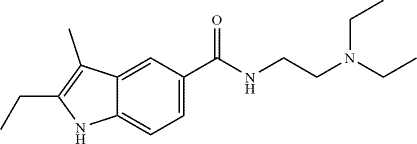 [SMILES: CCc1[nH]c2ccc(C(=O)NCCN(CC)CC)cc2c1C]

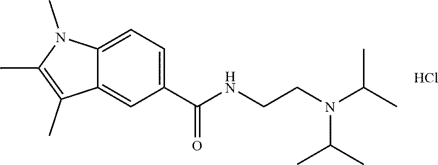 [SMILES: Cc1c(C)n(C)c2ccc(C(=O)NCCN(C(C)C)C(C)C)cc12 ; Cl]

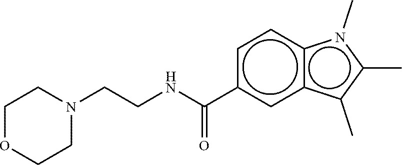

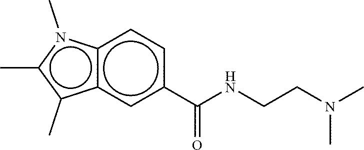

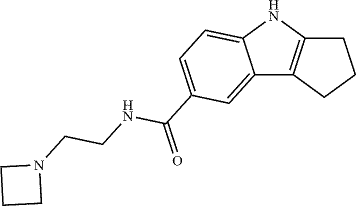 [SMILES: O=C(NCCN1CCC1)c1ccc2[nH]c3c(c2c1)CCC3]

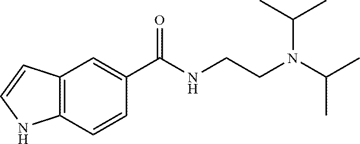 [SMILES: CC(C)N(CCNC(=O)c1ccc2[nH]ccc2c1)C(C)C]

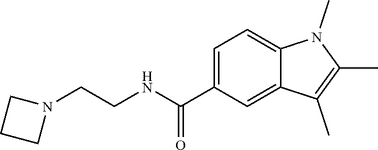 [SMILES: Cc1c(C)n(C)c2ccc(C(=O)NCCN3CCC3)cc12]

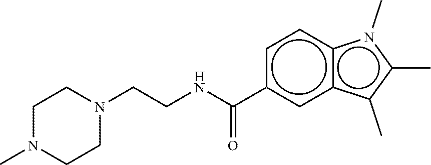

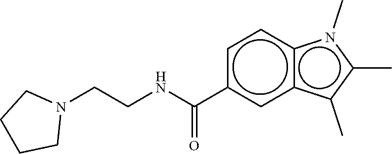

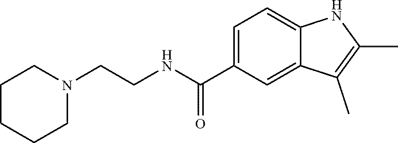 [SMILES: Cc1[nH]c2ccc(C(=O)NCCN3CCCCC3)cc2c1C]

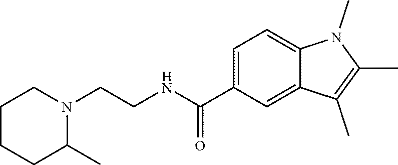 [SMILES: Cc1c(C)n(C)c2ccc(C(=O)NCCN3CCCCC3C)cc12]

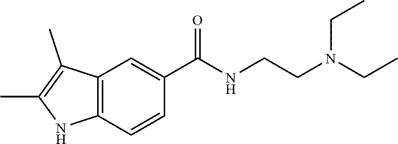 [SMILES: CCN(CC)CCNC(=O)c1ccc2[nH]c(C)c(C)c2c1]

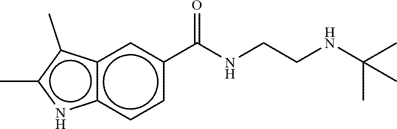

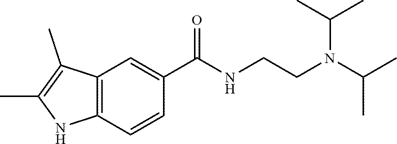 [SMILES: Cc1[nH]c2ccc(C(=O)NCCN(C(C)C)C(C)C)cc2c1C]

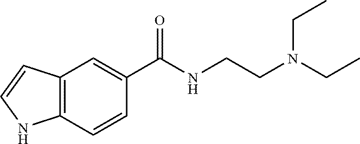 [SMILES: CCN(CC)CCNC(=O)c1ccc2[nH]ccc2c1]

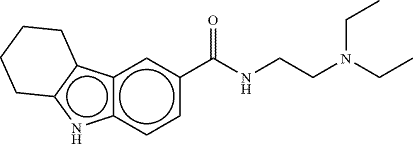

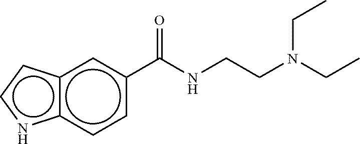

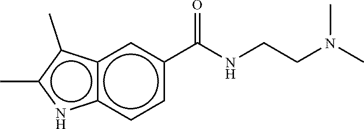

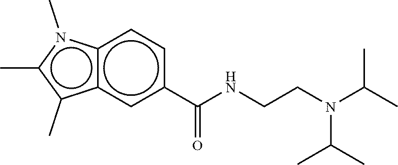

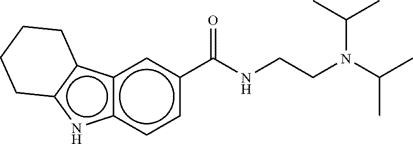

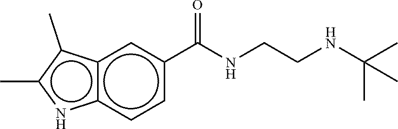

In some embodiments of any of the above aspects, the compound of Formula I partially reverses the conformational change of mutated huntingtin. In some embodiments, the compound of Formula I completely reverses the conformational change of mutated huntingtin.

In some embodiments, the conformational change results in improved autophagy. In some embodiments, the improved autophagy is improved autophagic flux.

In some embodiments, the polyglutamine-expansion (polyQ) disease is Huntington’s disease. In some embodiments the polyglutamine-expansion (polyQ) disease is spinocerebellar ataxias SCA1, SCA2, SCA3, SCA6, SCA7 and SCA17; DRPLA (Dentatorubropallidoluysian atrophy) or SBMA (Spinal and bulbar muscular atrophy).

In some embodiments, the mutated or misfolded protein is reduced in vivo. In some embodiments, the mutated or misfolded protein is reduced in the brain.

In some embodiments, the mutated or misfolded protein is reduced by autophagy. In some embodiments, the mutated or misfolded protein is reduced by increasing autophagic flux. In some embodiments, the mutated or misfolded protein is reduced by increasing degradation.

In some embodiments, the mutated or misfolded protein is huntingtin.

In another aspect, the present disclosure provides a method of treating Huntington’s Disease; reversing a conformational change of mutated huntingtin; treating a polyQ disease; reducing mutated or misfolded proteins; or inducing autophagy, comprising administering to a subject in need thereof a compound selected from:

Structure

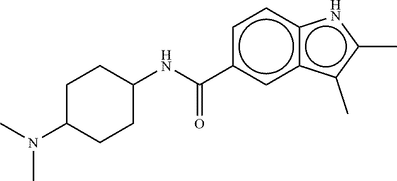

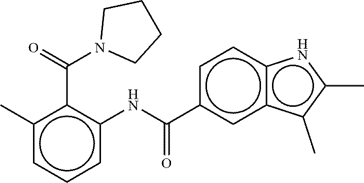

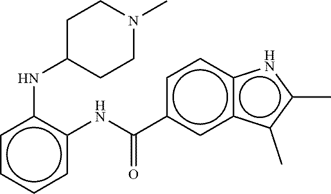

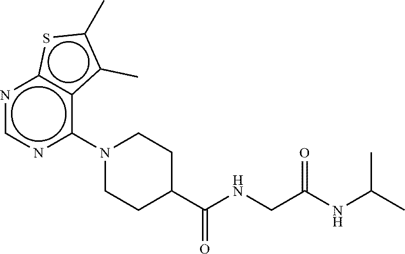

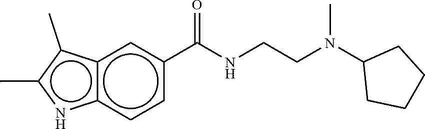

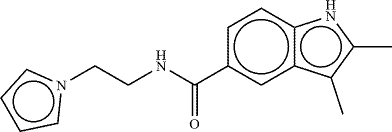

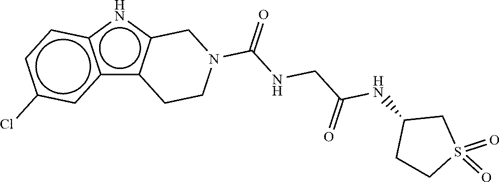

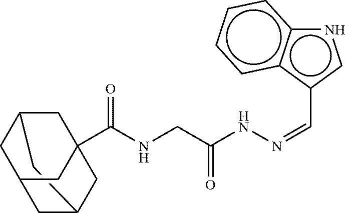

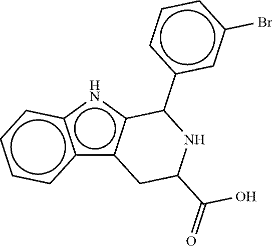

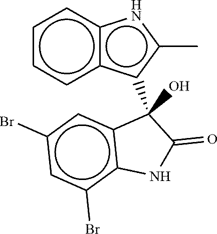

In another aspect, the present disclosure provides a compound selected from the group consisting of:

Structure

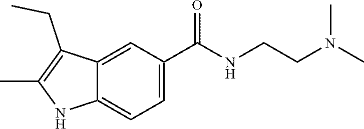

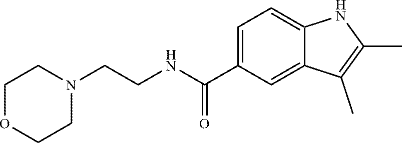 [SMILES: Cc1[nH]c2ccc(C(=O)NCCN3CCOCC3)cc2c1C]

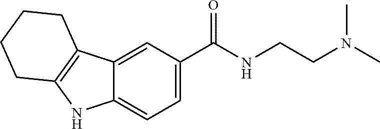 [SMILES: CN(C)CCNC(=O)c1ccc2[nH]c3c(c2c1)CCCC3]

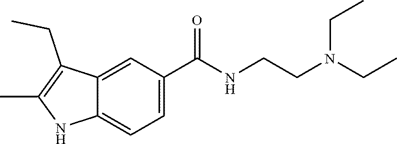 [SMILES: CCc1c(C)[nH]c2ccc(C(=O)NCCN(CC)CC)cc12]

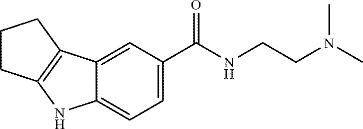 [SMILES: CN(C)CCNC(=O)c1ccc2[nH]c3c(c2c1)CCC3]

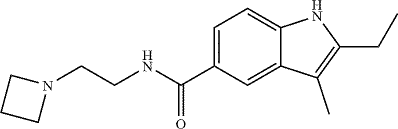 [SMILES: CCc1[nH]c2ccc(C(=O)NCCN3CCC3)cc2c1C]

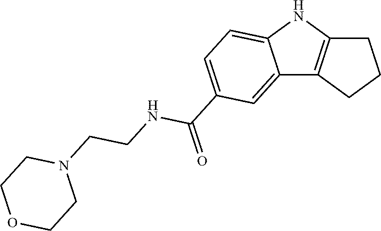 [SMILES: O=C(NCCN1CCOCC1)c1ccc2[nH]c3c(c2c1)CCC3]

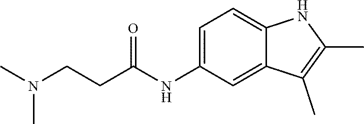

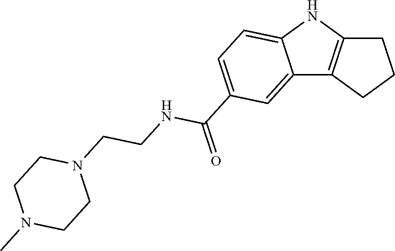 [SMILES: CN1CCN(CCNC(=O)c2ccc3[nH]c4c(c3c2)CCC4)CC1]

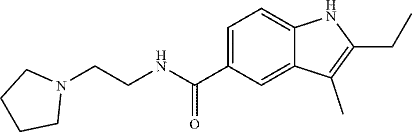 [SMILES: CCc1[nH]c2ccc(C(=O)NCCN3CCCC3)cc2c1C]

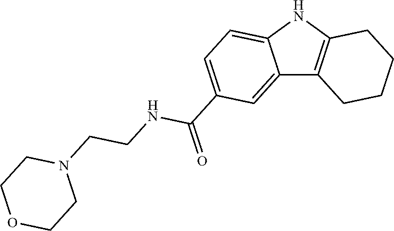 [SMILES: O=C(NCCN1CCOCC1)c1ccc2[nH]c3c(c2c1)CCCC3]

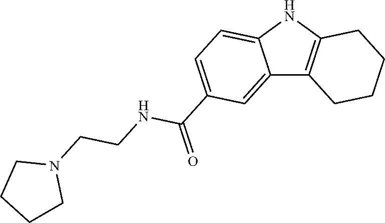 [SMILES: O=C(NCCN1CCCC1)c1ccc2[nH]c3c(c2c1)CCCC3]

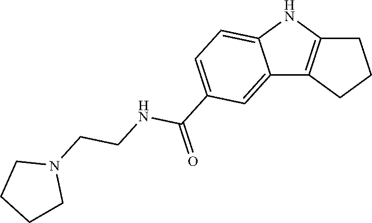 [SMILES: O=C(NCCN1CCCC1)c1ccc2[nH]c3c(c2c1)CCC3]

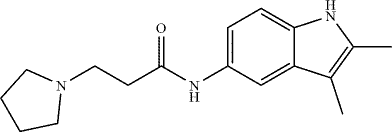 [SMILES: Cc1[nH]c2ccc(NC(=O)CCN3CCCC3)cc2c1C]

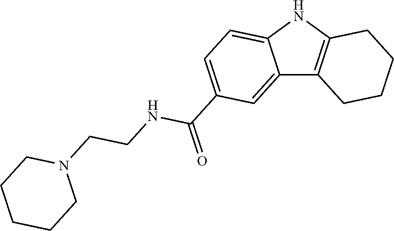 [SMILES: O=C(NCCN1CCCCC1)c1ccc2[nH]c3c(c2c1)CCCC3]

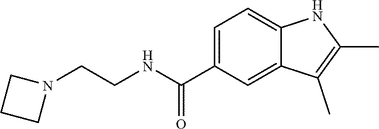 [SMILES: Cc1[nH]c2ccc(C(=O)NCCN3CCC3)cc2c1C]

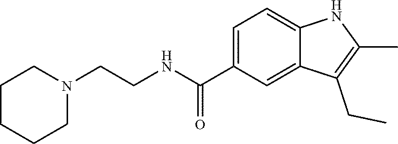 [SMILES: CCc1c(C)[nH]c2ccc(C(=O)NCCN3CCCCC3)cc12]

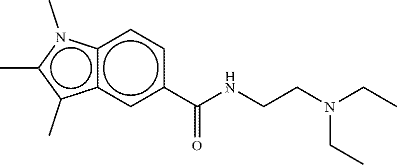

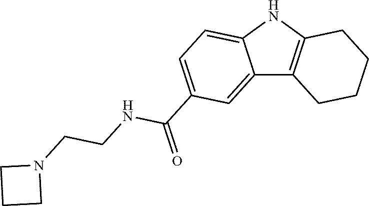 [SMILES: O=C(NCCN1CCC1)c1ccc2[nH]c3c(c2c1)CCCC3]

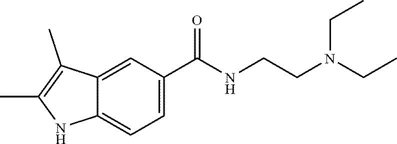 [SMILES: CCN(CC)CCNC(=O)c1ccc2[nH]c(C)c(C)c2c1]

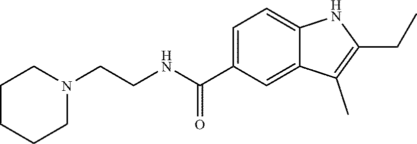 [SMILES: CCc1[nH]c2ccc(C(=O)NCCN3CCCCC3)cc2c1C]

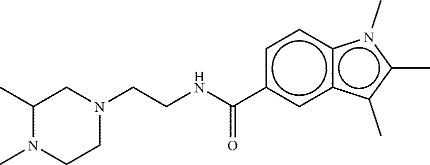

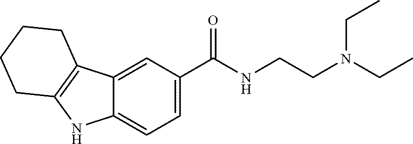 [SMILES: CCN(CC)CCNC(=O)c1ccc2[nH]c3c(c2c1)CCCC3]

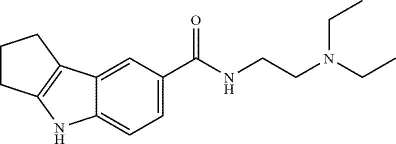 [SMILES: CCN(CC)CCNC(=O)c1ccc2[nH]c3c(c2c1)CCC3]

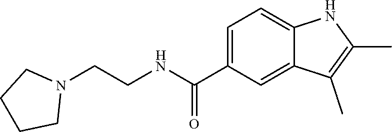 [SMILES: Cc1[nH]c2ccc(C(=O)NCCN3CCCC3)cc2c1C]

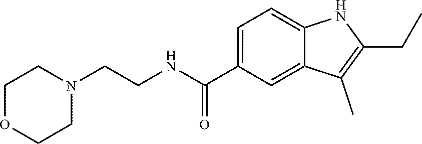 [SMILES: CCc1[nH]c2ccc(C(=O)NCCN3CCOCC3)cc2c1C]

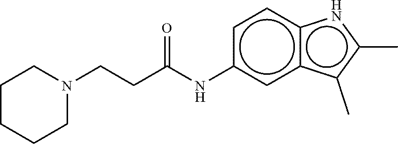

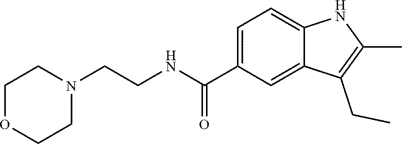 [SMILES: CCc1c(C)[nH]c2ccc(C(=O)NCCN3CCOCC3)cc12]

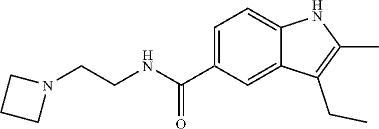 [SMILES: CCc1c(C)[nH]c2ccc(C(=O)NCCN3CCC3)cc12]

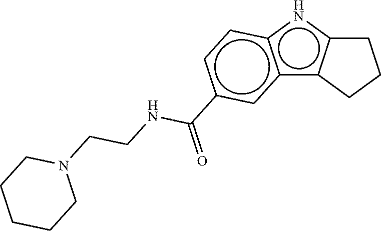

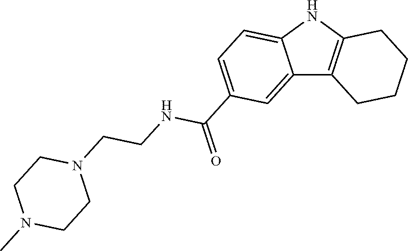 [SMILES: CN1CCN(CCNC(=O)c2ccc3[nH]c4c(c3c2)CCCC4)CC1]

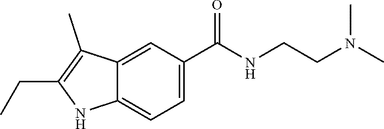 [SMILES: CCc1[nH]c2ccc(C(=O)NCCN(C)C)cc2c1C]

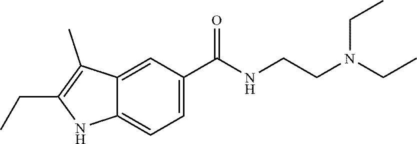 [SMILES: CCc1[nH]c2ccc(C(=O)NCCN(CC)CC)cc2c1C]

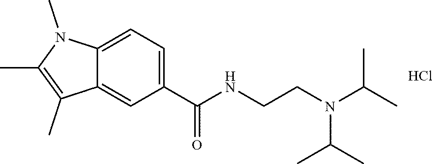 [SMILES: Cc1c(C)n(C)c2ccc(C(=O)NCCN(C(C)C)C(C)C)cc12 ; Cl]

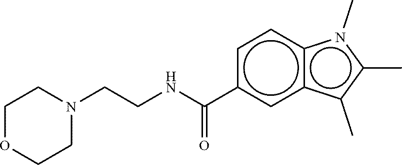

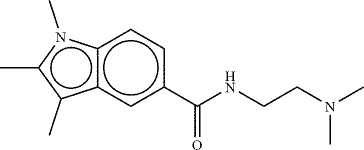

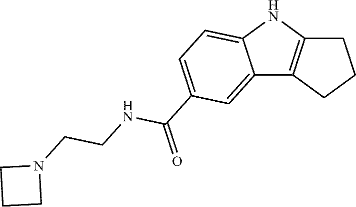 [SMILES: O=C(NCCN1CCC1)c1ccc2[nH]c3c(c2c1)CCC3]

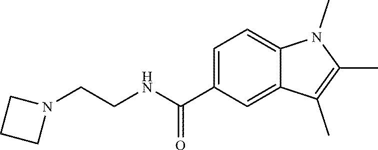 [SMILES: Cc1c(C)n(C)c2ccc(C(=O)NCCN3CCC3)cc12]

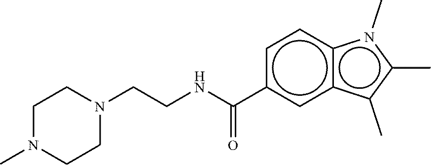

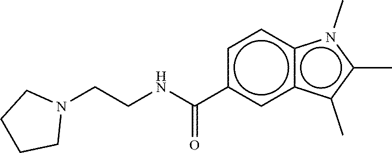

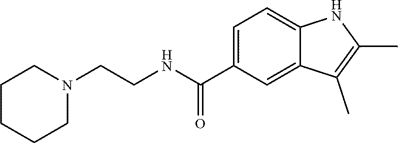 [SMILES: Cc1[nH]c2ccc(C(=O)NCCN3CCCCC3)cc2c1C]

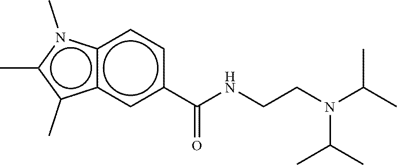

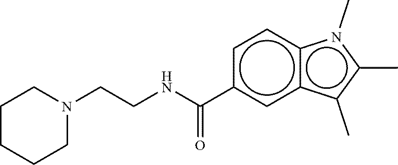

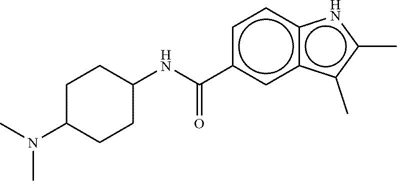

or a pharmaceutically acceptable salt thereof.

In some embodiments, the present disclosure provides a pharmaceutical composition comprising a compound selected from compound number 1, 2, 3, 4, 5, 6, 7, 9, 10, 11, 12, 13, 14, 15, 16, 17, 18, 19, 21, 22, 23, 24, 27, 29, 30, 31, 32, 33, 34, 36, 37, 38, 39, 42, 43, 45, 46, 47, 48, 49, 50, 53, 55, 57, 58, 60, 62, 63, 64, 71, 76, 80, 83, 84, 87, 92, 93, and/or 94, or a pharmaceutically acceptable salt thereof.

In another aspect, the present disclosure provides a method of treating Huntington’s Disease; reversing a conformational change of mutated huntingtin; treating a polyQ disease; or reducing mutated or misfolded proteins, comprising administering to a subject in need thereof an effective amount of a compound selected from compound number 1, 2, 3, 4, 5, 6, 7, 9, 10, 11, 12, 13, 14, 15, 16, 17, 18, 19, 21, 22, 23, 24, 27, 29, 30, 31, 32, 33, 34, 36, 37, 38, 39, 42, 43, 45, 46, 47, 48, 49, 50, 53, 55, 57, 58, 60, 62, 63, 64, 71, 76, 80, 83, 84, 87, 92, 93, and/or 94, or a pharmaceutically acceptable salt thereof.

In another aspect, the present disclosure provides compounds of Formula (I):

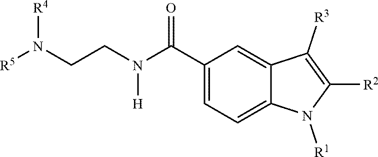

or a pharmaceutically acceptable salt thereof, wherein:
R¹ is independently hydrogen or methyl;
R² and R³ are each independently cyano, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl; wherein R² and R³ cannot both simultaneously be methyl;
R⁴ and R⁵ are each independently hydrogen, cyano, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl; or R⁴ and R⁵ can optionally combine, together with the atom to which they are attached, to form an optionally substituted 4 to 6-membered heterocyclyl, wherein the heterocyclyl is not imidazole substituted with a methyl.

In some embodiments, R¹ is hydrogen. In some embodiments, R¹ is methyl.

In some embodiments, R² and R³ are each independently substituted or unsubstituted alkyl, substituted or unsubstituted alkenyl, substituted or unsubstituted alkynyl, or substituted or unsubstituted carbocyclyl. In some embodiments, R² and R³ are each independently substituted or unsubstituted alkyl. In some embodiments, R² and R³ are each independently substituted or unsubstituted C₁-C₆ alkyl. In some embodiments, R² is methyl. In some embodiments, R³ is ethyl. In some embodiments, R² is methyl and R³ is ethyl.

In some embodiments, R⁴ and R⁵ are each independently hydrogen, cyano, substituted or unsubstituted alkyl, substituted or unsubstituted carbocyclyl, substituted or unsubstituted heterocyclyl, substituted or unsubstituted aryl, or substituted or unsubstituted heteroaryl. In some embodiments, R⁴ and R⁵ are each independently substituted or unsubstituted alkyl. In some embodiments, R⁴ and R⁵ are each independently substituted or unsubstituted C₁-C₆ alkyl. In some embodiments, R⁴ and R⁵ are each independently methyl, ethyl, or propyl. In some embodiments, R⁴ and R⁵ are both ethyl.

In some embodiments, R⁴ and R⁵ combine and together with the atom to which they are attached form a six member heterocyclyl with 1, 2, or 3 heteroatoms, wherein the heteroatoms are either O or N. In some embodiments, the heterocyclyl is optionally substituted with a C₁-C₆ alkyl. In some embodiments, the C₁-C₆ alkyl is methyl. In some embodiments, R⁴ and R⁵ combine and together with the atom to which they are attached form a five member heterocyclyl. In some embodiments, the heterocyclyl is optionally substituted with a C₁-C₆ alkyl. In some embodiments, he C₁-C₆ alkyl is methyl. In some embodiments, R⁴ and R⁵ combine and together with the atom to which they are attached form a four member heterocyclyl. In some embodiments, the heterocyclyl is optionally substituted with a C₁-C₆ alkyl. In some embodiments, the C₁-C₆ alkyl is methyl..

In some embodiments, the compound is selected from the group consisting of:

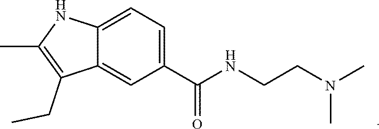

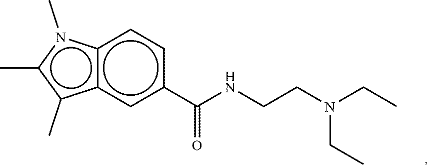

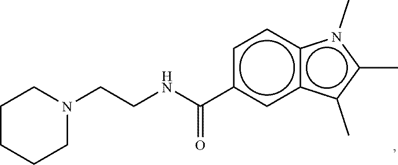

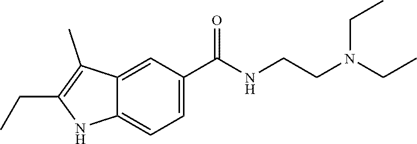 [SMILES: CCc1[nH]c2ccc(C(=O)NCCN(CC)CC)cc2c1C]

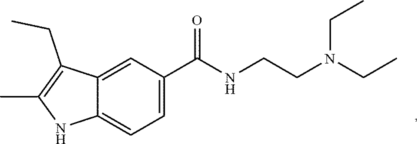

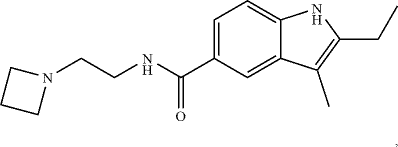

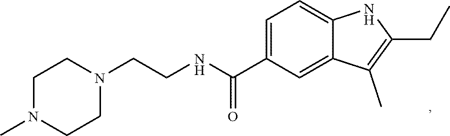

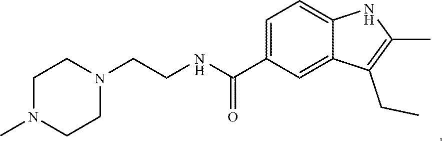

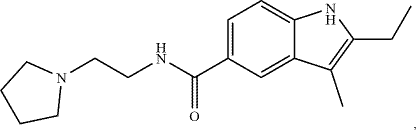

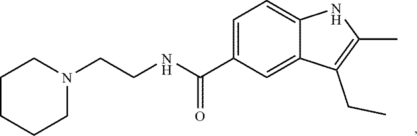

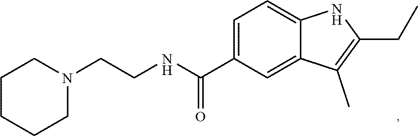

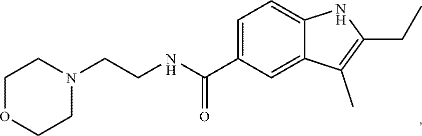

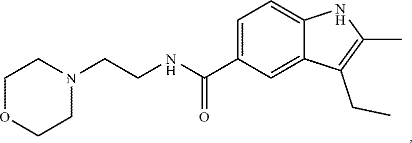

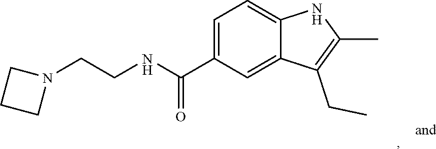

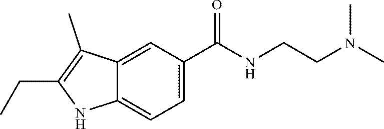 [SMILES: CCc1[nH]c2ccc(C(=O)NCCN(C)C)cc2c1C]

, or a pharmaceutically acceptable salt.

In some embodiments, the compound is

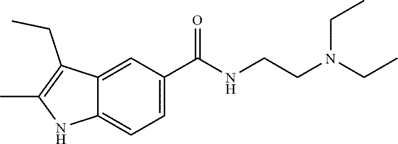 [SMILES: CCc1c(C)[nH]c2ccc(C(=O)NCCN(CC)CC)cc12]

In some embodiments, the compound is

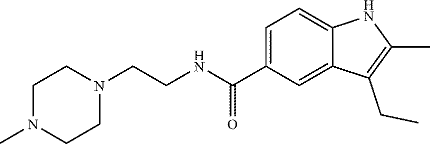

In some embodiments, the compound is

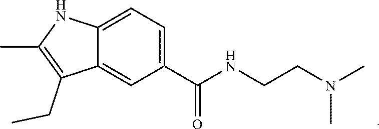

In another aspect, the present disclosure provides compounds of Formula (I):

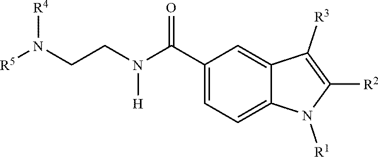

or a pharmaceutically acceptable salt thereof, wherein:
R¹ is independently hydrogen or methyl;
R² and R³ combine, together with the atoms to which they are attached, to form a substituted or unsubstituted carbocyclyl, substituted or unsubstituted heterocyclyl, substituted or unsubstituted aryl, or substituted or unsubstituted heteroaryl.
R⁴ and R⁵ are each independently hydrogen, cyano, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl; or R⁴ and R⁵ can optionally combine, together with the atom to which they are attached, to form an optionally substituted 4 to 6-membered heterocyclyl, wherein the heterocyclyl is not imidazole substituted with a methyl.

In some embodiments, R¹ is hydrogen. In some embodiments, R¹ is methyl.

In some embodiments, R₂ and R₃ combine, together with the atoms to which they are attached, to form a substituted or unsubstituted carbocyclyl. In some embodiments, R₂ and R₃ combine, together with the atoms to which they are attached, to form an unsubstituted carbocyclyl. In some embodiments, R₂ and R₃ combine, together with the atoms to which they are attached, to form a 5 or 6 member unsubstituted carbocyclyl.

In some embodiments, R₂ and R₃ combine, together with the atoms to which they are attached, to form a 5 member unsubstituted carbocyclyl. In some embodiments, the carbocyclyl is partially unsaturated.

In some embodiments, R₂ and R₃ combine, together with the atoms to which they are attached to form a 6 member unsubstituted carbocyclyl. In some embodiments, the carbocyclyl is partially unsaturated.

In some embodiments, R⁴ and R⁵ are each independently hydrogen, cyano, substituted or unsubstituted alkyl, substituted or unsubstituted carbocyclyl, substituted or unsubstituted heterocyclyl, substituted or unsubstituted aryl, or substituted or unsubstituted heteroaryl. In some embodiments, R⁴ and R⁵ are each independently substituted or unsubstituted alkyl. In some embodiments, R⁴ and R⁵ are each independently substituted or unsubstituted C₁-C₆ alkyl. In some embodiments, R⁴ and R⁵ are each independently methyl, ethyl, or propyl. In some embodiments, R⁴ and R⁵ are both methyl.

In some embodiments, R⁴ and R⁵ combine and together with the atom to which they are attached form a six member heterocyclyl with 1, 2, or 3 heteroatoms, wherein the heteroatoms are either O or N. In some embodiments, the heterocyclyl is optionally substituted with a C₁-C₆ alkyl. In some embodiments, the C₁-C₆ alkyl is methyl. In some embodiments, R⁴ and R⁵ combine and together with the atom to which they are attached form a five member heterocyclyl. In some embodiments, the heterocyclyl is optionally substituted with a C₁-C₆ alkyl. In some embodiments, the C₁-C₆ alkyl is methyl. In some embodiments, R⁴ and R⁵ combine and together with the atom to which they are attached form a four member heterocyclyl. In some embodiments, the heterocyclyl is optionally substituted with a C₁-C₆ alkyl. In some embodiments, the C₁-C₆ alkyl is methyl.

In some embodiments, the compound is selected from the group consisting of:

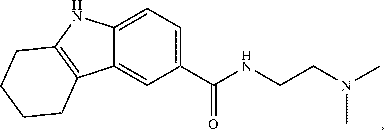

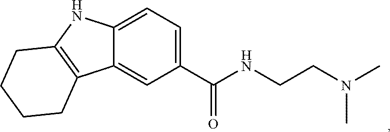

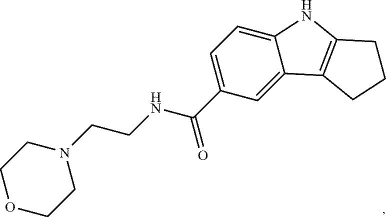

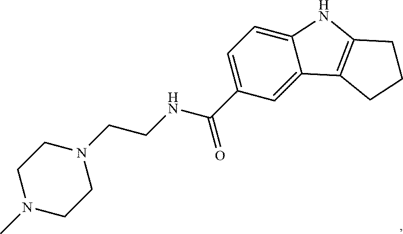

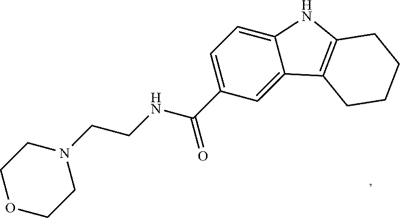

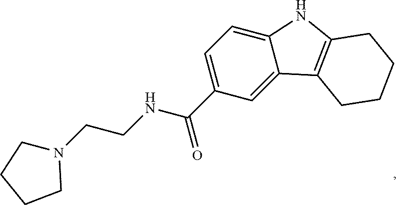

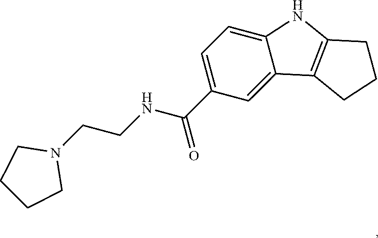

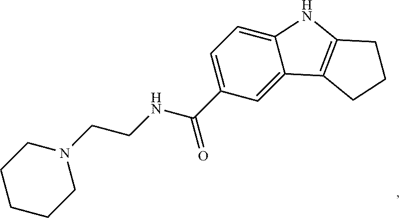

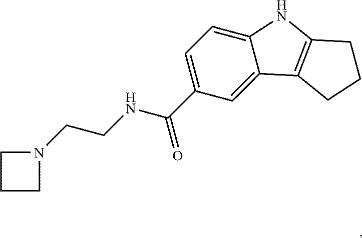

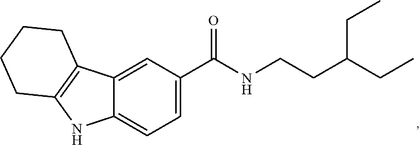

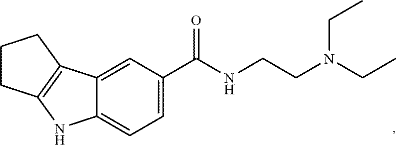

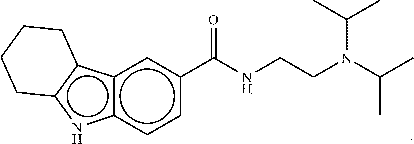

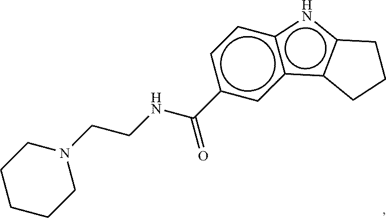

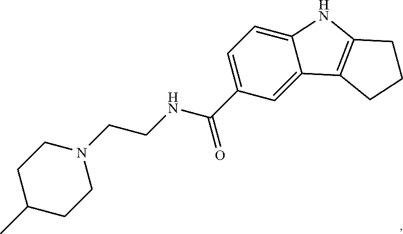

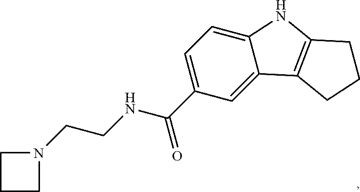

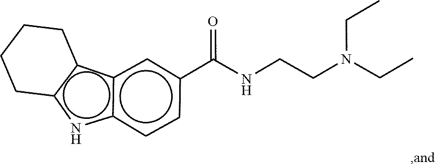

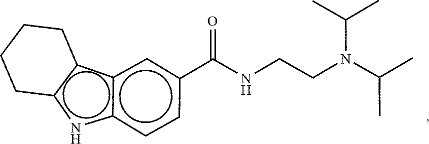

or a pharmaceutically acceptable salt thereof.

In some embodiments, the compound is

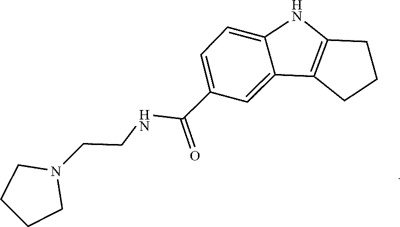

In some embodiments, the compound is

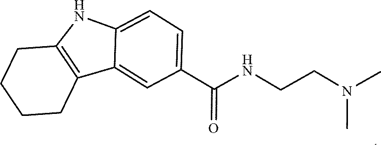

In some embodiments, the compound is

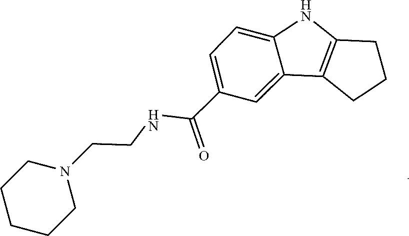

In some embodiments, the compound is

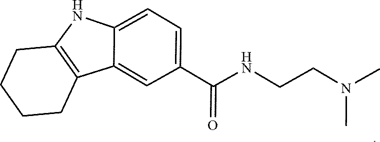

In some embodiments, the compound is

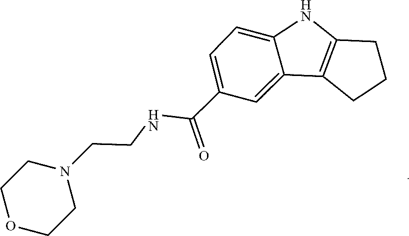

In some embodiments, the compound is

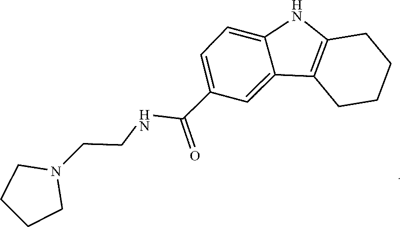

In some embodiments, the compound is

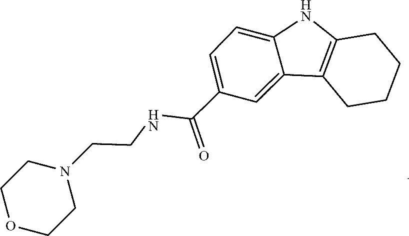

The details of the invention are set forth in the accompanying description below. Although methods and materials similar or equivalent to those described herein can be used in the practice or testing of the present invention, illustrative methods and materials are now described. Other features, objects, and advantages of the invention will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs. All patents and publications cited in this specification are incorporated herein by reference in their entireties.

### BRIEF DESCRIPTION OF THE FIGURES

FIG. 1 is a western blot that shows MurTRX and Mur16 protein expression. MurTRX and Mur16 proteins were expressed in E.coli. Four colonies of each (MurTRX -colony #2, 3, 6, 7; Mur16 - colony #1, 2, 3, 5) were selected and pellets were re-suspended in 150uL of lysis buffer. Mur16 colony #2 had the best expression level of those screened.

FIG. 2 is a western blot that shows Mur46 and MurTRX protein expression. Mur46 and MurTRX proteins were expressed in E.coli. Five colonies of each (Mur46 - colony #1, 2, 3, 4, 5; MurTRX - colony #1, 2, 3, 4, 5) were selected and pellets were re-suspended in 50 uL of lysis buffer. Proteins were detected for each of the Mur46 colonies. Low protein expression was observed for each of the MurTRX colonies.

FIG. 3 is a graph showing the saturation kinetics of 3B5H10 MAb on Mur46 and Mur16 coupled flow cells. 3B5H10 MAb (Sigma Aldrich, Saint Louis, USA) was diluted 1:600 and flowed over all four flow cells to the point of saturation using injections. The y-axis shows the Response Units (RU). The x-axis shows time (seconds).

FIG. 4 is a graph showing averaged densitometric measures of filter retardation following 24 hour treatment of Htt14A1.6 PC12 cells with different concentrations of Compound 7. A reduction of aggregation of Htt14A2.6 PC12 was not observed following treatment with Compound 7.

FIG. 5 is a graph showing averaged densitometric measures of filter retardation following 24 hour treatment of Htt14A1.6 PC12 cells with different concentrations of Compound 22. A reduction of aggregation of Htt14A2.6 PC12 was observed following treatment with Compound 22.

FIG. 6 is a graph showing the concentration of Compound 22 that penetrated the blood brain barrier, in vivo. Male CD1 mice were treated with Compound 28 at a dose of 33.3 mg/kg body weight. Following isolation of proteins precipitated from homogenized mouse brain tissue, the concentration of Compound 22 was determined using Ultra-High Performance Liquid Chromatography combined with Time-of-Flight Mass Spectrometry (UHPLC-TOF). Compound 22 penetrates the blood brain area.

FIG. 7 is a bar graph showing the nuclear diffuse mHTT levels of STHdh 111/111 cells and StHdh 7/7 cells treated with Compound 22. Nuclear diffuse mHTT was significantly reduced by 32% (p = 0.02) in STHdh 111/111 cells treated with Compound 22 in comparison to untreated STHdh 111/111 cells. But wildtype huntingtin was not reduced in STHdh 7/7 cells treated with Compound 22 in comparison to non-treated STHdh 7/7.

FIG. 8 is a line graph showing the cell viability of STHdh7/7 and STHdh 111/111 cells treated with Compound 22. Heat shock triggers accumulation of misfolded proteins which are degraded. Cell viability was in Compound 22 treated striatal neurons with mHTT almost at wildtype level in the first 24 h ctr control.

FIG. 9 is a bar graph showing the cell viability of STHdh 7/7 striatal cells following treatment with heat shock and small molecule compounds at 10 µM.

FIG. 10 is a schematic diagram of the study of Compound 22 on motor behavior in R6/2 mice of Huntington’s disease. Motor function testing using rotarod were commenced at 4 weeks (pre-treatment baseline) and continued at 6, 8 and 10 weeks of age, accompanied with grip strength at 4 weeks (pre-treatment baseline), 10 and 12 weeks of age. At the end point of 12 weeks of age, the mice were subjected to tissue collection.

FIG. 11 is a line graph showing the effects of chronic administration of Compound 22 (33 mg/kg) on body weight of pooled genders of R6/2 mice aged 3 to 12 weeks. Data are presented as mean ± SEM (WT Vehicle, n = 10; R6/2 Vehicle, n = 10; R6/2 Compound 22 33 mg/kg, n = 10). # p < 0.05, R6/2 Vehicle vs. WT Vehicle.

FIG. 12 is a line graph showing the effects of chronic administration of Compound 22 (33 mg/kg) on body weight of female R6/2 mice aged 3 to 12 weeks. Data are presented as mean ± SEM (WT Vehicle, n = 5; R6/2 Vehicle, n = 5; R6/2 Compound 22 33 mg/kg, n = 5). # p < 0.05, R6/2 Vehicle vs. WT Vehicle.

FIG. 13 is a line graph showing the effects of chronic administration of Compound 22 (33 mg/kg) on body weight of male R6/2 mice aged 3 to 12 weeks. Data are presented as mean ± SEM (WT Vehicle, n = 5; R6/2 Vehicle, n = 5; R6/2 Compound 22 33 mg/kg, n = 5). # p < 0.05, R6/2 Vehicle vs. WT Vehicle.

FIG. 14 is a graph showing the effects of chronic administration of Compound 22 (33 mg/kg) on rotarod latency of pooled genders of R6/2 mice at 4 to 10 weeks of age. Data are presented as mean ± SEM (WT Vehicle, n = 10; R6/2 Vehicle, n = 10; R6/2 Compound 22 33 mg/kg, n = 10). # p < 0.05, R6/2 Vehicle vs. WT Vehicle.

FIG. 15 is a bar graph showing the effects of chronic administration of Compound 22 (33 mg/kg) on rotarod latency of pooled genders of R6/2 mice at 4 to 10 weeks of age. Data are presented as percentage from 4-week pre-treatment baseline (mean + SEM) (WT Vehicle, n = 10; R6/2 Vehicle, n = 10; R6/2 Compound 22 33 mg/kg, n = 10). # p < 0.05, R6/2 Vehicle vs. WT Vehicle.

FIG. 16 is a graph showing the effects of chronic administration of Compound 22 (33 mg/kg) on rotarod latency of female R6/2 mice at 4 to 10 weeks of age. Data are presented as mean ± SEM (WT Vehicle, n = 10; R6/2 Vehicle, n = 10; R6/2 Compound 22 33 mg/kg, n = 10). # p < 0.05, R6/2 Vehicle vs. WT Vehicle.

FIG. 17 is a bar graph showing the effects of chronic administration of Compound 22 (33 mg/kg) on rotarod latency of female R6/2 mice at 4 to 10 weeks of age. Data are presented as percentage from 4-week pre-treatment baseline (mean + SEM) (WT Vehicle, n = 10; R6/2 Vehicle, n = 10; R6/2 Compound 22 33 mg/kg, n = 10). # p < 0.05, R6/2 Vehicle vs. WT Vehicle; * p < 0.05, R6/2 Compound 22 33 mg/kg vs. R6/2 Vehicle.

FIG. 18 is a graph showing the effects of chronic administration of Compound 22 (33 mg/kg) on rotarod latency of male R6/2 mice at 4 to 10 weeks of age. Data are presented as mean ± SEM (WT Vehicle, n = 10; R6/2 Vehicle, n = 10; R6/2 Compound 22 33 mg/kg, n = 10). # p < 0.05, R6/2 Vehicle vs. WT Vehicle.

FIG. 19 is a bar graph showing the effects of chronic administration of Compound 22 (33 mg/kg) on rotarod latency of male R6/2 mice at 4 to 10 weeks of age. Data are presented as percentage from 4-week pre-treatment baseline (mean + SEM) (WT Vehicle, n = 10; R6/2 Vehicle, n = 10; R6/2 Compound 22 33 mg/kg, n = 10). # p < 0.05, R6/2 Vehicle vs. WT Vehicle.

FIG. 20 is a bar graph showing the effects of chronic administration of Compound 22 (33 mg/kg) on grip strength of pooled genders of R6/2 mice from 4-12 weeks of age. Data are presented as mean ± SEM (WT Vehicle, n = 10; R6/2 Vehicle, n = 10; R6/2 Compound 22 33 mg/kg, n = 10). # p < 0.05, R6/2 Vehicle vs. WT Vehicle.

FIG. 21 is a bar graph showing the effects of chronic administration of Compound 22 (33 mg/kg) on grip strength of female R6/2 mice from 4-12 weeks of age. Data are presented as mean ± SEM (WT Vehicle, n = 10; R6/2 Vehicle, n = 10; R6/2 Compound 22 33 mg/kg, n = 10). # p < 0.05, R6/2 Vehicle vs. WT Vehicle.

FIG. 22 is a bar graph showing the effects of chronic administration of Compound 22 (33 mg/kg) on grip strength of male R6/2 mice from 4-12 weeks of age. Data are presented as mean ± SEM (WT Vehicle, n = 10; R6/2 Vehicle, n = 10; R6/2 Compound 22 33 mg/kg, n = 10). # p < 0.05, R6/2 Vehicle vs. WT Vehicle

FIGS. 23A-23F are a series of confocal fluorescence images of striatum region from W from WT vehicle, TG vehicle and TG Compound 22 samples labelled with EM48 mHTT antibody and detected with fluorophore in the 488 nm channel. Grayscale images were pseudocolored with LUT “green” (top row) and “Green Fire Blue” (bottom row) in Fiji to visualize gradient in signal intensities. For the latter LUT, increasing signal intensities are represented from blue to green to white. Overall, the strongest mHTT signal is seen in TG vehicle while both WT vehicle and TG Compound 22 displayed a decreased signal. Scale bars = 10 µm.

FIGS. 23A and 23D - WT vehicle sample showing basal binding of the antibody to endogenous mouse HTT. Single bright foci of homogenous size were seen, which were thought to represent spontaneous self-aggregation of the antibody resulting from the absence of specific antigen.

FIGS. 23B and 23E - TG vehicle sample displaying a substantial raise in total signal over the WT. Bright, large and more heterogeneous foci were described as IBs, with surrounding diffuse, oligomeric mHTT (see especially green-colored areas in lower depiction).

FIGS. 23C and 23F - TG Compound 22 sample with an apparent slump in mHTT signal compared to the TG vehicle control. Both intensities of IBs and diffuse mHTT seemed to be substantially lowered in the treated sample. In general, the intensity level appeared to resemble the intensity of the WT control.

FIG. 24 is a bar graph of the nuclear diffuse mHTT levels in the cortex of R6/2 mice treated with Compound 22. Nuclear diffuse mHTT was reduced by 40% in treated animals (n= 9) in comparison to vehicle treated TG mice (p = 0.01). TG transgenic R6/2 mice, wt wildtype, mHTT mutated huntingtin.

FIG. 25 is a graph of the rotarod correlation with diffuse mHTT in R6/2 mice treated with Compound 22. Pearson r: -0.8, p= 0.01, n=9 (all transgenic female mice), confidence interval of r= -0.9561 to -0.2896 for nuclear diffuse mHTT. TG, transgenic R6/2 mice; mHTT mutated huntingtin.

FIG. 26 is a bar graph showing the rotarod latency until mice fall in R6/2 mice treated with Compound 22. R6/2 mice treated with Compound 22 showed significantly improved rotarod performance at 10 weeks of age compared to vehicle treated R6/2 mice. The mean latency to fall was 108.3 s ± 32.9 s in treated transgenic R6/2 mice and 55.7 s ± 19.1 s (p < 0.05). Compound 22 improved the latency to fall 94% in comparison to untreated model mice.

FIG. 27 is a bar graph showing CREB-binding protein (CBP) colocalization with mHTT. 4-fold reduction of colocalization in treated animals showed an improvement in motor symptoms in comparison to untreated TG mice (p = 0.02)

FIG. 28 is a graph showing the circular dichroism spectra for long Q length (Q46) Exon1 and Compound 22. Increasing doses of Compound 22 decreases the proportion of a predominantly α-helical state of Exon1 Q46.

FIG. 29 is a graph showing the circular dichroism spectra for short Q length(Q16) Exon 1, Exon1 Q46 and Exon 1Q46 bound to Compound 4.

FIG. 30 is a graph showing the circular dichroism spectra of TRX exposed to different doses of Compound 4.

FIG. 31 is a graph showing the circular dichroism spectra between 206 nm - 210 nm of Exon1 Q16, Exon 1 Q46 and Exon1 Q46 bound to Compound 9.

FIG. 32 is a bar graph showing the autophagic flux of STHdh 111/111 neurons treated with 5 µM Compound 22 compared with untreated STHdh 111/111 neurons and with STHdh 7/7 cells expressing wildtype huntingtin

FIG. 33 is a bar graph showing that the area stained with LC3 antibody is reduced in treated STHdh 111/111 cells compared to untreated cells. LC3 area is area stained with LC3 antibody. ** p < 0.001

FIG. 34 is a graph showing that STHdh neurons exposed to autophagy inhibitor NH4CL did not show reduction of mHTT upon treatment of Compound 22. FI - fluorescence intensity of mHTT stained with 3B5H10 primary antibody against mHTT.

FIG. 35 is a graph showing that STHdh neurons exposed to 125 nM of Ubiquitin Proteasome System (UPS) inhibitor MG132, had a reduction of mHTT upon treatment of Compound 22. FI - fluorescence intensity of mHTT stained with 3B5H10 primary antibody against mHTT.

FIG. 36 is a graph showing that Slc32al is downregulated in STHdh 111/111 (Q111-0) neurons in comparison to STHdh 7/7 (Q7-0). Treatment with Compound 22 improves SLC32al expression in STHdh 111/111 cells (Q111-10).

FIG. 37 is a graph showing that Rasgrpl is upregulated in STHdh 111/111 (Q111-0) neurons in comparison to STHdh 7/7 (Q7-0). Treatment with Compound 22 improves Rasgrpl expression in STHdh 111/111 cells (Q111-10).

FIG. 38 is a graph showing that Olig2 is downregulated in STHdh 111/111 (Q111-0) neurons in comparison to STHdh 7/7 (Q7-0). Treatment with Compound 22 improves Olig2 expression in STHdh 111/111 cells (Q111-10).

FIG. 39 is a graph showing that NGFR is downregulated in STHdh 111/111 (Q111-0) neurons in comparison to STHdh 7/7 (Q7-0). Treatment with Compound 22 improves NGFR expression in STHdh 111/111 cells (Q111-10).

FIG. 40 is a graph showing that Kcna is downregulated in STHdh 111/111 (Q111-0) neurons in comparison to STHdh 7/7 (Q7-0). Treatment with Compound 22 improves Kcna expression in STHdh 111/111 cells (Q111-10).

FIG. 41 is a graph showing that wildtype huntingtin was not reduced in STHdh 7/7 cells treated with Compound 4, in comparison to non-treated STHdh 7/7.

FIG. 42 is a graph showing that the EC 50 valuefor mHTT reduction in Example 41 was 130 nM.

FIG. 43 is a graph showing that the cell viability of STHdh 111/111 treated with Compound 22, with 125% in comparison to pre-heat shock cell viability, was higher in comparison to untreated cells, with 25% in comparison to pre-heat shock cell viability.

FIG. 44 is a graph showing that Compound 4 is both soluble and it can cross the blood brain barrier. Table 14 describes physicochemical properties of Compound 4.

FIG. 45 is a graph showing Normalized fluorescence intensity versus compound 20 concentration.

FIG. 46 is a graph showing that 10 µM Compound 4 treatment could reduce in STHdh 7/7 exposed to ammonium chloride 15.2% phospho-Tau (Ser396) in comparison to non-treated STHdh 7/7.

FIG. 47 is a graph showing that Phospho-Tau (Ser404) was significantly reduced by 40% (p = 0.003, Welch’s t-test) in STHdh 7/7 cells treated with 10 µM Compound 4 in comparison to untreated STHdh 7/7 cells. In STHdh 7/7 cells exposed to 10 mM autophagy inhibitor ammonium chloride (Sigma Aldrich) Compound 4 treatment could not reduce phospho-Tau (Ser404) in Compound 4 STHdh 7/7 neurons.

FIG. 48 shows that STHdh 111/111 treated with Compound 4 depicted improved cell viability, dendrite outgrowth and increased cell size.

FIG. 49 is a graph showing that Slc1a1, Ctse, Atp6vlh, Atp6v0dl, Ap3sl, Lamp1, Cd68, Gsub, Gba, Man2bl, Pptl, Hexb, Npcl and Ggal were equal to or greater than 1 time the standard deviation from the mean upregulated in STHdh 111/111 neurons treated with 20 µM Compound 22.

FIG. 50 is a timeline showing that zebrafish embryos were treated with Compound 4 prior to MPP+ exposure at 0 hpf, and as co-incubation with MPP+ at 24 hpf onwards.

FIG. 51 is a graph showing that larvae exposed to MPP+ alone moved significantly less frequently with 13.3 movement bouts in 30 minutes than naïve larvae with 40.8 movement bouts in 30 minutes (p < 0.001), and larvae exposed to 1 µM Compound 4 moved significantly less frequently with 21.0 movement bouts in 30 minutes than naïve larvae (p < 0.01), whereas no significant difference was observed for the drug treatment groups compared to MPP+

FIG. 52 is a graph showing effect of Compound 4 on sleep parameters following co-incubation with MPP+.

FIG. 53 is a graph showing effect of Compound 4 on sleep parameters following co-incubation with MPP+.

FIG. 54 is a graph showing effect of Compound 4 on sleep parameters following co-incubation with MPP+.

FIG. 55 is a graph showing effect of Compound 4 on sleep parameters following co-incubation with MPP+.

FIG. 56 is a graph showing effect of Compound 4 on sleep parameters following co-incubation with MPP+.

FIG. 57 is a graph showing a separation between the movement of larvae treated with MPP+ alone and 10 µM Compound 4 with MPP+ during lights-on phases is visible.

FIG. 58 is a graph showing the effects of 1 µM Compound 4 seems to be centered around the latter part of the photomotor assay and the first half of the daytime recording.

FIG. 59 is a proton NMR spectrum of Compound 4 (Example 28).

### DETAILED DESCRIPTION OF THE INVENTION

The present disclosure provides compounds that are capable of reducing misfolded and/or mutated proteins (e.g., huntingtin; “HTT”). Moreover, the compounds disclosed herein can ameliorate the pathological interaction of mutated and/or misfolded proteins with other proteins.

As noted above, Huntington’s Disease is characterized by the expansion of the poly-glutamine (“polyQ”) portion of HTT above 36 glutamine (“Q”) residues, while wild-type huntingtin contains fewer than 36 glutamine residues in a row. Above the threshold of 36 glutamine residues, the huntingtin protein is considered to be mutated (mHTT). The consequences of the poly-glutamine expansion above 36 residues include the misfolding, loss of conformational flexibility, and/or aggregation of mHTT (see Fodale V et al. (2014). PLoS One 9(12):e112262; Cui X et al. (2014). Sci Rep 4:5601). Without wishing to be bound by theory, the conformational rigidity (i.e., reduced flexibility) is caused by an increase of alpha-helical content and mHTT and may be responsible for the pathological interactions of mHTT with different proteins in cells. This pathological protein-protein interactions can lead to the symptoms of Huntington’s Disease (HD).

Without wishing to be bound by theory, previous therapeutics which target single protein pathways downstream of mHTT pathological protein-protein interactions have been ineffective at significantly ameliorating the symptoms of HD. There is a need for compounds that interact with mHTT in order to disrupt pathological interactions at an early stage. The present disclosure provides compounds that can bind directly to the polyQ segment of proteins such as mHTT. As a consequence of that direct binding, the compounds disclosed herein can at least partially reverse the conformation of mutated huntingtin (mHTT) back to wild-type huntingtin (HTT). Furthermore, the present disclosure provides compounds that can penetrate the blood brain barrier, which is an advantageous feature over existing therapeutics (e.g., antisense nucleotides) which without wishing to be bound by theory only penetrate efficiently to the outer layers of the brain and penetrate less efficiently to deep brain regions such as the striatum. The conformational changes induced by the compounds of the present disclosure can also facilitate increased autophagy of the mHTT proteins, enabling the cell to remove and/or break down mutated and/or misfolded proteins such as mHTT, for instance before they exert their pathological effects. Accordingly, the compounds of the present disclosure can be used to treat diseases of the central nervous system (CNS), such as Huntington’s Disease (HD). Additional features and advantages of the present disclosure will be apparent as set forth herein.

### Compounds

### R¹

In some embodiments, R¹ is selected from the group consisting of —H, —C₁—C₆alkyl, —C₂—C₆alkenyl, and —C₂—C₆alkynyl, wherein each alkyl, alkenyl and alkynyl is optionally substituted with one or more substituents independently selected from the group consisting of —Br and —F. In some embodiments, R¹ is selected from the group consisting of —H and —C₁—C₆alkyl. In some embodiments, R¹ is selected from the group consisting of —H, methyl, and benzyl. In some embodiments, R¹ is selected from the group consisting of —H and methyl. In some embodiments R¹ is —H. In some embodiments, R¹ is methyl.

### R²

In some embodiments, R² is —H, —C₁—C₆alkyl, —C₂—C₆alkenyl, or —C₂—C₆alkynyl, wherein each alkyl, alkenyl and alkynyl is optionally substituted with one or more substituents independently selected from the group consisting of —Br and —F. In some embodiments, R² is selected from the group consisting of —H and —Ci—C₆alkyl. In some embodiments, R² is selected from the group consisting of —H, methyl, and ethyl. In some embodiments, R² is selected from the group consisting of —H and methyl. In some embodiments R² is —H. In some embodiments, R² is methyl. In some embodiments, R² is ethyl.

### R³

In some embodiments, R³ is —H, —C1—C₆alkyl, —C₂—C₆alkenyl, or —C₂—C₆alkynyl, wherein each alkyl, alkenyl and alkynyl is optionally substituted with one or more substituents independently selected from the group consisting of —Br and —F. In some embodiments, R³ is selected from the group consisting of —H and —Ci—C₆alkyl. In some embodiments, R³ is selected from the group consisting of —H, methyl, and ethyl. In some embodiments, R³ is selected from the group consisting of —H and methyl. In some embodiments R³ is —H. In some embodiments, R³ is methyl. In some embodiments, R³ is ethyl.

### R² and R³

In some embodiments, R² and R³ can optionally combine, together with the atoms to which they are attached, to form a C₄-C₈carbocycle or a C₄-C₈heterocycle, wherein the carbocycle or heterocycle is optionally substituted with one or more substituents independently selected from the group consisting of —Br, —F, —C1—C₆alkyl, —C₂—C₆alkenyl, and —C₂—C₆alkynyl, and wherein each alkyl, alkenyl, and alkynyl is optionally substituted with one or more —Br or —F. In some embodiments, R² and R³ can optionally combine, together with the atoms to which they are attached, to form a unsubstituted C₄-C₈carbocycle. In some embodiments, R² and R³ can optionally combine, together with the atoms to which they are attached, to form an unsubstituted 5-6-membered carbocycle. In some embodiments, R² and R³ can optionally combine, together with the atoms to which they are attached, to form an unsubstituted 5-membered carbocycle. In some embodiments, R² and R³ can optionally combine, together with the atoms to which they are attached, to form an unsubstituted 6-membered carbocycle.

### R⁴

In some embodiments, R⁴ is —H, —C1—C₆alkyl, —C₂—C₆alkenyl, or —C₂—C₆alkynyl, wherein each alkyl, alkenyl and alkynyl is optionally substituted with one or more substituents independently selected from the group consisting of —Br and —F. In some embodiments, R⁴ is selected from the group consisting of —H and —Ci—C₆alkyl. In some embodiments, R⁴ is selected from the group consisting of —H, methyl, ethyl, i-propyl, cyclopropyl, cyclopentyl, and t-butyl. In some embodiments, R⁴ is selected from the group consisting of —H, methyl, ethyl, and i-propyl. In some embodiments R⁴ is —H. In some embodiments, R⁴ is methyl. In some embodiments, R⁴ is ethyl. In some embodiments, R⁴ is i-propyl.

### R⁵

In some embodiments, R⁵ is —H, —C1—C₆alkyl, —C₂—C₆alkenyl, or —C₂—C₆alkynyl, wherein each alkyl, alkenyl and alkynyl is optionally substituted with one or more substituents independently selected from the group consisting of —Br and —F. In some embodiments, R⁵ is selected from the group consisting of —H and —Ci—C₆alkyl. In some embodiments, R⁵ is selected from the group consisting of —H, methyl, ethyl, i-propyl, cyclopropyl, cyclopentyl, and t-butyl. In some embodiments, R⁵ is selected from the group consisting of —H, methyl, ethyl, and i-propyl. In some embodiments R⁵ is —H. In some embodiments, R⁵ is methyl. In some embodiments, R⁴ is ethyl. In some embodiments, R⁵ is i-propyl.

### R⁴ and R⁵

In some embodiments, R⁴ and R⁵ can optionally combine, together with the nitrogen atom to which they are attached, to form a C₃-C₈heterocycle, wherein the heterocycle is optionally substituted with one or more substituents independently selected from the group consisting of —Br, —F, —C1—C₆alkyl, —C₂—C₆alkenyl, and —C₂—C₆alkynyl, and wherein each alkyl, alkenyl and alkynyl is optionally substituted with one or more —Br or —F. In some embodiments, R⁴ and R⁵ can optionally combine, together with the nitrogen atom to which they are attached, to form a heterocycle selected from the group consisting of azetidine, pyrrolidine, piperidine, morpholine, and piperazine, wherein the heterocycle is optionally substituted with one or more substituents independently selected from the group consisting of —Br, —F, —C₁—C₆alkyl, —C₂—C₆alkenyl, and —C₂—C₆alkynyl, and wherein each alkyl, alkenyl and alkynyl is optionally substituted with one or more —Br or —F.

### Definitions

“Alkyl” refers to a straight or branched chain saturated hydrocarbon containing 1-12 carbon atoms. C₁—C₆alkyl groups contain 1 to 6 carbon atoms. Examples of a C₁—C₆alkyl group include, but are not limited to, methyl, ethyl, propyl, butyl, pentyl, isopropyl, isobutyl, sec-butyl and tert-butyl, isopentyl and neopentyl.

“Alkenyl” refers to a straight or branched chain unsaturated hydrocarbon containing 2-12 carbon atoms. The “alkenyl” group contains at least one double bond in the chain. The double bond of an alkenyl group can be unconjugated or conjugated to another unsaturated group. Examples of alkenyl groups include ethenyl, propenyl, n-butenyl, iso-butenyl, pentenyl, or hexenyl. An alkenyl group can be unsubstituted or substituted. Alkenyl, as herein defined, may be straight or branched.

“Alkynyl” refers to a straight or branched chain unsaturated hydrocarbon containing 2-12 carbon atoms. The “alkynyl” group contains at least one triple bond in the chain. Examples of alkenyl groups include ethynyl, propanyl, n-butynyl, iso-butynyl, pentynyl, or hexynyl. An alkynyl group can be unsubstituted or substituted.

The terms “heterocyclyl” or “heterocycloalkyl” or “heterocycle” refer to monocyclic or polycyclic 3 to 8-membered non-aromatic rings containing carbon and heteroatoms taken from oxygen, phosphorous, nitrogen, or sulfur and wherein there are not delocalized π electrons (aromaticity) shared among the ring carbon or heteroatoms. Heterocyclyl rings include, but are not limited to, oxetanyl, azetadinyl, tetrahydrofuranyl, pyrrolidinyl, oxazolinyl, oxazolidinyl, thiazolinyl, thiazolidinyl, pyranyl, thiopyranyl, tetrahydropyranyl, dioxalinyl, piperidinyl, morpholinyl, thiomorpholinyl, thiomorpholinyl S-oxide, thiomorpholinyl S-dioxide, piperazinyl, azepinyl, oxepinyl, diazepinyl, tropanyl, and homotropanyl. A heterocyclyl or heterocycloalkyl ring can also be fused or bridged, e.g., can be a bicyclic ring.

The term “cycloalkyl” or “carbocycle” or “carbocyclyl” means monocyclic or polycyclic saturated or unsaturated carbon rings containing 3-8 carbon atoms, wherein the rings may be fused to an aromatic group. Examples of cycloalkyl groups include, without limitations, cyclopropyl, cyclobutyl, cyclopentyl, cyclohexyl, cycloheptanyl, cyclooctanyl, norboranyl, norborenyl, bicyclo[2.2.2]octanyl, or bicyclo[2.2.2]octenyl. A C₃-C₈ cycloalkyl is a cycloalkyl group containing between 3 and 8 carbon atoms. A cycloalkyl group can be fused (e.g., decalin) or bridged (e.g., norbornane).

“Aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C10 aryl”; e.g., naphthyl such as 1-naphthyl and 2-naphthyl). In some embodiments, an aryl group has fourteen ring carbon atoms (“C14 aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, and trinaphthalene. Particularly aryl groups include phenyl, naphthyl, indenyl, and tetrahydronaphthyl. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

“Heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

Alkyl, alkenyl, alkynyl, carbocyclyl, heterocyclyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety.

As used herein, the term “halo” or “halogen” means a fluoro, chloro, bromo, or iodo group.

The term “tautomers” refers to a set of compounds that have the same number and type of atoms, but differ in bond connectivity and are in equilibrium with one another. A “tautomer” is a single member of this set of compounds. Typically, a single tautomer is drawn but it is understood that this single structure is meant to represent all possible tautomers that might exist. Examples include enol-ketone tautomerism. When a ketone is drawn it is understood that both the enol and ketone forms are part of the invention.

The term “prodrug,” as used in this disclosure, means a compound which is convertible in vivo by metabolic means (e.g., by hydrolysis) to a disclosed compound. Furthermore, as used herein a prodrug is a drug which is inactive in the body, but is transformed in the body typically either during absorption or after absorption from the gastrointestinal tract into the active compound. The conversion of the prodrug into the active compound in the body may be done chemically or biologically (i.e., using an enzyme).

The term “solvate” refers to a complex of variable stoichiometry formed by a solute and solvent. Such solvents for the purpose of the invention may not interfere with the biological activity of the solute. Examples of suitable solvents include, but are not limited to, water, MeOH, EtOH, and AcOH. Solvates wherein water is the solvent molecule are typically referred to as hydrates. Hydrates include compositions containing stoichiometric amounts of water, as well as compositions containing variable amounts of water.

The term “isomer” refers to compounds that have the same composition and molecular weight but differ in physical and/or chemical properties. The structural difference may be in constitution (geometric isomers) or in the ability to rotate the plane of polarized light (stereoisomers). With regard to stereoisomers, the compounds of Formula I may have one or more asymmetric carbon atom and may occur as racemates, racemic mixtures and as individual enantiomers or diastereomers.

The term “stereoisomers” refers to the set of compounds which have the same number and type of atoms and share the same bond connectivity between those atoms, but differ in three dimensional structure. The term “stereoisomer” refers to any member of this set of compounds. For instance, a stereoisomer may be an enantiomer or a diastereomer.

The term “enantiomers” refers to a pair of stereoisomers which are non-superimposable mirror images of one another. The term “enantiomer” refers to a single member of this pair of stereoisomers. The term “racemic” refers to a 1:1 mixture of a pair of enantiomers.

The term “diastereomers” refers to the set of stereoisomers which cannot be made superimposable by rotation around single bonds. For example, cis- and trans- double bonds, endo- and exo- substitution on bicyclic ring systems, and compounds containing multiple stereogenic centers with different relative configurations are considered to be diastereomers. The term “diastereomer” refers to any member of this set of compounds. In some examples presented, the synthetic route may produce a single diastereomer or a mixture of diastereomers. In some cases these diastereomers were separated and in other cases a wavy bond is used to indicate the structural element where configuration is variable.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

The disclosure also includes pharmaceutical compositions comprising an effective amount of a disclosed compound and a pharmaceutically acceptable carrier. Representative “pharmaceutically acceptable salts” include, e.g., water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, sethionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts.

An “effective amount” when used in connection with a compound is an amount effective for treating or preventing a disease in a subject as described herein.

The term “carrier”, as used in this disclosure, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

The term “treating” with regard to a subject, refers to improving at least one symptom of the subject’s disorder. Treating includes curing, improving, or at least partially ameliorating the disorder.

As used herein, the term “prevent,” “prevention,” or “preventing” refers to any method to partially or completely prevent or delay the onset of one or more symptoms or features of a disease, disorder, and/or condition. Prevention treatment may be administered to a subject who does not exhibit signs of a disease, disorder, and/or condition.

The term “disorder” is used in this disclosure to mean, and is used interchangeably with, the terms disease, condition, or illness, unless otherwise indicated.

The term “administer”, “administering”, or “administration” as used in this disclosure refers to either directly administering a disclosed compound or pharmaceutically acceptable salt of the disclosed compound or a composition to a subject, or administering a prodrug derivative or analog of the compound or pharmaceutically acceptable salt of the compound or composition to the subject, which can form an equivalent amount of active compound within the subject’s body.

A “patient” or “subject” is a mammal, e.g., a human, mouse, rat, guinea pig, dog, cat, horse, cow, pig, or non-human primate, such as a monkey, chimpanzee, baboon or rhesus.

In some embodiments of the invention, the compounds of the present disclosure are enantiomers. In some embodiments the compounds are the (S)-enantiomer. In other embodiments the compounds are the (R)-enantiomer. In yet other embodiments, the compounds of Formula I may be (+) or (-) enantiomers. It should be understood that all isomeric forms are included within the present invention, including mixtures thereof. If the compound contains a double bond, the substituent may be in the E or Z configuration. If the compound contains a disubstituted cycloalkyl, the cycloalkyl substituent may have a cis- or trans- configuration. All tautomeric forms are also intended to be included.

### Methods of Using the Disclosed Compounds

Without wishing to be bound by theory, the compounds of the present disclosure can interact with the polyQ segment of a mutated protein (e.g., via electrostatic attraction, hydrogen bonding, and/or van der Walls forces) and at least partially reverse the conformation of mHTT back to wild-type HTT. For example, the compounds of the present disclosure can increase the proportion of predominantly random coil conformation of mHTT and reduce the proportion of predominantly alpha-helix conformation of mHTT as measured by methods such as circular dichroism, electron paramagnetic resonance (EPR), and nuclear magnetic resonance (NMR) spectroscopy. In some embodiments, the at least partial reversal of the conformation of mHTT back to wild-type HTT (i.e., increased proportion of predominantly random coil conformation of mHTT and reduced proportion of predominantly alpha-helix conformation) takes place while the compounds are engaged with the target site on mHTT. Without wishing to be bound by theory, there is an equilibrium between mHTT bound to compound with reversal of the pathological conformation and mHTT unbound to compound and with no reversal of the pathological conformation of mHTT. Subsequently, drug residence time can determine the percentage of reversed mHTT.

In some embodiments, the compounds of the present disclosure can result in at least about 1% reversal of mHTT; about 2% reversal of mHTT; about 3% reversal of mHTT; about 4% reversal of mHTT; about 5% reversal of mHTT; about 6% reversal of mHTT; about 7% reversal of mHTT; about 8% reversal of mHTT; about 9% reversal of mHTT; about 10% reversal of mHTT; about 15% reversal of mHTT; about 20% reversal of mHTT; about 25% reversal of mHTT; about 30% reversal of mHTT; about 35% reversal of mHTT; about 40% reversal of mHTT; about 45% reversal of mHTT; about 50% reversal of mHTT; about 55% reversal of mHTT; about 60% reversal of mHTT; about 65% reversal of mHTT; about 70% reversal of mHTT; about 75% reversal of mHTT; about 80% reversal of mHTT; about 85% reversal of mHTT; about 90% reversal of mHTT; about 95% reversal of mHTT; about 99% reversal of mHTT; or about 100% reversal of mHTT.

Aspects of the invention relate to the treatment of a polyQ disease such as HD. The method can involve administering to a patient in need thereof an effective amount of a compound of the present disclosure or a pharmaceutically acceptable salt, hydrate, solvate, prodrug, stereoisomer, or tautomer thereof. Another aspect of the present disclosure relates to a compound of the present disclosure, or a pharmaceutically acceptable salt, hydrate, solvate, prodrug, stereoisomer, or tautomer thereof, for use in treating a polyQ disease such as HD. In another aspect, the present disclosure relates to the use of a compound of the disclosure, or a pharmaceutically acceptable salt, hydrate, solvate, prodrug, stereoisomer, or tautomer thereof, in the manufacture of a medicament for treating a polyQ disease such as HD. The present disclosure also relates to pharmaceutical compositions. In some embodiments, the pharmaceutical compositions can be used for the treatment of a polyQ disease such as HD.

Aspects of the invention relate to the prevention of a polyQ disease such as HD. The method can involve administering to a patient in need thereof an effective amount of a compound of the present disclosure or a pharmaceutically acceptable salt, hydrate, solvate, prodrug, stereoisomer, or tautomer thereof. Another aspect of the present disclosure relates to a compound of the present disclosure, or a pharmaceutically acceptable salt, hydrate, solvate, prodrug, stereoisomer, or tautomer thereof, for use in preventing a polyQ disease such as HD. In another aspect, the present disclosure relates to the use of a compound of the disclosure, or a pharmaceutically acceptable salt, hydrate, solvate, prodrug, stereoisomer, or tautomer thereof, in the manufacture of a medicament for preventing a polyQ disease such as HD.

In some embodiments, the compounds of the present disclosure can be used to treat a polyQ disease. In some embodiments, the polyQ disease can be Huntington’s Disease. In some embodiments, the polyQ disease can be a spinocerebellar ataxia such as spinocerebellar ataxia 1, spinocerebellar ataxia 2, spinocerebellar ataxia 3 (i.e., MJD), spinocerebellar ataxia 6 (CACNL1A4), and/or spinocerebellar ataxia 7. In some embodiments, the polyQ disease can be spinal bulbar muscular atrophy, Kennedy’s Disease, and/or dentatorubral-pallidoluysian atrophy.

In some embodiments, the compounds of the present disclosure can be used to treat neurodegenerative diseases such as Alzheimer’s disease (AD), Parkinson’s disease (PD), Frontotemporal dementia, Multiple system atrophy (MSA), Amyotrophic lateral sclerosis (ALS), Friedreich’s ataxia, Motor neurone diseases, and/or Spinal muscular atrophy (SMA).

In some embodiments, the compounds of the disclosure can be used after strokes to reduce the penumbra. In some embodiments, the compounds of the disclosure can inhibit neuronal apoptosis following ischemia.

Depending on the intended mode of administration, the disclosed compositions can be in solid, semi-solid or liquid dosage form, such as, for example, injectables, tablets, suppositories, pills, time-release capsules, elixirs, tinctures, emulsions, syrups, powders, liquids, suspensions, or the like, sometimes in unit dosages and consistent with conventional pharmaceutical practices. Likewise, they can also be administered in intravenous (both bolus and infusion), intraperitoneal, subcutaneous or intramuscular form, all using forms well known to those skilled in the pharmaceutical arts.

Illustrative pharmaceutical compositions are tablets and gelatin capsules comprising a Compound of the Invention and a pharmaceutically acceptable carrier, such as a) a diluent, e.g., purified water, triglyceride oils, such as hydrogenated or partially hydrogenated vegetable oil, or mixtures thereof, corn oil, olive oil, sunflower oil, safflower oil, fish oils, such as EPA or DHA, or their esters or triglycerides or mixtures thereof, omega-3 fatty acids or derivatives thereof, lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, algiic acid or its sodium salt, or effervescent mixtures; e) absorbent, colorant, flavorant and sweetener; f) an emulsifier or dispersing agent, such as Tween 80, Labrasol, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption of the compound such as cyclodextrin, hydroxypropyl-cyclodextrin, PEG400, PEG200.

Liquid, particularly injectable, compositions can, for example, be prepared by dissolution, dispersion, etc. For example, the disclosed compound is dissolved in or mixed with a pharmaceutically acceptable solvent such as, for example, water, saline, aqueous dextrose, glycerol, ethanol, and the like, to thereby form an injectable isotonic solution or suspension. Proteins such as albumin, chylomicron particles, or serum proteins can be used to solubilize the disclosed compounds.

The disclosed compounds can be also formulated as a suppository that can be prepared from fatty emulsions or suspensions; using polyalkylene glycols such as propylene glycol, as the carrier.

The disclosed compounds can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described in U.S. Pat. No. 5,262,564.

Disclosed compounds can also be delivered by the use of monoclonal antibodies as individual carriers to which the disclosed compounds are coupled. The disclosed compounds can also be coupled with soluble polymers as targetable drug carriers. Such polymers can include polyvinylpyrrolidone, pyran copolymer, polyhydroxypropylmethacrylamide-phenol, polyhydroxyethylaspanamidephenol, or polyethyleneoxidepolylysine substituted with palmitoyl residues. Furthermore, the disclosed compounds can be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels. In one embodiment, disclosed compounds are not covalently bound to a polymer, e.g., a polycarboxylic acid polymer, or a polyacrylate.

Parental injectable administration is generally used for subcutaneous, intramuscular or intravenous injections and infusions. Injectables can be prepared in conventional forms, either as liquid solutions or suspensions or solid forms suitable for dissolving in liquid prior to injection.

Another aspect of the invention relates to a pharmaceutical composition comprising a compound of the present disclosure and a pharmaceutically acceptable carrier. The pharmaceutically acceptable carrier can further include an excipient, diluent, or surfactant.

Compositions can be prepared according to conventional mixing, granulating or coating methods, respectively, and the present pharmaceutical compositions can contain from about 0.1% to about 99%, from about 5% to about 90%, or from about 1% to about 20% of the disclosed compound by weight or volume.

The dosage regimen utilizing the disclosed compound is selected in accordance with a variety of factors including type, species, age, weight, sex and medical condition of the patient; the severity of the condition to be treated; the route of administration; the renal or hepatic function of the patient; and the particular disclosed compound employed. A physician or veterinarian of ordinary skill in the art can readily determine and prescribe the effective amount of the drug required to prevent, counter or arrest the progress of the condition.

Effective dosage amounts of the disclosed compounds, when used for the indicated effects, range from about 0.5 mg to about 5000 mg of the disclosed compound as needed to treat the condition. Compositions for in vivo or in vitro use can contain about 0.5, 5, 20, 50, 75, 100, 150, 250, 500, 750, 1000, 1250, 2500, 3500, or 5000 mg of the disclosed compound, or, in a range of from one amount to another amount in the list of doses. In one embodiment, the compositions are in the form of a tablet that can be scored.

### EXAMPLES

The disclosure is further illustrated by the following examples and synthesis examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims. Unless otherwise noted, all materials were obtained from commercial suppliers and were used without further purification.

Example 1- Preparation of 2,3-dimethyl-1H-indole-5-carboxylic acid (Intermediate 1)

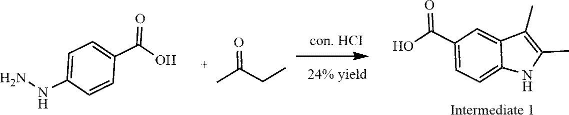

To a stirred solution of 4-hydrazinobenzoic acid (10.0 g, 65.7 mmol) in 1,4-dioxane was added butan-2-one (5.21 g, 72.3 mmol) and 2.00 mL con. HCl. The reaction mixture was stirred at 120° C. for 96 h. LC-MS check showed 87% area of Intermediate 1 and 5% area of hydrazone intermediate at 254 nm. The reaction was diluted with 300 mL H₂O, extracted with DCM (100 mL * 3). The organic phases were combined and dried over Na₂SO₄, concentrated. The residue was washed with 20.0 mL EtOAc to give pure Intermediate 1 (DCM/MeOH = 10/1, R_{ƒ}= 0.60). Yield: 3.00 g (24%).

Example 2 - Preparation of 2,3-dimethyl-1H-indol-5-amine (Intermediate 2)

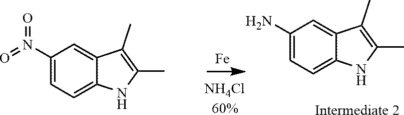

To a stirred solution of 2,3-dimethyl-5-nitro-1H-indole (1.90 g, 10.0 mmol) in 40.0 mL MeOH and 10.0 mL H₂O was added Fe powder (2.23 g, 40.0 mmol) and NH₄Cl (2.14 g, 40.0 mmol). The reaction mixture was stirred at 65° C. for 1 h. TLC check (EtOAc/petroleum = 1/1) showed the reaction was completed, a new spot was observed (R_{f}= 0.30). The reaction was filtered through a thick pad of celite. The filter cake was washed with MeOH (3* 40.0 mL). The filtrate was concentrated to remove the solvent of MeOH. The residue was diluted with 20.0 mL brine, extracted with DCM (3* 50.0 mL). The organic phase was combined, dried over Na₂SO₄, filtered and concentrated to give desired product. Yield: 1.00 g (60%).

Example 3 - Preparation of 1,2,3-Trimethyl-1H-indole-5-carboxylic acid (Intermediate 3)

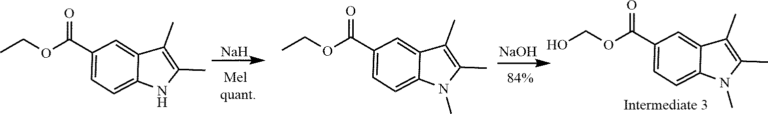

To a stirred solution of ethyl 2,3-Dimethyl-1H-indole-5-carboxylate (1.40 g, 6.44 mmol) in 5.00 mL DMF was added NaH (387 mg, 9.67 mmol, 60% in mineral oil) and MeI (2.74 g, 19.3 mmol, 3.0 eq.) at 0° C. The reaction mixture was stirred at 25° C. for 2 h. TLC (petroleum Ether/ EtOAc = 5/1) check showed all starting material (R_{ƒ} = 0.40) was consumed, a major new spot (R_{f}= 0.50) was observed. The reaction was diluted by 25.0 mL cold H₂O, extracted with DCM (10.0 mL *3). The combined organic layers were dried over Na₂SO₄, concentrated to give desired product. Yield: 1.60 g (quant.).

To a stirred solution of 2,3-Dimethyl-1H-indole-5-carboxylate (1.49 g, 6.44 mmol) in 20.0 mL EtOH and 5.00 mL H₂O was added NaOH (773 mg, 19.3 mmol). The reaction mixture was stirred at 25° C. for 16 h. TLC check (petroleum Ether/ EtOAc = 5/1) showed half of 2,3-Dimethyl-1H-indole-5-carboxylate still remained. The reaction was heated to 60° C. for 7 h till full consumption of 2,3-Dimethyl-1H-indole-5-carboxylate by TLC check. The reaction was concentrated to remove most of the EtOH. The residue was diluted with 25.0 mL brine, acidified to pH = 3-4 and extracted with DCM (10.0 mL * 3). The organic phase was combined and dried over Na₂SO₄, concentrated to give desired product. Yield: 1.10 g (84%).

Example 4 - Preparation of 2,3-Dimethyl-5-nitro-1-(phenylsulfonyl)-1H-indole (Intermediate 4A)

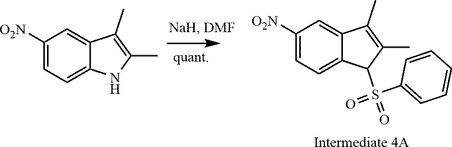

To a stirred solution of NaH (2.27 g, 56.8 mmol) in 20.0 mL DMF was added 2,3-Dimethyl-5-nitro-1H-indole (5.40 g, 28.4 mmol) in portions at 0° C. The reaction mixture was stirred for 30 minutes, benzenesulfonyl chloride (6.02 g, 34.1 mmol) was added and the mixture was allowed to stir for 2 h. The reaction was diluted by 60.0 mL H₂O, extracted with EtOAc (30.0 mL * 3). The combined organic layers were dried over Na₂SO₄, concentrated to give desired product. Yield: 9.58 g (quant.).

Example 5 - Preparation of 2,3-Dimethyl-1-(phenylsulfonyl)-1H-indol-5-amine (Intermediate 4B)

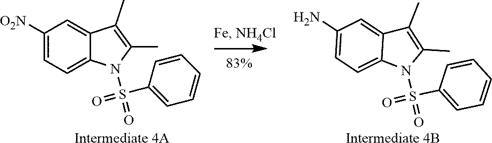

To a stirred solution of Intermediate 4A (9.58 g, 29.0 mmol) in 20.0 mL EtOH and 5.00 mL H₂O was added NH₄Cl (4.65 g, 87.0 mmol). The reaction mixture was heated to 85° C., then Fe powder (9.72 g, 174 mmol) was added in portions. After the addition, the reaction was heated to 85° C. for 2 h. the reaction mixture was cooled to 40° C. and filtered through a pad of celite, washed with a large amount of EtOH. The filtrate was concentrated. The residue was diluted with water and EtOAc, separated. The aqueous phase was extracted with EtOAc (100 mL * 3). The combined organic phases were washed with brine, dried over Na₂SO₄, concentrated. The residue was purified by silica gel column (EtOAc/petroleum ether = ⅒ to ⅛) to give pure desired product. Yield: 7.46 g (83%).

Example 6 - Preparation of /V-(2-(Diethylamino)ethyl)-2,3-dimethyl-1-(phenylsulfonyl)-1H-indole-5-sulfonamide (Intermediate 4C)

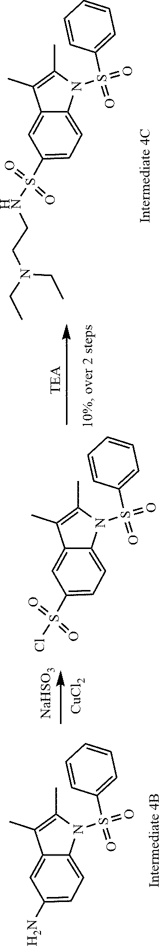

To conc. HCl (36%, 0.30 mL) at -5° C. was added Intermediate 4B (200 mg, 0.67 mmol). The mixture was stirred under -5° C. for 30 min. Then a solution of NaNO₂ (50.5 mg, 0.73 mmol) in H₂0 (4.00 mL) was added to the mixture under -5° C. The resulting mixture (A) was further stirred at -5° C. for 30 min. In parallel, to a solution of CuCl₂•2H₂O (25.2 mg, 0.17 mmol) in conc. HCl (36%, 0.30 mL) at -5° C. was added NaHSO₃ (277 mg, 2.66 mmol) to form mixture B. The resulting mixture (B) was stirred under -5° C. for 30 min. After that, the mixture (A) was added into mixture (B) and the reaction was stirred at - 5° C. for 45 min. The reaction was quenched with ice (1.00 g) and after 10 minutes, water (10.0 mL) was added. The resulting mixture was extracted with EtOAc (5.00 mL * 3). The organic solvents were concentrated and dried over vacuo to afford crude desired product, which was used directly in next step without any purification.

The crude product was dissolved in 5.00 mL DCM was added N¹,N¹-diethylethane-1,2-diamine (11.7 mg, 0.10 mmol), TEA (13.9 mg, 0.14 mmol). The reaction mixture was stirred at 25° C. for 2 h. TLC check (DCM/MeOH = 10/1) showed a major new spot (R_{f}= 0.60) formed. The reaction was diluted with 5.00 mL DCM and 10.0 mL H₂O. The organic phase was separated, the aqueous layer was extracted with DCM (5.00 mL * 2). The organic phase was combined, dried over Na₂SO₄, filtered and concentrated. The residue was purified by preparative TLC (DCM/MeOH = 10/1) to give desired product. Yield: 24.0 mg (10%, over 2 steps).

Example 7 - Preparation of Ethyl 2,3-dimethyl-1-(phenylsulfonyl)-1H-indole-5-carboxylate (Intermediate 4D)

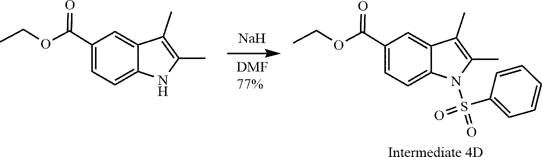

To a stirred solution of NaH (2.76 g, 69.0 mmol) in 80.0 mL DMF was added ethyl 2,3-dimethyl-1H-indole-5-carboxylate (10.0 g, 46.0 mmol) at 0° C. The reaction mixture was stirred 30 minutes at 0° C., benzenesulfonyl chloride (10.6 g, 59.8 mmol) was added drop wise and the mixture was allowed to stir for 16 h at 0-28° C. TLC check (EtOAc/ petroleum ether = ⅕) showed roughly 10% of the starting material (R_{f}= 0.30) remained, one new spot (R_{f}= 0.40) formed. The reaction was quenched with 150 mL H₂O at 0° C., extracted with EtOAc (50.0 mL * 3). The combined organic phases were washed with brine, dried over Na₂SO₄, concentrated. The residue was purified by silica gel column (EtOAc/petroleum ether = ⅛ to ¼) to give pure desired product. Yield: 12.6 g (77%).

Example 8 - Preparation of (2,3-Dimethyl-1-(phenylsulfonyl)-1H-indol-5-yl)methanol (Intermediate 4E)

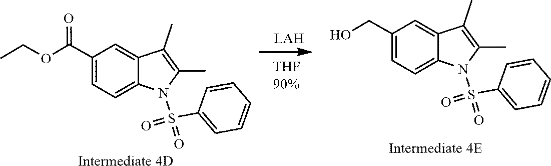

To Intermediate 4D (12.6 g, 35.3 mmol) in 100 mL dry THF was added LAH (1.61 g, 42.4 mmol) in portions at 0° C. The reaction mixture was stirred 45 minutes at 0-25° C. TLC check (EtOAc/ petroleum ether = ½) showed complete conversion of the starting material (R_{f} = 0.60), one new spot (R_{f}= 0.30) could be observed. The reaction was quenched and diluted with 200 mL H₂O at 0° C., extracted with EtOAc (70.0 mL * 3). The combined organic phases were washed with brine, dried over Na₂SO₄, concentrated. The residue was purified by silica gel column (EtOAc/petroleum ether = ½) to give pure desired product. Yield: 9.97 g (90%).

Example 9 - Preparation of 2,3-Dimethyl-1-(phenylsulfonyl)-1H-indole-5-carbaldehyde (Intermediate 4F)

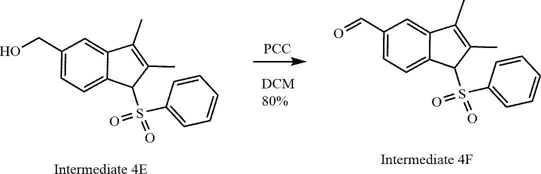

To Intermediate 4E (5.00 g, 15.9 mmol) in 30.0 mL DCM was added silica gel (20.0 g) and PCC (5.13 g, 23.8 mmol). The reaction mixture was stirred at 28° C. for 10 min. TLC check (EtOAc/ petroleum ether = ½) showed complete conversion of the starting material (R_{ƒ} = 0.30), one new spot (R_{f}= 0.50) could be observed. The reaction was filtered through a pad of celite, and the filtrate was diluted with 100 mL H₂O, extracted with DCM (20.0 mL * 3). The combined organic phases were washed with brine, dried over Na₂SO₄, concentrated. The residue was purified by silica gel column (EtOAc/petroleum ether = ⅙) to give pure desired product. Yield: 4.00 g (80%).

Example 10 - Preparation of N-((2,3-Dimethyl-1-(phenylsulfonyl)-1H-indol-5-yl)methyl)-2-(pyrrolidin-1-yl)ethan-1-amine (Intermediate 4G)

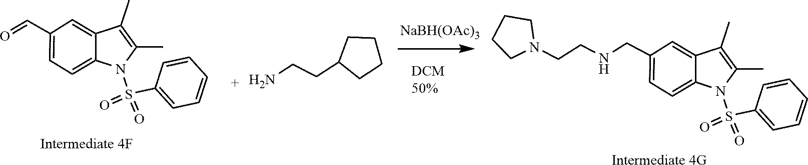

To a solution of Intermediate 4F (300 mg, 0.96 mmol) and 2-(pyrrolidin-1-yl)ethan-1-amine (114 mg, 1.00 mmol) in 15.0 mL DCM was added NaBH(OAc)₃ (305 mg, 1.44 mmol) and AcOH (57.6 mg, 0.96 mmol) at 0° C. The reaction was stirred at 0-28° C. for 16 h. TLC check (MeOH/ DCM = 1/20) showed complete conversion of the starting material (R_{f}= 0.95), one new spot (R_{f}= 0.50) could be observed. The reaction was diluted with 50.0 mL H₂O, extracted with DCM (15.0 mL * 3). The combined organic phases were washed with brine, dried over Na₂SO₄, concentrated. The residue was purified by Chromatotron (MeOH/DCM = 1/100 to 1/30) to give pure desired product. Yield: 192 mg (50%).

Example 11 - Preparation of N¹-((2,3-Dimethyl-1-(phenylsulfonyl)-1H-indol-5-yl)methyl)-N²,N²-diethylethane-1,2-diamine (Intermediate 4H)

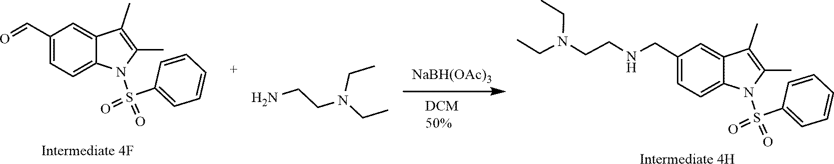

To a solution of Intermediate 4F (300 mg, 0.96 mmol) and N¹,N¹-diethylethane-1,2-diamine (116 mg, 1.00 mmol) in 15.0 mL DCM was added NaBH(OAc)₃ (305 mg, 1.44 mmol) and AcOH (57.6 mg, 0.96 mmol) at 0° C. The reaction was stirred at 0-28° C. for 16 h. TLC check (MeOH/ DCM = 1/20) showed complete conversion of the starting material (R_{f}= 0.95), one new spot (R_{f}= 0.50) formed. The reaction was diluted with 50.0 mL H₂O, extracted with DCM (15.0 mL * 3). The combined organic phases were washed with brine, dried over Na₂SO₄, concentrated. The residue was purified by Chromatotron (MeOH/DCM = 1/100 to 1/30) to give pure desired product. Yield: 195 mg (50%).

Example 12- Preparation of 2,3-dimethyl-N-(2-morpholinoethyl)-1H-indole-5-carboxamide (Compound 2)

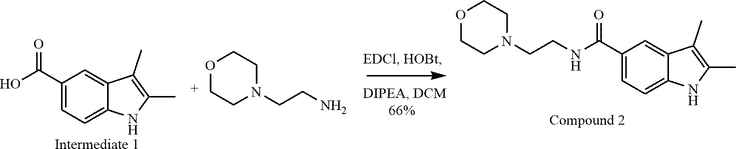

To a stirred solution of Intermediate 1 (50.0 mg, 0.26 mmol) in 5.00 mL DCM was added HOBt (42.8 mg, 0.32 mmol), EDC (49.2 mg, 0.32 mmol), 2-morpholinoethan-1-amine (41.3 mg, 0.32 mmol) and DIEA (68.3 mg, 0.53 mmol). The reaction mixture was stirred at 25° C. for 16 h. The reaction mixture was stirred at 25° C. for 16 h. TLC check (DCM/MeOH = 10/1, UV) indicated complete consumption of the starting material (R_{f}= 0.50), one main new spot (R_{ƒ}= 0.30) could be observed. The reaction was diluted with brine and extracted with DCM (3* 20.0 mL). The organic phase was combined and dried over Na₂SO₄, filtered and concentrated. The residue was purified by preparative TLC (DCM/MeOH = 10/1) to give pure target compound. Yield: 53.0 mg (66%).

Example 13- Preparation of N-(4-(dimethylamino)cyclohexyl)-2,3-dimethyl-1H-indole-5-carboxamide (Compound 101)

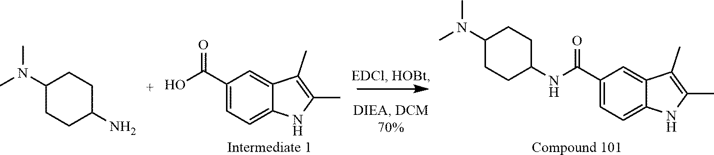

To a solution of Intermediate 1 (50.0 mg, 0.25 mmol) in 8.00 mL DCM at 0° C. was added N¹,N¹-dimethylcyclohexane-1,4-diamine (27.0 mg, 0.27 mmol), DIPEA (64.0 mg, 0.49 mmol), HOBt (49.0 mg, 0.37 mmol) and EDCI (70.0 mg, 0.37 mmol). Then the reaction was stirred at 20° C. for 16 h. TLC check (DCM/MeOH = 10/1, UV) indicated complete consumption of the starting material (R_{ƒ}= 0.50), one main new spot (R_{ƒ}= 0.20) could be observed. The mixture was cooled and partitioned between DCM and water, separated. The aqueous phase was extracted with DCM. The combined organic layers were washed with brine, dried over Na₂SO₄, filtered and concentrated. The residue was purified by PTLC (DCM/MeOH = 10/1) to give 55.0 mg pure target compound. Yield: 55.0 mg (70%).

Example 14 - Preparation of N-(2,3-Dimethyl-1H-indol-5-yl)-3-(dimethylamino)propanamide (Compound 9)

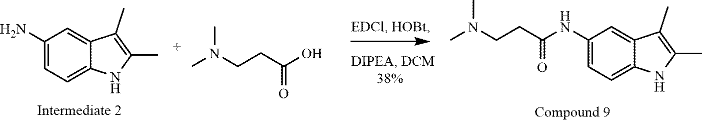

To a stirred solution of Intermediate 2 (50.0 mg, 0.31 mmol) in 5.00 mL DCM was added HOBt (46.0 mg, 0.34 mmol), EDCI (52.9 mg, 0.34 mmol), 3-(dimethylamino)propanoic acid (33.2 mg, 0.28 mmol) and DIPEA (73.3 mg, 0.57 mmol). The reaction mixture was stirred at 25° C. for 16 h. TLC check (DCM/MeOH = ⅒, UV) indicated complete consumption of the starting material (R_{f}= 0.50), one main new spot (R_{f}= 0.30) could be observed. The reaction was diluted with brine and extracted with DCM (3* 20.0 mL). The organic phase was combined and dried over Na₂SO₄, filtered and concentrated. The residue was purified by preparative TLC (DCM/MeOH = ⅒) to give pure title compound. Yield: 28.0 mg (38%).

Example 15 - Preparation of N-(2-(Azetidin-1-yl)ethyl)-2,3-dimethyl-1H-indole-5-carboxamide (Compound 17)

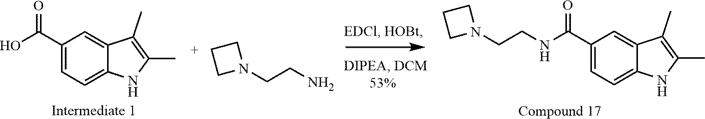

To a stirred solution of Intermediate 1 (50.0 mg, 0.26 mmol) in 5.00 mL DCM was added HOBt (42.8 mg, 0.32 mmol), EDC (49.2 mg, 0.32 mmol), 2-(azetidin-1-yl)ethan-1-amine (31.8 mg, 0.32 mmol) and DIEA (68.3 mg, 0.53 mmol). The reaction mixture was stirred at 25° C. for 16 h. The reaction mixture was stirred at 25° C. for 16 h under Ar. TLC check (DCM/MeOH = 10/1) showed full consumption of Intermediate 1 (R_{f}= 0.60), a major new spot (R_{f}= 0.30) was observed. The reaction was diluted with 15.00 mL DCM and 15.0 mL H₂O. The organic phase was separated, the aqueous layer was extracted with DCM (10.0 mL * 2). The organic phase was combined, dried over Na₂SO₄, filtered and concentrated. The residue was purified by preparative TLC (DCM/MeOH = 10/1) to give pure target compound. Yield: 38.0 mg (53%).

Example 16 - Preparation of N-(2-(Diethylamino)ethyl)-1,2,3-dimethyl-1H-indole-5-carboxamide (Compound 19)

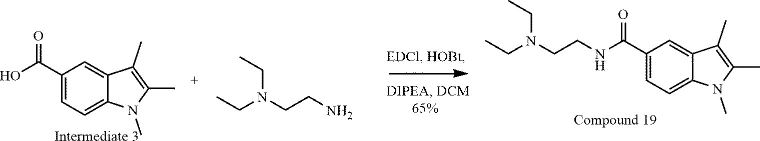

To a stirred solution of Intermediate 3 (50.0 mg, 0.25 mmol) in 5.00 mL DCM was added HOBt (39.9 mg, 0.30 mmol), EDCI (45.8 mg, 0.30 mmol), N¹,N¹-diethylethane-1,2-diamine (34.3 mg, 0.30 mmol) and DIEA (63.6 mg, 0.49 mmol). The reaction mixture was stirred at 25° C. for 16 h. The reaction mixture was stirred at 25° C. for 16 h. TLC check (DCM/MeOH = 10/1) showed full consumption of Intermediate 3 (R_{f}= 0.50), a major new spot (R_{f}= 0.30) was observed. The reaction was diluted with 5.00 mL DCM and 10.0 mL H₂O. The organic phase was separated, the aqueous layer was extracted with DCM (5.00 mL * 2). The organic phase was combined, dried over Na₂SO₄, filtered and concentrated. The residue was purified by preparative TLC (DCM/MeOH = 10/1) to give target compound. Yield: 49.0 mg (65%).

Example 17 - Preparation of N-(2-(Diethylamino)ethyl)-2,3-dimethyl-1H-indole-5-carboxamide (Compound 21)

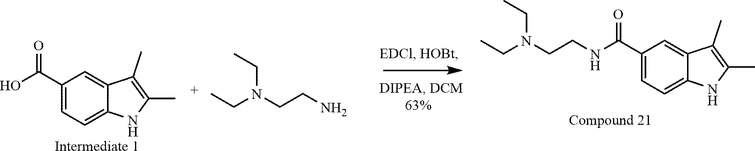

To a stirred solution of Intermediate 1 (50.0 mg, 0.26 mmol) in 5.00 mL DCM was added HOBt (42.8 mg, 0.32 mmol), EDCI (49.2 mg, 0.32 mmol), N1,N1-diethylethane-1,2-diamine (36.8 mg, 0.32 mmol) and DIEA (68.3 mg, 0.53 mmol). The reaction mixture was stirred at 25° C. for 16 h. The reaction mixture was stirred at 25° C. for 16 h under Ar. TLC check (DCM/MeOH = 10/1) showed full consumption of Intermediate 1 (R_{f}= 0.60), a major new spot (R_{f}= 0.30) was observed. The reaction was diluted with 10.0 mL DCM and 10.0 mL H₂O. The organic phase was separated, the aqueous layer was extracted with DCM (5.00 mL * 2). The organic phase was combined, dried over Na₂SO₄, filtered and concentrated. The residue was purified by preparative TLC (DCM/MeOH = 10/1) 2 times to give pure target compound. Yield: 48.0 mg (63%).

Example 18- Preparation of 3-Ethyl-2-methyl-N-(2-(piperidin-1-yl)ethyl)-1H-indole-5-carboxamide (Compound 24)

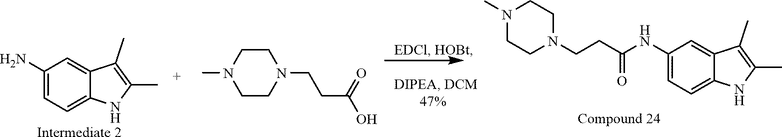

To a stirred solution of Intermediate 2 (50.0 mg, 0.31 mmol) in 5.00 mL DCM was added HOBt (46.0 mg, 0.34 mmol), EDCI (52.9 mg, 0.34 mmol), 3-(4-methylpiperazin-1-yl)propanoic acid (48.9 mg, 0.28 mmol) and DIPEA (73.3 mg, 0.57 mmol). The reaction mixture was stirred at 25° C. for 16 h. TLC check (DCM/MeOH = ⅒, UV) indicated complete consumption of the starting material (R_{ƒ}= 0.50), one main new spot (R_{ƒ}= 0.30) could be observed. The reaction was diluted with brine and extracted with DCM (3* 20.0 mL). The organic phase was combined and dried over Na₂SO₄, filtered and concentrated. The residue was purified by preparative TLC (DCM/MeOH = ⅒) to give pure target compound. Yield: 42.0 mg (47%).

Example 19 - Preparation of N-(2-(3,4-Dimethylpiperazin-1-yl)ethyl)-1,2,3-trimethyl-1H-indole-5-carboxamide (Compound 25)

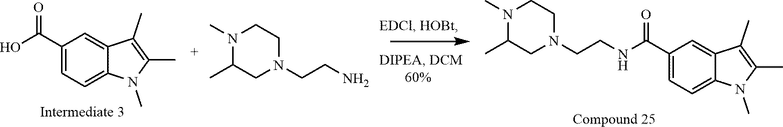

To a stirred solution of Intermediate 3 (50.0 mg, 0.25 mmol) in 5.00 mL DCM was added HOBt (39.9 mg, 0.30 mmol), EDCI (45.8 mg, 0.30 mmol), 2-(3,4-dimethylpiperazin-1-yl)ethan-1-amine (47.2 mg, 0.30 mmol) and DIEA (63.6 mg, 0.49 mmol). The reaction mixture was stirred at 25° C. for 16 h. The reaction mixture was stirred at 25° C. for 16 h. TLC check (DCM/MeOH = 10/1) showed full consumption of Intermediate 3 (R_{f}= 0.50), a major new spot (R_{f}= 0.30) was observed. The reaction was diluted with 5.00 mL DCM and 10.0 mL H₂O. The organic phase was separated, the aqueous layer was extracted with DCM (5.00 mL * 2). The organic phase was combined, dried over Na₂SO₄, filtered and concentrated. The residue was purified by preparative TLC (DCM/MeOH = 8/1) to give target compound. Yield: 50.0 mg (60%).

Example 20 - Preparation of 2,3-Dimethyl-N-(2-(pyrrolidin-1-yl)ethyl)-1H-indole-5-carboxamide (Compound 29)

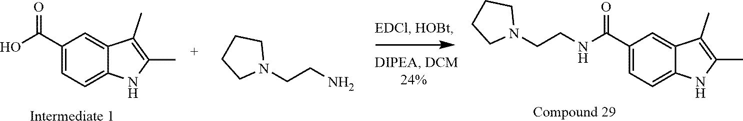

To a stirred solution of Intermediate 1 (50.0 mg, 0.26 mmol) in 5.00 mL DCM was added HOBt (42.8 mg, 0.32 mmol), EDCI (49.2 mg, 0.32 mmol), 2-(pyrrolidin-1-yl)ethan-1-amine (36.2 mg, 0.32 mmol) and DIEA (68.3 mg, 0.53 mmol). The reaction mixture was stirred at 25° C. for 16 h. The reaction mixture was stirred at 25° C. for 16 h. TLC check (DCM/MeOH = 10/1, UV) indicated complete consumption of the starting material (R_{f}= 0.50), one main new spot (R_{ƒ}= 0.30) could be observed. The reaction was diluted with brine and extracted with DCM (3* 20.0 mL). The organic phase was combined and dried over Na₂SO₄, filtered and concentrated. The residue was purified by preparative TLC (DCM/MeOH = 10/1) 2 times to give pure target compound. Yield: 18.0 mg (24%).

Example 21 - Preparation of N-(2-(Dimethylamino)ethyl)-1,2,3-trimethyl-1H-indole-5-carboxamide (Compound 41)

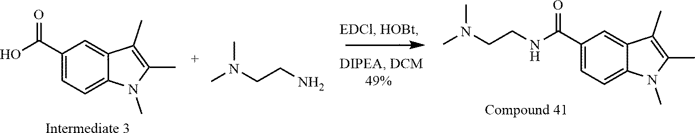

To a stirred solution of Intermediate 3 (50.0 mg, 0.25 mmol) in 5.00 mL DCM was added HOBt (39.9 mg, 0.30 mmol), EDC (49.2 mg, 0.30 mmol), N¹,N¹-dimethylethane-1,2-diamine (26.0 mg, 0.30 mmol) and DIEA (63.6 mg, 0.49 mmol). The reaction mixture was stirred at 25° C. for 16 h. TLC check (DCM/MeOH = 10/1) showed full consumption of Intermediate 3 (R_{f}= 0.50), a major new spot (R_{f}= 0.30) was observed. The reaction was diluted with 5.00 mL DCM and 10.0 mL H₂O. The organic phase was separated, the aqueous layer was extracted with DCM (5.00 mL * 2). The organic phase was combined, dried over Na₂SO₄, filtered and concentrated. The residue was purified by preparative TLC (DCM/MeOH = 10/1) to give pure target compound. Yield: 33.0 mg (49%).

Example 22 - Preparation of N-(2-(Azetidin-1-yl)ethyl)-1,2,3-dimethyl-1H-indole-5-carboxamide (Compound 44)

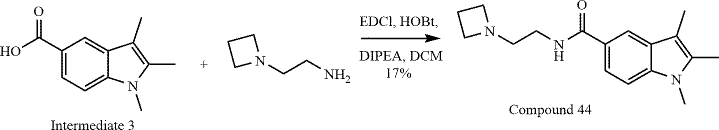

To a stirred solution of Intermediate 3(50.0 mg, 0.25 mmol) in 5.00 mL DCM was added HOBt (39.9 mg, 0.30 mmol), EDCI (45.8 mg, 0.30 mmol), 2-(azetidin-1-yl)ethan-1-amine (29.6 mg, 0.30 mmol) and DIEA (63.6 mg, 0.49 mmol). The reaction mixture was stirred at 25° C. for 16 h. The reaction mixture was stirred at 25° C. for 16 h. TLC check (DCM/MeOH = 10/1) showed full consumption of Intermediate 3 (R_{f}= 0.50), a major new spot (R_{f}= 0.30) was observed. The reaction was diluted with 5.00 mL DCM and 10.0 mL H₂O. The organic phase was separated, the aqueous layer was extracted with DCM (5.00 mL * 2). The organic phase was combined, dried over Na₂SO₄, filtered and concentrated. The residue was purified by preparative TLC (DCM/MeOH = 10/1) 3 times to give pure target compound. Yield: 12.0 mg (17%).

Example 23 - Preparation of 1,2,3-Trimethyl-N-(2-morpholinoethyl)-1H-indole-5-carboxamide (Compound 45)

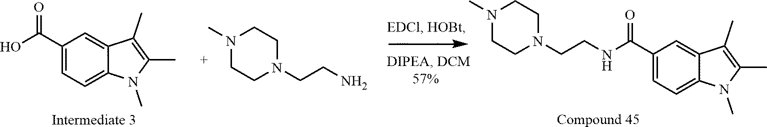

To a stirred solution of Intermediate 3(50.0 mg, 0.25 mmol) in 5.00 mL DCM was added HOBt (39.9 mg, 0.30 mmol), EDCI (45.8 mg, 0.30 mmol), 2-(4-methylpiperazin-1-yl)ethan-1-amine (42.3 mg, 0.30 mmol) and DIEA (63.6 mg, 0.49 mmol). The reaction mixture was stirred at 25° C. for 16 h. The reaction mixture was stirred at 25° C. for 16 h. TLC check (DCM/MeOH = 10/1) showed full consumption of Intermediate 3 (R_{f}= 0.50), a major new spot (R_{f}= 0.30) was observed. The reaction was diluted with 5.00 mL DCM and 10.0 mL H₂O. The organic phase was separated, the aqueous layer was extracted with DCM (5.00 mL * 2). The organic phase was combined, dried over Na₂SO₄, filtered and concentrated. The residue was purified by preparative TLC (DCM/MeOH = 10/1) to give target compound. Yield: 46.0 mg (57%).

Example 24 - Preparation of 1,2,3-Trimethyl-N-(2-(pyrrolidin-1-yl)ethyl)-1Hindole-5-carboxamide (Compound 46)

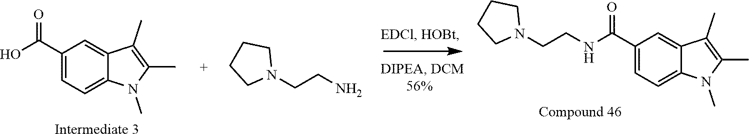

To a stirred solution of Intermediate 3 (50.0 mg, 0.25 mmol) in 5.00 mL DCM was added HOBt (39.9 mg, 0.30 mmol), EDCI (45.8 mg, 0.30 mmol), 2-(pyrrolidin-1-yl)ethan-1-amine (33.7 mg, 0.30 mmol) and DIEA (63.6 mg, 0.49 mmol). The reaction mixture was stirred at 25° C. for 16 h. The reaction mixture was stirred at 25° C. for 16 h. TLC check (DCM/MeOH = 10/1) showed full consumption of Intermediate 3 (R_{f}= 0.50), a major new spot (R_{f}= 0.30) was observed. The reaction was diluted with 5.00 mL DCM and 10.0 mL H₂O. The organic phase was separated, the aqueous layer was extracted with DCM (5.00 mL * 2). The organic phase was combined, dried over Na₂SO₄, filtered and concentrated. The residue was purified by preparative TLC (DCM/MeOH = 10/1) to give target compound. Yield: 41.0 mg (56%).

Example 25 - Preparation of 2,3-Dimethyl-N-(2-(piperidin-1-yl)ethyl)-1H-indole-5-carboxamide (Compound 47)

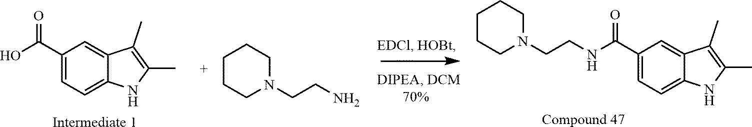

To a stirred solution of Intermediate 1 (50.0 mg, 0.26 mmol) in 5.00 mL DCM was added HOBt (42.8 mg, 0.32 mmol), EDC (49.2 mg, 0.32 mmol), 2-(piperidin-1-yl)ethan-1-amine (40.7 mg, 0.32 mmol) and DIEA (68.3 mg, 0.53 mmol). The reaction mixture was stirred at 25° C. for 16 h. The reaction mixture was stirred at 25° C. for 16 h. The reaction mixture was stirred at 25° C. for 16 h. TLC check (DCM/MeOH = 10/1, UV) indicated complete consumption of the starting material (R_{ƒ}= 0.50), one main new spot (R_{ƒ}= 0.30) could be observed. The reaction was diluted with brine and extracted with DCM (3* 20.0 mL). The organic phase was combined and dried over Na₂SO₄, filtered and concentrated. The residue was purified by preparative TLC (DCM/MeOH = 10/1) to give pure target compound. Yield: 56.0 mg (70%).

Example 26 - Preparation of 2,3-Dimethyl-N-(2-morpholinoethyl)-1H-indole-5-carboxamide (Compound 24)

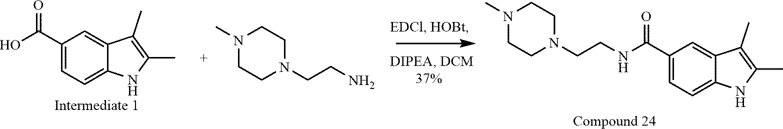

To a stirred solution of Intermediate 1 (50.0 mg, 0.26 mmol) in 5.00 mL DCM was added HOBt (42.8 mg, 0.32 mmol), EDC (49.2 mg, 0.32 mmol), 2-(4-methylpiperazin-1-yl)ethan-1-amine (45.4 mg, 0.32 mmol) and DIEA (68.3 mg, 0.53 mmol). The reaction mixture was stirred at 25° C. for 16 h. The reaction mixture was stirred at 25° C. for 16 h. The reaction mixture was stirred at 25° C. for 16 h. TLC check (DCM/MeOH = 10/1, UV) indicated complete consumption of the starting material (R_{ƒ}= 0.50), one main new spot (R_{ƒ}= 0.30) could be observed. The reaction was diluted with brine and extracted with DCM (3* 20.0 mL). The organic phase was combined and dried over Na₂SO₄, filtered and concentrated. The residue was purified by preparative TLC (DCM/MeOH = 10/1) to give pure target compound. Yield: 31.0 mg (37%).

Example 27 - Preparation of 1,2,3-Trimethyl-N-(2-(piperidin-1-yl)ethyl)-1H-indole-5-carboxamide (Compound 31)

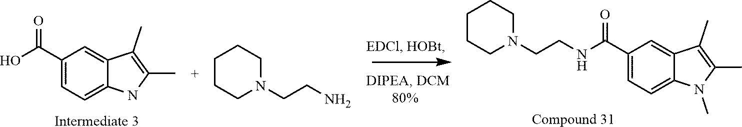

To a stirred solution of Intermediate 3 (50.0 mg, 0.25 mmol) in 5.00 mL DCM was added HOBt (39.9 mg, 0.30 mmol), EDCI (45.8 mg, 0.30 mmol), 2-(piperidin-1-yl)ethan-1-amine (37.9 mg, 0.30 mmol) and DIEA (63.6 mg, 0.49 mmol). The reaction mixture was stirred at 25° C. for 16 h. The reaction mixture was stirred at 25° C. for 16 h. TLC check (DCM/MeOH = 10/1) showed full consumption of Intermediate 3 (R_{f}= 0.50), a major new spot (R_{f}= 0.30) was observed. The reaction was diluted with 5.00 mL DCM and 10.0 mL H₂O. The organic phase was separated, the aqueous layer was extracted with DCM (5.00 mL * 2). The organic phase was combined, dried over Na₂SO₄, filtered and concentrated. The residue was purified by preparative TLC (DCM/MeOH = 10/1) to give target compound. Yield: 62.0 mg (80%).

Example 28 - Preparation of N-(2-(diethylamino)ethyl)-3-ethyl-2-methyl-1H-indole-5-carboxamide (Compound 4)

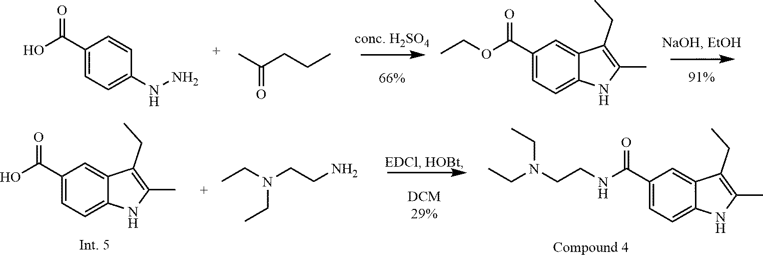

To a stirred solution of Intermediate 5 (100 mg) was added N,N-dimethylethane-1,2-diamine, EDCI, HOBt, and IDEA. The reaction mixture was stirred at 26° C. for 16 h to afford 43.0 mg (29% yield) after purification by PTLC.

### Example 29 - In-Silico Screen for Compounds That Interact With Mutant Huntingtin (mHTT)

Using a mutant Huntingtin protein (mHTT), a structure-based virtual screening was performed to determine the structures of small molecules that can interact with the surface of the polyQ peptide of the mHTT. Interaction of small molecules with this surface is predicted to reverse the conformational changes of mHTT to wildtype HTT. A final set of 39 compounds were selected for further testing.

### Example 30 - Mutant Huntingtin (mHTT) Protein Expression

Exon 1 of the HTT huntingtin (NCBI Gene ID 3064) was expressed as a fusion protein with (i) TRX only (MurTRX); (ii) 16Q (Mur16); and (iii) 46Q (Mur46). MurTRX, Mur16, and Mur46 were produced and purified by HIS-tag and size exclusion chromatography (SEC). The protocol for MurTRX, Mur16 and Mur46 expression was adopted from Bennett, M.J. et al. Proc. Natl. Acad. Sci. USA 99, 11634-11639.2002.

Generation of MurTRX, Mur 16 and Mur46 Expression Vectors - The TRX-linker-histag (MurTRX), TRX-linker-16Q-histag (Mur16), TRX-linker-46Q-histag (Mur46) were synthesized by IDT DNA (Integrated DNA Technologies, Coralville, Iowa USA). These fragments were cloned into the NcoI and BamHI sites of a pET28a+ expression vector (Novagen part of Merck-Millipore) and overexpressed in BL21(DE3) plysE cells (Bioline Ltd, London, UK). The linker segment had the sequence GSGSGERQHMDSPDLGTDDDDK (SEQ ID NO: 1). Sequence alignments for selected colonies are shown below.

MurTRX Sequence Alignment:

70HG41 MIAPILDEIADEYQGKLTVAKLNIDQNPGTAPKYGIRGIPTLLLFKNGEVAATKVGALSK
Designed MIAPILDEIADEYQGKLTVAKLNIDQNPGTAPKYGIRGIPTLLLFKNGEVAATKVGALSK
************************************************************
70HG41 GQLKEFLDANLAGSGSGERQHMDSPDLGTDDDDKGSGHHHHHH (SEQ ID NO: 2)
Designed GQLKEFLDANLAGSGSGERQHMDSPDLGTDDDDKGSGHHHHHH (SEQ ID NO: 3)
*******************************************

Mur16 Sequence Alignment:

sequenced MSDKIIHLTDDSFDTDVLKADGAILVDFWAEWCGPCKMIAPILDEIADEYQGKLTVAKLN
designed MSDKIIHLTDDSFDTDVLKADGAILVDFWAEWCGPCKMIAPILDEIADEYQGKLTVAKLN
************************************************************
sequenced IDQNPGTAPKYGIRGIPTLLLFKNGEVAATKVGALSKGQLKEFLDANLAGSGSGERQHMD
designed IDQNPGTAPKYGIRGIPTLLLFKNGEVAATKVGALSKGQLKEFLDANLAGSGSGERQHMD
************************************************************
sequenced SPDLGTDDDDKMATLEKLMKAFESLKSFQQQQQQQQQQQQQQQQPPPPPPPPPPPQLPQP
designed SPDLGTDDDDKMATLEKLMKAFESLKSFQQQQQQQQQQQQQQQQPPPPPPPPPPPQLPQP
************************************************************
sequenced PPQAQPLLPQPQPPPPPPPPPPGPAVAEEPLHRGSGHHHHHH (SEQ ID NO: 4)
designed PPQAQPLLPQPQPPPPPPPPPPGPAVAEEPLHRGSGHHHHHH (SEQ ID NO: 5)
******************************************

Mur46 Sequence Alignment:

sequenced XPSXNILFTLRRRYTMSDKIIHLTDDSFDTDVLKADGAILVDFWAEWCGPCKMIAPILDE
designed ---K-FCLTLRRRYTMSDKIIHLTDDSFDTDVLKADGAILVDFWAEWCGPCKMIAPILDE
: :****************************************************
sequenced IADEYQGKLTVAKLNIDQNPGTAPKYGIRGIPTLLLFKNGEVAATKVGALSKGQLKEFLD
designed IADEYQGKLTVAKLNIDQNPGTAPKYGIRGIPTLLLFKNGEVAATKVGALSKGQLKEFLD
************************************************************
sequenced ANLAGSGSGERQHMDSPDLGTDDDDKMATLEKLMKAFESLKSFQQQQQQQQQQQQQQQQQ
designed ANLAGSGSGERQHMDSPDLGTDDDDKMATLEKLMKAFESLKSFQQQQQQQQQQQQQQQQQ
************************************************************
sequenced QQQQQQQQQQQQQQQQQQQQQQQQQQQQQPPPPPPPPPPPQLPQPPPQAQPLLPQPQPPP
designed QQQQQQQQQQQQQQQQQQQQQQQQQQQQQPPPPPPPPPPPQLPQPPPQAQPLLPQPQPPP
************************************************************
sequenced PPPPPPPGPAVAEEPLHRGSGHHHHHH--AAALEHHHHHH-DPAANKARKEAELAAATAE
designed PPPPPPPGPAVAEEPLHRGSGHHHHHHAA-------------------------------
*************************** *
sequenced Q-LA-PLGASKRVLRGFLLKGGTISGLANGTRPVAAH-ARRVWWLRAA-PLHLPAP-RPL
designed ------------------------------------------------------------
* * * * *
sequenced LSLSSLPXXXXSPAFPVKL-IGGSL-XS (SEQ ID NO: 6)
designed ---------------------------- (SEQ ID NO: 7)
* *

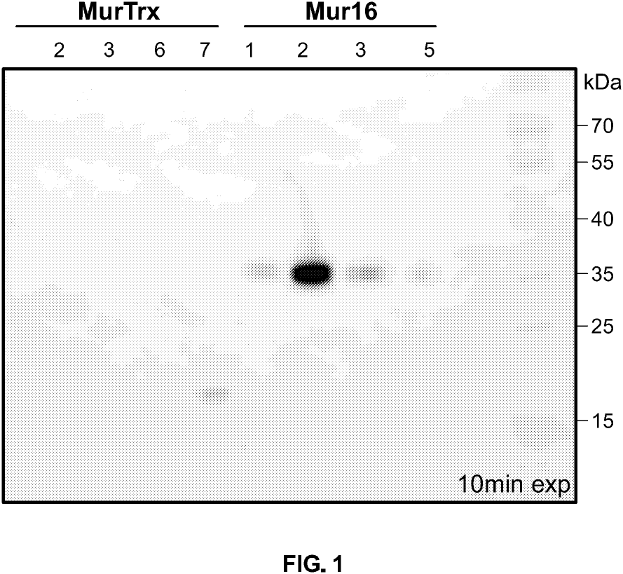

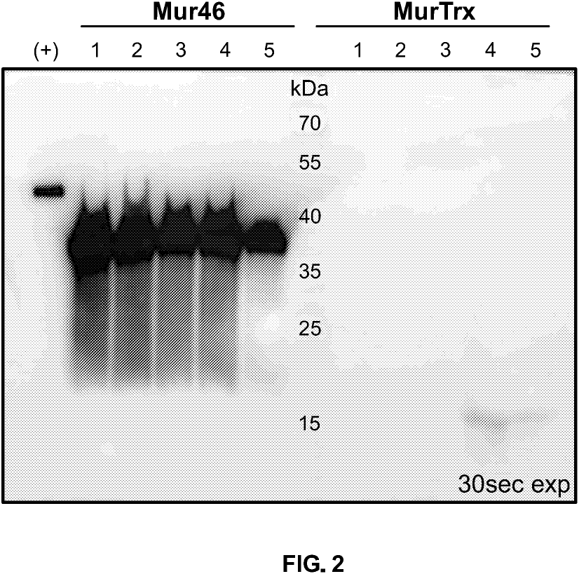

mHTT Protein Expression - Colonies were picked and 5 mL cultures were grown. Cells were induced at Optical Density (OD) 0.7-0.8 for four hours at 37° C. 1 mL of the post-induction culture was spun down and the pellet resuspended in lysis buffer. This was spun down at 10,000 RPM for 10 minutes and the soluble fraction loaded on a 4-20% polyacrylamide gel (NuSep). An anti-His western blot was performed to detect protein expression. Mur16 protein was expressed (FIG. 1). Mur46 protein was expressed (FIG. 2). Weaker protein expression signal was observed for MurTRX (FIG. 2). An ELISA assay was performed to confirm the expression and detection of the proteins. MurTRX, Mur16 and Mur46 are recognized by anti-His antibodies. Mur16 and Mur46 are recognized by anti-polyQ antibody. The anti-polyQ antibody, 3B5H10 MAb (Sigma Aldrich, Saint Louis, USA), was used in the ELISA assay.

### Example 32 - Determination of mHTT and Compound Interactions by Surface Plasmon Resonance (SPR)

A Biacore Surface Plasmon Resonance (SPR) assay was used as a label-free method to determine the interaction of small molecule compounds with the mHTT target.

MurTRX, Mur 16 and Mur46 Protein Purification - Proteins were loaded onto IMAC resin (ThermoFisher Scientific HisPur) and eluted in 200 mM imidazole, purified by gel filtration FPLC (HiLoad superdex-200, 26/60; GE Life sciences) and concentrated using 3 kDa cut off Vivaspin 20 PES centrifugal concentrators (Sartorius AG). Proteins were biotinylated using a 1:0.5 molar ratio of EZ-link™ Sulfo-NHS-LC-LC-Biotin (ThermoFisher Scientific) and thoroughly dialysed against PBS prior to Biacore coupling.

Neutravidin CM5 amine coupling immobilisation - It was determined that the ligand density of streptavidin found on a standard streptavidin (SA) Chip was not high enough for the assay (~3,000 RU). A customized biotin capture chip using NeutrAvidin as the capture ligand was created for this assay. To obtain high enough ligand density on the sensor chip surface, NeutrAvidin (Thermoscientific Pierce, Waltham, MA USA) was immobilised on all four sensor chip surfaces to a level of approximately 20,000 RU.

Capture of the Mur ligands to NeutrAvidin - Biotinylated proteins were diluted in HBS EP+ running buffer and captured on the sensor chip surface. Mur46 was coupled to the sensor chip first. After saturating FC3 with Mur46, target response levels for Mur16 and MurTRX were calculated based on their respective molecular weights. This ensured that the same molarity of each target was coupled to the sensor chip surface, ensuring that the same proportion of non-specific binding to TRX for all analytes was the same across all flow cells.

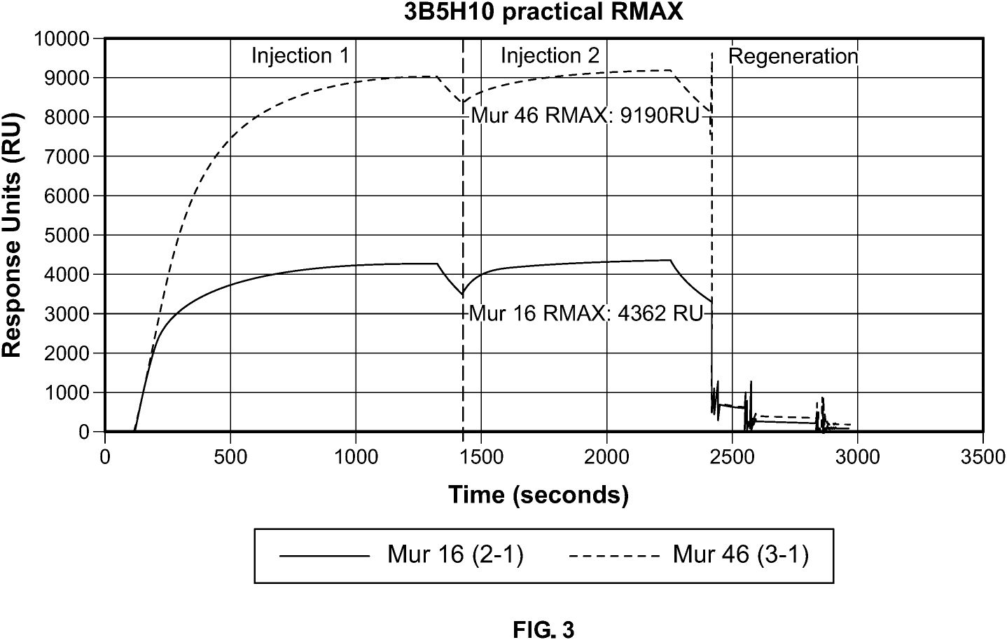

Sensor chip validation Poly-Q 3B5H10 MAb binding - To investigate the binding properties of the coupled chip, 3B5H10 MAb (Sigma Aldrich, Saint Louis, USA) was diluted 1:600 and flowed over all four flow cells to the point of saturation using injections (FIG. 3).

TABLE 1

Practical RMAX

| Ligand | 3B5H10 practical RMAX (RU) | |
|---|---|---|
| Mur 16 | 9,190 | 19 |
| Mur 46 | 4,362 | 15.5 |

The molecular weight of the MAb is ~150kDa, which is ~5x larger than the largest ligand, Mur46. Interestingly, with the same molarity of Mur 16/46 coupled to the flow cells, double the level of MAb binding was observed with Mur46 in comparison to Mur 16 (Table 1). Assuming that the proportion of ligand bound to both flow cells is active, this would indicate that there are double the MAb accessible epitopes on the 46Q than the 16Q.

Primary Screen -Methods - Briefly, compounds were diluted in 100% DMSO to a final concentration of 100 mM. These were diluted wherever possible to 1 mM, 0.1 mM and 0.01 mM in 10% DMSO in PBS+ running buffer. The entire assay was run in 10% DMSO in the BIACore T200 (GE, Little Chalfont, United Kingdom) and an appropriate solvent correction window was selected for the assay. Where compounds were found to precipitate at 1 mM, compounds were prepared in 10% DMSO at 2-fold lower concentrations e.g. 0.5 mM, 0.05 mM and 0.005 mM. Samples were run by identity, from the lowest to the highest concentration with one concentration per cycle. Low-pH regeneration was performed after each sample injection and an extra-wash with 50% DMSO was performed on the machine to eliminate sample carry-over. The assay was run with a 10 HZ data collection frequency in a multi (4-1, 3-1, 2-1) configuration and compounds were in contact with the ligands for 60 seconds. Blank samples (required for double referencing) were passed over the sensor surface every 21 cycles in triplicates, as was the positive MAb control and solvent correction.

Primary Screen - Results - 39 compounds determined from the in-silico screen were tested. Following solvent correction and blank subtraction, double referenced binding levels for samples were plotted against binding levels on both Mur 16 and Mur 46. 4 compounds showed binding to the target structures Mur16 and Mur46 (Table 2). The 4 compounds demonstrated higher binding to the drug targets with increasing concentration of the compound. The 4 compounds consistently bind with a higher response on the 16 Q surface than the 46 Q repeat surface, despite the MAb positive control showing the opposite trend.

TABLE 2

Compounds that demonstrated binding to Mur16 and/or Mur46

| Compound Number | Biacore affinity (RU²) | Chemical Structure |
|---|---|---|
| 109 | 3 | 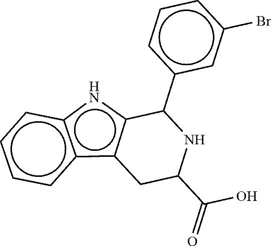 |
| 107 | 92 | 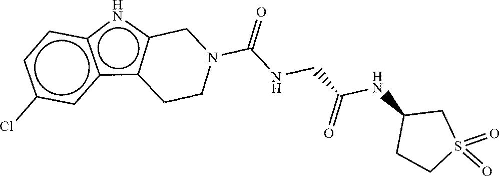 |
| 113 | 160 | 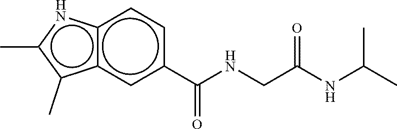 |
| 110 | 80 | 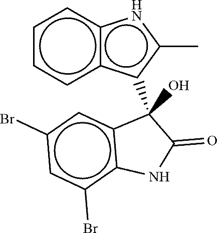 |

Analog Screening-Methods - Analog or derivative compounds from the secondary screen were determined and were used in an analog Biacore SPR screen. Compounds were stored at 100% DMSO with a concentration of 100 mM. For the screening the compounds were diluted to 1 mM in 5% DMSO in PBS+ running buffer. This was serially diluted to 0.1 and 0.01 mM (retaining 5% DMSO). Compounds which did not dissolve at 1 mM in 5% DMSO were diluted 10 fold to 0.1 mM in 5% PBS+ and run in the assay at 0.1, 0.01 and 0.001 mM concentrations MAb 3B5H10 (1:5000) was used as a positive control whilst running buffer was used as a negative. Solvent correction was carried out every 20 cycles as recommended by BIACore. Samples were flowed over the sensor chip surface for 60 seconds and regeneration was carried out with 0.1 M glycine, pH 3.0 for 30 seconds. A system wash with 50% DMSO was carried out after each compound injection to reduce the chance of compounds cross contamination across the length of the run.

First Analog Screening - Results - The results of the first analog screen are shown in Table 4.

Second Analog Screening - Results - The results of the second analog screen are shown in Table 4. Table 4 lists the compound identification numbers and the binding of the compounds to the target structure.

Third Analog Screening - Results - The results of the third analog screen are shown in Table 4. Table 3 lists the compound identification numbers and the binding of the compounds to the target structure in comparison to the parent compound.

TABLE 3

Binding and Solubility of Analog Compounds

| Compound | Binding properties | Solubility |
|---|---|---|
| 57 | ++ | +/- |
| 102 | + (Mur16) ++ (Mur46) | +/- |
| 106 | + (Mur16) ++ (Mur46) | +/- |
| 105 | ++ | +/- |
| 103 | ++ | +/- |
| 114 | +/ | +/ |

++ very improved in comparison to parent compound
+ improved in comparison to parent compound
- reduced in comparison to parent compound
+/- in par with parent compound

Table 4 shows a summary of the compounds and analog compounds that were tested using the Biacore screening method.

Table 4: Affinity of Compounds to Mur46 Determined by Biacore Screening. Biacore affinity was measured at 0.1 mM compound concentration; * means 1 mM concentration of the compound was used for Biacore measurement in case there was no binding detected at 0.1 mM; ** means 0.01 mM concentration for measurement was used in case the compound was not soluble at 0.1 mM; *** means 0.001 mM concentration for measurement was used in case the compound was not soluble at 0.1 mM and at 0.01 mM

TABLE 4

Biacore Affinity Values for Binding of Compounds to Mur46

| Compound Number | Biacore affinity (RU²) | Chemical Structure |
|---|---|---|
| 102 | 28 | 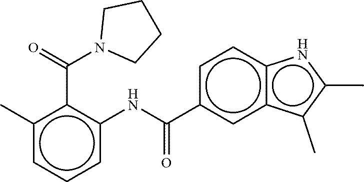 |
| 103 | 124 | 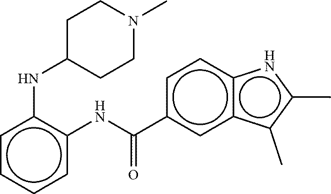 |
| 104 | 116 | 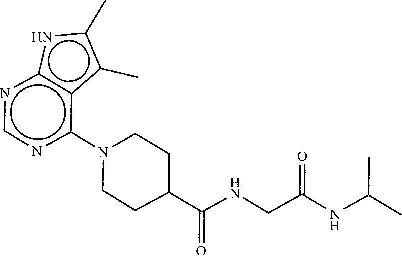 |
| 57 | 214 | 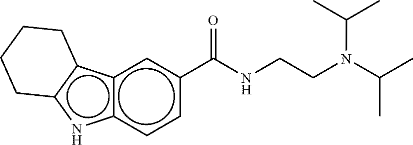 |
| 105 | 118 | 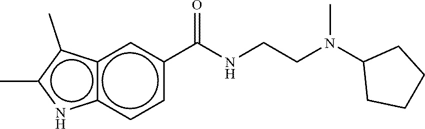 |
| 106 | 93 | 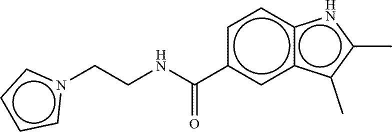 |
| 108 | 6** | 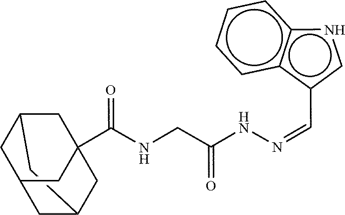 |
| 112 | 130 | 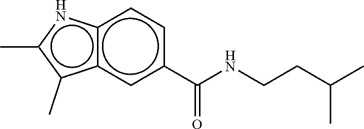 |
| 7 | 122 | 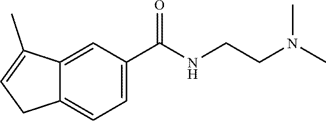 [SMILES: CC1=CCc2ccc(C(=O)NCCN(C)C)cc21] |
| 114 | 20 | 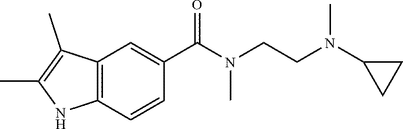 [SMILES: Cc1[nH]c2ccc(C(=O)N(C)CCN(C)C3CC3)cc2c1C] |

Compounds from the Biacore screening were analyzed in a modified Parallel Artificial Permeation Assay (PAMPA) assay for their ability to permeate the blood brain barrier. The filter material of a 96-well microplate is impregnated with brain lipids (Di, L. et al., Eur. J. Med. Chem. 2003 Mar;38(3):223-232). The test compounds were added to the donor compartment. After diffusion, the concentration of the compounds in the acceptor compartment were determined by Liquid Chromatography with tandem mass spectrometry (LC-MS-MS) to calculate the flux rate. Based on these permeation data, the drugs were classified as blood brain barrier permeating (BBB+) with a flux rate >50% or non-permeating (BBB-) with a flux rate <50%. Table 5 shows the results of this experiment for Compound 53 and Compound 7.

TABLE 5

Assessment of Blood Brain Barrier Permeation by LC-MS-MS

| Compound number | Flux [%] | SD | SE | Mean Recovery [%] | SD | Classification |
|---|---|---|---|---|---|---|
| 53 | 76.7 | 1.1 | 0.6 | 103.5 | 1.7 | BBB+ |
| 7 | 70.8 | 4.7 | 2.7 | 88.5 | 2.6 | BBB+ |

Compounds from the Biacore screening were selected for measurement of aggregation in a cell model Htt14A2.6 PC12. Htt14A2.6 PC12 cells express a GFP- tagged 97Q mHtt exon 1 fusion protein (mHttex1-GFP) in the presence of ponasterone A. Htt14A2.6 PC12 cells were grown in medium containing 5 µM ponasterone A for 20 hours prior to treatment with the compounds. Htt14A2.6 PC12 cells were plated at 50~60% confluency. Compound 22 was tested at a final concentration of 80 nM, 400 nM and 10 µM in cell culture medium for 24 hours. Htt14A2.6 PC12 cells were harvested, rinsed, homogenized in cold phosphate-buffered saline containing protease inhibitor cocktail, and centrifuged at 16000 g for 30 min at 4° C. twice. About 25 µg total protein of each sample was resolved in 8% SDS PAGE, transferred onto PVDF membrane, blotted with an anti-GFP antibody, and visualized by ECL detection. mHTT aggregates were quantified from the cell lysates by filter retardation assay (Wanker, E.E., et al., Methods Enzymol (1999); 309: 375-386).

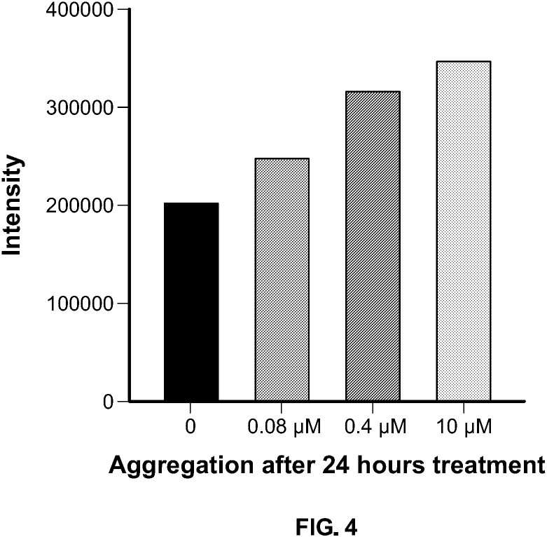

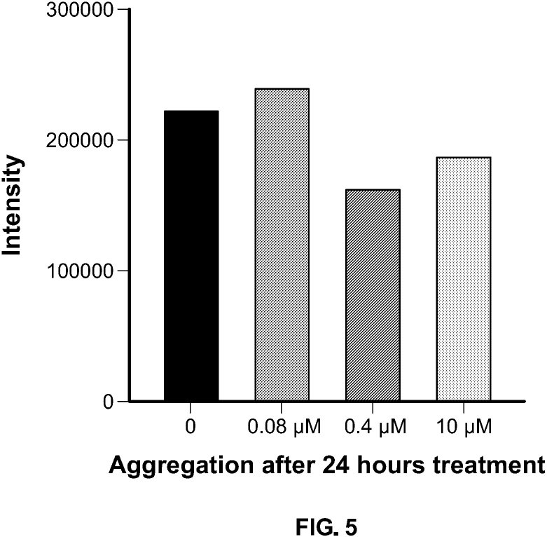

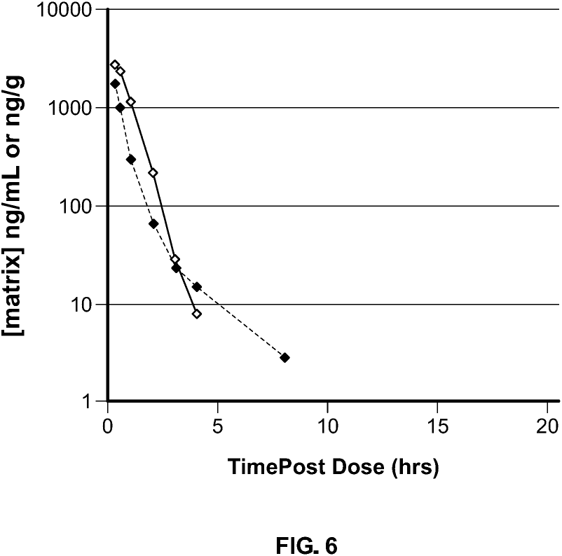

Compound 7 did not show a reduction of aggregation in Htt14A2.6 PC12 (FIG. 4). Compound 22 is an analog of Compound 7. A reduction of mHTT in Htt14A2.6 PC12 cells was observed following treatment with Compound 22 (FIG. 5). Compound 22 pharmacokinetic properties was measured in three male CD1 mice at several timepoints. Compound 22 was dissolved in 10% DMSO, 10%, cremaphor, and 80% saline. The tested dose was 33.3 mg/kg body weight. Brain tissue was homogenized and protein precipitated with acetonitrile. The concentration of Compound 22 was measured in plasma and brain with UltraHigh Performance Liquid Chromatography combined with Time-of-Flight Mass Spectrometry (UHPLC - TOF). The results of this experiment show that Compound 22 can penetrate the blood brain area in vivo (FIG. 6). As such, Compound 22 was selected for testing in an animal model of HD the R6/2 model. As described below, Compound 22 and its analogs were further tested for their effects on striatal cell viability and effects of mHTT levels.

### Example 33 - Effects of Compounds on Striatal Cell Viability and mHTT Levels

94 compounds were tested for their ability to bind mHTT and reduce diffuse levels of mHTT. Diffuse mHTT can be both monomeric and or oligomeric. Diffuse mHTT is correlated to pathological parameters in Huntington’s disease.

Cell culture - ST HDH Q111/111 (CH00095, Coriell Institute) striatal derived cell line were grown at 33° C. in DMEM (Sigma-Aldrich), supplemented with 10% fetal bovine serum (FBS), 1% Penicillin-Streptomycin (ThermoFisher Scientific), and 0.4 mg/ml G418 (Geneticin; Invitrogen). ST HDH Q111/111 are mouse striatal cells with polyQ length of 1111.

Pre-Treatment of striatal cell line - First the medium was removed and new DMEM (DMEM, high glucose, HEPES, no phenol red) medium, supplemented with 10% fetal bovine serum (FBS), 1% Penicillin-Streptomycin, containing 10 µM compound was added. The cells were incubated at 33° C. for 24 hours.

Heat shock - Cells were heat shocked for 3 hours at 41° C.

Treatment - Medium was removed and new DMEM (DMEM, high glucose, HEPES, no phenol red) medium, supplemented with 1% fetal bovine serum (FBS), 1% Penicillin-Streptomycin, containing 10 µM compound was added. The cells were incubated at 33° C. for 48 hours.

Cell Viability - Cell viability was measured according to manufacturer recommendations with CellTiter Glo (Promega). 96-well plates for used either for cell viability determination or cell staining.

Staining - Cells were permeabilized with 0.3% Triton X-100 in 4° C. PBS (pH 7.4 from ThermoFisher) for 10 minutes. Then the cells were washed two times with 4° C. PBS. Subsequently, cells are blocked with 3% NGS (normal goat serum from Thermo Fisher) in 4° C. PBS for 1 hour. Cells were incubated with primary antibodies in blocking buffer (PBS, 3% NGS, 0.02% NaN3) over night at 4° C. Primary antibodies were mouse antipolyglutamine monoclonal antibody 3B5H10 (Sigma-Aldrich) at 1:250 dilution and Guinea Pig anti-MAP2 polyclonal antibody (Synaptic Systems) at 1:500 dilution. 3B5H10 binds to diffuse mHTT. Diffuse mHTT is a monomeric and small oligomeric mHTT. Diffuse mHTT is correlated to pathological parameters in Huntington’s disease. Compounds that bind the mHTT will reduce the levels of diffuse mHTT detected.

Cells were washed 2 times with 4° C. PBS, and then incubated with secondary antibodies at 1:2000 dilution in blocking buffer for 1 hour at room temperature. Secondary antibodies were Goat anti-Guinea Pig IgG (H+L) Highly Cross-Adsorbed Secondary Antibody, Alexa Fluor 647 (ThermoFisher Invitrogen) and Goat anti-Mouse IgG (H+L) Cross-Adsorbed Secondary Antibody, Alexa Fluor 488 (ThermoFisher Invitrogen). Cells were washed 2 times with 4° C. PBS, and then the nuclei were counterstain with DAPI (ThermoFisher) for 10 min at room temperature in the dark. Cells were washed 2 times with 4° C. PBS, and then 4° C. PBS was added to cells and then images were acquired. Table 6 shows the antibodies used in immunocytochemistry studies.

TABLE 6

Antibodies Used in Immunocytochemistry Studies
Antibodies

| | | |
|---|---|---|
| Mouse anti-human-mHtt mab (clone mEM48) | Merck | MAB5374 |
| Guinea Pig anti-MAP2 pab | Synaptic Systems | 188 004 |
| Alexa Fluor 488 Goat anti-Mouse IgG (H+L) | ThermoFisher Invitrogen | A-11001 |
| Alexa Fluor 647 Goat anti-Rabbit IgG (H+L) | ThermoFisher Invitrogen | A-21244 |
| Alexa Fluor 647 Goat anti-Guinea Pig IgG (H+L) | ThermoFisher Invitrogen | A-21450 |

Fluorescence Imaging - Imaging sessions were performed on a confocal microscope system (Carl Zeiss Microscopy) equipped with a dual spinning disk unit (Yokogawa). All components of the imaging system were controlled via the ZEN 2 software suite (Carl Zeiss Microscopy). The laser lines used were 405 nm, 488 nm and 639 nm to excite DAPI or the respective fluorophores. The fluorescence images obtained of the immunofluorescence labelled tissue sections were quantified with the help of the “Image Processing and Analysis in Java” or short ImageJ software distributed under the GNU General Public License by the NIH (Rueden, C.T. et al., BMC Bioinformatics. 2017 Nov 29;18(1):529), i.e. the edition used was the Fiji distribution (Schindelin, J. et al., Nature methods. 2012 Jun 28;9(7):676-82). Nested t test was performed to calculate the significance amongst repeated measurements using GraphPad Prism software.

RESULTS - Table 7a summarizes the effect of the compounds on the levels of mHTT in striatal cells. STHdh 111/111 were heat shocked for 3 hours and treated for 48 hours with small molecule compounds. Table 7 lists the percentage of mHTT and the cell viability in striatal cells in comparison to untreated STHdh 111/111 striatal cells after 48 hours. The compounds were ranked by efficacy.

TABLE 7a

Effect of Compounds on mHTT levels and cell viability of STHdh 111/111 striatal cells

| Compound Number | mHTT after treatment | Cell Viability striatal cells 3h heat shocked | Chemical Structure |
|---|---|---|---|
| 1 | 30 | 111 | 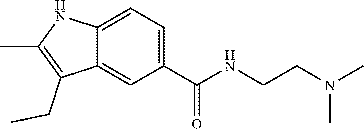 |
| 2 | 32 | 100 | 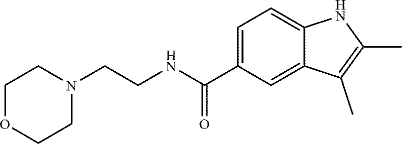 [SMILES: Cc1[nH]c2ccc(C(=O)NCCN3CCOCC3)cc2c1C] |
| 3 | 33 | 86 | 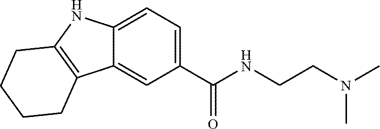 [SMILES: CN(C)CCNC(=O)c1ccc2[nH]c3c(c2c1)CCCC3] |
| 4 | 34 | 125 | 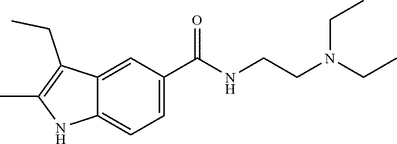 [SMILES: CCc1c(C)[nH]c2ccc(C(=O)NCCN(CC)CC)cc12] |
| 5 | 36 | 104 | 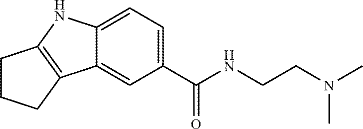 [SMILES: CN(C)CCNC(=O)c1ccc2[nH]c3c(c2c1)CCC3] |
| 6 | 37 | 113 | 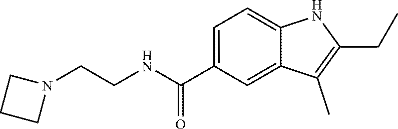 [SMILES: CCc1[nH]c2ccc(C(=O)NCCN3CCC3)cc2c1C] |
| 7 | 45 | 96 | 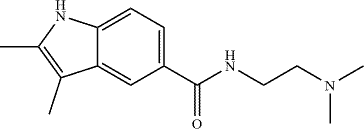 |
| 9 | 37 | 88 | 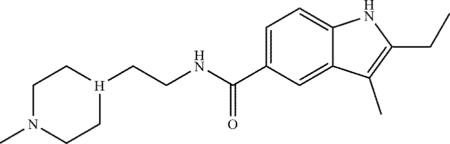 |
| 101 | 38 | 110 | 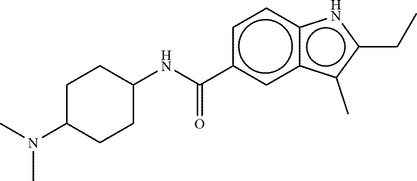 |
| 8 | 38 | 115 | 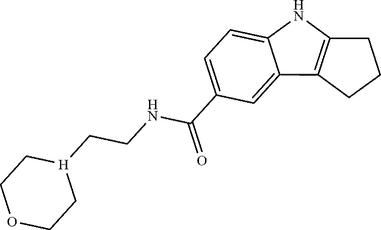 |
| 10 | 40 | 96 | 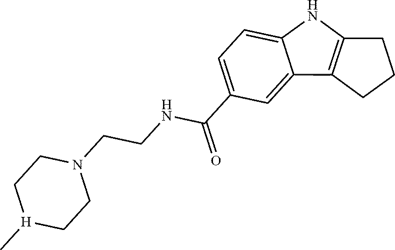 |
| 11 | 40 | 114 | 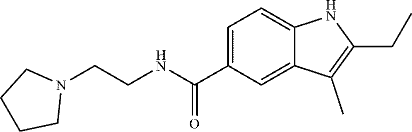 [SMILES: CCc1[nH]c2ccc(C(=O)NCCN3CCCC3)cc2c1C] |
| 12 | 40 | 106 | 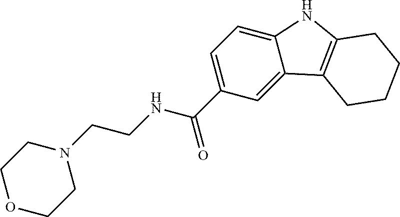 [SMILES: O=C(NCCN1CCOCC1)c1ccc2[nH]c3c(c2c1)CCCC3] |
| 13 | 41 | 117 | 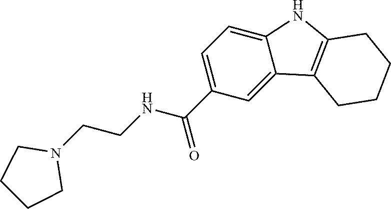 [SMILES: O=C(NCCN1CCCC1)c1ccc2[nH]c3c(c2c1)CCCC3] |
| 14 | 41 | 113 | 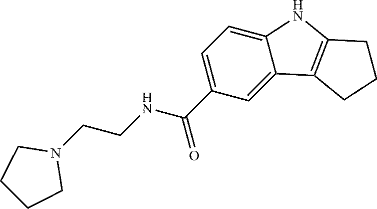 [SMILES: O=C(NCCN1CCCC1)c1ccc2[nH]c3c(c2c1)CCC3] |
| 15 | 42 | 104 | 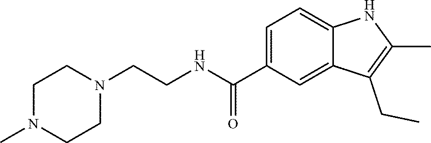 |
| 16 | 43 | 117 | 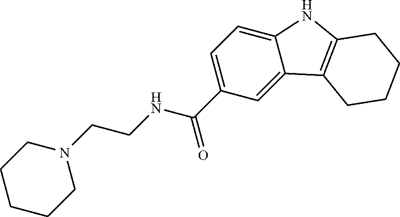 [SMILES: O=C(NCCN1CCCCC1)c1ccc2[nH]c3c(c2c1)CCCC3] |
| 17 | 44 | 99 | 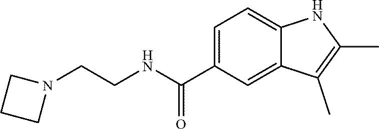 [SMILES: Cc1[nH]c2ccc(C(=O)NCCN3CCC3)cc2c1C] |
| 18 | 44 | 103 | 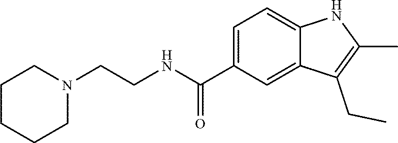 [SMILES: CCc1c(C)[nH]c2ccc(C(=O)NCCN3CCCCC3)cc12] |
| 19 | 45 | 76 | 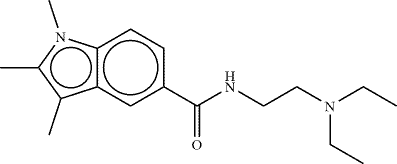 |
| 20 | 45 | 118 | 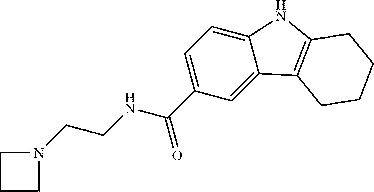 [SMILES: O=C(NCCN1CCC1)c1ccc2[nH]c3c(c2c1)CCCC3] |
| 21 | 46 | 87 | 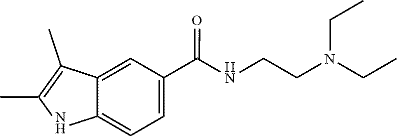 [SMILES: CCN(CC)CCNC(=O)c1ccc2[nH]c(C)c(C)c2c1] |
| 22 | 46 | 102 | 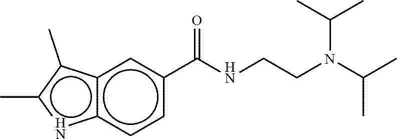 |
| 23 | 47 | 84 | 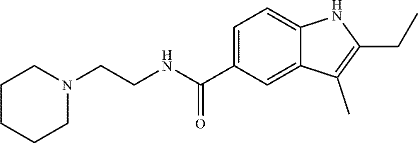 [SMILES: CCc1[nH]c2ccc(C(=O)NCCN3CCCCC3)cc2c1C] |
| 24 | 47 | 103 | |
| 25 | 48 | 109 | 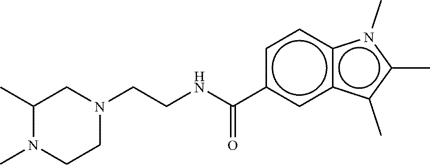 |
| 26 | 49 | 82 | 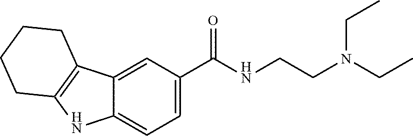 [SMILES: CCN(CC)CCNC(=O)c1ccc2[nH]c3c(c2c1)CCCC3] |
| 27 | 50 | 66 | 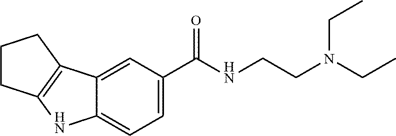 [SMILES: CCN(CC)CCNC(=O)c1ccc2[nH]c3c(c2c1)CCC3] |
| 28 | 51 | 18 | 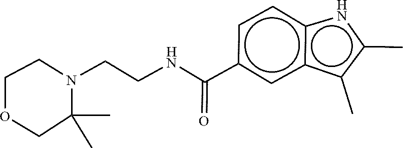 |
| 29 | 51 | 103 | 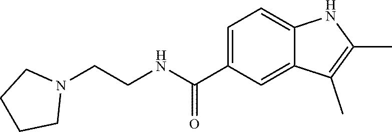 [SMILES: Cc1[nH]c2ccc(C(=O)NCCN3CCCC3)cc2c1C] |
| 30 | 51 | 55 | 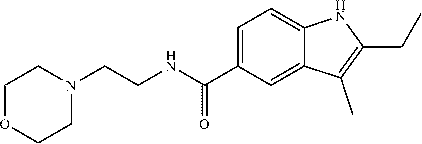 [SMILES: CCc1[nH]c2ccc(C(=O)NCCN3CCOCC3)cc2c1C] |
| 31 | 52 | 116 | |
| 32 | 53 | 98 | 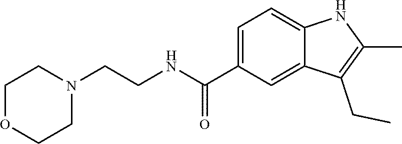 [SMILES: CCc1c(C)[nH]c2ccc(C(=O)NCCN3CCOCC3)cc12] |
| 33 | 55 | 79 | 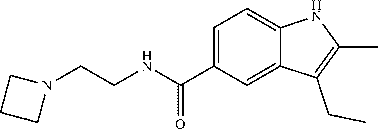 [SMILES: CCc1c(C)[nH]c2ccc(C(=O)NCCN3CCC3)cc12] |
| 34 | 56 | 60 | 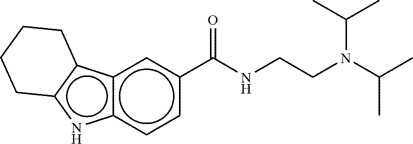 |
| 35 | 56 | 103 | 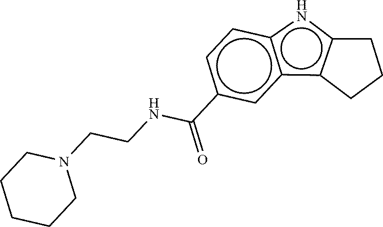 |
| 36 | 56 | 84 | 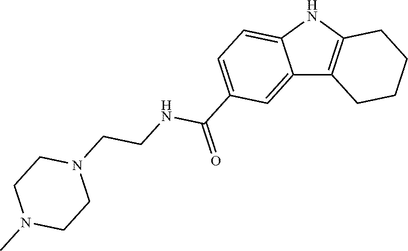 [SMILES: CN1CCN(CCNC(=O)c2ccc3[nH]c4c(c3c2)CCCC4)CC1] |
| 37 | 56 | 78 | 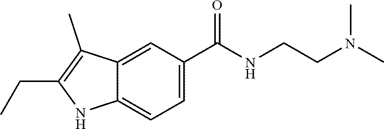 [SMILES: CCc1[nH]c2ccc(C(=O)NCCN(C)C)cc2c1C] |
| 38 | 57 | 81 | 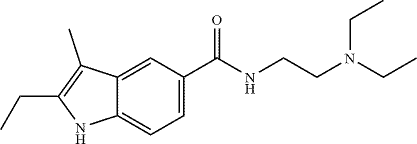 [SMILES: CCc1[nH]c2ccc(C(=O)NCCN(CC)CC)cc2c1C] |
| 39 | 58 | 117 | 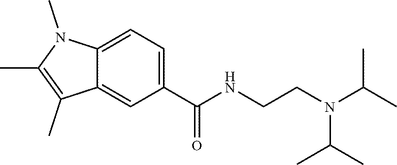 [SMILES: Cc1c(C)n(C)c2ccc(C(=O)NCCN(C(C)C)C(C)C)cc12] |
| 40 | 62 | 85 | 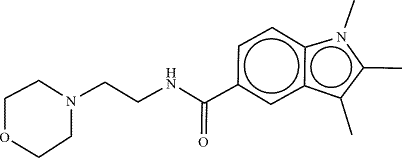 |
| 41 | 65 | 75 | 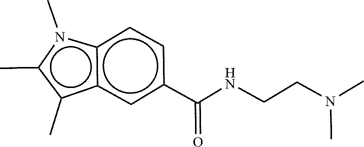 |
| 42 | 65 | 88 | 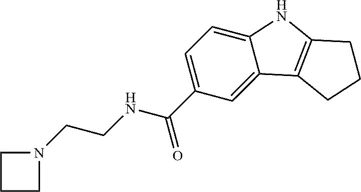 [SMILES: O=C(NCCN1CCC1)c1ccc2[nH]c3c(c2c1)CCC3] |
| 43 | 65 | 32 | 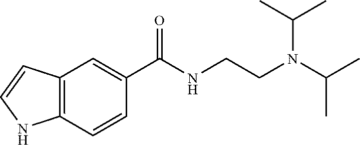 [SMILES: CC(C)N(CCNC(=O)c1ccc2[nH]ccc2c1)C(C)C] |
| 44 | 66 | 75 | 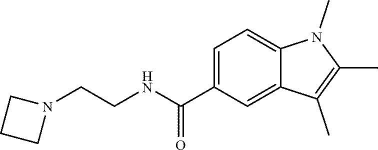 [SMILES: Cc1c(C)n(C)c2ccc(C(=O)NCCN3CCC3)cc12] |
| 45 | 66 | 81 | 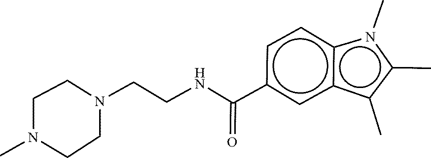 |
| 46 | 66 | 96 | 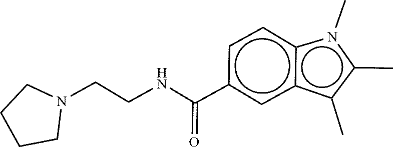 |
| 47 | 66 | 66 | 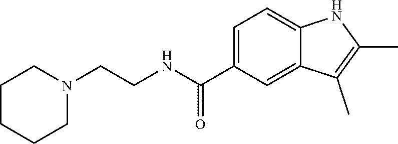 [SMILES: Cc1[nH]c2ccc(C(=O)NCCN3CCCCC3)cc2c1C] |
| 48 | 68 | 108 | 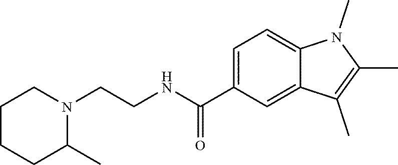 [SMILES: Cc1c(C)n(C)c2ccc(C(=O)NCCN3CCCCC3C)cc12] |
| 50 | 72 | 67 | 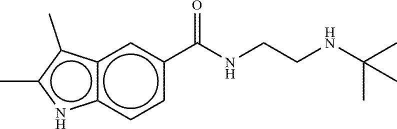 |
| 52 | 73 | 59 | 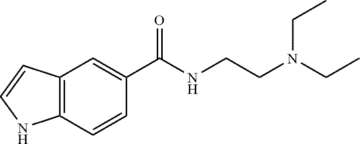 [SMILES: CCN(CC)CCNC(=O)c1ccc2[nH]ccc2c1] |
| 53 | 75 | 37 | 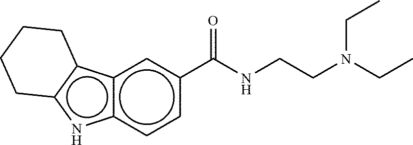 |
| 54 | 90 | 13 | 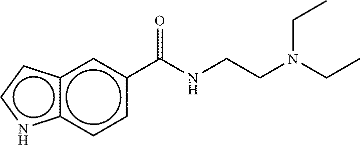 |
| 55 | 91 | 67 | |
| 56 | 109 | 27 | 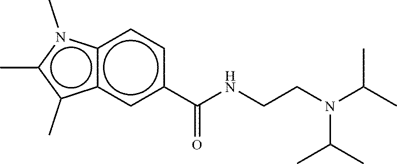 |

RESULTS - Table 7b summarizes the effect of the compounds on cell viability. STHdh 7/7 were heat shocked for 3 hours and treated for 48 hours with small molecule compounds. Table 2 lists the cell viability in striatal cells after 48 hours.

TABLE 7b

Effects of Compounds on cell viability of STHdh 7/7 striatal cells

| Compound Number | Cell Viability striatal cells 3h heat shocked | Chemical Structure |
|---|---|---|
| | 29.2 | No treatment |
| 21 | 82.6 | 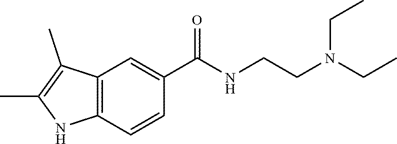 [SMILES: CCN(CC)CCNC(=O)c1ccc2[nH]c(C)c(C)c2c1] |
| 22 | 34.9 | 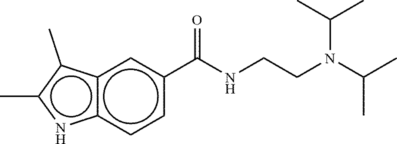 |
| 29 | 131.1 | 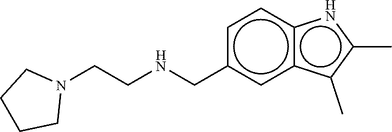 |
| 50 | 40.9 | 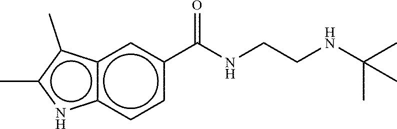 |
| 55 | 40.7 | |
| 56 | 64.1 | 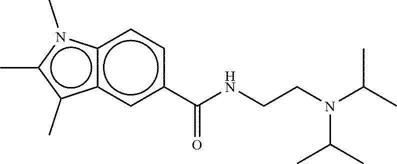 |
| 59 | 153.6 | 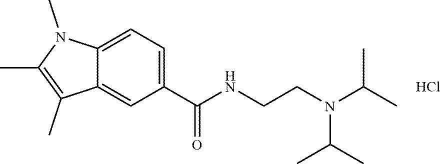 [SMILES: Cc1c(C)n(C)c2ccc(C(=O)NCCN(C(C)C)C(C)C)cc12 ; Cl] |
| 72 | 45.7 | 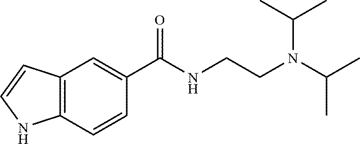 [SMILES: CC(C)N(CCNC(=O)c1ccc2[nH]ccc2c1)C(C)C] |

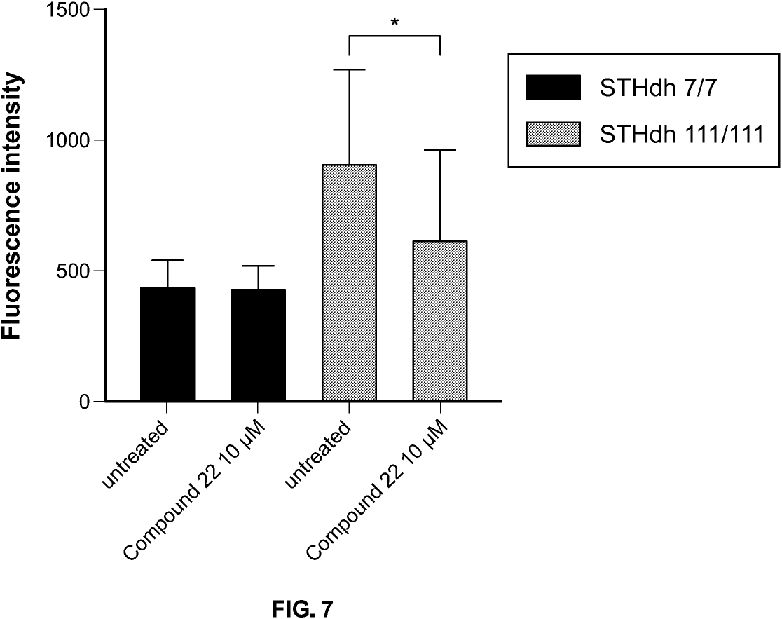

Effect of Compound 22 on Diffuse mHTT levels - ST HDH Q111/111 (CH00095, Coriell Institute) striatal derived cell line from a knock in transgenic mouse containing homozygous HTT loci with a humanized Exon 1 with 111 polyglutamine repeats. ST HDH Q7/7 (CH00097, Coriell Institute) striatal derived cell line from a knock in transgenic mouse containing HTT loci with a humanized Exon 1 containing 7 polyglutamine repeats. Nuclear diffuse mHTT was significantly reduced by 32% (p = 0.02) in STHdh 111/111 cells treated with Compound 22 in comparison to untreated STHdh 111/111 cells (FIG. 7). But wildtype huntingtin was not reduced in STHdh 7/7 cells treated with Compound 22, in comparison to non-treated STHdh 7/7.

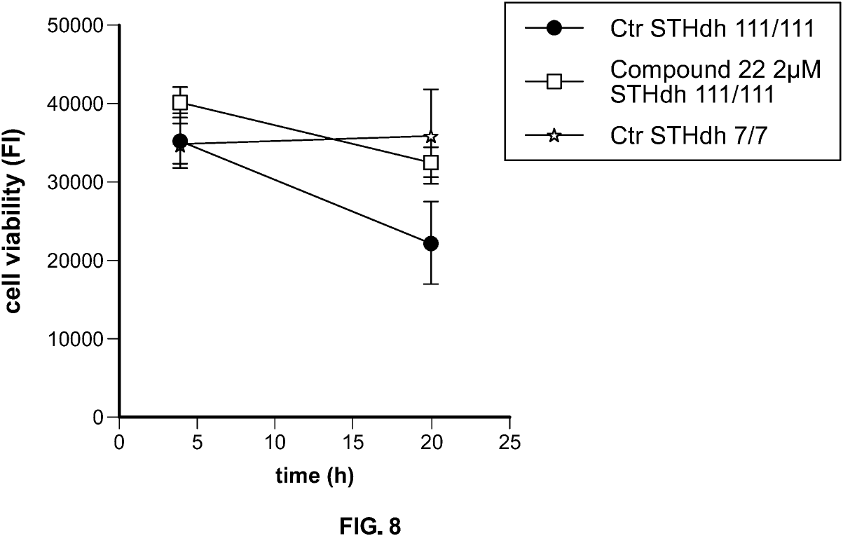

Effect of Compound 22 on Cell Viability - Heat shock triggers accumulation of misfolded proteins, which are degraded. At 24 hours after treatment, the cell viability of Compound 22 treated STHdh 111/111 cells was almost equivalent to the cell viability of STHdh 7/7 control cells with wildtype huntingtin (FIG. 8). The cell viability of the control STHdh 111/111 after 24 hours was lower than the cell viability of STHdh 111/111 cells treated with Compound 22.

Physicochemical properties of Compound 22 - Compound 22 is both soluble and it can cross the blood brain barrier. Significant concentrations can accumulate in the brain as indicated by comparison to the EC₅₀ value. The oral bioavailability in mice is 10%. This is due to the short microsomal stability in mice. In humans, the microsomal stability is 20 times higher in comparison to mice. Table 8 describes physicochemical properties of Compound 22.

TABLE 8

Brain Exposure and Physicochemical Properties of Compound 22

| | |
|---|---|
| | Compound 22 |
| Thermodynamic Solubility | 2.1 mM |
| Kinetic solubility | 188 µM |
| Log D | 1.21 (pH 7.4) |
| Brain exposure | Cₘₐₓ = 5.56 µM with i.p. administration and formulation |
| Blood Brain Barrier Penetrability | AUC_{brain}/AUCₚₗₐₛₘₐ = 2.24 |

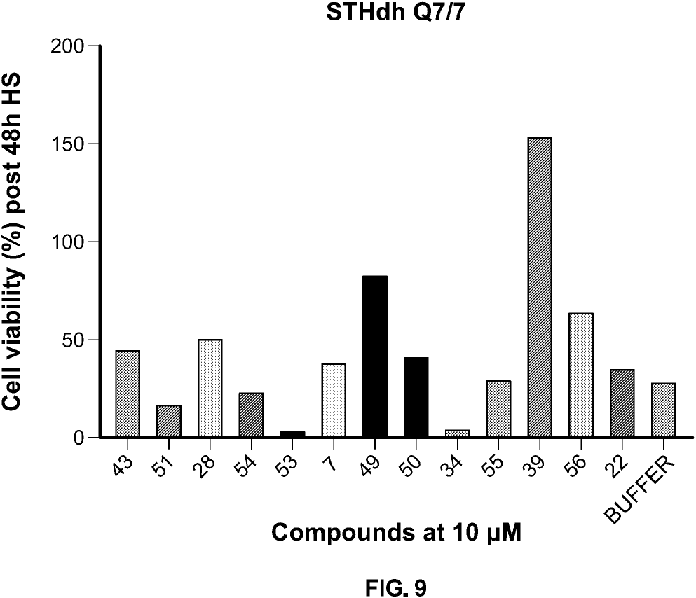

Effect of Compound 22 on Cell Viability of Wildtype Striatal Neurons - STHdh 7/7 cells are striatal cells that express wildtype huntingtin. These cells were cultured in the same conditions as described above for STHdh 111/111. The neurons were first heat shocked for 3 hours at 41° C. as described above. After 48 hours of treatment with the small molecule Compounds, cell viability was determined using the method described above. The “buffer” control is represents cells treated with a buffer only, without any compounds added. Some of the compounds show higher cell viability in comparison to control heat shocked STHdh 7/7 neurons with buffer and no treatment (FIG. 9). These compounds possess neuroprotective properties and protect striatal neurons from heat shock.

### Example 34 - Effects of Compound 22 on Motor Behavior in R6/2 Mice of Huntington’s Disease

### 44.1 Summary

Purpose - The objective of this study was to investigate the effects of Compound 22 on body weight and motor deficits in transgenic R6/2 mice of Huntington’s disease.

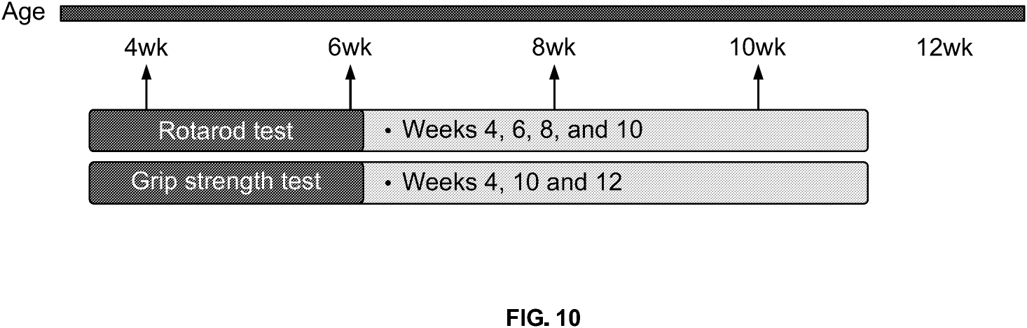

Methods - Total of 20 female and male R6/2 mice and 10 female and male wild-type littermate control mice (WT) were used in the study. The mice were genotyped and the R6/2 mice were divided into different treatment groups based on their litter and baseline body weight. The treatment with Vehicle or Compound 22(33 mg/kg; 5 ml/kg, i.p. QD) was started at 4 weeks of age after the baseline behavioral tests (FIG. 10). Body weights were measured at 3 weeks of age and twice a week until the end of the study. Motor function testing using rotarod were commenced at 4 weeks (pre-treatment baseline) and continued at 6, 8 and 10 weeks of age, accompanied with grip strength at 4 (pre-treatment baseline), 10 and 12 weeks of age. At the end point of 12 weeks of age the mice were subjected to tissue collection.

Results - Body Weight: There were no significant differences between the Compound 22and vehicle treated R6/2 mice. Rotarod: Within females R6/2 mice treated with Compound 22 showed significantly improved rotarod performance at 10 weeks of age compared to vehicle treated R6/2 mice. This effect was seen namely when the data was analyzed as normalizing the data of subsequent age points to that of the 4-week baseline data of each group. Grip Strength: There were no significant differences between the Compound 22 and vehicle treated R6/2 mice.

Conclusions - The current findings are well in line with previously reported results in studies produced at CRL Finland in terms of the phenotype progression of the R6/2 mice. Reviewing the data of the pooled genders in this study the genotype difference in body weight was significant starting from 10 weeks whereas in a previous study the body weight difference was significant from 9 weeks although the weekly averages were close to the same level (Beaumont, V. et al. Neuron. 2016 Dec 21;92(6):1220-1237). The rotarod latency of the R6/2 mice was significantly decreased from 6 weeks onwards both in the current study and in the previously reported one (Beaumont V et al., 2016). Also the grip strength data were close to similar in both studies at 12 weeks of age.

Regarding the efficacy of the Compound 22 treatment, the most notable finding in the current study was that the chronic treatment with Compound 22 (33 mg/kg, i.p., QD) significantly improved the rotarod performance of female R6/2 mice at 10 weeks of age, however showing no similar enhancement within males when comparing to vehicle treated R6/2 mice. Body weight loss was significantly improved in female R6/2 mice treated with Compound 22 at week 12 in comparison to untreated female R6/2 mice. This improvement was not observed in male R6/2 mice treated with Compound 22 at week 12 in comparison to untreated male R6/2 mice. Compound 22 treatment had no significant effects in grip.

### 44.2 Purpose of the Study

The objective of this study was to investigate the effects of Compound 22 on body weight and motor deficits in transgenic R6/2 mice of Huntington’s disease.

Total of 20 female and male R6/2 mice and 10 female and male wild-type littermate control mice (WT) were used in the study. The mice were genotyped and the R6/2 mice were divided into different treatment groups based on their litter and baseline body weight. The treatment with Vehicle or Compound 22 (33 mg/kg; 5 ml/kg, i.p. QD) was started at 4 weeks of age after the baseline behavioral tests. Body weights were measured at 3 weeks of age and twice a week until the end of the study. Motor function testing using rotarod were commenced at 4 weeks (pre-treatment baseline) and continued at 6, 8 and 10 weeks of age, accompanied with grip strength at 4 (pre-treatment baseline), 10 and 12 weeks of age. At the end point of 12 weeks of age the mice were subjected to tissue collection.

### 44.3 Materials and Methods

44.3.1 - Animals - All animal experiments were carried out according to the National Institute of Health (NIH) guidelines for the care and use of laboratory animals, and approved by the National Animal Experiment Board, Finland. The animal facility at site is accredited by the Association for Assessment and Accreditation of Laboratory Animal Care (AAALAC), International.

20 female and male R6/2 mice and 10 female and male wild-type littermate control mice were bred at Charles River, Germany. Mice derived from two consecutive rounds of breeding were randomly entered into the treatment plan below, using an equal number of males and females, and allocating them equally to the different treatment groups.

The experimental groups were:
Group 1) 10 wild-type mice (mixed gender) treated with Vehicle (5 ml/kg, i.p., QD) starting at 4 weeks of age
Group 2) 10 R6/2 mice (mixed gender) treated with Vehicle (5 ml/kg, i.p., QD) starting at 4 weeks of age
Group 3) 10 R6/2 mice (mixed gender) treated with Compound 22 (33 mg/kg; 5 ml/kg, i.p. QD) starting at 4 weeks of age

44.3.2 - Husbandry - All mice were housed in groups of up to 5 per cage, in a temperature (22±1° C.) and humidity (30-70%) controlled environment with a normal light-dark cycle (7:00-20:00 h light). All mice were housed in cages with clean bedding covering the ground that was changed as frequently as needed, at least once a week to provide the animals with dry bedding. This basic environment was enriched with the addition of a red mouse igloo (K3327), shredded paper and a wooden chewing stick. Food and water were available ad libitum to the mice in their home cages. The water spouts were fitted with extensions to allow mice to easily access from floor level. Each cage contained mice of only one gender and treatment group. In each cage was included also a wild-type mouse to provide normal social stimulation to R6/2 mice.

44.3.3 - Breeding and Weaning - 20 female and male R6/2 mice and 10 female and male wild-type littermate control mice (F₁ generation) were bred by Charles River Laboratories, Sulzfeld, Germany by mating (F₀ generation) WT males (C57BL/6J; systematically re-infused with pedigreed JAX mice, stock 000664) with ovarian transferred (OT) TG females (JAX, stock 006494). After weaning mice were sent from Germany to Charles River, Kuopio, Finland at an age of 3 weeks. Following genotyping and acclimation, the mice were enrolled in the study.

44.3.4 - Genotyping - Mice were ear marked at the age of 15-21 days and tail samples were collected at the same time for genotyping with PCR. Genotyping was performed at Charles River Discovery Services, Kuopio. DNA was isolated from tail or ear samples with Phire Animal Tissue Direct PCR-kit (Thermo Scientific, ref. F140WH) according to the kit’s instructions. Then 1 µl of DNA was multiplied in the PCR reaction with mouse specific (Gapdh) primers and human specific (Htt) primers.

Primers sequences and final working concentrations are listed below. After the PCR, multiplied DNA was separated by agarose gel electrophoresis. The expected products were 272bp (human specific product) and 372bp (mouse specific product). Thus wild-type (WT) mouse has one 372bp band while transgenic (TG) mouse has both the 272bp and 372bp bands.

Human specific 25 pmol/ µl, (5′-3′): TCATCAGCTTTTCCAGGGTCGCCAT (SEQ ID NO: 8)

Human specific 25 pmol/µl, (5′-3′): CGCAGGCTAGGGCTGTCAATCATGCT (SEQ ID NO: 9)

Mouse specific 5 pmol/µl, (5′-3′): ACTCCACTCACGGCAAATTCAACGGCAC (SEQ ID NO : 10)

Mouse specific 5 pmol/µl, (5′-3′): GGTCATGAGCCCTTCCACAATGCCAAAG (SEQ ID NO : 11)

Tail samples of all mice were taken at the end of the study for possible CAG repeat analysis.

44.3.5 - Plasma Sample Collection for Bile Acid Analysis - At 4 weeks of age blood samples for bile acid analysis were collected from saphenous vein into pre-cooled (ice bath) Li-Hep-tubes. The tubes were kept on ice and plasma was separated by centrifugation at 2000 g (+4° C.). About 50 µl of plasma from each mouse was aliquoted into pre-cooled polypropylene tubes and stored at -80° C. until analyzed. Plasma samples were analyzed using Thermofisher Konelab Xti 20 according to manufactures instructions. The mice having abnormally high bile acid levels in the plasma (more than 10 µmol /l) were removed from the study.

44.3.6 - General Health Status and Humane End-Points - Animals were monitored daily by laboratory personnel. The following humane end-points applied to all animals, unless otherwise mentioned in the experimental license granted by the National Ethics Committee. If the animal reached the humane end-points, it was euthanized.

The animals’ welfare was assessed by observing the following signs: general appearance (dehydration, weight loss, abnormal posture, condition of skin and fur, signs of pain); ambulation (reluctance or difficulties to move); behavior (apathy, abnormal behavior); clinical signs (eating, drinking, urinating, defecating). In addition, the mouse was euthanized if the mouse was not able to right itself within 20 sec when put on one side.

If there was a deviation from normal, the animal was closely monitored and treated, when possible (e.g. hydration, analgesia, warming). As a general rule, the animal was monitored no longer than 24-48 hours, after which the animal was euthanized, if its condition had not markedly improved.

44.3.7 - Compound Delivery and Dosing - Treatment with Compound 22 (33 mg/kg; 5 ml/kg, i.p. QD) or Vehicle was started at age week 4 and continued until the 12 weeks of age. The test articles were handled and stored and the dose formulations were prepared according to detailed instructions provided by the Sponsor.

44.3.8 - Body Weight - Body weight was measured starting at 3 weeks of age before treatment onset and two times per week on the same day (on Monday and Friday) until the end of the study.

44.3.9 - Motor Function and Cognition - The behavioral tests were conducted during the diurnal phase, between 8 a.m. - 5 p.m. The mice were transported to the behavioral test rooms from the animal housing rooms in their home cages. The mice were allowed to acclimate in the behavioral test room conditions at least for an hour before the tests. The behavioral tests were conducted under normal white light conditions.

44.3.9.1 - Rotarod - The Rotarod test was perfomed at 4 (pre-treatment baseline), 6, 8 and 10 weeks of age. Each testing day included a training trial of 5 min at 4 RPM on the Rotarod apparatus (AccuScan Instruments, Columbus, USA). 30 minutes later, the animals were tested for 3 consecutive accelerating trials of 6 min with the speed changing from 0 to 40 RPM over 360 seconds and with an inter-trial interval of at least 30 min. The latency to fall from the rod was recorded. Mice remaining on the rod for more than 360 s were removed and their time scored as 360 sec.

44.3.9.2 - Grip Strength - Mice were tested at 4 (pre-treatment baseline), 10 and 12 weeks of age. Mice were taken to the experimental room and, one at a time, were placed on the grip strength apparatus (San Diego Instruments, San Diego, USA) in such a way that the animal grabbed a small mesh grip with its forepaws. The entire apparatus was placed on a table top for testing. Animals were lowered to the platform and then slowly pulled away from the handle by the tail until the animal released the handle. The equipment automatically measures the strength of the animal’s grip in grams. Five scores were recorded per animal in consecutive sequence, and the average of three best scores for each animal was used for the results. Mice were returned to their home cage after testing.

44.3.10 - End-Point and Tissue Processing - Approximately one hour after the last dose the mice were terminally anesthetized with pentobarbital. A blood sample was collected via cardiac puncture in EDTA coated tubes on ice and plasma was separated by centrifugation (2000 g for 10 min). The plasma was aliquoted in two samples and fresh frozen on liquid nitrogen. Thereafter the mice were transcardially perfused with ice cold heparinized saline (Heparin 2.5 IU/ml) 25 ml)), followed by perfusion with ice cold 4 % PFA (80 ml). The brains were fixed by immersion in 4 % paraformaldehyde for minimum of 24 h after which brain samples were cryoprotected by 30 % sucrose solution for 72 h after which the brain samples were frozen in liquid nitrogen. Frozen brain specimens were stored at -80° C.

44.3.11 - Statistical Analysis - All values are presented as mean ± standard error of mean (SEM), and differences are considered to be statistically significant at the P<0.05 level. Statistical analyses were performed using GraphPad Prism statistical software. Differences among means were analyzed by using unpaired t-test.

### 44.4 Results

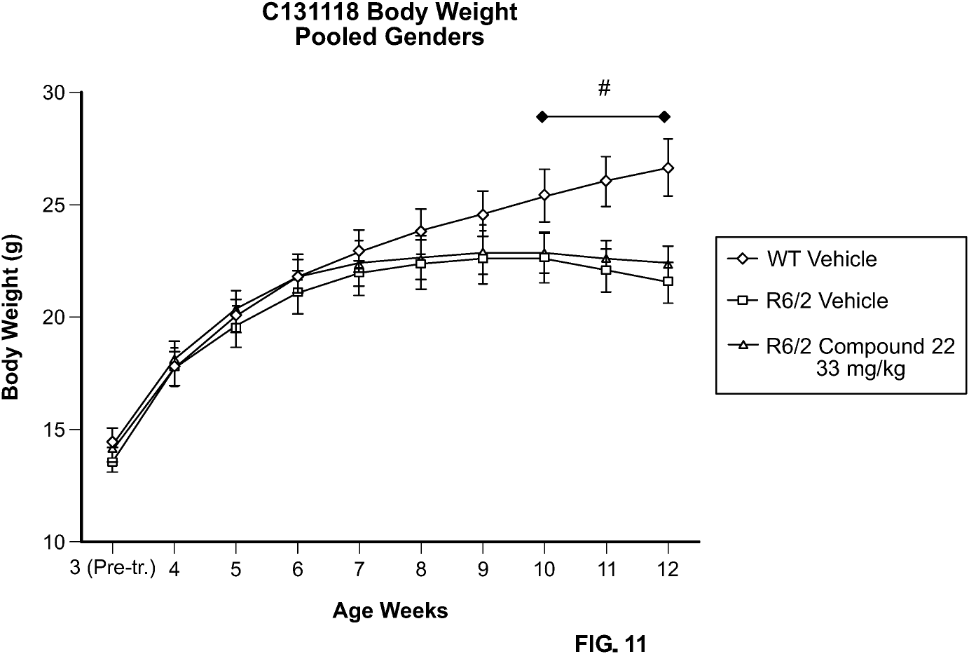

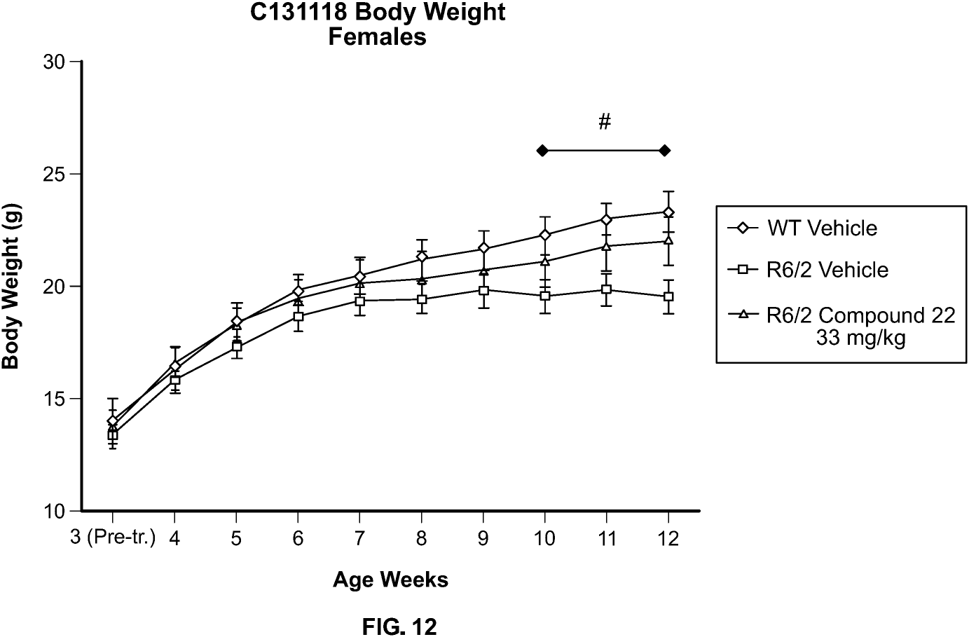

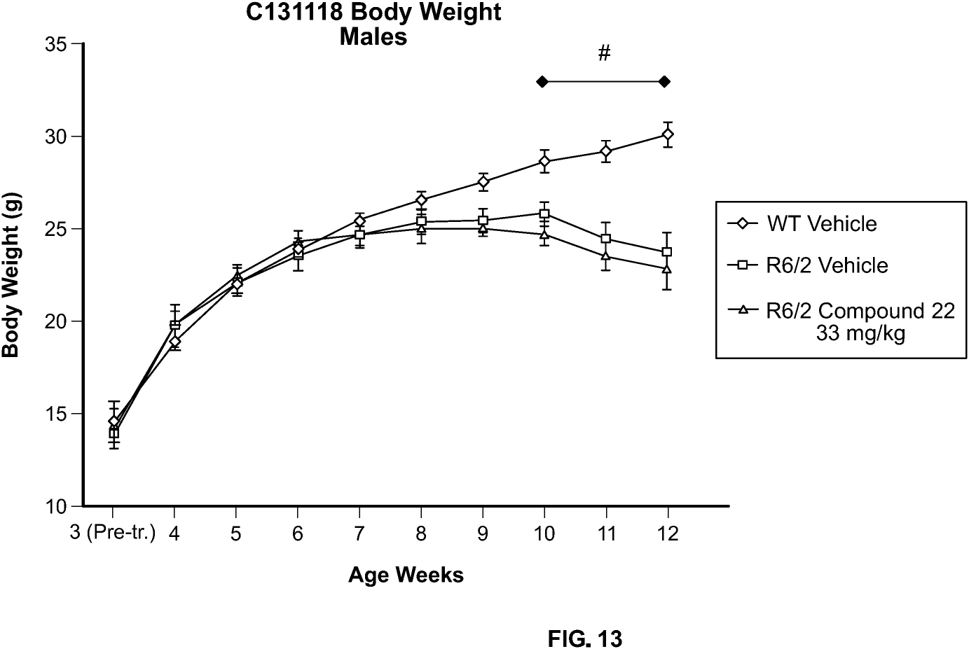

44.4.1 Body Weight - The effects of chronic administration of Compound 22 (33 mg/kg) on body weight of R6/2 mice from 3 to 12 weeks are presented in FIGS. 11-13. There were no significant differences between the vehicle and Compound 22 treated R6/2 mice in body weight (unpaired t-test, p > 0.05) (FIGS. 11-13). The vehicle treated R6/2 mice had decreased body weight compared to wild-type mice at 10-12 weeks of age within pooled genders and females, and at 9-12 weeks of age within males (unpaired t-test, # p < 0.05) (FIGS. 11-13).

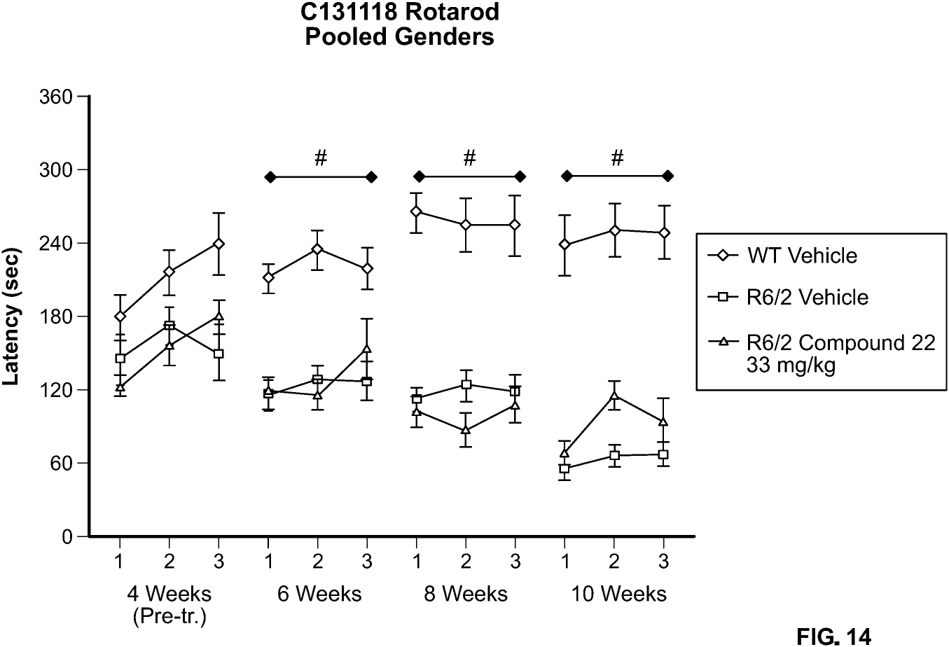

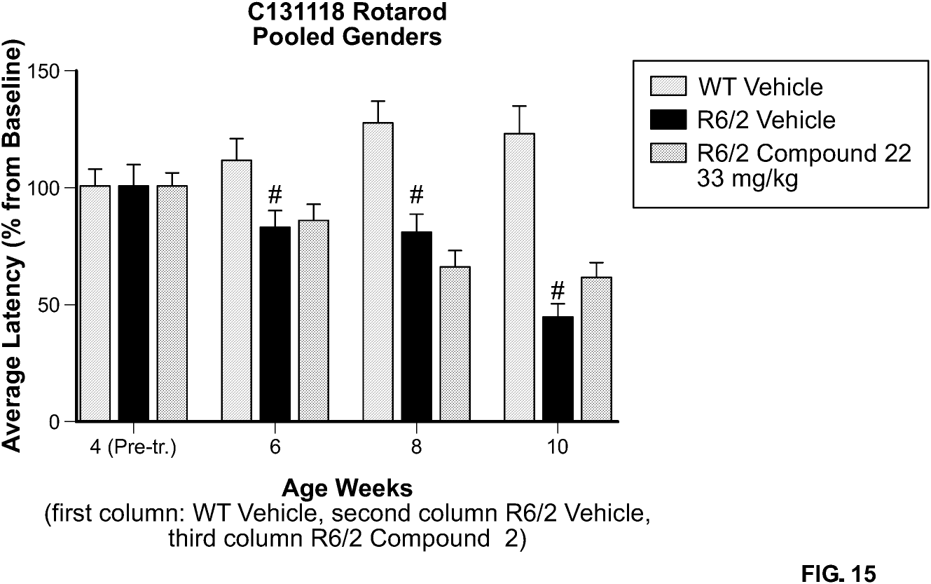

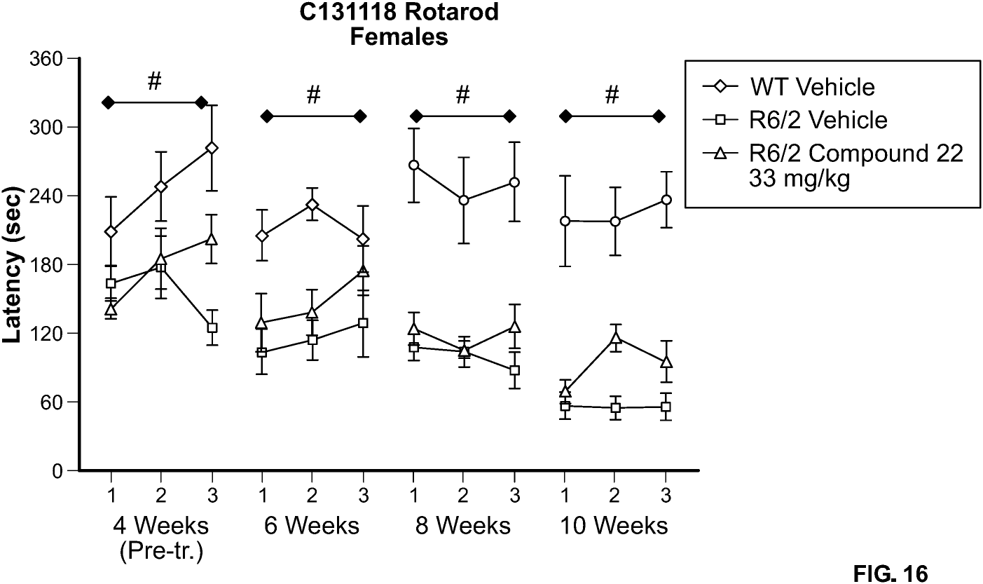

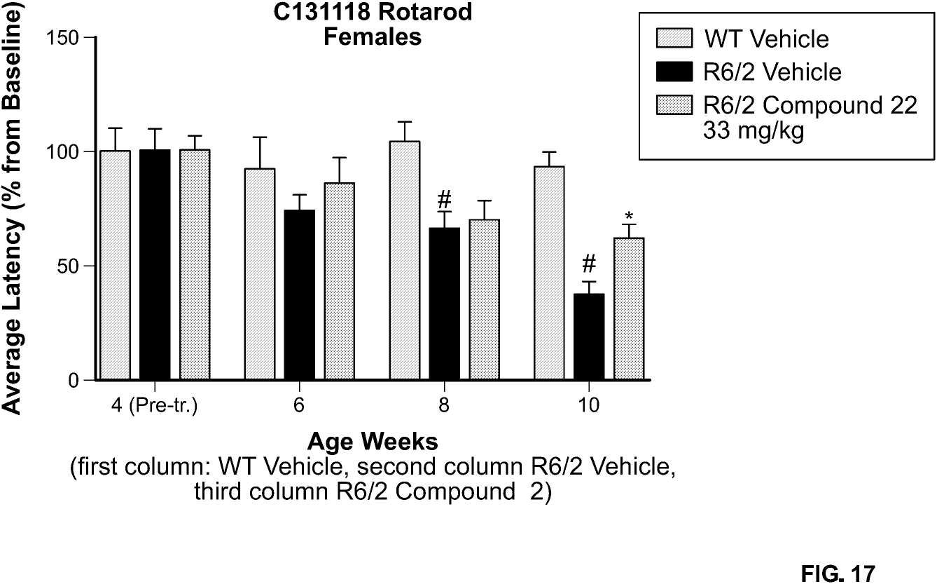

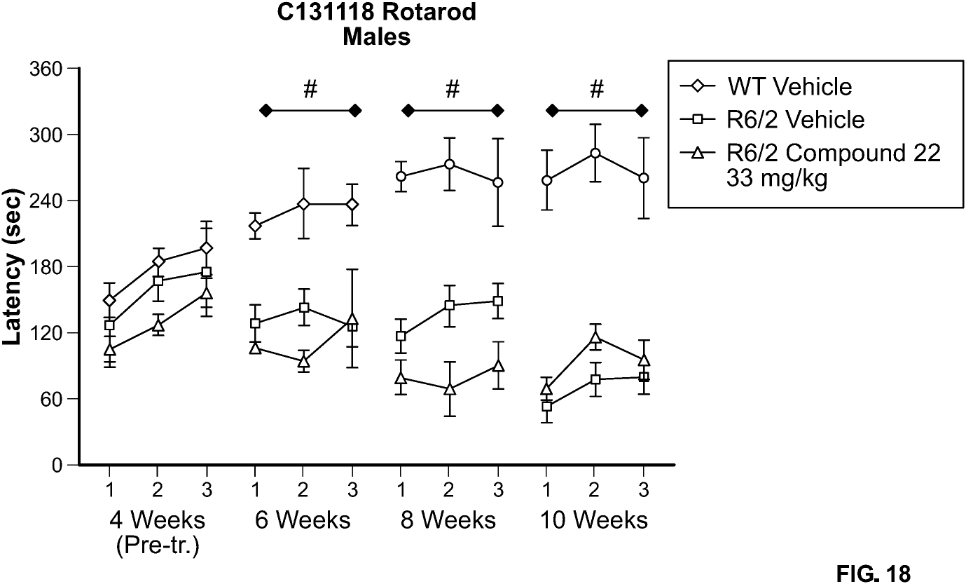

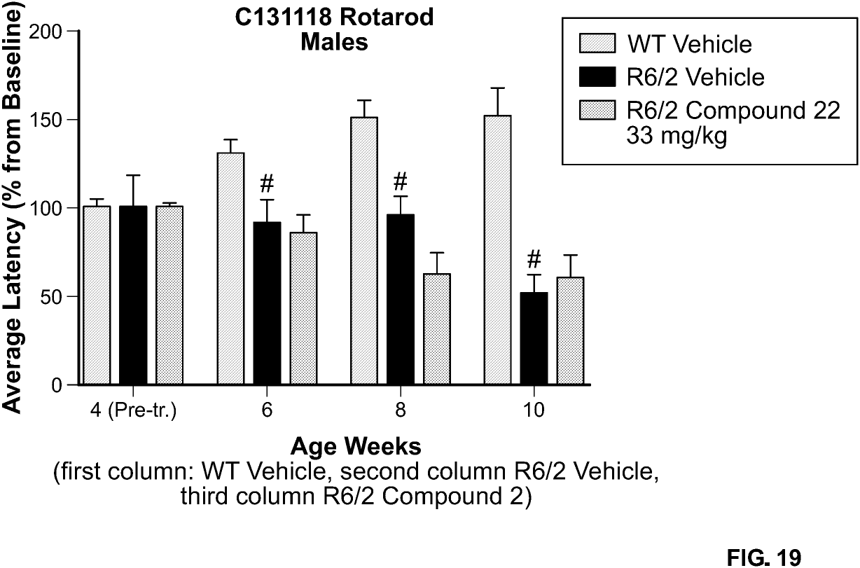

44.4.2 Rotarod - The effects of chronic administration of Compound 22 (33 mg/kg) on rotarod performance of R6/2 mice are presented in FIGS. 14-19. Within females R6/2 mice treated with Compound 22 showed significantly improved rotarod performance at 10 weeks of age compared to vehicle treated R6/2 mice (unpaired t-test, * p < 0.05) (FIG. 17). This effect was seen namely when the data was analyzed as normalizing the data of subsequent age points to that of the 4-week baseline data of each group. However, there were no significant differences between the Compound 22 and vehicle treated R6/2 mice within pooled genders or males (unpaired t-test, p > 0.05) (FIGS. 14-15 and 18-19). Vehicle treated R6/2 mice had decreased rotarod latency at 4-10 weeks within pooled genders and females, and at 6-10 weeks of age within males compared to wild-type mice (# p < 0.05, unpaired t-test) (FIGS. 14-19).

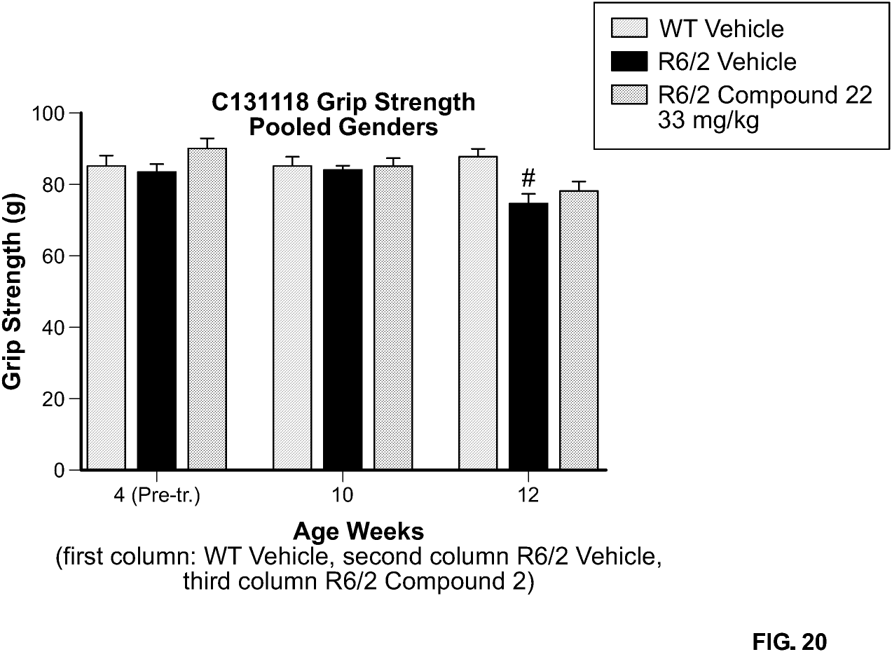

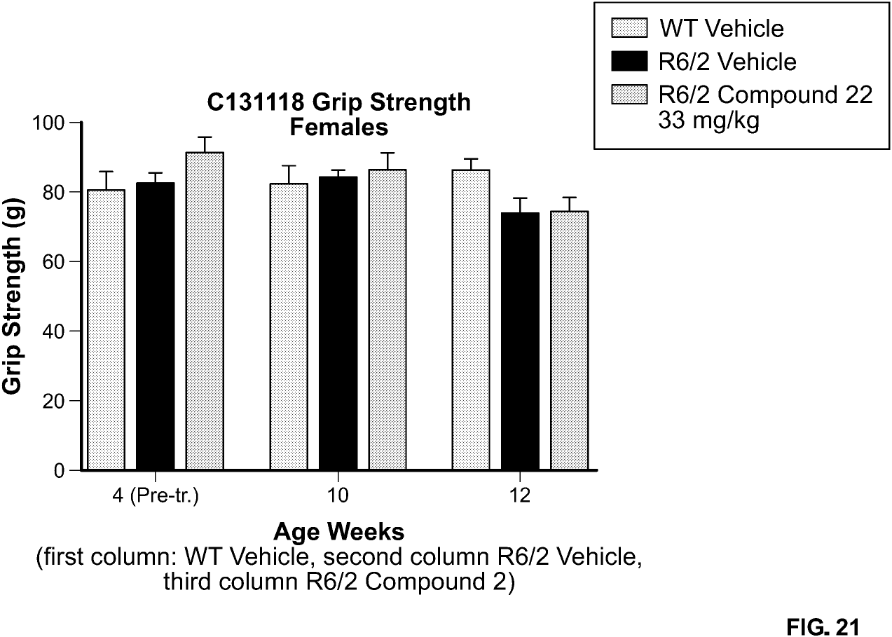

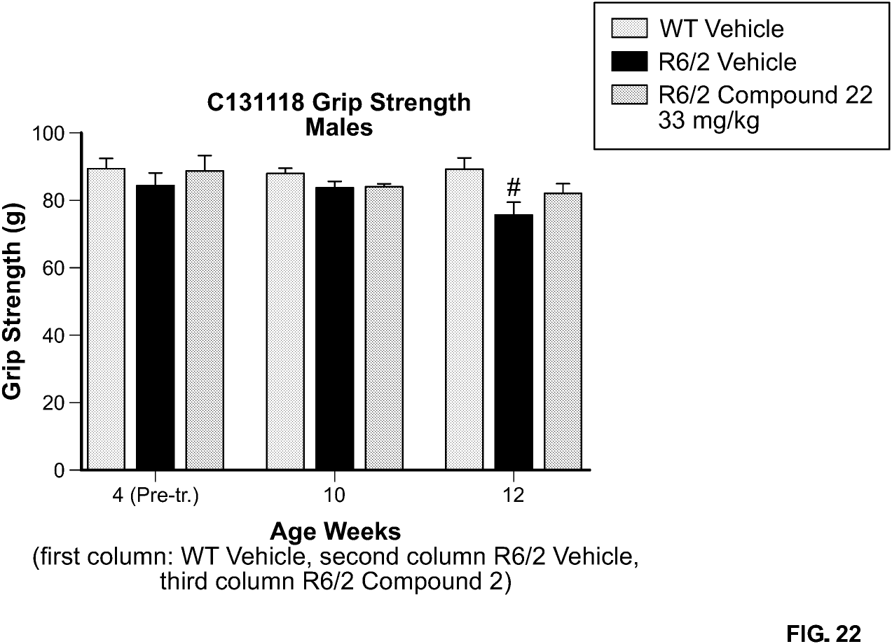

44.4.3 Grip Strength - The effects of chronic administration of Compound 22 (33 mg/kg) on grip strength of R6/2 mice are presented in FIGS. 17-19. There were no significant differences between the vehicle and Compound 22 treated R6/2 mice in grip strength (unpaired t-test, p > 0.05) (FIGS. 20-22). The vehicle treated R6/2 mice had lower grip strength at 12 weeks of age within pooled genders and males compared to wild-type mice (unpaired t-test, # p < 0.05) (FIGS. 20 and 22).

### 44.5 Conclusions

The current findings are well in line with previously reported results in studies produced at CRL Finland in terms of the phenotype progression of the R6/2 mice. Reviewing the data of the pooled genders in this study the genotype difference in body weight was significant starting from 10 weeks whereas in a previous study the body weight difference was significant from 9 weeks although the weekly averages were close to the same level (Beaumont, V. et al. Neuron. 2016 Dec 21;92(6):1220-1237). The rotarod latency of the R6/2 mice was significantly decreased from 6 weeks onwards both in the current study and in the previously reported one (Beaumont V et al., 2016). Also the grip strength data were close to similar in both studies at 12 weeks of age.

Regarding the efficacy of the Compound 22 treatment, the most notable finding in the current study was that the chronic treatment with Compound 22 (33 mg/kg, i.p., QD) significantly improved the rotarod performance of female R6/2 mice at 10 weeks of age, however showing no similar enhancement within males when comparing to vehicle treated R6/2 mice. Compound 22 treatment had no significant effects in body weight development or grip strength of the R6/2 mice.

### Example 35 - Immunohistochemistry Study With Tissue Isolated From R6/2 Study

Preparation of Immunohistology slides - Brain tissue collected within the animal study was prepared for IHC studies by Charles River staff. The brains were fixed by immersion in 4% paraformaldehyde for at least 24 h after which brain samples were cryoprotected by 30% sucrose solution for 72 h. Finally, the brain samples were flash-frozen in liquid nitrogen and stored at -80° C. The brain samples were cut using a microtome cryostat system, producing coronal brain tissue sections of 40 µm thickness. Those were mounted on individual adhesive-coated microscope glass slides with frosted ends.

Staining - Goat anti-Rabbit IgG (H+L) Alexa Fluor 647 (1:500, ThermoFisher Invitrogen) was used as a secondary antibody for binding to CBP antibody. DAPI (Sigma-Aldrich) was used to identify the nuclei. Goat anti-Mouse IgG (H+L), Alexa Fluor 488 (1:500, A-11034, ThermoFisher Invitrogen) was used as a secondary antibody for binding to EM48 antibody. Mouse anti-human-mHTT (EM48, 1:500, Sigma-Aldrich), was used to stain huntingtin. Rabbit anti-CBP (1:100, Sigma-Aldrich) was used a primary antibody to stain CBP.

The blocking buffer was freshly prepared and consisted of PBS (Sigma-Aldrich) with 5% normal goat serum (NGS), 0.2% BSA, 0.2% lysine and 0.2% glycine. Samples were covered with 750 µL of blocking buffer per sealing chamber and incubated at 4° C. for 24 hours. Subsequently, on day two samples were washed three times 10 min each in PBS, before working dilutions of primary antibodies were applied in 750 µL primary antibody buffer per chamber. The primary buffer consisted of PBS with 2% BSA/0.3% Triton X-100 (Sigma-Aldrich) and 0.02% NaN3 as preservative agent. The samples were incubated with primary antibodies at 4° C. for 73 hours. On day five, samples were washed like described previously and then incubated at 4° C. for 24 hours with secondary antibody in 750 µL secondary antibody buffer at 1:500 working dilutions per chamber. The secondary buffer consisted of PBS with 3% NGS/0.3% Triton X-100/0.02% NaN3. On day six, samples were washed and then incubated with DAPI containing mounting medium Fluoroshield (Sigma-Aldrich), in order to counterstain the nuclei and preserve the fluorescence. Therefore, one drop of mounting medium (Dako) was added per tissue section and the sample carefully coverslipped avoiding introduction of air bubbles. The samples were stored for 24 hours at room temperature shielded from light before being stored at 4° C. until imaging.

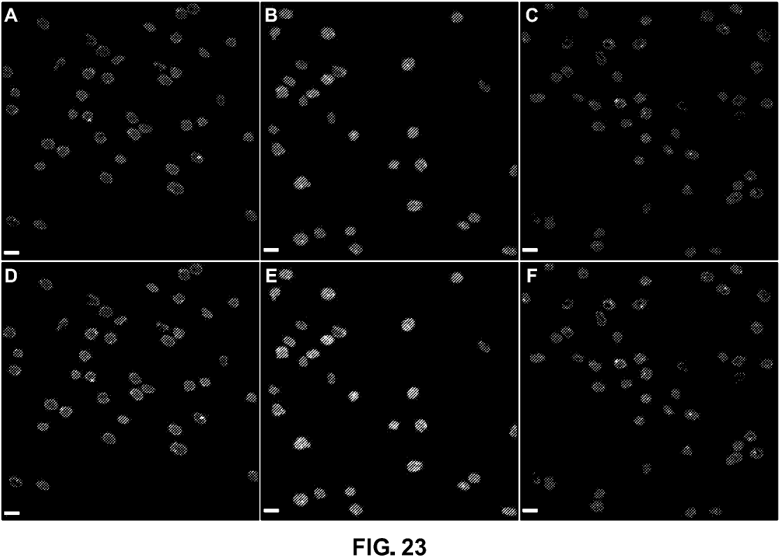

Results - FIGS. 23A-23F show the distinction of inclusion bodies (IB) and diffuse species of mHTT by confocal fluorescence imaging. FIGS. 23A-23F represent images taken of either the striatum or the cortex for the analysis of mHTT aggregation within nuclei of R6/2 mice. In the transgenic samples it can be seen that large IBs are clearly visible in a multitude of nuclei. Moreover, most IBs appear to be surrounded by diffuse protein, visibly as a hazy signal in the nucleus. Consequently, this distinction of diffuse mHTT and IBs was implemented in the analysis macro to quantify the different species of mHTT within striatum and cortex of R6/2 mice. To this end both an upper threshold of diffuse protein fluorescence signal and an adjacent lower threshold of IB fluorescence intensity were set and the images quantified appropriately.

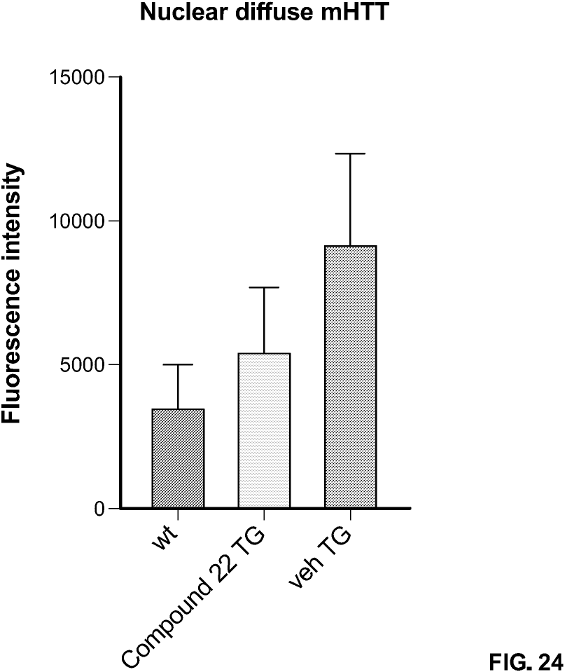

Diffuse mHTT was reduced in the cortex of animals treated with Compound 22. Diffuse mHTT consists of monomers and oligomers. Diffuse forms of mHTT are highly toxic in comparison to mHTT aggregates. Nuclear diffuse mHTT was reduced by 40% in treated animals (n= 9) in comparison to vehicle treated TG mice (p = 0.01) (FIG. 24). Compound 22 lowers mHTT in the cortex of R6/2 model mice.

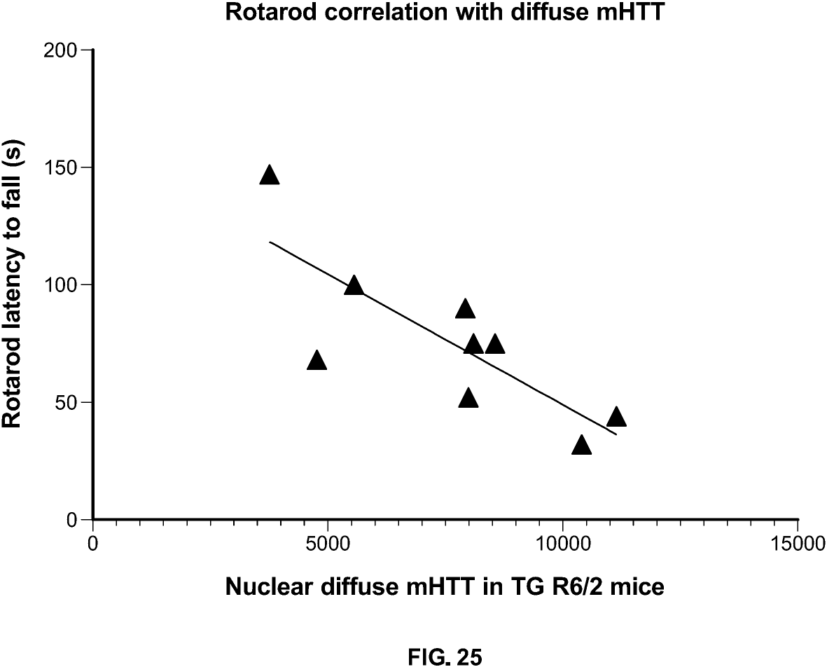

The reduction of diffuse mHTT correlates with motor symptoms. FIG. 25 shows that the motor symptoms strongly negatively correlate with diffuse mHTT in the nuclei of the cortex (Pearson r: -0.8, p= 0.01, n=9 (all transgenic female mice), confidence interval of r= -0.9561 to -0.2896 for nuclear diffuse mHTT). mHTT can be used as a biomarker for clinical efficacy as measured by motor symptoms. Therefore, lowering mHTT is a strategy to treat symptoms of Huntington’s disease.

### Example 36 - Compound 22 Improves Motor Symptoms in R6/2 Mice

10 female R6/2 mice and 10 female wild-type littermate control mice (F1 generation) were bred by Charles River Laboratories, Sulzfeld, Germany by mating (F0 generation) WT males (C57BL/6J; systematically re-infused with pedigreed JAX mice, stock 000664) with ovarian transferred (OT) TG females (JAX, stock 006494). After weaning mice were sent from Germany to Charles River, Kuopio, Finland at an age of 3 weeks. Following genotyping and acclimation, the mice were enrolled in the study.

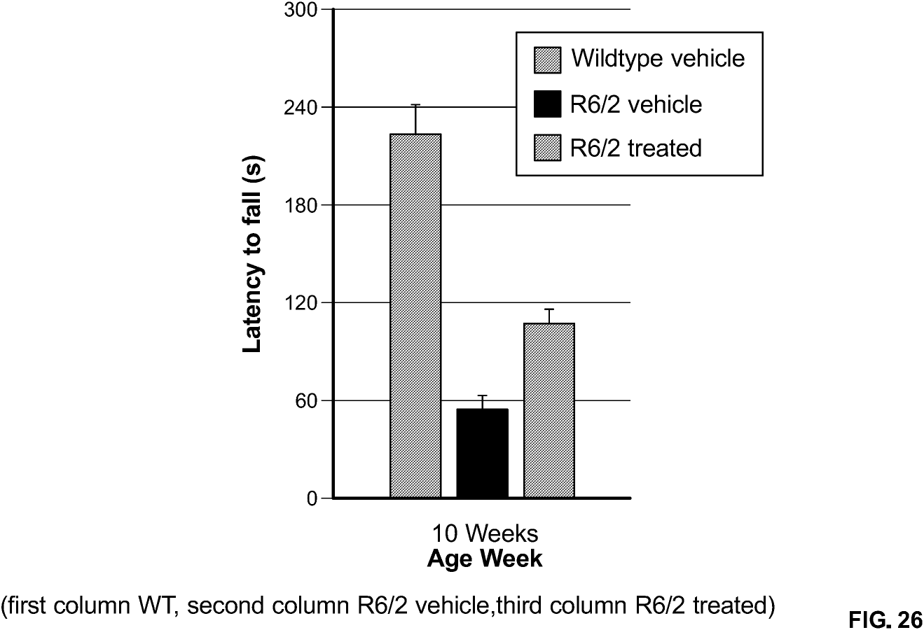

The treatment with Vehicle or Compound 22 (33 mg/kg; 5 ml/kg, intraperitoneal once daily was started at 4 weeks of age after the baseline behavioral tests. Motor function testing using rotarod were commenced at 4 weeks (pre-treatment baseline) and continued at 6, 8 and 10 weeks of age. R6/2 mice treated with Compound 22 showed significantly improved rotarod performance at 10 weeks of age compared to vehicle treated R6/2 mice. The mean latency to fall was 108.3 s ± 32.9 s in treated transgenic R6/2 mice and 55.7 s ± 19.1 s (p < 0.05) (FIG. 26). The R6/2 model is a very aggressive model and an improvement by 20% is regarded as a positive outcome. Compound 22 almost doubled the latency to fall in treated transgenic model mice indicating that Compound 22 improves motor symptoms in R6/2 mice.

### Example 37 - Co-Localization of CREB-Binding Protein (CBP) with mHTT and Quantification of CBP Abundance

CREB-binding protein (CBP) is a central coactivator of gene transcription. CBP serves as a molecular scaffold, facilitating interaction of CREB and other transcriptional regulators by enhancing the formers transcriptional activity toward cAMP-responsive genes. Additionally, it shows histone acetyltransferase (HAT) activity, thereby regulating gene transcription activity through epigenetic modifications, i.e. the modulation of histone acetylation status as well as of other transcriptional factors (Jiang, H. et al., Neurobiol Dis. 2006 Sep;23(3):543-551). In HD, the homeostatic functions of CBP are disrupted, since the mHTT shows aberrant interaction with CBP. CBP nuclear depletion analysis was performed based on scientific evidence, showing enhanced CBP degradation via the UPS pathway mediated by mHTT binding resulting in nuclear depletion of CBP (La Spada, A.R. et al., Nat Rev Genet. 2011 Apr;11(4):247-2589; Cong, S.Y. et al., Mol Cell Neurosci. 2005 Dec;30(4):560-571).

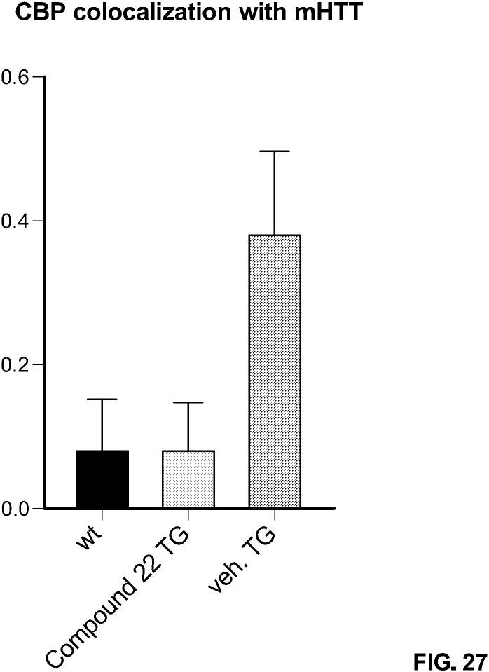

CBP colocalization with mHTT and quantitative assessment of the images were measured (Lee, J. et al., Acta Neuropathol. 2017 Nov;13(5):729-748; Steffan, J.S. et al., Nature. 2001 Oct 18;413(6857):739-743; Nucifora, F.C. et al., Science. 2001 Mar 23;291(5512):2423-2428). In brief, regions of interest (colocalized areas) in multi-channel images from two or three of the overlapping fluorescence dyes were selected using the Plugin CoLoc2 of FIJI. The colocalization score of identified areas was calculated. We used the FIJI plugin Coloc2 for measuring colocalization of CBP with diffuse mHTT. We detected reduced colocalization of CBP with mHTT in treated TG mice (FIG. 27).

### Example 38 - Circular Dichroism Study

### Protocol

Protein samples were diluted with buffer to 7.5 µM prior to Circular Dichroism (CD) measurements. Measurements were conducted at room temperature.

Four spectra at each measurement were taken every 1 nm from 200 to 260 nm, scanning at 50 nm/min with an averaging time of 1 sec. Spectra were obtained from samples in 20 mM phosphate, pH 7.4. Ten scans were averaged for each sample spectrum

Single-wavelength readings at 222 nm were obtained. After subtraction of the Trx contribution, the α-helical content of Httex1 was estimated from its mean residue ellipticity (MREHttex1) at 222 nm. This calculation was conducted as described by Bravo-Arredondo (Bravo-Arredondo, J.M. et al., J Biol Chem. 2018 Dec 21;293(51):19613-19623).

MRE Exon-1 Q16 and Q46 (Mean Residue Ellipticity) was measured every 1 sec for 300 sec; the 301 readings for each sample were averaged and MRE Trx subtracted. The number of amino acids in each sample to experience a change in helicity was estimated using a previously developed helix-coil transition model, which gave the change in fraction of helicity (Δf_{Helix}) by the following equation:

$${f}_{H e l i x} = \frac{M R {E}_{H t t e x 1} - M R {E}_{C o i l}}{M R {E}_{H e l i x} - M R {E}_{C o i l}}$$

where MRE Exon1 Q26 or Q46 (MRE_{Httex1} in the formula) describes the helix-coil transition, obtained from the difference between MRE Eon1 Q26 or Q46 at 222 nm of the fusion protein at the given temperature (in our case 37° C.) and the product of MRE Trx at 222 nm at the same temperature and the fraction of residues comprised by Trx in that particular construct, and

$$\begin{matrix}M R {E}_{C o i l} = 2220 - 53 T , M R {E}_{H e l i x} = \\ \left(- 44 , 000 + 250 T\right) \left(1 - \frac{3}{{N}_{r}}\right)\end{matrix}$$

given T as the temperature in °C and Nᵣ as the number of residues.

In order to calculate an equilibrium of the two states model, percent α-helicity was calculated using the relation of MRE_{Httex1} = 0 and -34700 (deg cm² dmol⁻¹) for 0% and 100% helicity, respectively.

TABLE 9

Comparison between Kd, reversal of conformational changes of Exon1 Q46 and reduction of mHTT in comparison to untreated striatal neurons

| Compound ID | Kd (µM) | Reduction of the proportion of a predominantly α-helical state to wildtype levels with 2 µM (Compound 22) or 1.6 µM (Compound 4 and Compound 101) compound exposure | mHTT after 3 h heat shock and following 48 hours of compound treatment in comparison to untreated STHdh 111/111 |
|---|---|---|---|
| Compound 4 | 0.06 | Complete reversal | 34% |
| Compound 101 | 0.07 | 59.3% | 38% |
| Compound 22 | 0.7 | 36.6% | 46% |

### Compound 22

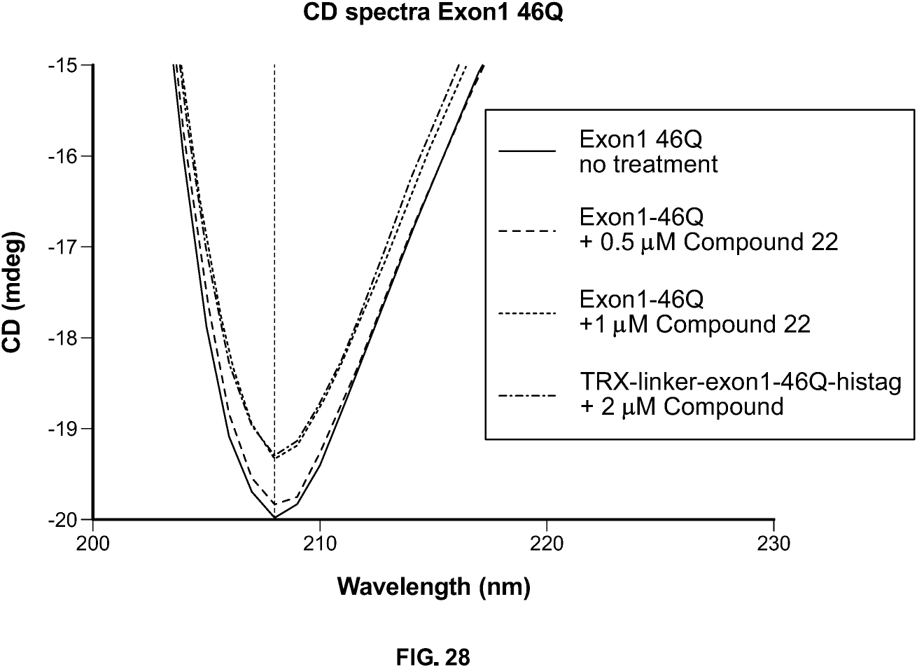

In this CD measurement the predominately α-helical state of Exon1 Q46 mHTT was 23.6% and thus 6% higher than the 17.6% proportion of predominantly α-helical state of Exon1 Q16 mHTT. Treatment with 2 µM Compound 22 reduced the proportion of a predominantly α-helical state to 21.4% of Exon1 Q46 and thus reduced the proportion of a predominantly α-helical state by 36.7% in comparison to short Q length Exon1 (FIG. 28).

### Compound 4

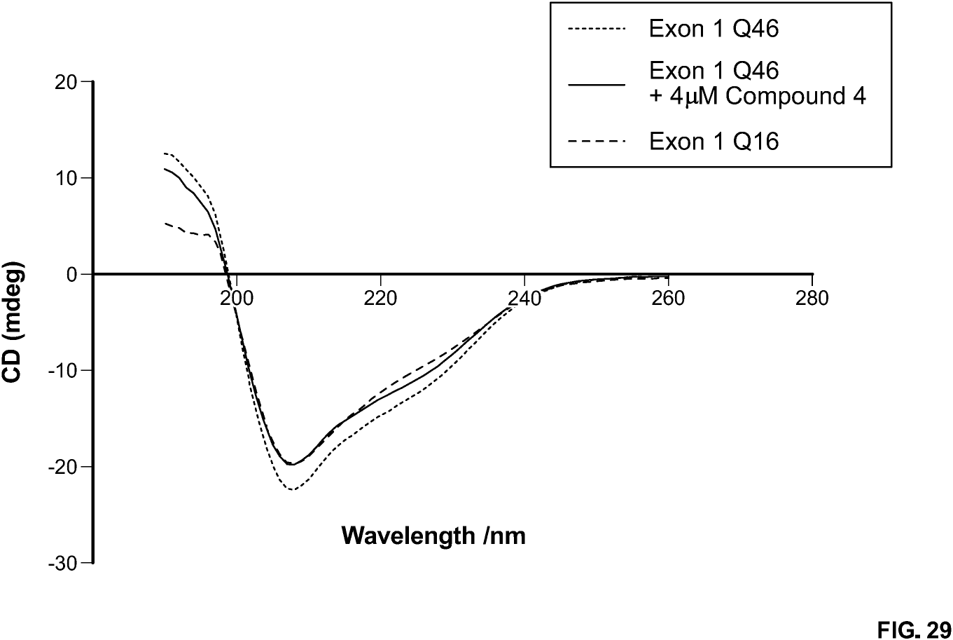

Compound 4 was used for CD measurements. Compound 4 exhibited a K_{d} of 60 nM to Exon1 Q46 protein. Compound 22 exhibited a K_{d} of 701 nM. Thus, Compound 4 more efficiently reversed the conformational change in mutated Exon1 Q46 than Compound 22 (Table 9). In this CD measurement the predominately α-helical state of Exon1 Q46 mHTT was 23.2% and thus 3.4% higher than the 19.9% proportion of predominantly α-helical state of Exon1 Q16 mHTT (Table 10). Treatment with 1.6 µM Compound 4 reduced in Exon1 Q46 the proportion of a predominantly α-helical state to 19.5% and thus reversed the proportion of a predominantly α-helical state completely back to short Q length Exon1 (FIG. 29).

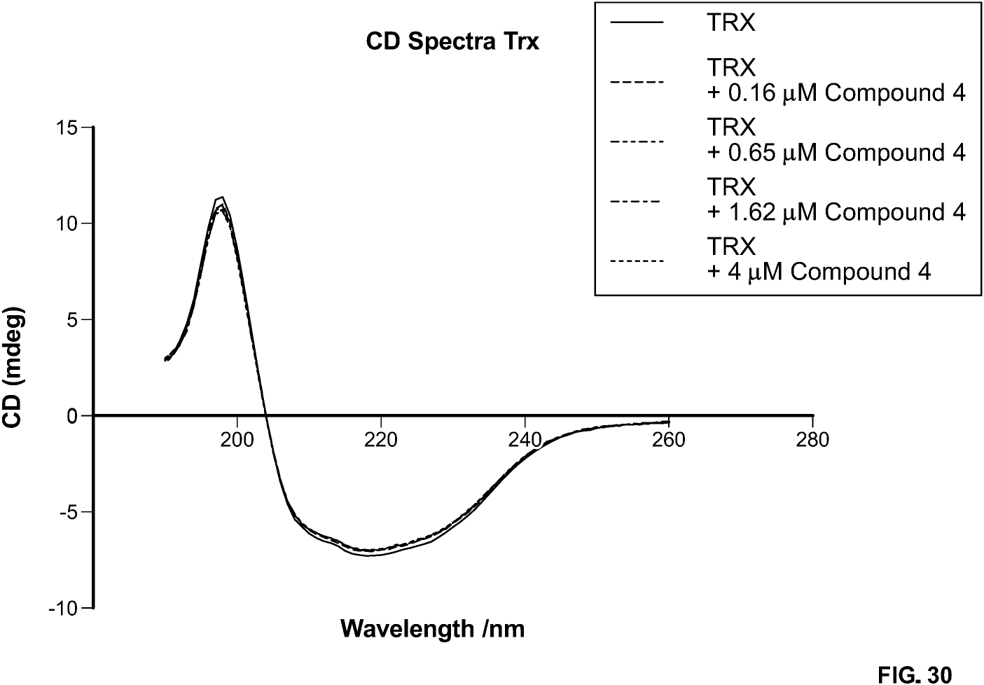

Thioredoxin (Trx) was used as a control to demonstrate that exposure to the compounds have no impact on the α-helix structure of proteins which are not mutated huntingtin (FIG. 30). Trx contains 31% α-helix.

TABLE 10

Percentage of Predominantly α-Helical Status of Exon1 Q46 Exposed to Different Doses of Compound 4.

| conc. Compound (µM) | Fraction Helix fH (%) |
|---|---|
| 0 | 23.2 |
| 0.01 | 22.4 |
| 0.059 | 20.9 |
| 0.158 | 21.1 |
| 0.647 | 20.0 |
| 1.62 | 19.5 |
| 4.04 | 19.3 |
| 13.68 | 18.3 |
| 37.77 | 18.1 |

### Compound 101

The CD measurements protocol for Compound 101 was the same as outlined above except that from 206 nm - 210 nm and 220 nm - 224 nm, the step size was 0.2 nm, and the bandwidth was 1 nm.

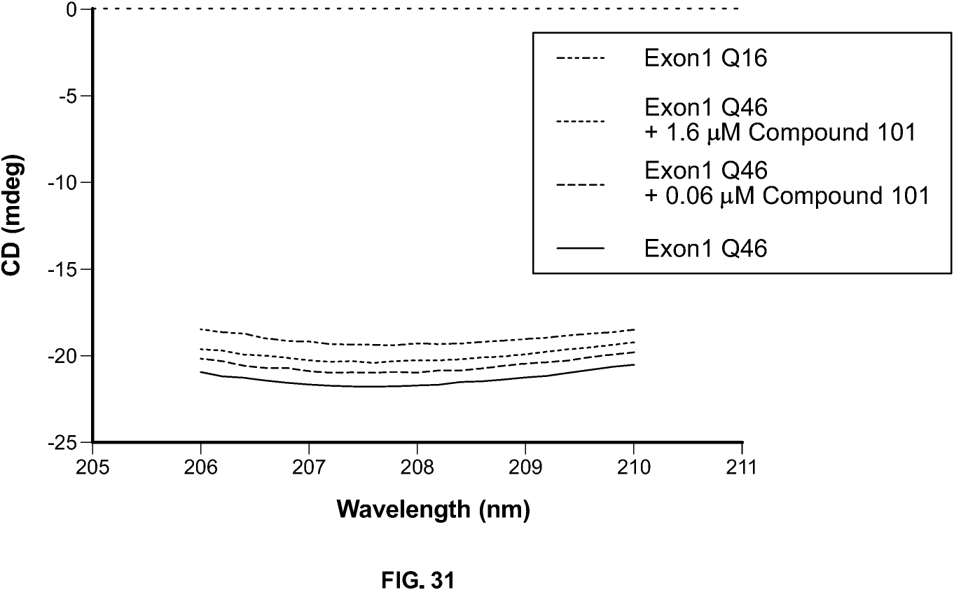

The long Q length (Q46) Exon1 increase the proportion of a predominantly α-helical state about 2.3% from 18.1% in short Q length Exon1 to 20.4% in long length Exon1 Q46. Treatment with 1.6 µM Compound 101 reduced the proportion of a predominantly α-helical state to 19.0% of Exon1 Q46 and thus reduced the proportion of a predominantly α-helical state by 59.3% in comparison to short Q length Exon1 (FIG. 31 and Table 11).

TABLE 11

Percentage of Predominately α-helical Status of Exon1 Q46 Exposed to Different Doses of Compound 101

| conc. Compound (µM) | Fraction Helix fH (%) |
|---|---|
| 0 | 20.4 |
| 0.01 | 20.5 |
| 0.059 | 20.1 |
| 0.158 | 19.9 |
| 0.647 | 19.1 |
| 1.62 | 19.0 |
| 4.04 | 18.5 |
| 13.68 | 18.7 |
| 37.77 | 18.2 |

### Example 39 - Autophagy Flux Increase Is Responsible for mHTT Reduction in Compound 22 Treated Neurons

Cell culture - ST HDH Q111/111 (CH00095, Coriell Institute) striatal derived cell line were grown at 33° C. in DMEM (Sigma-Aldrich), supplemented with 10% fetal bovine serum (FBS), 1% Penicillin-Streptomycin (ThermoFisher Scientific), and 0.4 mg/ml G418 (Geneticin; Invitrogen).

Pre-Treatment of striatal cell line - First the medium was removed and new DMEM (DMEM, high glucose, HEPES, no phenol red) medium, supplemented with 10% fetal bovine serum (FBS), 1% Penicillin-Streptomycin, containing 10 µM compound was added. The cells were incubated at 33° C. for 24 hours.

Heat shock - Cells were heat shocked for 3 hours at 41° C.

Treatment - Medium was removed and new DMEM (DMEM, high glucose, HEPES, no phenol red) medium, supplemented with 1% fetal bovine serum (FBS), 1% Penicillin-Streptomycin, containing no compound, or 10 µM Compound 22, or 10 mM NH4Cl (Sigma Aldrich), or 125 nM MG132 (Sigma Aldrich), or 10 µM Compound 22 with 10 mM NH4Cl, or 10 µM Compound 22 with 125 nM MG132 was added. The cells were incubated at 33° C. for 48 hours.

Cell Viability - Cell viability was measured according to manufacturer recommendations with CellTiter Glo (Promega). 96-well plates for used either for cell viability determination or cell staining.

Staining - Cells were permeabilized with 0.3% Triton X-100 in 4° C. PBS (pH 7.4 from ThermoFisher) for 10 minutes. Then the cells were washed two times with 4° C. PBS. Subsequently, cells are blocked with 3% NGS (normal goat serum from Thermo Fisher) in 4° C. PBS for 1 hour. Cells were incubated with primary antibodies in blocking buffer (PBS, 3% NGS, 0.02% NaN3) over night at 4° C. Primary antibodies were mouse antipolyglutamine monoclonal antibody 3B5H10 (Sigma-Aldrich) at 1:250 dilution and Guinea Pig anti-MAP2 polyclonal antibody (Synaptic Systems) at 1:500 dilution. For LC3 staining primary antibodies were mouse anti-LC3 mab (5F10) (nanoTools) at 1:500 dilution and Guinea Pig anti-MAP2 polyclonal antibody (Synaptic Systems) at 1:500 dilution.

Cells were washed 2 times with 4° C. PBS, and then incubated with secondary antibodies at 1:2000 dilution in blocking buffer for 1 hour at room temperature. Secondary antibodies were Goat anti-Guinea Pig IgG (H+L) Highly Cross-Adsorbed Secondary Antibody, Alexa Fluor 647 (ThermoFisher Invitrogen) and Goat anti-Mouse IgG (H+L) Cross-Adsorbed Secondary Antibody, Alexa Fluor 488 (ThermoFisher Invitrogen). Cells were washed 2 times with 4° C. PBS, and then the nuclei were counterstain with DAPI (ThermoFisher) for 10 min at room temperature in the dark. Cells were washed 2 times with 4° C. PBS, and then 4° C. PBS was added to cells and then images were acquired.

Fluorescence Imaging - Imaging sessions were performed on a confocal microscope system (Carl Zeiss Microscopy) equipped with a dual spinning disk unit (Yokogawa). All components of the imaging system were controlled via the ZEN 2 software suite (Carl Zeiss Microscopy). The laser lines used were 405 nm, 488 nm and 639 nm to excite DAPI or the respective fluorophores. The fluorescence images obtained of the immunofluorescence labelled tissue sections were quantified with the help of the “Image Processing and Analysis in Java” or short ImageJ software distributed under the GNU General Public License by the NIH (Rueden, C.T. et al., BMC Bioinformatics. 2017 Nov 29;18(1):529), i.e. the edition used was the Fiji distribution (Schindelin, J. et al., Nature methods. 2012 Jun 28;9(7):676-82). Nested t test was performed to calculate the significance amongst repeated measurements using GraphPad Prism software.

RESULTS - Compound 22 improves autophagic flux - An increased level of LC3-II or an accumulation of GFP-LC3 puncta is not always indicative of autophagy induction and may represent a blockade in autophagosome maturation (Fass, E. et al., J Biol Chem. 2006 Nov 24;281(47):36303-16). Autophagic flux is generally defined as a measure of the autophagic system’s degradation activity (Klionsky, D.J. et al., Autophagy. 2012 Nov:7(11):1273-94). If autophagic flux is occurring, the level of LC3-II will be increased in the presence of a lysosomal degradation inhibitor because the transit of LC3-II through the autophagic pathway will be blocked (Tanida, I. et al., Autophagy. 2005 Jun;273(11):2553-62).

Autophagic flux was calculated as the area stained with LC3 antibody per cell in STHdh 111/111 with autophagy inhibitor NH4Cl minus area stained with LC3 antibody per cell in STHdh 111/111 without the autophagy inhibitor NH4Cl.

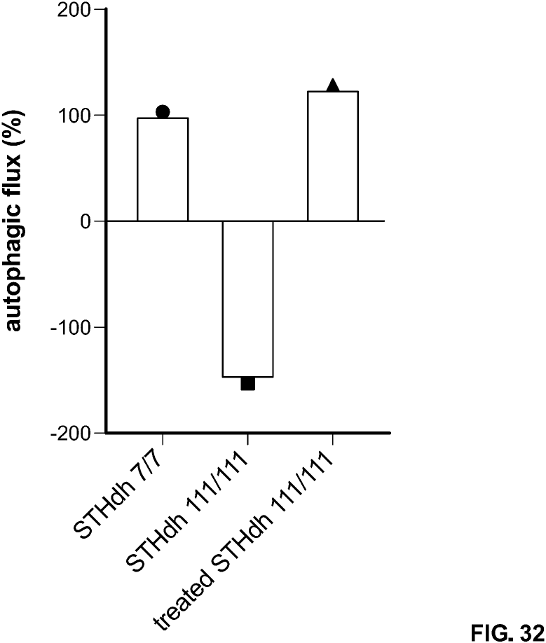

The Compound 22 treated STHdh 111/111 cells have a positive autophagic flux and the untreated STHdh Q111/111 cells have a negative autophagic flux (FIG. 32).

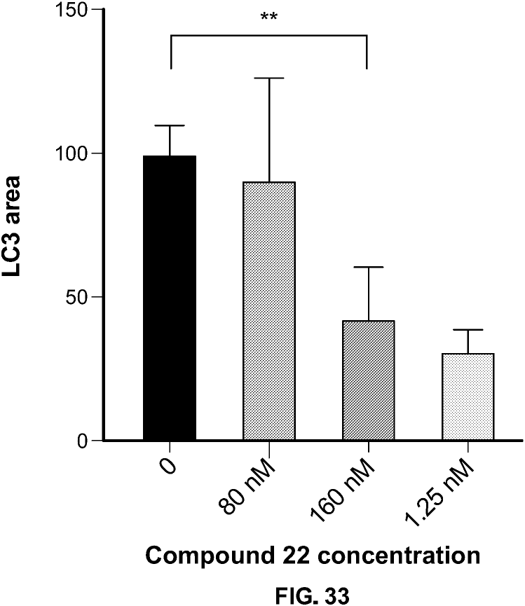

This explains that in striatal neurons treated with Compound 22 LC3 stained area in STHdh 111/111 neurons is decreased and in untreated neurons LC3 is increased (p < 0.001) (FIG. 33). Several research groups observed that the autophagosome marker LC3 expression is increased in STHdh Q111/111 neurons expressing mutated huntingtin compared to wild type (WT) controls (Walter, C. et al., Neuropharmacology. 2016 Sep;108:24-38).

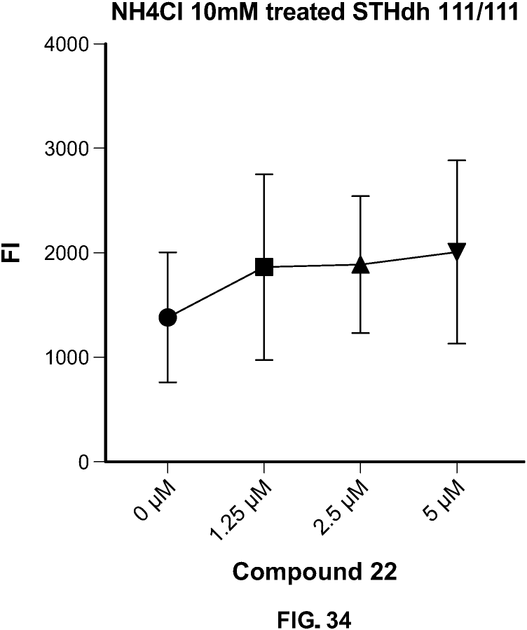

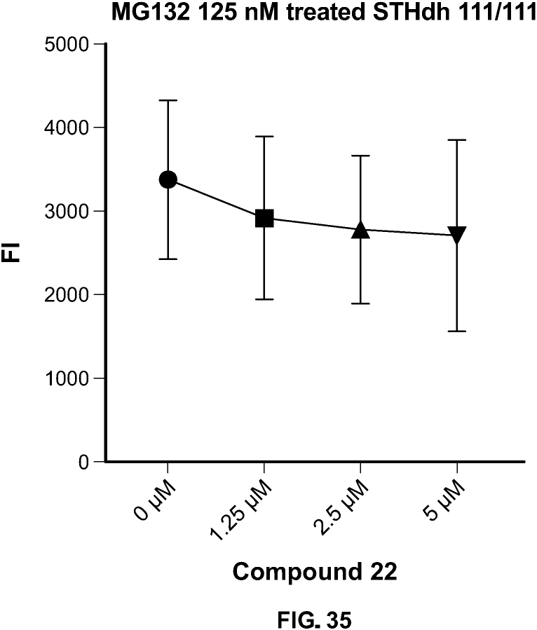

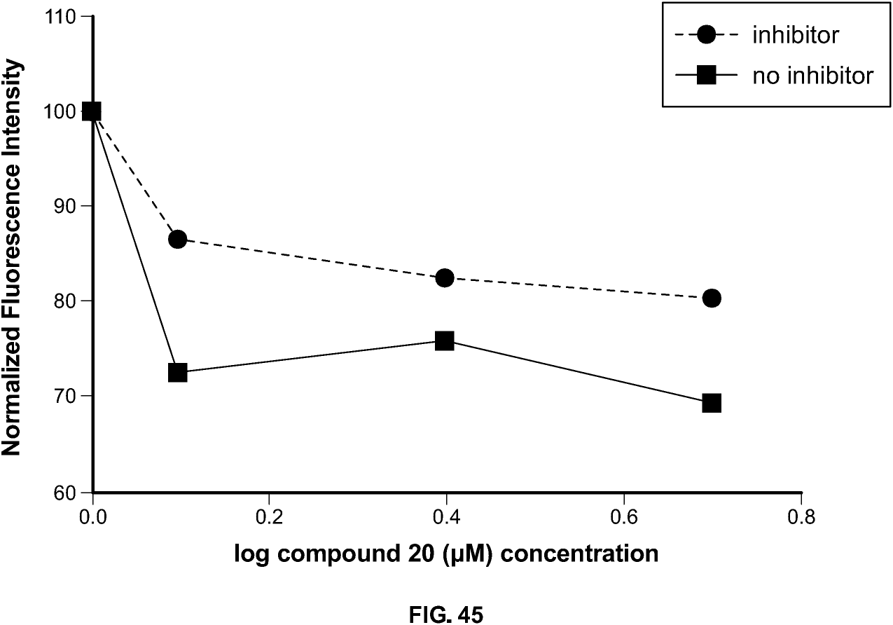

Autophagosomal degradation is responsible for mHTTreduction in neurons treated with Compound 22 - Striatal neurons STHdh 111/111 which express mHTT were exposed to 10 mM of the autophagy inhibitor NH4Cl. The autophagy inhibitor NH4Cl blocked the mHTT lowering effects of Compound 22 completely (FIG. 34). The ubiquitin proteasome system (UPS) is one of the main pathways for the degradation of misfolded proteins. Compound 22 could reduce mHTT in STHdh 111/111 cells which were exposed to the UPS inhibitor MG132 (FIG. 35). But the mHTT reduction in Compound 20 treated striatal cells exposed to MG132 is less in comparison striatal cells only treated with Compound 20 without exposure to MG132. This observation indicates that the mHTT reduction in Compound 20 treated cells is in part due to the UPS (FIG. 45).

Example 40 - Effects of Compound 22 on Gene Expression

Gene expression of certain transcripts of interest were measured using Nanostring Technology. STHdh 7/7 and STHdh 111/111 cells were used. Some STHdh 7/7 and STHdh 111/111 cell samples were not heat shocked and some wells were treated with Compound 22 at the indicated concentration on for 48 hours (Table 12). Some STHdh 7/7 and STHdh 111/111 cell samples were heat shocked for 3 hours some wells were treated with Compound 22 at the indicated concentration on for 48 hours (Table 13). Cells are washed with PBS, trypsinised, and gathered in a 15 ml falcon tube. After a short spin down for 3 minutes at 300 g, the cell pellet was washed with PBS once, before it was frozen down in liquid nitrogen. RNA was extracted with a standard protocol. The nCounter® Mouse Neuropathology Panel (Nanostring, Seattle) was used for gene expression analysis according to the manufacturer’s protocols.

TABLE 12

Cell samples that were not subject to heat shock treatment

| SLOT | Cell Line | Compound 22 (µM) | Treatment time |
|---|---|---|---|
| 1 | STHdh7/7 | 0 | 48 h |
| 2 | STHdh7/7 | 10 | 48 h |
| 3 | STHdh7/7 | 20 | 48 h |
| 4 | STHdh111/111 | 0 | 48 h |
| 5 | STHdh111/111 | 10 | 48 h |
| 6 | STHdh111/111 | 20 | 48 h |

TABLE 13

Cell samples subject to heat shock treatment

| SLOT | Cell Line | Compound 22 (µM) | Time | Heat Shock |
|---|---|---|---|---|
| 1 | STHdh7/7 | 0 | 48 h | 42° C. / 3 h |
| 2 | STHdh7/7 | 10 | 48 h | 42° C. / 3 h |
| 3 | STHdh7/7 | 20 | 48 h | 42° C. / 3 h |
| 4 | STHdh111/111 | 0 | 48 h | 42° C. / 3 h |
| 5 | STHdh111/111 | 10 | 48 h | 42° C. / 3 h |
| 6 | STHdh111/111 | 20 | 48 h | 42° C. / 3 h |

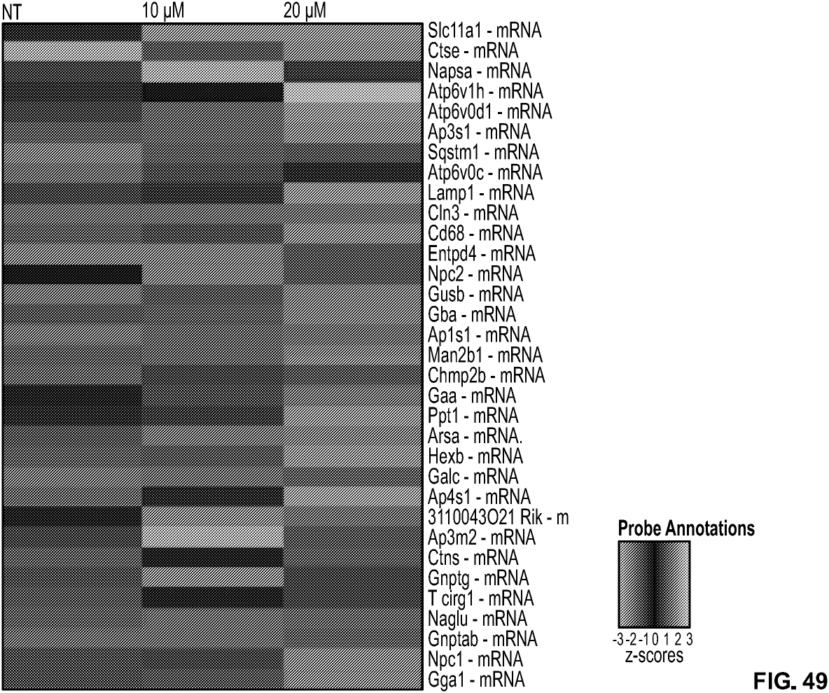

Genes associated with the autophagy pathway were upregulated in STHdh 111/111 neurons treated with 10 µM or 20 µM Compound 22 in comparison to untreated cells. Slc1a1, Ctse, Atp6vlh, Atp6v0d1, Ap3s1, Lamp1, Cd68, Gsub, Gba, Man2bl, Ppt1, Hexb, Npc1 and Gga1 were equal to or greater than 1 time the standard deviation from the mean upregulated in STHdh 111/111 neurons treated with 20 µM Compound 22 (FIG. 49).

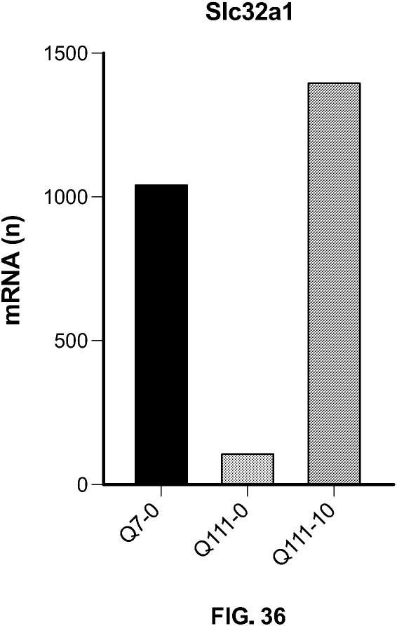

SLC32A1 (member 1 of the solute carrier family 32) is involved in the filling of vesicles at GABAergic and glycinergic synapses. GABA and glycine are the main inhibitory neurotransmitters in the brainstem and spinal cord (Reinus, R. at al., Front Behav Neurosci. 2015 Mar 27;9:71). SLC32A1 expression is downregulated in STHdh Q111/111 neurons expressing mHTT by 89% in comparison to wildtype STHdh Q7/7 level (FIG. 36). In Compound 22 treated STHdh 111/111 cells the Slc32a1 expression is upregulated 12-fold in comparison to untreated wildtype STHdh 111/111 neurons. Slc32a1 is downregulated in STHdh 111/111 (Q111-0) neurons in comparison to STHdh 7/7 (Q7-0). Treatment with Compound 22 improves SLC32a1 expression in STHdh 111/111 cells (Q111-10).

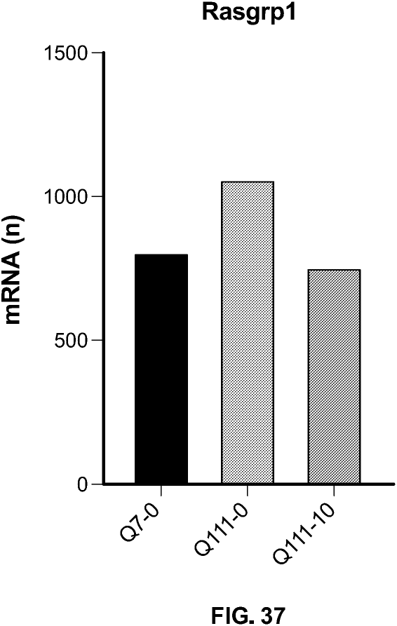

The GTPase Ras homolog-enriched in the striatum (Rhes) inhibits dopaminergic signaling in the striatum. Rhes is involved in the motor control of the stratum. Rhes is implicated in HD. It was described that the guanine nucleotide exchange factor (GEF) RasGRP1 inhibits Rhes role in striatal motor activity (Shahani, N. et al., Sci Signal. 2016 Nov 15;9(454):ra111). Rasgrp1 expression is upregulated in STHdh Q111/111 neurons expressing mHTT by 31% in comparison to wildtype STHdh Q7/7 level (FIG. 37). In Compound 22 treated STHdh 111/111 Rasgrp1 expression is downregulated by 29% in comparison to untreated STHdh 111/111 neurons.

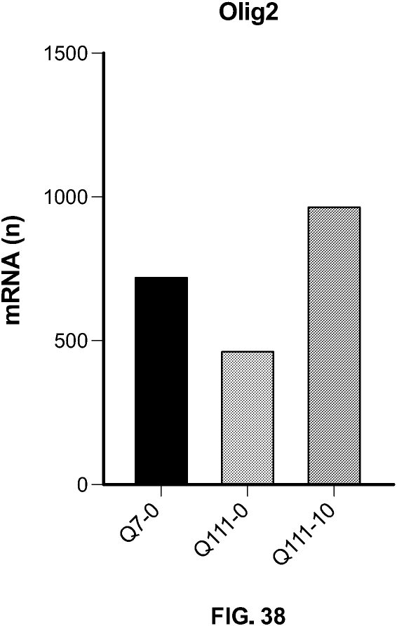

White matter abnormalities are prominent neuropathological features in Huntington’s disease (HD). They noted that the first group, which are sharply downregulated by mHTT, includes well-known oligodendrocyte lineage transcription factors OLIG2 (Osipovitch, M. et al., Cell Stem Cell. 2019 Jan 3;24(1):107-122.e7). Olig2 expression is downregulated in STHdh Q111/111 neurons expressing mHTT by 35% in comparison to wildtype STHdh Q7/7 level (FIG. 38). In Compound 22 treated STHdh 111/111 Olig2 expression is doubled in comparison to untreated STHdh 111/111 neurons. Olig2 is downregulated in STHdh 111/111 (Q111-0) neurons in comparison to STHdh 7/7 (Q7-0). Treatment with Compound 22 improves Olig2 expression in STHdh 111/111 cells (Q111-10).

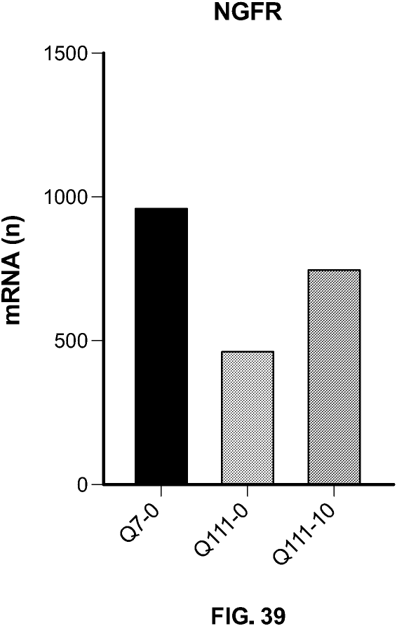

Intranuclear mutated huntingtin decreases the expression of nerve growth factor receptor (NGFR) in HD models (Li, S.H. et al. Mol Cell Biol. 2002 Mar;22(5): 1277-87). NGFR expression is downregulated in STHdh Q111/111 neurons expressing mHTT by 51% in comparison to wildtype STHdh Q7/7 level (FIG. 39). In Compound 22 treated STHdh 111/111 NGFR expression is increased by 60% in comparison to untreated STHdh 111/111 neurons. NGFR is downregulated in STHdh 111/111 (Q111-0) neurons in comparison to STHdh 7/7 (Q7-0). Treatment with Compound 22 improves NGFR expression in STHdh 111/111 cells (Q111-10).

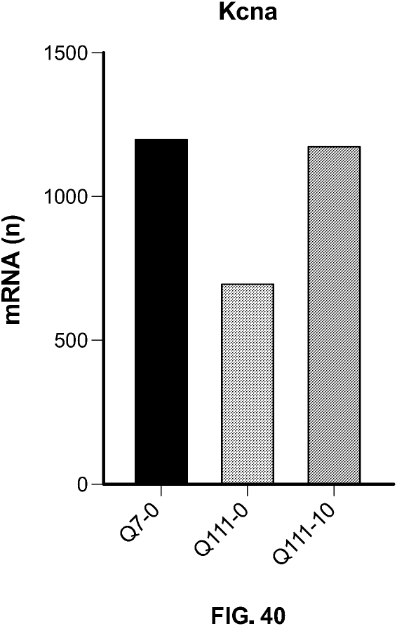

The KCNA1 gene encodes the alpha subunit of the potassium channel Kv1.1 which is found in brain tissue where it transports potassium ions into neurons. NGFR expression is downregulated in STHdh Q111/111 neurons expressing mHTT by 42% in comparison to wildtype STHdh Q7/7 level (FIG. 40). In Compound 22 treated STHdh 111/111 NGFR expression is increased by 68% in comparison to untreated STHdh 111/111 neurons. Kcna is downregulated in STHdh 111/111 (Q111-0) neurons in comparison to STHdh 7/7 (Q7-0). Treatment with Compound 22 improves Kcna expression in STHdh 111/111 cells (Q111-10).

### Example 41- Effects of Compound 4 on Striatal mHTT Levels

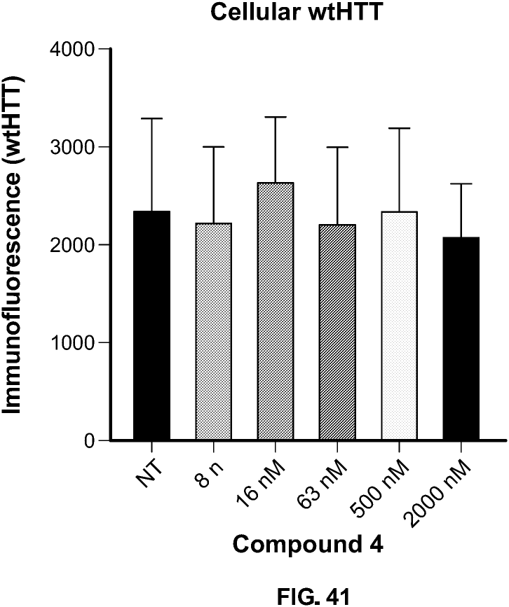

Effect of Compound 4 on Diffuse mHTT levels - ST HDH Q111/111 (CH00095, Coriell Institute) striatal derived cell line from a knock in transgenic mouse containing homozygous HTT loci with a humanized Exon 1 with 111 polyglutamine repeats. ST HDH Q7/7 (CH00097, Coriell Institute) striatal derived cell line from a knock in transgenic mouse containing HTT loci with a humanized Exon 1 containing 7 polyglutamine repeats. Diffuse mHTT quantified with immunocytochemistry as described in Example 33. Diffuse mHTT was significantly reduced by 68% (p < 0.0001, Welch’s t-test) in STHdh 111/111 cells treated with Compound 4 in comparison to untreated STHdh 111/111 cells. But wildtype huntingtin was not reduced in STHdh 7/7 cells treated with Compound 4, in comparison to non-treated STHdh 7/7 (FIG. 41).

### EC50 Value of Compound 4 for Reducing Mutated Huntingtin (mHTT)

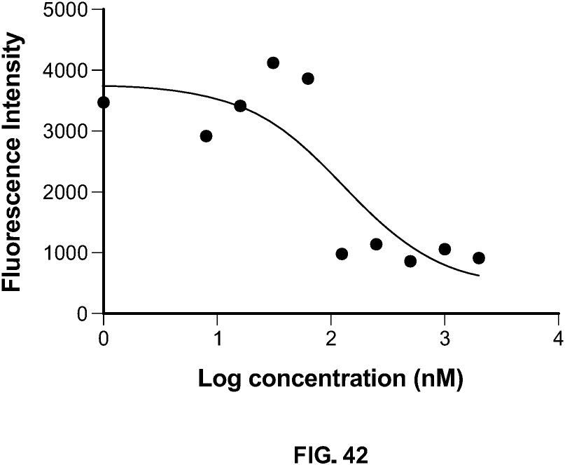

Diffuse mHTT quantified with immunocytochemistry as described in Example 33 with the exception that instead of 3B5H10 antibody for staining mHTT, MW1 (mouse anti-polyQ specific mab, Merck, catalog number: MABN2427) was used as primary antibody to stain mutated huntingtin. The EC 50 value for mHTT reduction was 130 nM (FIG. 42). EC 50 value was determined with non-linear regression.

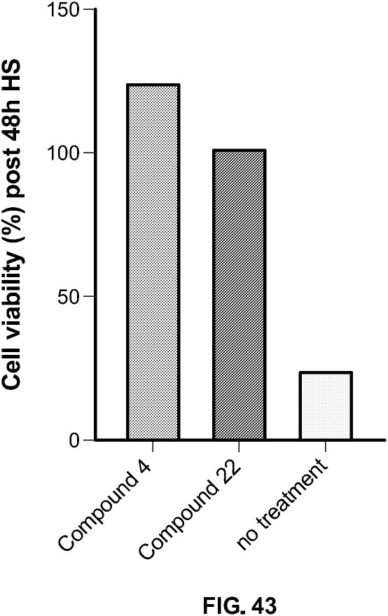

Effect of Compound 4 on Cell Viability - Heat shock triggers accumulation of misfolded proteins, which are degraded. At 48 hours after 3 hours heat shock the cell viability of STHdh 111/111 treated with Compound 4, with 125% in comparison to pre-heat shock cell viability, was higher in comparison to untreated cells, with 25% in comparison to pre-heat shock cell viability (FIG. 43).

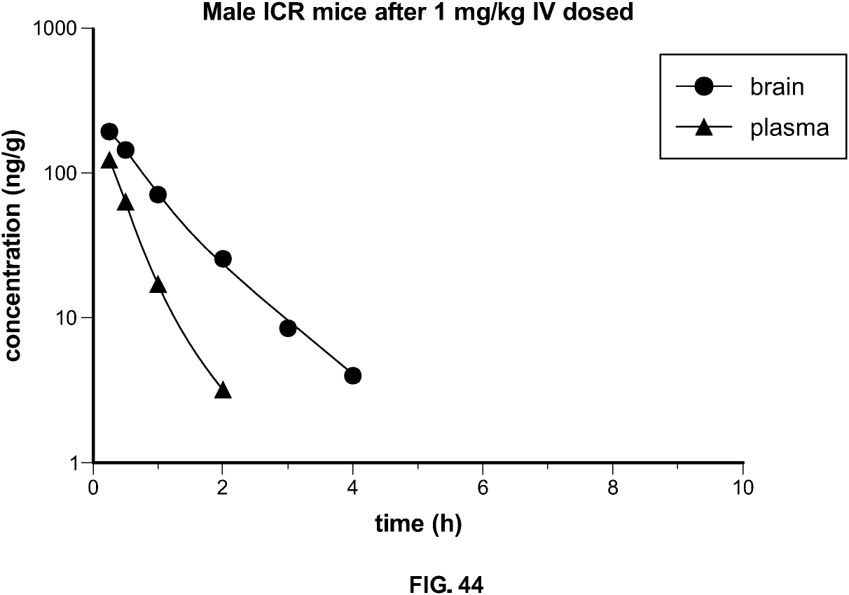

Physicochemical properties of Compound 4 - Compound 4 is both soluble and it can cross the blood brain barrier. Table 14 describes physicochemical properties of Compound 4 (FIG. 44).

TABLE 14

Brain Exposure and Physicochemical Properties of Compound 4

| | |
|---|---|
| | Compound 4 |
| Thermodynamic Solubility | 5.1 mM |
| Kinetic solubility | 328 µM |
| Log D | 2.02 (pH 7.4) |
| Blood Brain Barrier Penetrability | AUC_{brain}/AUCₚₗₐₛₘₐ = 2.27 |

### Example 42 - Phosphorylated Tau Reduction

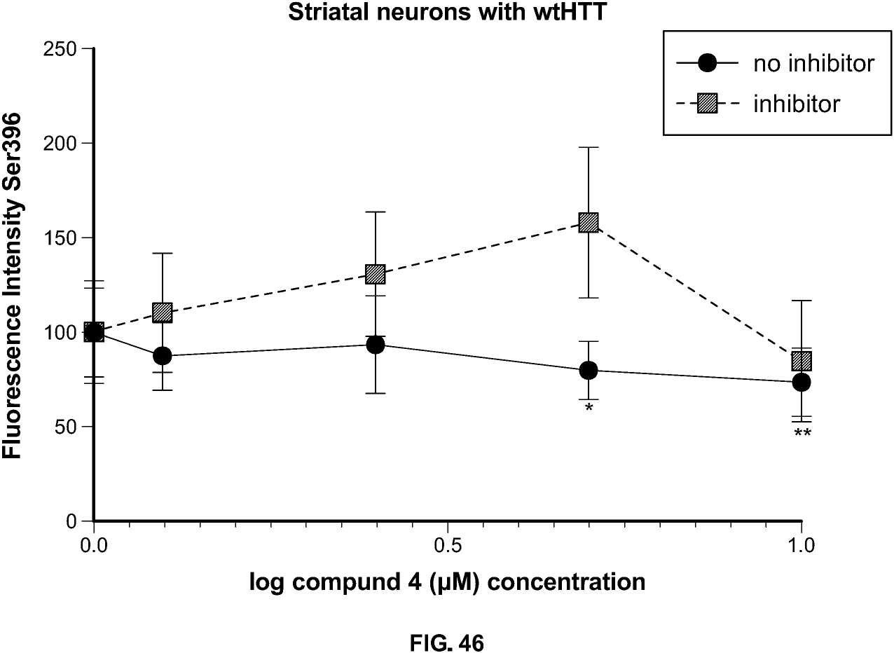

Effect of Compound 4 on Phospho-Tau (Ser396) levels - ST HDH Q7/7 (CH00097, Coriell Institute) striatal derived cell line from a knock in transgenic mouse containing HTT loci with a humanized Exon 1 containing 7 polyglutamine repeats. Immunocytochemistry was conducted as described in Example 33 with the exception that instead of 3B5H10 antibody for staining mHTT, phospho-Tau (Ser396) polyclonal antibody (ThermoFisher, Catalog 44-752G) was used to stain phospho-Tau (Ser396). Phospho-Tau (Ser396) was significantly reduced by 26.3% (p = 0.01, Welch’s t-test) in STHdh 7/7 cells treated with 10 µM Compound 4 in comparison to untreated STHdh 7/7 cells. In STHdh 7/7 cells exposed to 10 mM autophagy inhibitor ammonium chloride (Sigma Aldrich) Compound 4 treatment with 5 µM could not reduce phospho-Tau (Ser396) in comparison to non-treated STHdh 7/7. But 10 µM Compound 4 treatment could reduce in STHdh 7/7 exposed to ammonium chloride 15.2% phospho-Tau (Ser396) in comparison to non-treated STHdh 7/7 (FIG. 46).

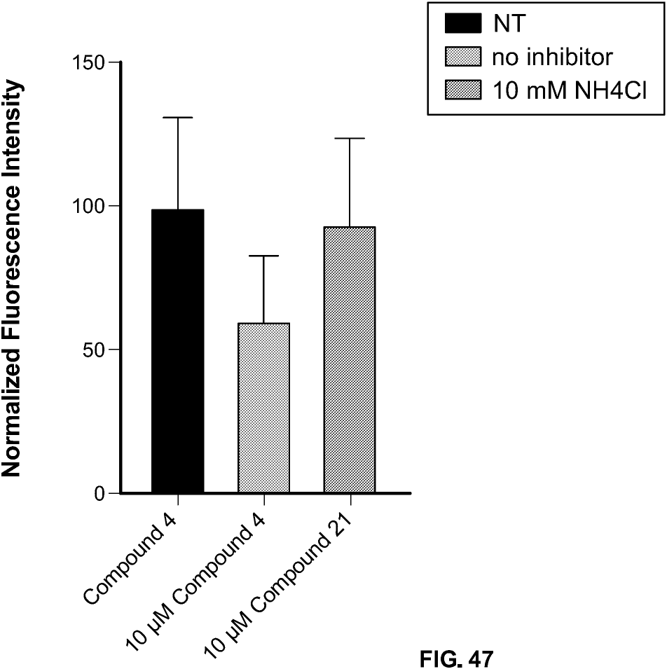

Effect of Compound 4 on Phospho-Tau (Ser396) levels - ST HDH Q7/7 (CH00097, Coriell Institute) striatal derived cell line from a knock in transgenic mouse containing HTT loci with a humanized Exon 1 containing 7 polyglutamine repeats. Diffuse mHTT quantified with immunocytochemistry as described in Example 33 with the exception that instead of 3B5H10 antibody for staining mHTT, phospho-Tau (Ser404) polyclonal antibody (ThermoFisher, Catalog 44-758G) was used to stain phospho-Tau (Ser396) and no heat shock was used. Phospho-Tau (Ser404) was significantly reduced by 40% (p = 0.003, Welch’s t-test) in STHdh 7/7 cells treated with 10 µM Compound 4 in comparison to untreated STHdh 7/7 cells. In STHdh 7/7 cells exposed to 10 mM autophagy inhibitor ammonium chloride (Sigma Aldrich) Compound 4 treatment could not reduce phospho-Tau (Ser404) in Compound 4 STHdh 7/7 neurons (FIG. 47).

### Example 43 - Striatal Cells Expressing Mutated Huntingtin Morphology Improvements

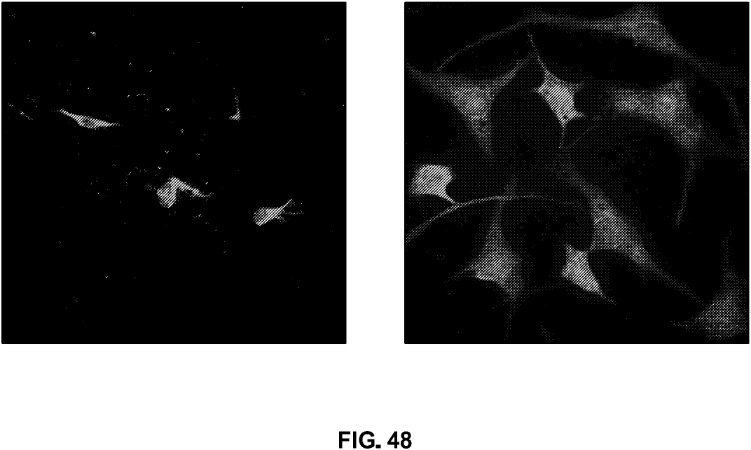

STHdh 111/111 are striatal cells expressing mutated huntingtin (Q111). STHdh 111/111 which were not treatment showed impaired morphology and high rate of cell death. STHdh 111/111 treated with Compound 4 depicted improved cell viability, dendrite outgrowth and increased cell size (FIG. 48).

### Example 44 - Motility Zebrafish Model

Exposure to the neurotoxin 1-methyl-4-phenylpyridinium (MPP+) has been shown to induce dopaminergic ablation (1) and locomotor perturbations symptomatic of Parkinson’s disease (2) in zebrafish larvae. MPP+ selectively targets dopaminergic neurons; the putative mechanism is induction of K+ efflux, cell membrane hyperpolarization and inhibition of neuronal firing. This ultimately leads to an inhibition of complex I, a decrease in ATP production and promotion of the generation of reactive oxygen species upon accumulation in the mitochondria (3). The observed phenotype following exposure to 500 µM from 24 hours post fertilization (hpf) to 120 hpf is characterized by a decrease in movement during lights-on phases of a photomotor assay. In unpublished data below, representative locomotor perturbations of the distance moved and movement frequency is shown.

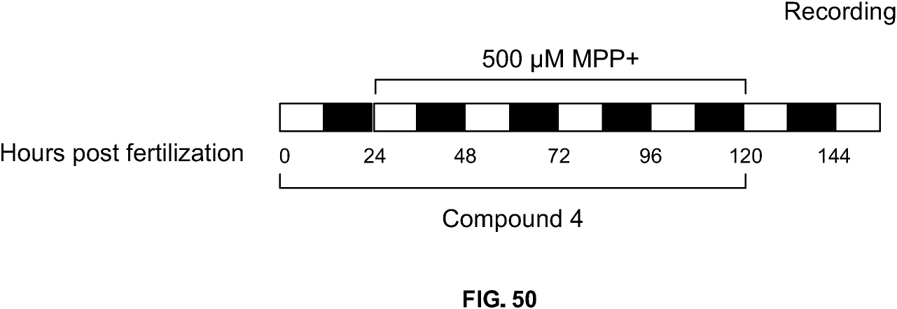

To study the potential for neuroprotection, zebrafish embryos were treated with Compound 4 prior to MPP+ exposure at 0 hpf, and as co-incubation with MPP+ at 24 hpf onwards (FIG. 50).

Six groups were included in each experiment. Immediately after spawning, 40 embryos per group were allocated to a 24-well plate and system water (solvent) was added to all wells. For assessment of neuroprotective potential, Compound 4 was added for a final concentration of 1, 10 and 20 µM (column 4-6). From the following day at 24 hpf, Compound 4 was co-incubated with 500 µM MPP+. MPP+ was prepared immediately prior to incubation and solutions were changed daily. At 120 hpf, drug treatment and MPP+ exposure was ceased and a 3X wash was performed with system water. Larvae were then relocated to 96-well plates for behavioral recording and placed in a separate recording room for 24-hour acclimation. Prior to behavioral recording, wells were fully replenished system water.

Naive group was exposed to solvent only. Vehicle group was exposed to solvent and vehicle corresponding to the vehicle of 20 µM drug solution (0.02% DMSO). MPP+ positive control was exposed to solvent and 500 µM MPP+. All compound groups were treated with the respective vehicle and respective concentration of compound 4.

| Compound | Concentration | Solvent | %DMSO |
|---|---|---|---|
| MPP+ | 500 µM | Water | 0 |
| Compound 4 | 100 mM | DMSO | 100 |
| Compound 4 | 10 mM (diluation of 100 mM) | Distilled Water | 10 |
| Compound 4 | 1 µM (dilution of 10 mM) | Water | 0.001 |
| Compound 4 | 10 µM (dilution of 10 mM) | Water | 0.01 |
| Compound 4 | 20 µM (dilution of 10 mM) | Water | 0.02 |
| Vehicle | 20 µM (DMSO content, no drug*) | Water | 0.02 |

*0.02% DMSO vehicle was included for control for effect of DMSO to naive larvae.

Behavioral data was recorded from 1 p.m. on 6 dpf until shortly after wakening on 7 dpf. The recording consisted of a photomotor assay in which lights switch off and on in 30-minute intervals between 1 p.m. and 6 p.m., producing a consistent photomotor response. Here, the larvae demonstrate a rapid increase in movement in response to lights-off followed by a gradual decrease. Upon lights being turned on again, the larvae cease movement followed by a return to baseline. This cycle is repeated five times. Following the last lights-off phase, constant illumination is presented until lights turn off at 10 p.m. for the night. Lights turn on again at 8 a.m. the following day.

Average movement frequency was measured during 30-minute lights-on phases of photomotor assay. Movement bouts were defined as initiated when velocity exceeded a threshold of 2 mm/s and ceased when velocity fell below 1 mm/s.

Distance moved (mm) was defined as the average distance moved during 30-minute lights-on phases of photomotor assay.

Data was obtained using EthoVision XT (Version 11.5.2016, Noldus) and exported to Microsoft Excel for data analysis. Statistical analysis was performed using GraphPad Prism Software (Version 5.01, GraphPad Software Inc.). All data are presented as mean ± standard error of the mean (sem). For analysis of differences between groups, a Kruskal-Wallis one-way ANOVA with Dunn’s multiple comparison post hoc test was performed. P < 0.05 was considered statistically significant.

The neuroprotective potential of Compound 4 was assessed following co-incubation with MPP+. A statistically significant difference in distance moved between the different treatment groups, χ2(5) = 29.20, p < 0.0001, was observed. Larvae exposed to MPP+ alone moved significantly shorter distances than naive larvae (p < 0.001), whereas no significant difference was observed for the drug treatment groups compared to MPP+.

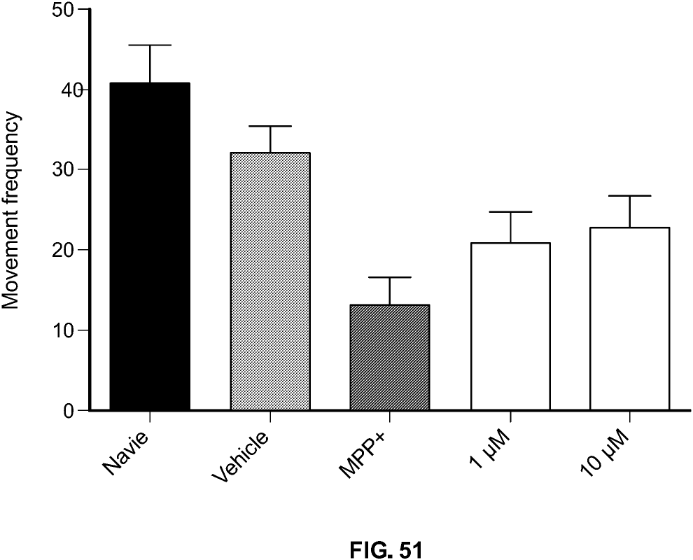

Similarly, a statistically significant difference in movement frequency between the different treatment groups, χ2(5) = 40.18, p < 0.0001, was observed. Larvae exposed to MPP+ alone moved significantly less frequently with 13.3 movement bouts in 30 minutes than naive larvae with 40.8 movement bouts in 30 minutes (p < 0.001), and larvae exposed to 1 µM Compound 4 moved significantly less frequently with 21.0 movement bouts in 30 minutes than naive larvae (p < 0.01), whereas no significant difference was observed for the drug treatment groups compared to MPP+ (FIG. 51).

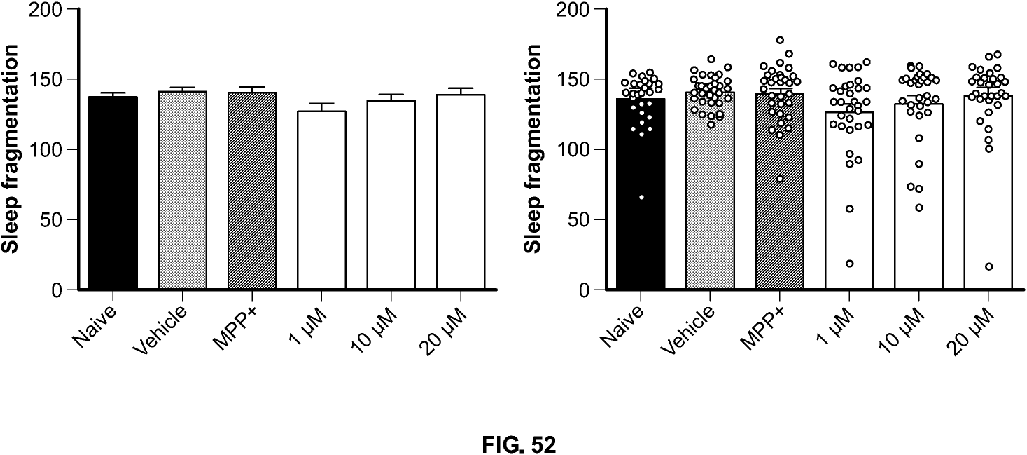

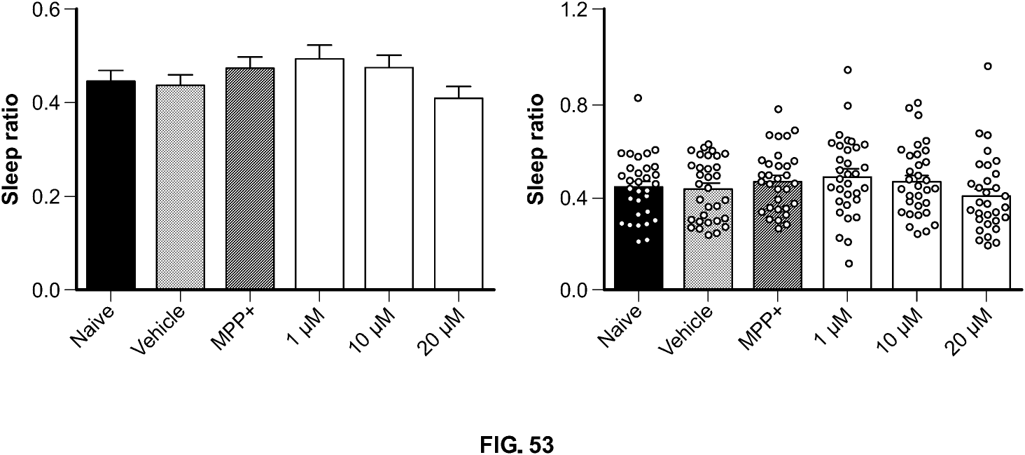

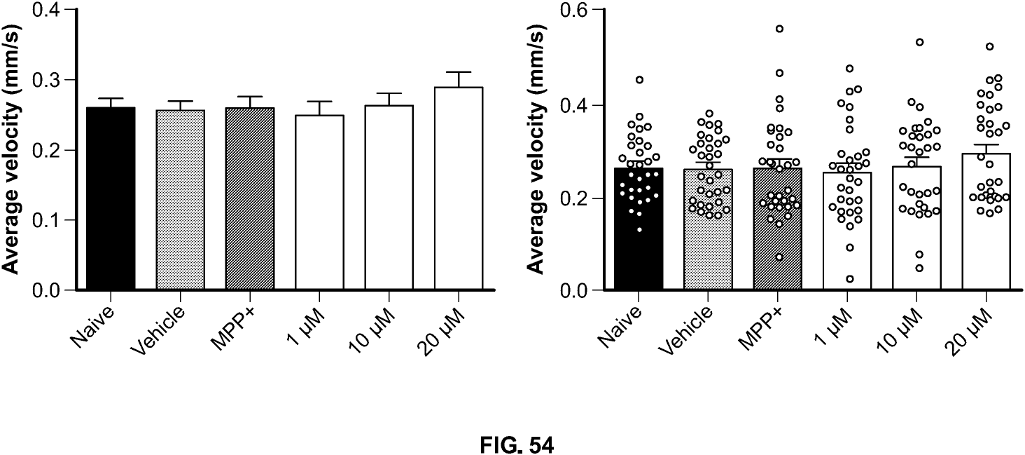

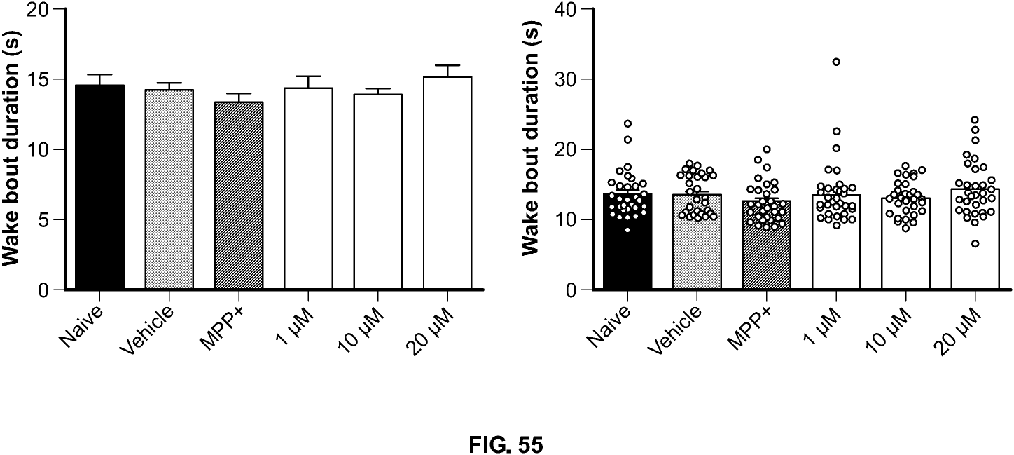

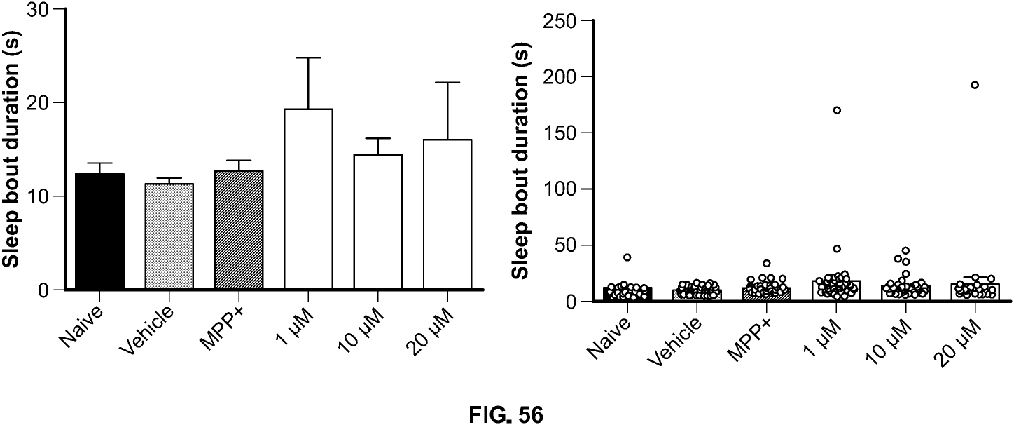

The effect of Compound 4 on sleep parameters was assessed following co-incubation with MPP+. No statistically significant difference between the different groups for either sleep fragmentation, χ2(5) = 6.573, p = 0.2544; sleep ratio, χ2(5) = 7.368, p = 0.1947; velocity, χ2(5) = 3.676, p = 0.5970; wake bout duration, χ2(5) = 5.015, p = 0.4141; or sleep bout duration, χ2(5) = 8.719, p = 0.1208, was observed (FIG. 52-FIG. 56).

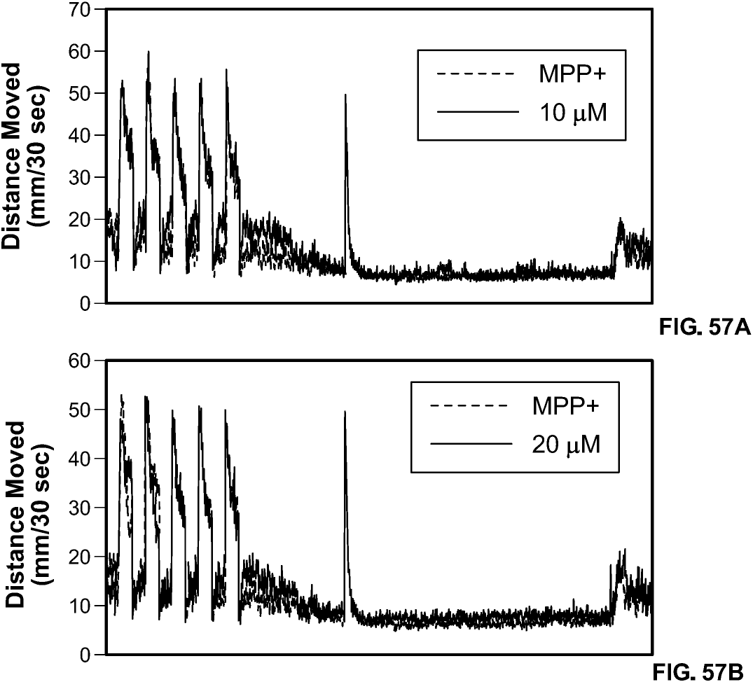

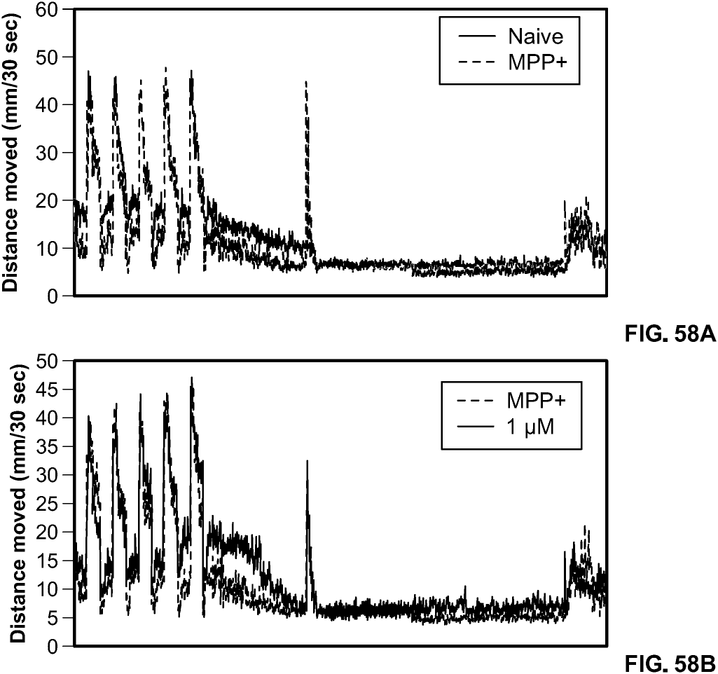

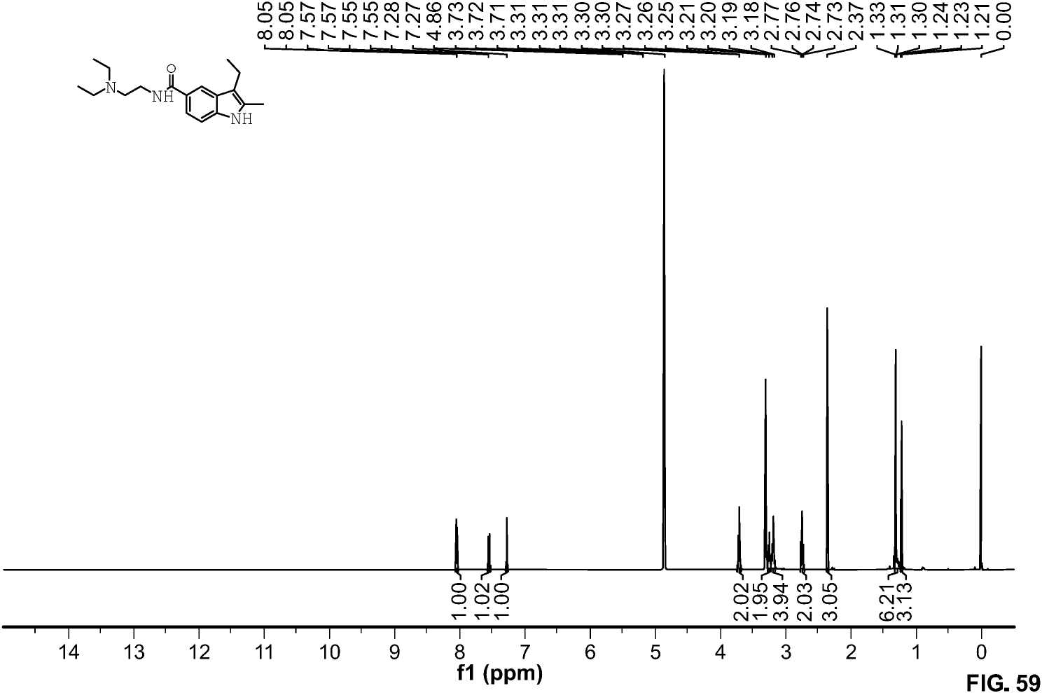

Despite no significant rescuing effects of Compound 4 in either of the performed experiments, the data indicates that concentrations ~10 µM and ~1 µM causes a subtle increase in overall movement during assessment of neuroprotection. This is supported by the plots depicting the movement during the entire recording sequence. In FIG. 57, a separation between the movement of larvae treated with MPP+ alone and 10 µM Compound 4 with MPP+ during lights-on phases is visible. In FIG. 58, the effects of 1 µM Compound 4 seems to be centered around the latter part of the photomotor assay and the first half of the daytime recording. The sleep phenotype of larvae treated with Compound 4, independent of concentration, appears similar to both naive and MPP+-treated larvae in both experiments. This suggests that Compound 4 does neither reduce nor increase sleep and consistencies in both velocity and sleep fragmentation indicates that spontaneous movement during sleep is retained.

### EQUIVALENTS

While the present disclosure has been described in conjunction with the specific embodiments set forth above, many alternatives, modifications and other variations thereof will be apparent to those of ordinary skill in the art. All such alternatives, modifications and variations are intended to fall within the spirit and scope of the present disclosure.

1. A method of treating Huntington’s Disease, comprising administering to a subject in need thereof an effective amount of a compound of Formula I: 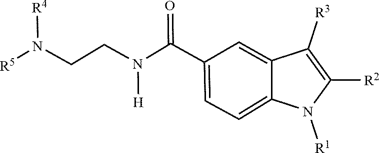
or a pharmaceutically acceptable salt, prodrug, solvate, hydrate, tautomer or isomer thereof, wherein:
R¹ is independently —H, —C₁—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
R² is independently —H, —C₁—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
or R¹ and R² can optionally combine, together with the atoms to which they are attached, to form a C₄-C₈heterocycle, wherein the heterocycle is optionally substituted with one or more substituents independently selected from the group consisting of —Br, —F, —Ci—C₆alkyl, —C₂—C₆alkenyl, and —C₂—C₆alkynyl, and wherein each alkyl, alkenyl, and alkynyl is optionally substituted with one or more —Br or —F;
R³ is independently —H, —C₁—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
or R² and R³ can optionally combine, together with the atoms to which they are attached, to form a C₄-C₈carbocycle or a C₄-C₈heterocycle, wherein the carbocycle or heterocycle is optionally substituted with one or more substituents independently selected from the group consisting of —Br, —F, —C₁—C₆alkyl, —C₂—C₆alkenyl, and —C₂—C₆alkynyl, and wherein each alkyl, alkenyl, and alkynyl is optionally substituted with one or more —Br or —F;
R⁴ is independently —H, —C₁—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
R⁵ is independently —H, —C₁—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
or R⁴ and R⁵ can optionally combine, together with the nitrogen atom to which they are attached, to form a C₃-C₈heterocycle, wherein the heterocycle is optionally substituted with one or more substituents independently selected from the group consisting of —Br, —F, —Ci—C₆alkyl, —C₂—C₆alkenyl, and —C₂—C₆alkynyl, and wherein each alkyl, alkenyl and alkynyl is optionally substituted with one or more —Br or —F.

2. A method of reversing a conformational change of mutated huntingtin, comprising administering to a subject in need thereof an effective amount of a compound of Formula I: 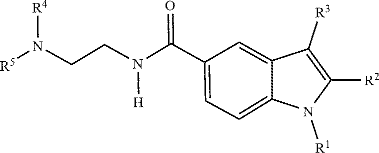
or a pharmaceutically acceptable salt, prodrug, solvate, hydrate, tautomer or isomer thereof, wherein:
R¹ is independently —H, —C₁—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
R² is independently —H, —C₁—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
or R¹ and R² can optionally combine, together with the atoms to which they are attached, to form a C₄-C₈heterocycle, wherein the heterocycle is optionally substituted with one or more substituents independently selected from the group consisting of —Br, —F, —Ci—C₆alkyl, —C₂—C₆alkenyl, and —C₂—C₆alkynyl, and wherein each alkyl, alkenyl, and alkynyl is optionally substituted with one or more —Br or —F;
R³ is independently —H, —C₁—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
or R² and R³ can optionally combine, together with the atoms to which they are attached, to form a C₄-C₈carbocycle or a C₄-C₈heterocycle, wherein the carbocycle or heterocycle is optionally substituted with one or more substituents independently selected from the group consisting of —Br, —F, —C₁—C₆alkyl, —C₂—C₆alkenyl, and —C₂—C₆alkynyl, and wherein each alkyl, alkenyl, and alkynyl is optionally substituted with one or more —Br or —F;
R⁴ is independently —H, —C₁—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
R⁵ is independently —H, —C₁—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
or R⁴ and R⁵ can optionally combine, together with the nitrogen atom to which they are attached, to form a C₃-C₈heterocycle, wherein the heterocycle is optionally substituted with one or more substituents independently selected from the group consisting of —Br, —F, —Ci—C₆alkyl, —C₂—C₆alkenyl, and —C₂—C₆alkynyl, and wherein each alkyl, alkenyl and alkynyl is optionally substituted with one or more —Br or —F.

3. A method of treating a polyQ disease, comprising administering to a subject in need thereof an effective amount of a compound of Formula I: 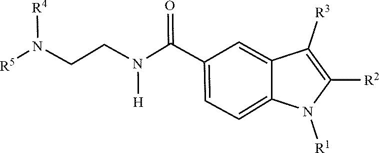
or a pharmaceutically acceptable salt, prodrug, solvate, hydrate, tautomer or isomer thereof, wherein:
R¹ is independently —H, —C₁—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
R² is independently —H, —C₁—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
or R¹ and R² can optionally combine, together with the atoms to which they are attached, to form a C₄-C₈heterocycle, wherein the heterocycle is optionally substituted with one or more substituents independently selected from the group consisting of —Br, —F, —Ci—C₆alkyl, —C₂—C₆alkenyl, and —C₂—C₆alkynyl, and wherein each alkyl, alkenyl, and alkynyl is optionally substituted with one or more —Br or —F;
R³ is independently —H, —C₁—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
or R² and R³ can optionally combine, together with the atoms to which they are attached, to form a C₄-C₈carbocycle or a C₄-C₈heterocycle, wherein the carbocycle or heterocycle is optionally substituted with one or more substituents independently selected from the group consisting of —Br, —F, —C₁—C₆alkyl, —C₂—C₆alkenyl, and —C₂—C₆alkynyl, and wherein each alkyl, alkenyl, and alkynyl is optionally substituted with one or more —Br or —F;
R⁴ is independently —H, —C₁—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
R⁵ is independently —H, —C₁—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
or R⁴ and R⁵ can optionally combine, together with the nitrogen atom to which they are attached, to form a C₃-C₈heterocycle, wherein the heterocycle is optionally substituted with one or more substituents independently selected from the group consisting of —Br, —F, —Ci—C₆alkyl, —C₂—C₆alkenyl, and —C₂—C₆alkynyl, and wherein each alkyl, alkenyl and alkynyl is optionally substituted with one or more —Br or —F.

4. A method of reducing mutated or misfolded proteins, comprising administering to a subject in need thereof an effective amount of a compound of Formula I: 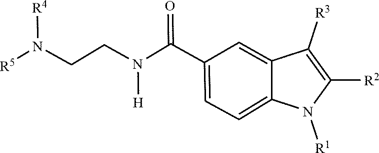
or a pharmaceutically acceptable salt, prodrug, solvate, hydrate, tautomer or isomer thereof, wherein:
R¹ is independently —H, —C₁—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
R² is independently —H, —C₁—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
or R¹ and R² can optionally combine, together with the atoms to which they are attached, to form a C₄-C₈heterocycle, wherein the heterocycle is optionally substituted with one or more substituents independently selected from the group consisting of —Br, —F, —Ci—C₆alkyl, —C₂—C₆alkenyl, and —C₂—C₆alkynyl, and wherein each alkyl, alkenyl, and alkynyl is optionally substituted with one or more —Br or —F;
R³ is independently —H, —C₁—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
or R² and R³ can optionally combine, together with the atoms to which they are attached, to form a C₄-C₈carbocycle or a C₄-C₈heterocycle, wherein the carbocycle or heterocycle is optionally substituted with one or more substituents independently selected from the group consisting of —Br, —F, —C₁—C₆alkyl, —C₂—C₆alkenyl, and —C₂—C₆alkynyl, and wherein each alkyl, alkenyl, and alkynyl is optionally substituted with one or more —Br or —F;
R⁴ is independently —H, —C₁—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
R⁵ is independently —H, —C₁—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
or R⁴ and R⁵ can optionally combine, together with the nitrogen atom to which they are attached, to form a C₃-C₈heterocycle, wherein the heterocycle is optionally substituted with one or more substituents independently selected from the group consisting of —Br, —F, —Ci—C₆alkyl, —C₂—C₆alkenyl, and —C₂—C₆alkynyl, and wherein each alkyl, alkenyl and alkynyl is optionally substituted with one or more —Br or —F.

5. A method of neuroprotection, comprising administering to a subject in need thereof an effective amount of a compound of Formula I: 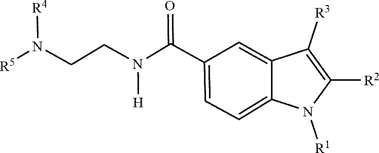
or a pharmaceutically acceptable salt, prodrug, solvate, hydrate, tautomer or isomer thereof, wherein:
R¹ is independently —H, —C₁—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
R² is independently —H, —C₁—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
or R¹ and R² can optionally combine, together with the atoms to which they are attached, to form a C₄-C₈heterocycle, wherein the heterocycle is optionally substituted with one or more substituents independently selected from the group consisting of —Br, —F, —Ci—C₆alkyl, —C₂—C₆alkenyl, and —C₂—C₆alkynyl, and wherein each alkyl, alkenyl, and alkynyl is optionally substituted with one or more —Br or —F;
R³ is independently —H, —C₁—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
or R² and R³ can optionally combine, together with the atoms to which they are attached, to form a C₄-C₈carbocycle or a C₄-C₈heterocycle, wherein the carbocycle or heterocycle is optionally substituted with one or more substituents independently selected from the group consisting of —Br, —F, —C₁—C₆alkyl, —C₂—C₆alkenyl, and —C₂—C₆alkynyl, and wherein each alkyl, alkenyl, and alkynyl is optionally substituted with one or more —Br or —F;
R⁴ is independently —H, —C₁—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
R⁵ is independently —H, —C₁—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
or R⁴ and R⁵ can optionally combine, together with the nitrogen atom to which they are attached, to form a C₃-C₈heterocycle, wherein the heterocycle is optionally substituted with one or more substituents independently selected from the group consisting of —Br, —F, —Ci—C₆alkyl, —C₂—C₆alkenyl, and —C₂—C₆alkynyl, and wherein each alkyl, alkenyl and alkynyl is optionally substituted with one or more —Br or —F.

6. A method of inducing autophagy, comprising administering to a subject in need thereof an effective amount of a compound of Formula I: 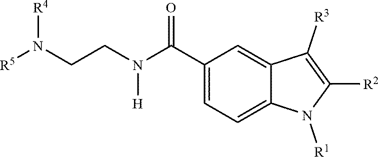
or a pharmaceutically acceptable salt, prodrug, solvate, hydrate, tautomer or isomer thereof, wherein:
R¹ is independently —H, —C₁—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
R² is independently —H, —C₁—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
or R¹ and R² can optionally combine, together with the atoms to which they are attached, to form a C₄-C₈heterocycle, wherein the heterocycle is optionally substituted with one or more substituents independently selected from the group consisting of —Br, —F, —Ci—C₆alkyl, —C₂—C₆alkenyl, and —C₂—C₆alkynyl, and wherein each alkyl, alkenyl, and alkynyl is optionally substituted with one or more —Br or —F;
R³ is independently —H, —C₁—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
or R² and R³ can optionally combine, together with the atoms to which they are attached, to form a C₄-C₈carbocycle or a C₄-C₈heterocycle, wherein the carbocycle or heterocycle is optionally substituted with one or more substituents independently selected from the group consisting of —Br, —F, —C₁—C₆alkyl, —C₂—C₆alkenyl, and —C₂—C₆alkynyl, and wherein each alkyl, alkenyl, and alkynyl is optionally substituted with one or more —Br or —F;
R⁴ is independently —H, —C₁—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
R⁵ is independently —H, —C₁—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
.

7. Or R⁴ and R⁵ can optionally combine, together with the nitrogen atom to which they are attached, to form a C₃-C₈heterocycle, wherein the heterocycle is optionally substituted with one or more substituents independently selected from the group consisting of —Br, —F, —Ci—C₆alkyl, —C₂—C₆alkenyl, and —C₂—C₆alkynyl, and wherein each alkyl, alkenyl and alkynyl is optionally substituted with one or more —Br or —F.

8. The method of claim 6, wherein the autophagy is induced by a small molecule interacting with huntingtin.

9. The method of any of claims 1-6, wherein R¹ is —H.

10. The method of any of claims 1-6, wherein R¹ is —C₁—C₆alkyl.

11. The method of any of claims 1-6, wherein R² is —C₁—C₆alkyl.

12. The method of any of claims 1-6, wherein R³ is —C₁—C₆alkyl.

13. The method of any of claims 1-6, wherein R¹ and R² are —C₁—C₆alkyl.

14. The method of any of claims 1-6, wherein R² and R³ are —C₁—C₆alkyl.

15. The method of any of claims 1-6, wherein R² and R³ combine to form a carbocycle.

16. The method of any of claims 1-6, wherein R² and R³ combine to form a heterocycle.

17. The method of any of claims 1-6, wherein R⁴ is —C₁—C₆alkyl.

18. The method of any of claims 1-6, wherein R⁵ is —C₁—C₆alkyl.

19. The method of any of claims 1-6, wherein R⁴ and R⁵ combine to form a heterocycle.

20. The method of any of claims 1-6, wherein the heterocycle is substituted with one or more —C₁—C₆alkyl.

21. The method of any of claims 1-6, wherein the compound has a structure of Formula I-A: 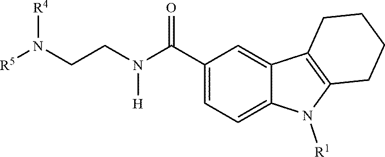
.

22. The method of any of claims 1-6, wherein the compound has a structure of Formula I-B: 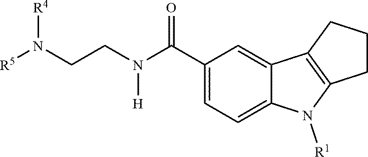
.

23. The method of any of claims 1-6, wherein the compound has a structure of Formula I-C: 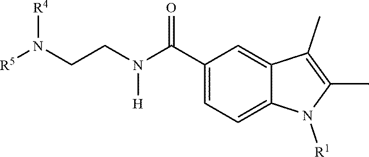
.

24. The method of any of claims 1-6, wherein the compound has a structure of Formula I-D: 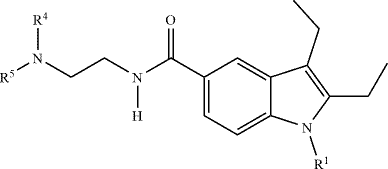
.

25. The method of any of claims 1-6, wherein the compound has a structure of Formula I-E: 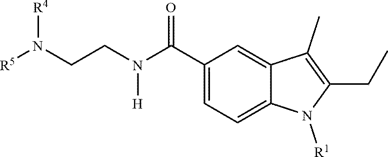
.

26. The method of any of claims 1-6, wherein the compound has a structure of Formula IF: 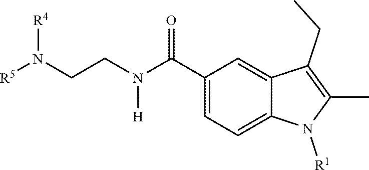
.

27. The method of any of claims 1-6, wherein the compound has a structure of Formula I-G: 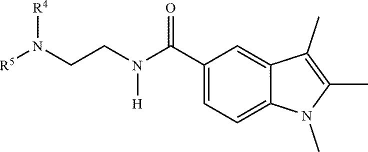
.

28. The method of any of claims 1-6, wherein the compound has a structure of Formula I-H: 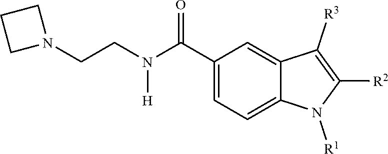
.

29. The method of any of claims 1-6, wherein the compound has a structure of Formula I-I: 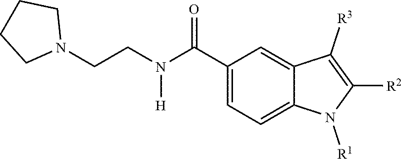
.

30. The method of any of claims 1-6, wherein the compound has a structure of Formula I-J: 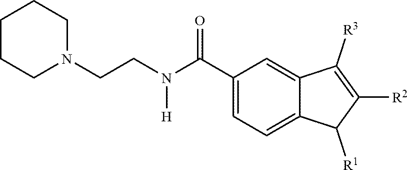
.

31. The method of any of claims 1-6, wherein the compound has a structure of Formula I-K: 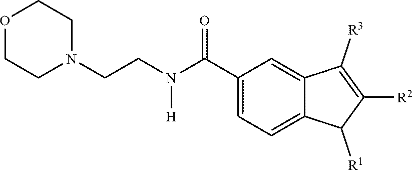
.

32. The method of any of claims 1-6, wherein the compound has a structure of Formula I-L: 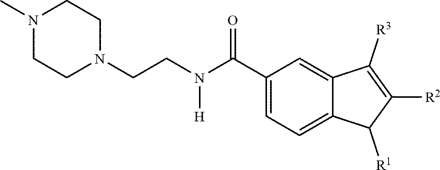
.

33. The method of any of claims 1-6, wherein the compound has a structure of Formula I-M: 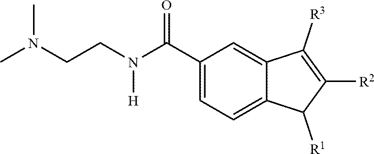
.

34. The method of any of claims 1-6, wherein the compound has a structure of Formula IN: 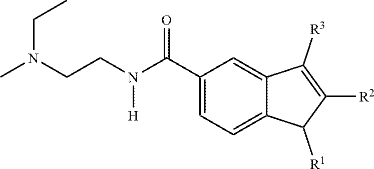
.

35. The method of any of claims 1-6, wherein the compound has a structure of Formula I-O: 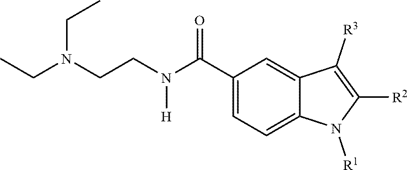
.

36. The method of any of claims 1-6, wherein the compound has a formula selected from the group consisting of:
Structure 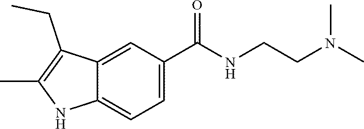 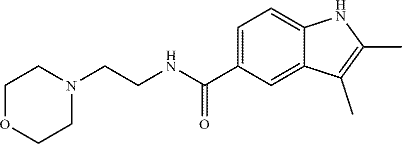 [SMILES: Cc1[nH]c2ccc(C(=O)NCCN3CCOCC3)cc2c1C] 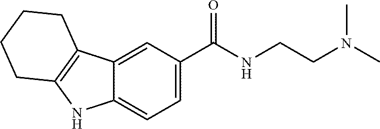 [SMILES: CN(C)CCNC(=O)c1ccc2[nH]c3c(c2c1)CCCC3] 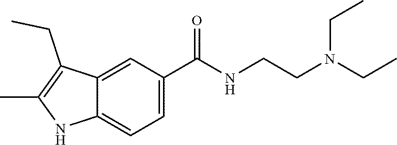 [SMILES: CCc1c(C)[nH]c2ccc(C(=O)NCCN(CC)CC)cc12] 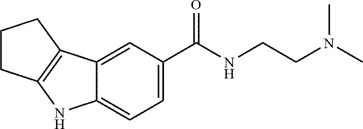 [SMILES: CN(C)CCNC(=O)c1ccc2[nH]c3c(c2c1)CCC3] 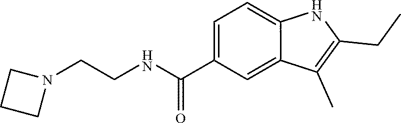 [SMILES: CCc1[nH]c2ccc(C(=O)NCCN3CCC3)cc2c1C] 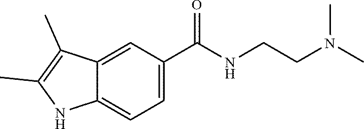 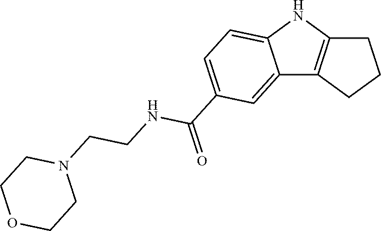 [SMILES: O=C(NCCN1CCOCC1)c1ccc2[nH]c3c(c2c1)CCC3] 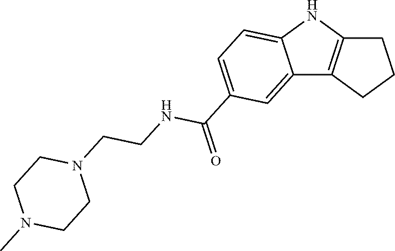 [SMILES: CN1CCN(CCNC(=O)c2ccc3[nH]c4c(c3c2)CCC4)CC1] 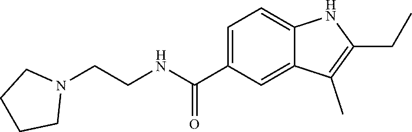 [SMILES: CCc1[nH]c2ccc(C(=O)NCCN3CCCC3)cc2c1C] 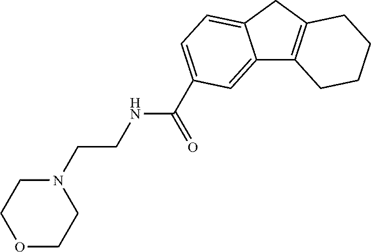 [SMILES: O=C(NCCN1CCOCC1)c1ccc2c(c1)C1=C(CCCC1)C2] 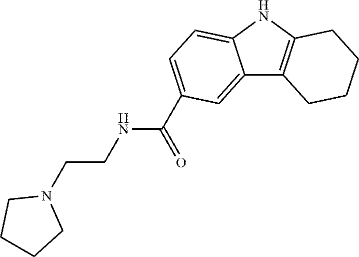 [SMILES: O=C(NCCN1CCCC1)c1ccc2[nH]c3c(c2c1)CCCC3] 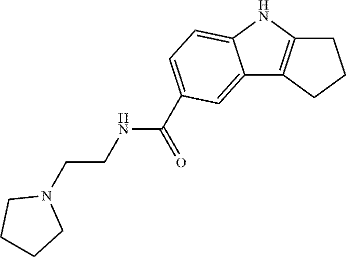 [SMILES: O=C(NCCN1CCCC1)c1ccc2[nH]c3c(c2c1)CCC3] 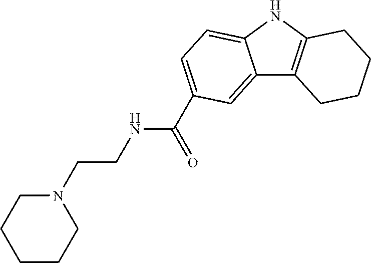 [SMILES: O=C(NCCN1CCCCC1)c1ccc2[nH]c3c(c2c1)CCCC3] 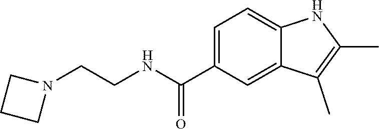 [SMILES: Cc1[nH]c2ccc(C(=O)NCCN3CCC3)cc2c1C] 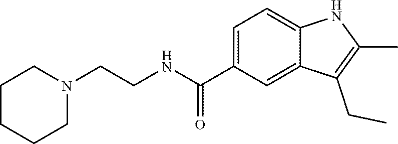 [SMILES: CCc1c(C)[nH]c2ccc(C(=O)NCCN3CCCCC3)cc12] 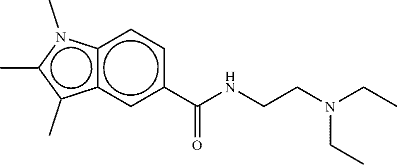 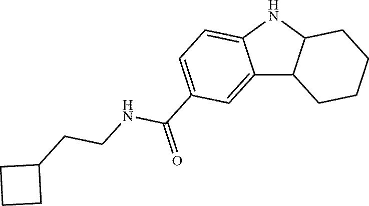 [SMILES: O=C(NCCC1CCC1)c1ccc2c(c1)C1CCCCC1N2] 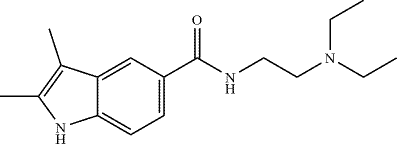 [SMILES: CCN(CC)CCNC(=O)c1ccc2[nH]c(C)c(C)c2c1] 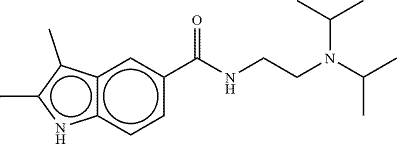 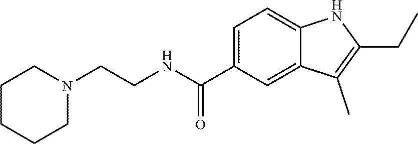 [SMILES: CCc1[nH]c2ccc(C(=O)NCCN3CCCCC3)cc2c1C] 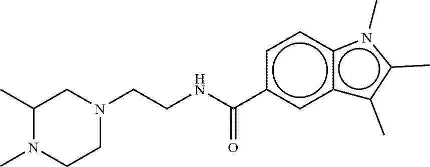 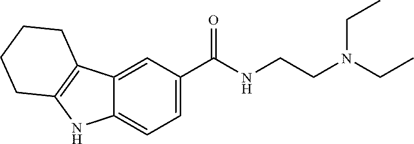 [SMILES: CCN(CC)CCNC(=O)c1ccc2[nH]c3c(c2c1)CCCC3] 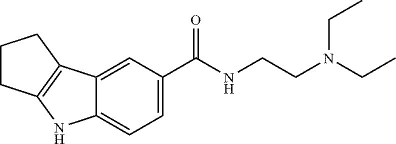 [SMILES: CCN(CC)CCNC(=O)c1ccc2[nH]c3c(c2c1)CCC3] 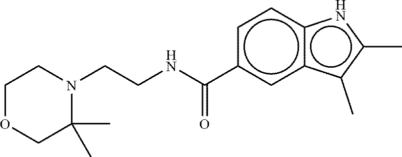 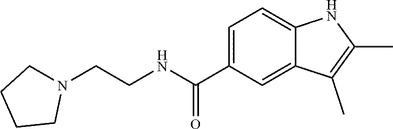 [SMILES: Cc1[nH]c2ccc(C(=O)NCCN3CCCC3)cc2c1C] 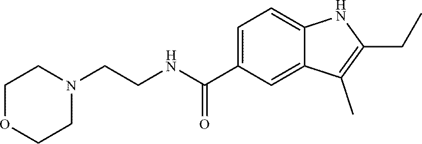 [SMILES: CCc1[nH]c2ccc(C(=O)NCCN3CCOCC3)cc2c1C] 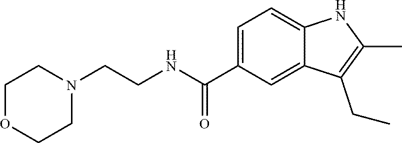 [SMILES: CCc1c(C)[nH]c2ccc(C(=O)NCCN3CCOCC3)cc12] 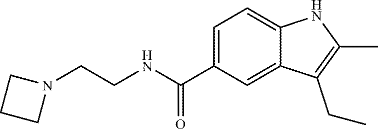 [SMILES: CCc1c(C)[nH]c2ccc(C(=O)NCCN3CCC3)cc12] 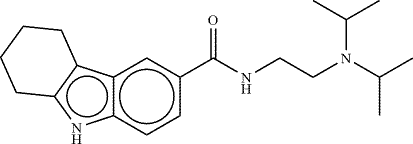 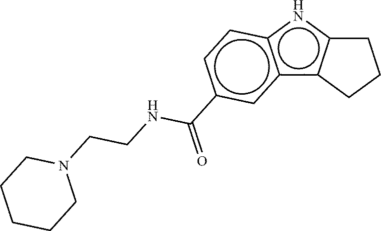 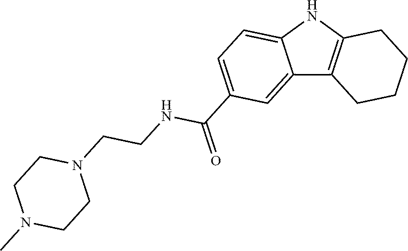 [SMILES: CN1CCN(CCNC(=O)c2ccc3[nH]c4c(c3c2)CCCC4)CC1] 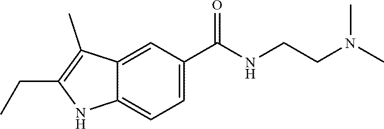 [SMILES: CCc1[nH]c2ccc(C(=O)NCCN(C)C)cc2c1C] 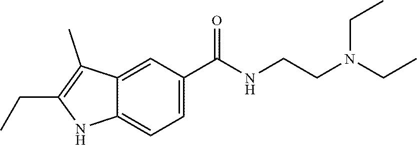 [SMILES: CCc1[nH]c2ccc(C(=O)NCCN(CC)CC)cc2c1C] 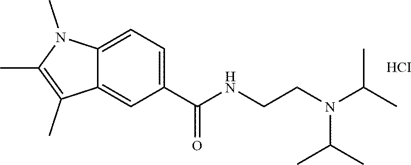 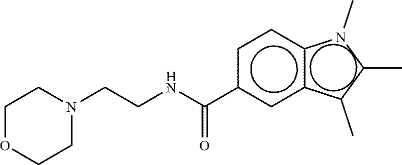 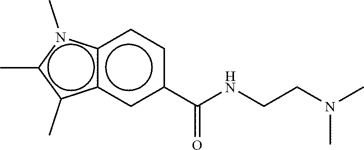 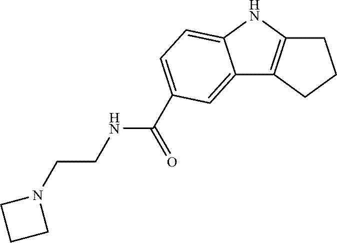 [SMILES: O=C(NCCN1CCC1)c1ccc2[nH]c3c(c2c1)CCC3] 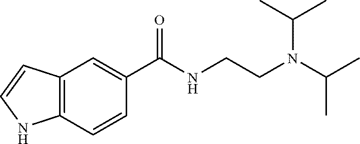 [SMILES: CC(C)N(CCNC(=O)c1ccc2[nH]ccc2c1)C(C)C] 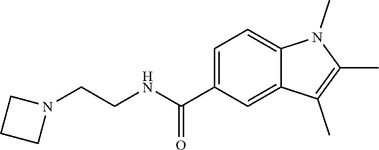 [SMILES: Cc1c(C)n(C)c2ccc(C(=O)NCCN3CCC3)cc12] 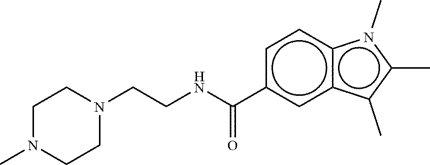 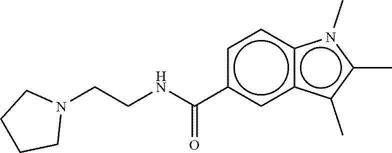 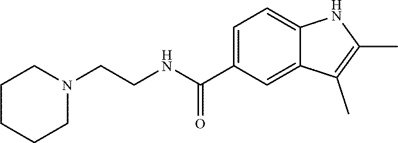 [SMILES: Cc1[nH]c2ccc(C(=O)NCCN3CCCCC3)cc2c1C] 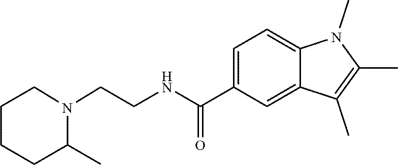 [SMILES: Cc1c(C)n(C)c2ccc(C(=O)NCCN3CCCCC3C)cc12] 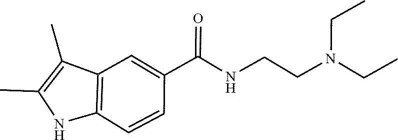 [SMILES: CCN(CC)CCNC(=O)c1ccc2[nH]c(C)c(C)c2c1] 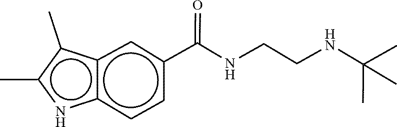 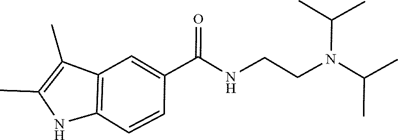 [SMILES: Cc1[nH]c2ccc(C(=O)NCCN(C(C)C)C(C)C)cc2c1C] 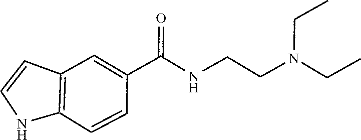 [SMILES: CCN(CC)CCNC(=O)c1ccc2[nH]ccc2c1] 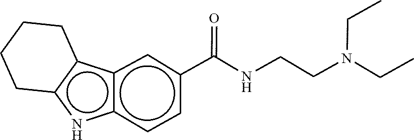 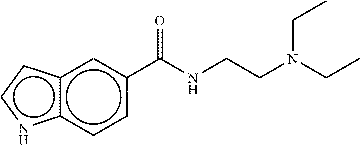 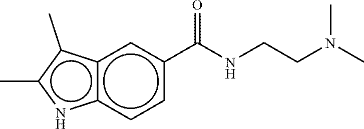 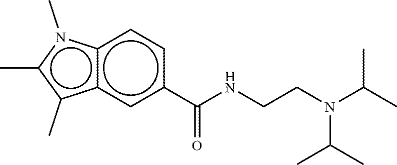 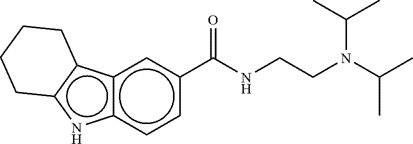
.

37. The method of any of claims 1-6, wherein the compound has the formula : 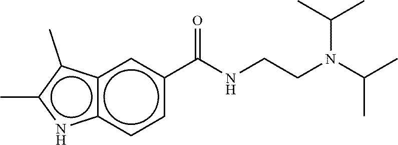
.

38. The method of claim 2, wherein the compound of Formula I partially reverses the conformational change of mutated huntingtin.

39. The method of claim 2, wherein the compound of Formula I completely reverses the conformational change of mutated huntingtin.

40. The method of claim 2, wherein the conformational change results in improved autophagy.

41. The method of claim 40, wherein the improved autophagy is improved autophagic flux.

42. The method of claim 3, wherein the polyQ disease is Huntington’s disease, Spinocerebellar ataxia 1, Spinocerebellar ataxia 2, Spinocerebellar ataxia 3, Spinocerebellar ataxia 6, Spinocerebellar ataxia 7, Spinocerebellar ataxia 17, Dentatorubropallidoluysian atrophy or Spinal and bulbar muscular atrophy.

43. The method of claim 4, wherein the mutated or misfolded protein is reduced in vivo.

44. The method of claim 4, wherein the mutated or misfolded protein is reduced in the brain.

45. The method of claim 4, wherein the mutated or misfolded protein is reduced by autophagy.

46. The method of claim 4, wherein the mutated or misfolded protein is reduced by increasing autophagic flux.

47. The method of claim 4, wherein the mutated or misfolded protein is reduced by increasing degradation.

48. The method of claim 4, wherein the mutated or misfolded protein is huntingtin.

49. A method of treating Huntington’s Disease; reversing a conformational change of mutated huntingtin; treating a polyQ disease; reducing mutated or misfolded proteins, or inducing autophagy, comprising administering to a subject in need thereof a compound selected from:

| Compound No. | Structure |
|---|---|
| 101 | 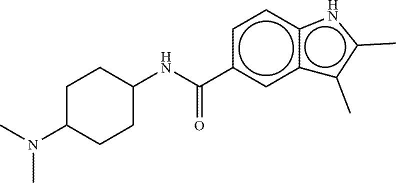 |
| 102 | 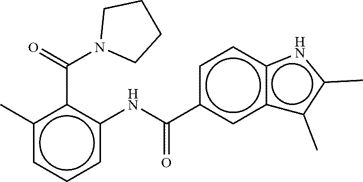 |
| 103 | 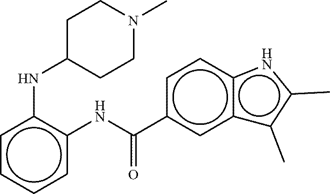 |
| 104 | 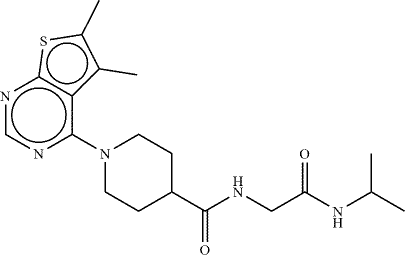 |
| 105 | 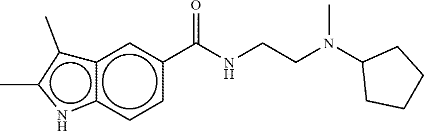 |
| 106 | 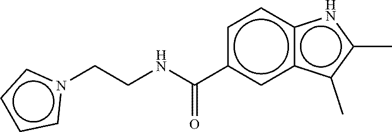 |
| 107 | 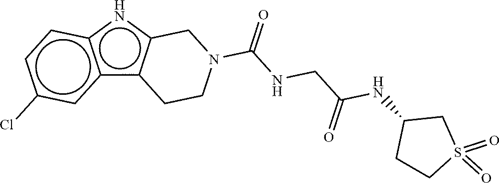 |
| 108 | 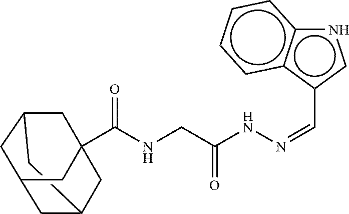 |
| 109 | 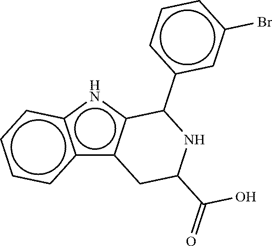 |
| 110 | 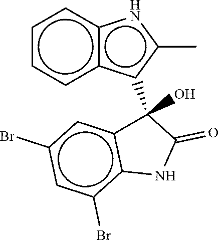 |

.

50. Use of a compound of Formula I in the manufacture of a medicament for treating Huntington’s Disease; reversing a conformational change of mutated huntingtin; treating a polyQ disease; or reducing mutated or misfolded proteins: 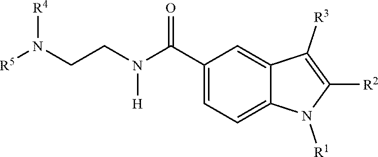
or a pharmaceutically acceptable salt, prodrug, solvate, hydrate, tautomer or isomer thereof, wherein:
R¹ is independently —H, —C₁—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
R² is independently —H, —C₁—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
or R¹ and R² can optionally combine, together with the atoms to which they are attached, to form a C₄-C₈heterocycle, wherein the heterocycle is optionally substituted with one or more substituents independently selected from the group consisting of —Br, —F, —Ci—C₆alkyl, —C₂—C₆alkenyl, and —C₂—C₆alkynyl, and wherein each alkyl, alkenyl, and alkynyl is optionally substituted with one or more —Br or —F;
R³ is independently —H, —C₁—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
or R² and R³ can optionally combine, together with the atoms to which they are attached, to form a C₄-C₈carbocycle or a C₄-C₈heterocycle, wherein the carbocycle or heterocycle is optionally substituted with one or more substituents independently selected from the group consisting of —Br, —F, —C₁—C₆alkyl, —C₂—C₆alkenyl, and —C₂—C₆alkynyl, and wherein each alkyl, alkenyl, and alkynyl is optionally substituted with one or more —Br or —F;
R⁴ is independently —H, —C₁—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
R⁵ is independently —H, —C₁—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
or R⁴ and R⁵ can optionally combine, together with the nitrogen atom to which they are attached, to form a C₃-C₈heterocycle, wherein the heterocycle is optionally substituted with one or more substituents independently selected from the group consisting of —Br, —F, —Ci—C₆alkyl, —C₂—C₆alkenyl, and —C₂—C₆alkynyl, and wherein each alkyl, alkenyl and alkynyl is optionally substituted with one or more —Br or —F.

51. Use of a compound of Formula I for treating Huntington’s Disease; reversing a conformational change of mutated huntingtin; treating a polyQ disease; or reducing mutated or misfolded proteins: 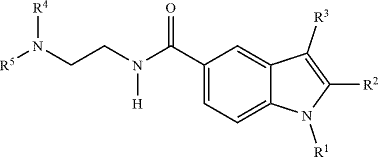
or a pharmaceutically acceptable salt, prodrug, solvate, hydrate, tautomer or isomer thereof, wherein:
R¹ is independently —H, —C₁—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
R² is independently —H, —C₁—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
or R¹ and R² can optionally combine, together with the atoms to which they are attached, to form a C₄-C₈heterocycle, wherein the heterocycle is optionally substituted with one or more substituents independently selected from the group consisting of —Br, —F, —Ci—C₆alkyl, —C₂—C₆alkenyl, and —C₂—C₆alkynyl, and wherein each alkyl, alkenyl, and alkynyl is optionally substituted with one or more —Br or —F;
R³ is independently —H, —C₁—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
or R² and R³ can optionally combine, together with the atoms to which they are attached, to form a C₄-C₈carbocycle or a C₄-C₈heterocycle, wherein the carbocycle or heterocycle is optionally substituted with one or more substituents independently selected from the group consisting of —Br, —F, —C₁—C₆alkyl, —C₂—C₆alkenyl, and —C₂—C₆alkynyl, and wherein each alkyl, alkenyl, and alkynyl is optionally substituted with one or more —Br or —F;
R⁴ is independently —H, —C₁—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
R⁵ is independently —H, —C₁—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
or R⁴ and R⁵ can optionally combine, together with the nitrogen atom to which they are attached, to form a C₃-C₈heterocycle, wherein the heterocycle is optionally substituted with one or more substituents independently selected from the group consisting of —Br, —F, —Ci—C₆alkyl, —C₂—C₆alkenyl, and —C₂—C₆alkynyl, and wherein each alkyl, alkenyl and alkynyl is optionally substituted with one or more —Br or —F.

52. A compound of Formula I or a pharmaceutically acceptable salt, prodrug, solvate, hydrate, tautomer or isomer thereof, 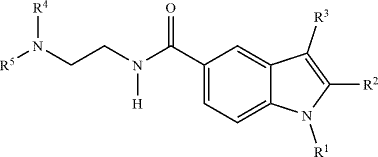
for use in treating Huntington’s Disease; reversing a conformational change of mutated huntingtin; treating a polyQ disease; or reducing mutated or misfolded proteins, wherein:
R¹ is independently —H, —C₁—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
R² is independently —H, —C₁—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
or R¹ and R² can optionally combine, together with the atoms to which they are attached, to form a C₄-C₈heterocycle, wherein the heterocycle is optionally substituted with one or more substituents independently selected from the group consisting of —Br, —F, —Ci—C₆alkyl, —C₂—C₆alkenyl, and —C₂—C₆alkynyl, and wherein each alkyl, alkenyl, and alkynyl is optionally substituted with one or more —Br or —F;
R³ is independently —H, —C₁—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
or R² and R³ can optionally combine, together with the atoms to which they are attached, to form a C₄-C₈carbocycle or a C₄-C₈heterocycle, wherein the carbocycle or heterocycle is optionally substituted with one or more substituents independently selected from the group consisting of —Br, —F, —C₁—C₆alkyl, —C₂—C₆alkenyl, and —C₂—C₆alkynyl, and wherein each alkyl, alkenyl, and alkynyl is optionally substituted with one or more —Br or —F;
R⁴ is independently —H, —C₁—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
R⁵ is independently —H, —C₁—C₆alkyl, —C₂—C₆alkenyl, or —C₂—C₆alkynyl, wherein each alkyl, alkenyl and alkynyl is optionally substituted with one or more substituents independently selected from the group consisting of —Br and —F;
or R⁴ and R⁵ can optionally combine, together with the nitrogen atom to which they are attached, to form a C₃-C₈heterocycle, wherein the heterocycle is optionally substituted with one or more substituents independently selected from the group consisting of —Br, —F, —Ci—C₆alkyl, —C₂—C₆alkenyl, and —C₂—C₆alkynyl, and wherein each alkyl, alkenyl and alkynyl is optionally substituted with one or more —Br or —F.

53. A compound selected from the group consisting of:

| Compound No. | Structure |
|---|---|
| 1 | 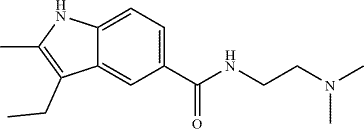 |
| 2 | 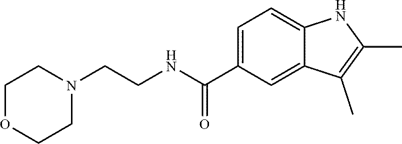 [SMILES: Cc1[nH]c2ccc(C(=O)NCCN3CCOCC3)cc2c1C] |
| 3 | 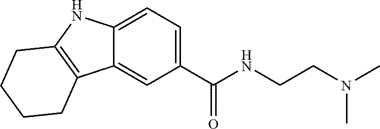 [SMILES: CN(C)CCNC(=O)c1ccc2[nH]c3c(c2c1)CCCC3] |
| 4 | 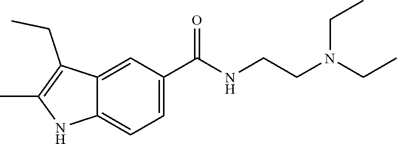 [SMILES: CCc1c(C)[nH]c2ccc(C(=O)NCCN(CC)CC)cc12] |
| 5 | 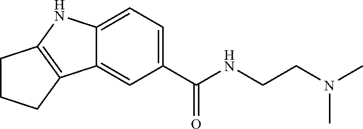 [SMILES: CN(C)CCNC(=O)c1ccc2[nH]c3c(c2c1)CCC3] |
| 6 | 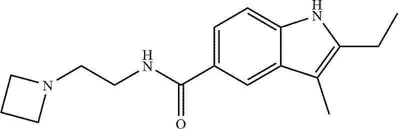 [SMILES: CCc1[nH]c2ccc(C(=O)NCCN3CCC3)cc2c1C] |
| 7 | 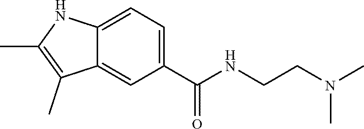 |
| 8 | 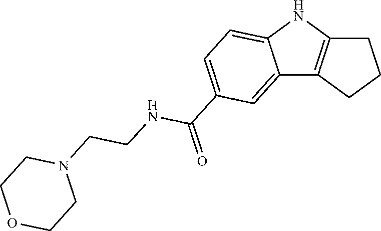 [SMILES: O=C(NCCN1CCOCC1)c1ccc2[nH]c3c(c2c1)CCC3] |
| 9 | 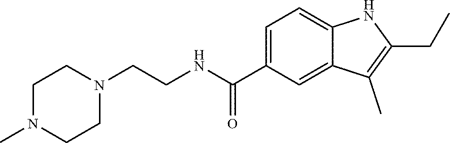 [SMILES: CCc1[nH]c2ccc(C(=O)NCCN3CCN(C)CC3)cc2c1C] |
| 10 | 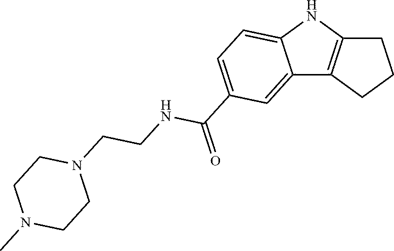 [SMILES: CN1CCN(CCNC(=O)c2ccc3[nH]c4c(c3c2)CCC4)CC1] |
| 11 | 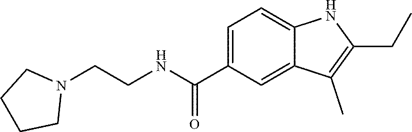 [SMILES: CCc1[nH]c2ccc(C(=O)NCCN3CCCC3)cc2c1C] |
| 12 | 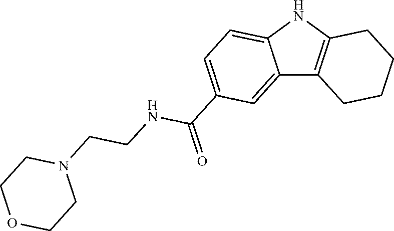 [SMILES: O=C(NCCN1CCOCC1)c1ccc2[nH]c3c(c2c1)CCCC3] |
| 13 | 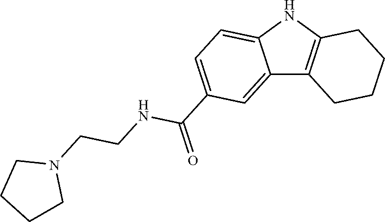 [SMILES: O=C(NCCN1CCCC1)c1ccc2[nH]c3c(c2c1)CCCC3] |
| 14 | 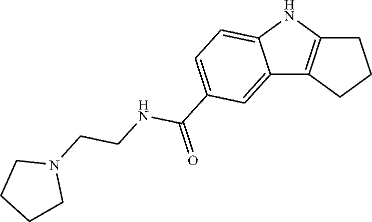 [SMILES: O=C(NCCN1CCCC1)c1ccc2[nH]c3c(c2c1)CCC3] |
| 15 | 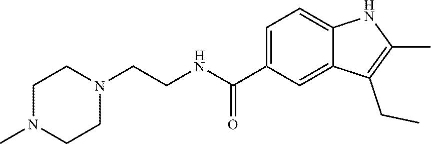 |
| 16 | 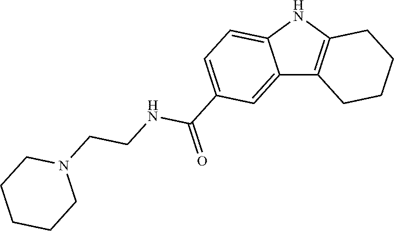 [SMILES: O=C(NCCN1CCCCC1)c1ccc2[nH]c3c(c2c1)CCCC3] |
| 17 | 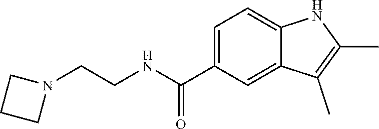 [SMILES: Cc1[nH]c2ccc(C(=O)NCCN3CCC3)cc2c1C] |
| 18 | 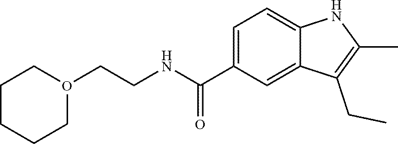 |
| 19 | 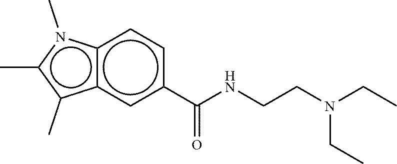 |
| 20 | 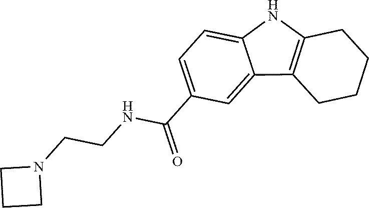 [SMILES: O=C(NCCN1CCC1)c1ccc2[nH]c3c(c2c1)CCCC3] |
| 21 | 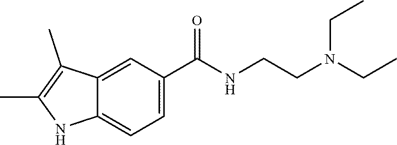 [SMILES: CCN(CC)CCNC(=O)c1ccc2[nH]c(C)c(C)c2c1] |
| 22 | 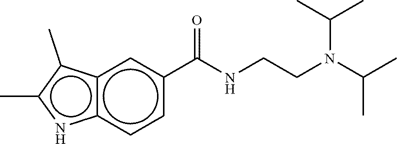 |
| 23 | 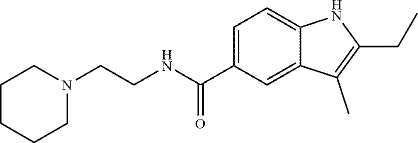 [SMILES: CCc1[nH]c2ccc(C(=O)NCCN3CCCCC3)cc2c1C] |
| 24 | |
| 25 | 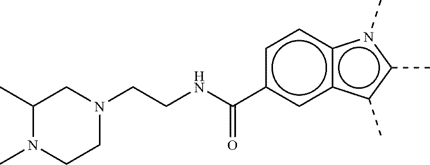 |
| 26 | 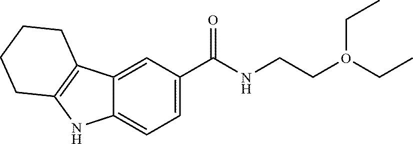 |
| 27 | 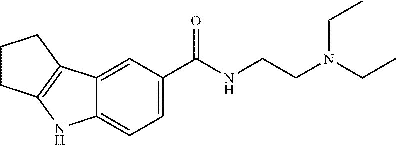 [SMILES: CCN(CC)CCNC(=O)c1ccc2[nH]c3c(c2c1)CCC3] |
| 28 | 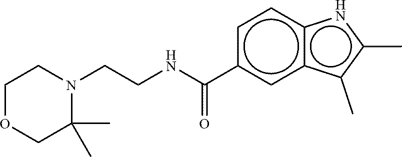 |
| 29 | 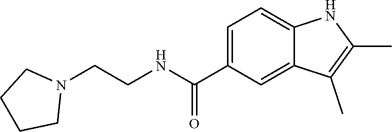 [SMILES: Cc1[nH]c2ccc(C(=O)NCCN3CCCC3)cc2c1C] |
| 30 | 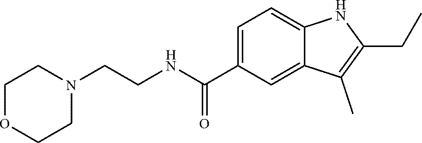 [SMILES: CCc1[nH]c2ccc(C(=O)NCCN3CCOCC3)cc2c1C] |
| 31 | |
| 32 | 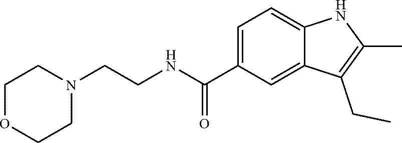 [SMILES: CCc1c(C)[nH]c2ccc(C(=O)NCCN3CCOCC3)cc12] |
| 33 | 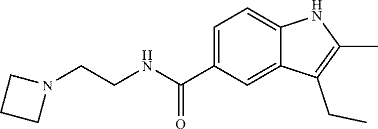 [SMILES: CCc1c(C)[nH]c2ccc(C(=O)NCCN3CCC3)cc12] |
| 34 | 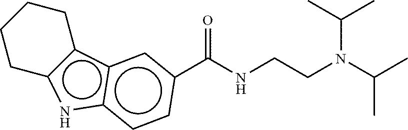 |
| 35 | 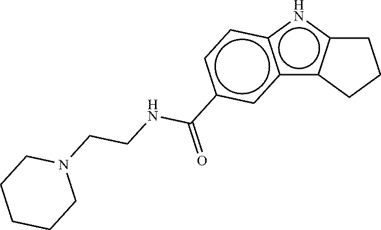 |
| 36 | 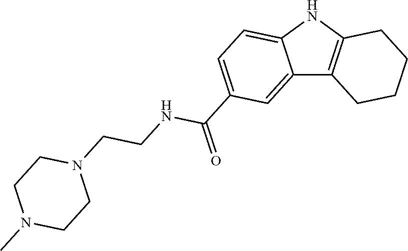 [SMILES: CN1CCN(CCNC(=O)c2ccc3[nH]c4c(c3c2)CCCC4)CC1] |
| 37 | 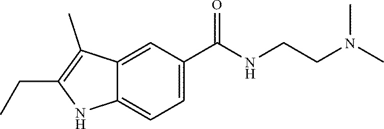 [SMILES: CCc1[nH]c2ccc(C(=O)NCCN(C)C)cc2c1C] |
| 38 | 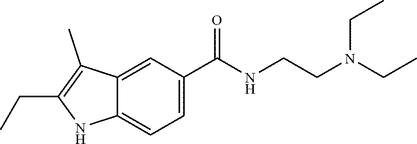 [SMILES: CCc1[nH]c2ccc(C(=O)NCCN(CC)CC)cc2c1C] |
| 39 | 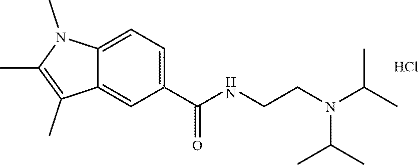 [SMILES: Cc1c(C)n(C)c2ccc(C(=O)NCCN(C(C)C)C(C)C)cc12 ; Cl] |
| 40 | 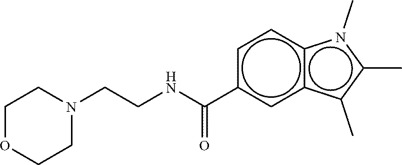 |
| 41 | 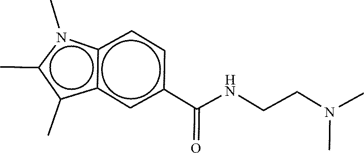 |
| 42 | 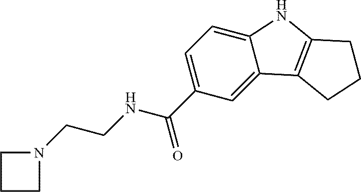 [SMILES: O=C(NCCN1CCC1)c1ccc2[nH]c3c(c2c1)CCC3] |
| 43 | 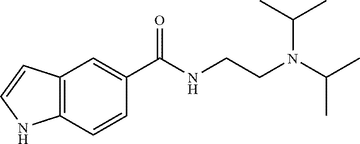 [SMILES: CC(C)N(CCNC(=O)c1ccc2[nH]ccc2c1)C(C)C] |
| 44 | 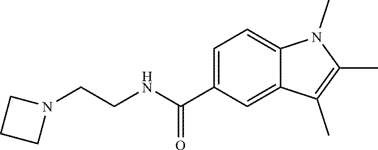 [SMILES: Cc1c(C)n(C)c2ccc(C(=O)NCCN3CCC3)cc12] |
| 45 | 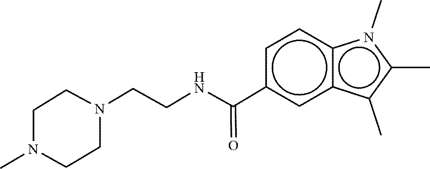 |
| 46 | 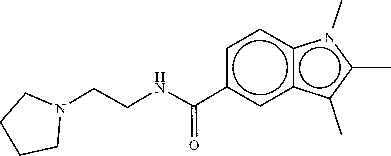 |
| 47 | 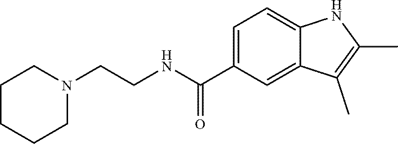 [SMILES: Cc1[nH]c2ccc(C(=O)NCCN3CCCCC3)cc2c1C] |
| 48 | 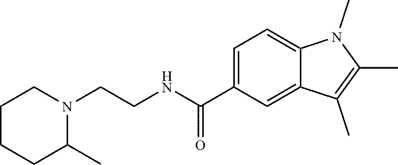 [SMILES: Cc1c(C)n(C)c2ccc(C(=O)NCCN3CCCCC3C)cc12] |
| 50 | 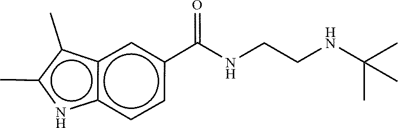 |
| 52 | 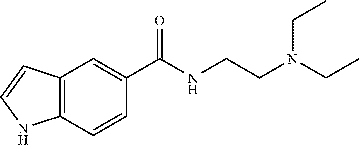 [SMILES: CCN(CC)CCNC(=O)c1ccc2[nH]ccc2c1] |
| 53 | 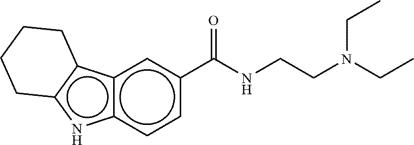 |
| 54 | 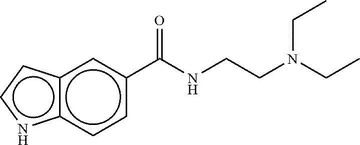 |
| 55 | |
| 56 | 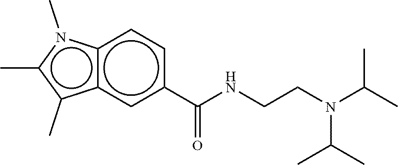 |
| 57 | 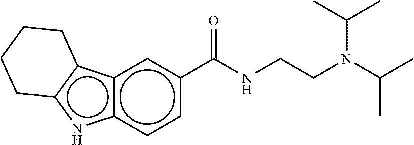 |
| 58 | |
| 59 | 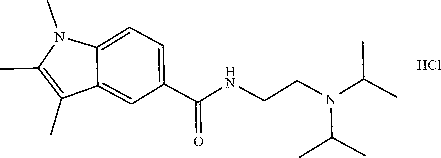 [SMILES: Cc1c(C)n(C)c2ccc(C(=O)NCCN(C(C)C)C(C)C)cc12 ; Cl] |
| 72 | 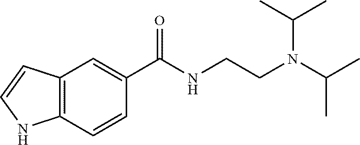 [SMILES: CC(C)N(CCNC(=O)c1ccc2[nH]ccc2c1)C(C)C] |
| 105 | 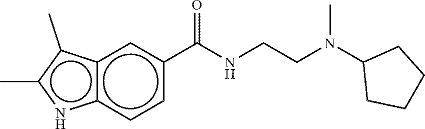 |
| 106 | 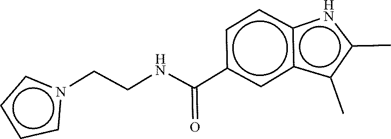 |
| 114 | 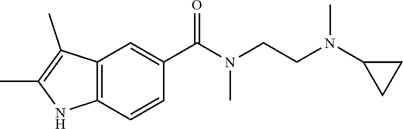 [SMILES: Cc1[nH]c2ccc(C(=O)N(C)CCN(C)C3CC3)cc2c1C] |

or a pharmaceutically acceptable salt thereof.

54. A pharmaceutical composition comprising a compound of claim 53 or a pharmaceutically acceptable salt thereof.

55. A method of treating a neurological disease or disorder, comprising administering to a subject in need thereof a therapeutically effective amount of a compound of any of claim 1-53 or pharmaceutical composition of claim 54.

56. The method of claim 55, wherein the neurological disease is a polyQ disease.

57. The method of claim 56, wherein the polyQ disease is selected from the group consisting of Huntington’s Disease, spinocerebellar ataxia, spinal bulbar muscular atrophy, Kennedy’s Disease, and dentatorubral-pallidoluysian atrophy.

58. The method of claim 57, wherein the polyQ disease is Huntington’s Disease.

59. The method of claim 57, wherein spinocerebellar ataxia is selected from the group consisting of spinocerebellar ataxia 1, spinocerebellar ataxia 2, spinocerebellar ataxia 3, spinocerebellar ataxia 6, and spinocerebellar ataxia 7.

60. The method of claim 57, wherein the polyQ disease is selected from spinal bulbar muscular atrophy, Kennedy’s Disease, and dentatorubral-pallidoluysian atrophy.

61. The method of claim 55, wherein the neurological disease or disorder is selected from the group consisting of Alzheimer’s disease, Parkinson’s disease, Frontotemporal dementia, Multiple system atrophy, Amyotrophic lateral sclerosis, Friedreich’s ataxia, Motor neurone diseases, and Spinal muscular atrophy.

62. The method of claim 55, wherein the neurological disease or disorder is Alzheimer’s disease.

63. The method of claim 55, wherein the neurological disease or disorder is Parkinson’s disease.

64. The method of claim 55, wherein the neurological disease or disorder is Frontotemporal dementia.

65. The method of claim 55, wherein the neurological disease or disorder is a delayed effect of stroke.

66. A method of reversing a conformational change of mutated huntingtin, comprising administering to a subject in need thereof an effective amount of a compound of claim 53.

67. A compound of Formula (I): 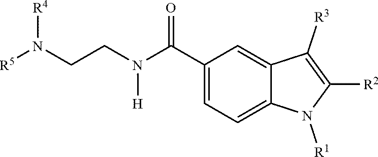
or a pharmaceutically acceptable salt thereof, wherein:
R¹ is independently hydrogen or methyl;
R² and R³ are each independently cyano, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
wherein R² and R³ cannot both simultaneously be methyl;
R⁴ and R⁵ are each independently hydrogen, cyano, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
or R⁴ and R⁵ can optionally combine, together with the atom to which they are attached, to form an optionally substituted 4 to 6-membered heterocyclyl, wherein the heterocyclyl is not imidazole substituted with a methyl.

68. The compound of claim 67, wherein R¹ is hydrogen.

69. The compound of claim 67, wherein R¹ is methyl.

70. The compound of claim 67, wherein R² and R³ are each independently substituted or unsubstituted alkyl, substituted or unsubstituted alkenyl, substituted or unsubstituted alkynyl, or substituted or unsubstituted carbocyclyl.

71. The compound of claim 67, wherein R² and R³ are each independently substituted or unsubstituted alkyl.

72. The compound of claim 67, wherein R² and R³ are each independently substituted or unsubstituted C₁-C₆ alkyl.

73. The compound of claim 67, wherein R² is methyl.

74. The compound of claim 67, wherein R³ is ethyl.

75. The compound of claim 67, wherein R² is methyl and R³ is ethyl.

76. The compound of claim 67, wherein R⁴ and R⁵ are each independently hydrogen, cyano, substituted or unsubstituted alkyl, substituted or unsubstituted carbocyclyl, substituted or unsubstituted heterocyclyl, substituted or unsubstituted aryl, or substituted or unsubstituted heteroaryl.

77. The compound of claim 67, wherein R⁴ and R⁵ are each independently substituted or unsubstituted alkyl.

78. The compound of claim 67, wherein R⁴ and R⁵ are each independently substituted or unsubstituted C₁-C₆ alkyl.

79. The compound of claim 67, wherein R⁴ and R⁵ are each independently methyl, ethyl, or propyl.

80. The compound of claim 67, wherein R⁴ and R⁵ are both ethyl.

81. The compound of claim 67, wherein R⁴ and R⁵ combine and together with the atom to which they are attached form a six member heterocyclyl with 1, 2, or 3 heteroatoms, wherein the heteroatoms are either O or N.

82. The compound of claim 81, where the heterocyclyl is optionally substituted with a C₁-C₆ alkyl.

83. The compound of claim 82, wherein the C₁-C₆ alkyl is methyl.

84. The compound of claim 67, wherein R⁴ and R⁵ combine and together with the atom to which they are attached form a five member heterocyclyl.

85. The compound of claim 84, where the heterocyclyl is optionally substituted with a C₁-C₆ alkyl.

86. The compound of claim 85, wherein the C₁-C₆ alkyl is methyl.

87. The compound of claim 67, wherein R⁴ and R⁵ combine and together with the atom to which they are attached form a four member heterocyclyl.

88. The compound of claim 87, where the heterocyclyl is optionally substituted with a C₁-C₆ alkyl.

89. The compound of claim 88, wherein the C₁-C₆ alkyl is methyl.

90. The compound of claim 67, wherein the compound is selected from the group consisting of: 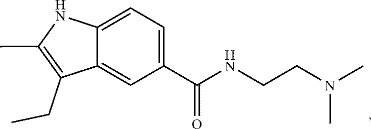 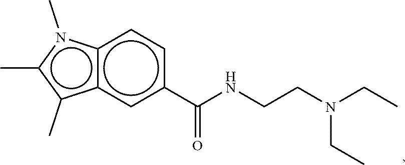 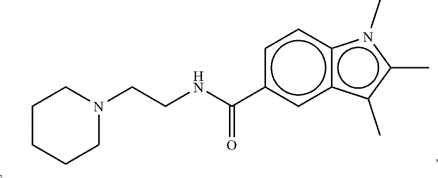 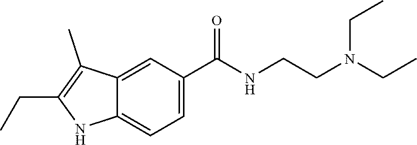 [SMILES: CCc1[nH]c2ccc(C(=O)NCCN(CC)CC)cc2c1C] 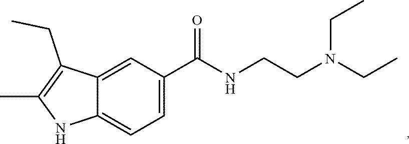 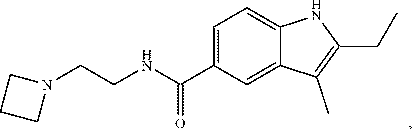 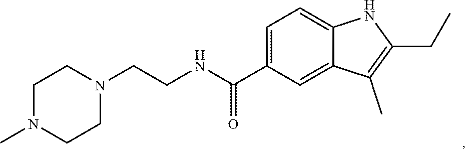 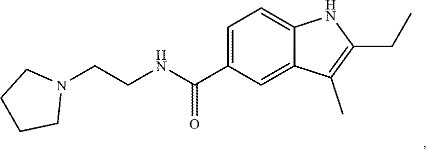 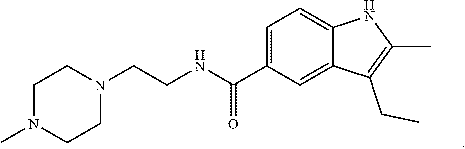 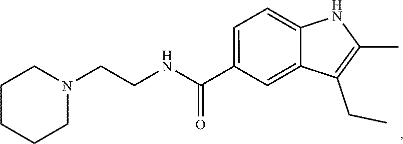 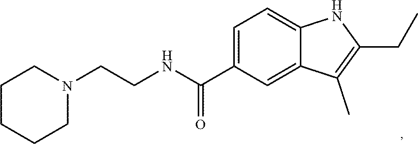 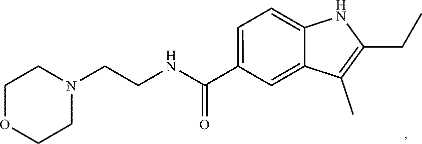 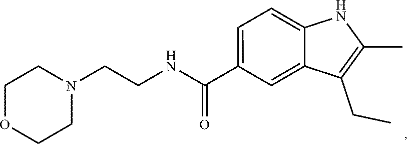 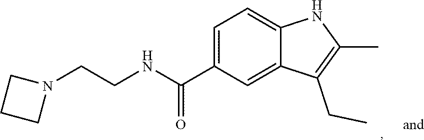 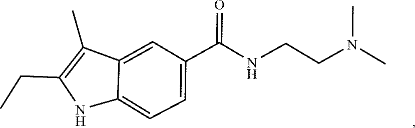
or a pharmaceutically acceptable salt.

91. The compound of claim 67, wherein the compound is 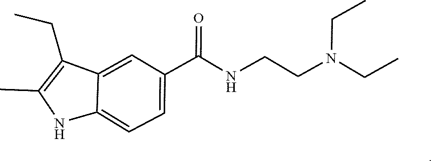
.

92. The compound of claim 67, wherein the compound is 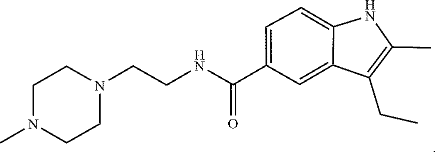
.

93. The compound of claim 67, wherein the compound is 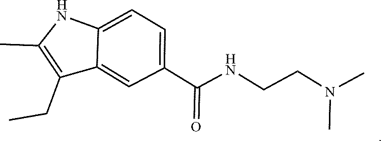
.

94. A compound of Formula (I): 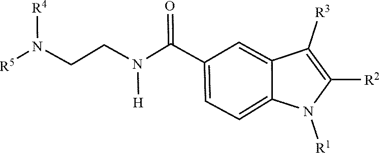
or a pharmaceutically acceptable salt thereof, wherein:
R¹ is independently hydrogen or methyl;
R² and R³ combine, together with the atoms to which they are attached, to form a substituted or unsubstituted carbocyclyl, substituted or unsubstituted heterocyclyl, substituted or unsubstituted aryl, or substituted or unsubstituted heteroaryl.
R⁴ and R⁵ are each independently hydrogen, cyano, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
or R⁴ and R⁵ can optionally combine, together with the atom to which they are attached, to form an optionally substituted 4 to 6-membered heterocyclyl, wherein the heterocyclyl is not imidazole substituted with a methyl.

95. The compound of claim 94, wherein R¹ is hydrogen.

96. The compound of claim 94, wherein R¹ is methyl.

97. The compound of claim 94, wherein R₂ and R₃ combine, together with the atoms to which they are attached, to form a substituted or unsubstituted carbocyclyl.

98. The compound of claim 94, wherein R₂ and R₃ combine, together with the atoms to which they are attached, to form an unsubstituted carbocyclyl.

99. The compound of claim 94, wherein R₂ and R₃ combine, together with the atoms to which they are attached, to form a 5 or 6 member unsubstituted carbocyclyl.

100. The compound of claim 94, wherein R₂ and R₃ combine, together with the atoms to which they are attached, to form a 5 member unsubstituted carbocyclyl.

101. The compound of claim 100, wherein the carbocyclyl is partially unsaturated.

102. The compound of claim 94, wherein R₂ and R₃ combine, together with the atoms to which they are attached to form a 6 member unsubstituted carbocyclyl.

103. The compound of claim 102, wherein the carbocyclyl is partially unsaturated.

104. The compound of claim 94, wherein R⁴ and R⁵ are each independently hydrogen, cyano, substituted or unsubstituted alkyl, substituted or unsubstituted carbocyclyl, substituted or unsubstituted heterocyclyl, substituted or unsubstituted aryl, or substituted or unsubstituted heteroaryl.

105. The compound of claim 94, wherein R⁴ and R⁵ are each independently substituted or unsubstituted alkyl.

106. The compound of claim 94, wherein R⁴ and R⁵ are each independently substituted or unsubstituted C₁-C₆ alkyl.

107. The compound of claim 94, wherein R⁴ and R⁵ are each independently methyl, ethyl, or propyl.

108. The compound of claim 94, wherein R⁴ and R⁵ are both methyl.

109. The compound of claim 94, wherein R⁴ and R⁵ combine and together with the atom to which they are attached form a six member heterocyclyl with 1, 2, or 3 heteroatoms, wherein the heteroatoms are either O or N.

110. The compound of claim 109, wherein the heterocyclyl is optionally substituted with a C₁-C₆ alkyl.

111. The compound of claim 110, wherein the C₁-C₆ alkyl is methyl.

112. The compound of claim 94, wherein R⁴ and R⁵ combine and, together with the atom to which they are attached, form a five member heterocyclyl.

113. The compound of claim 112, wherein the heterocyclyl is optionally substituted with a C₁-C₆ alkyl.

114. The compound of claim 113, wherein the C₁-C₆ alkyl is methyl.

115. The compound of claim 94, wherein R⁴ and R⁵ combine and together with the atom to which they are attached form a four member heterocyclyl.

116. The compound of claim 115 where the heterocyclyl is optionally substituted with a C₁-C₆ alkyl.

117. The compound of claim 116, wherein the C₁-C₆ alkyl is methyl.

118. The compound of claim 94, wherein the compound is selected from the group consisting of: 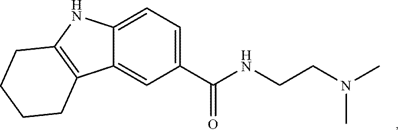 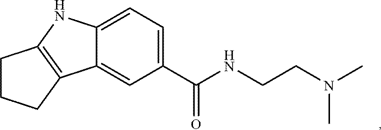 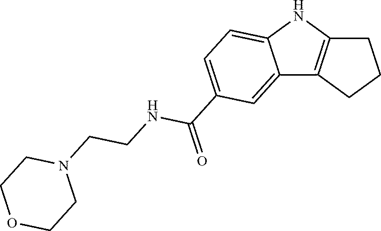 [SMILES: O=C(NCCN1CCOCC1)c1ccc2[nH]c3c(c2c1)CCC3] 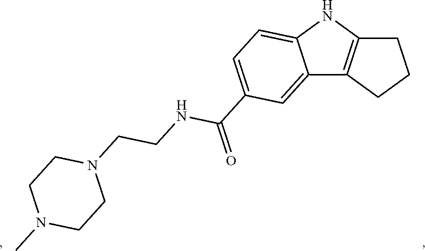 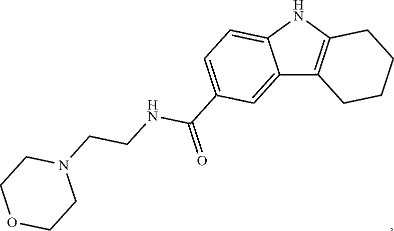 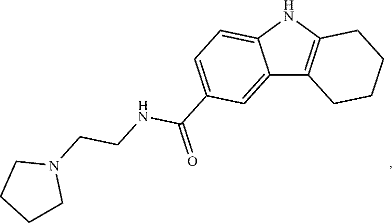 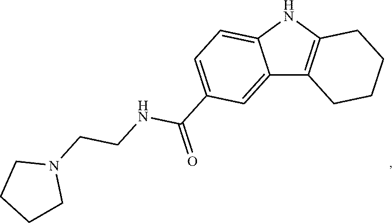 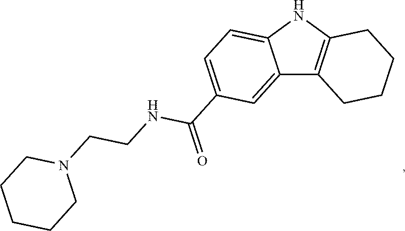 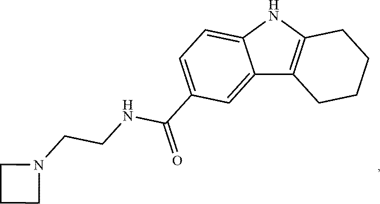 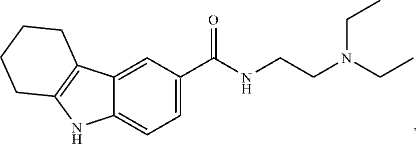 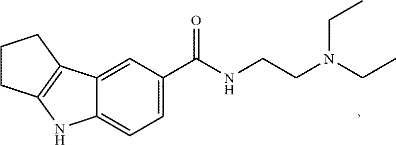 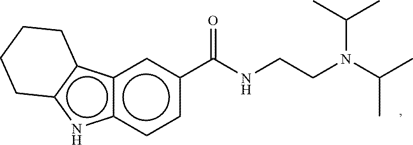 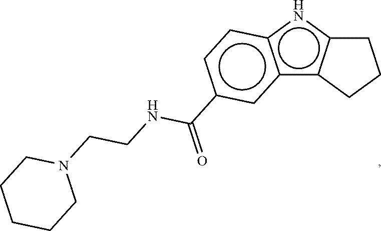 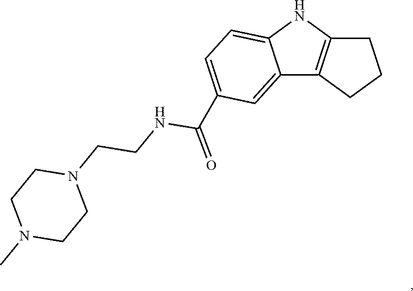 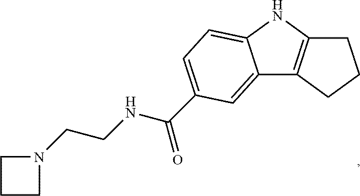 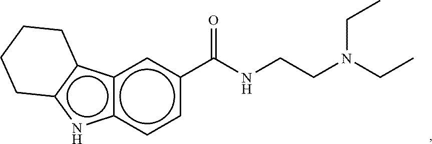
and 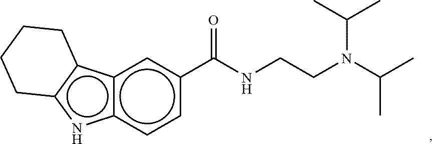
or a pharmaceutically acceptable salt thereof.

119. The compound of claim 94, wherein the compound is 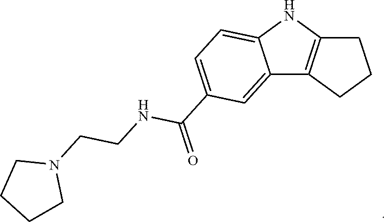
.

120. The compound of claim 94, wherein the compound is 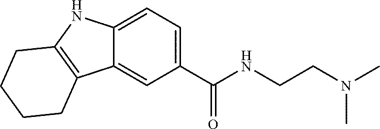 [SMILES: CN(C)CCNC(=O)c1ccc2[nH]c3c(c2c1)CCCC3]
.

121. The compound of claim 94, wherein the compound is 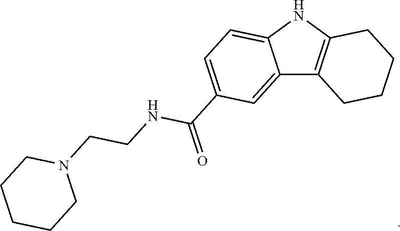
.

122. The compound of claim 94, wherein the compound is 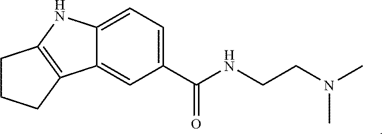
.

123. The compound of claim 94, wherein the compound is 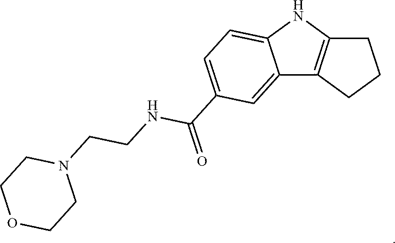
.

124. The compound of claim 94, wherein the compound is 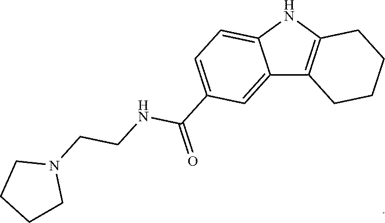
.

125. The compound of claim 94, wherein the compound is 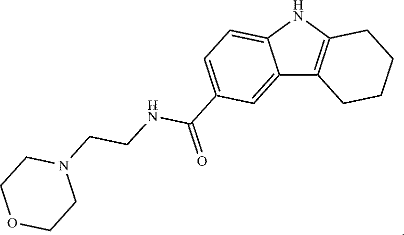
.


SEQUENCE LISTING (11 sequences)

SEQ ID NO: 1 - protein, 22 aa, Artificial Sequence
  Synthetic peptide
GSGSGERQHMDSPDLGTDDDDK

SEQ ID NO: 2 - protein, 103 aa, Artificial Sequence
  Synthetic polypeptide
MIAPILDEIADEYQGKLTVAKLNIDQNPGTAPKYGIRGIPTLLLFKNGEVAATKVGALSKGQLKEFLDANLAGSGSGERQHMDSPDLGTDDDDKGSGHHHHHH

SEQ ID NO: 3 - protein, 103 aa, Artificial Sequence; identical to SEQ ID NO: 2
  Synthetic polypeptide

SEQ ID NO: 4 - protein, 222 aa, Artificial Sequence
  Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGERQHMDSPDLGTDDDDKMATLEKLMKAFESLKSFQQQQQQQQQQQQQQQQPPPPPPPPPPPQLPQPPPQAQPLLPQPQPPPPPPPPPPGPAVAEEPLHRGSGHHHHHH

SEQ ID NO: 5 - protein, 222 aa, Artificial Sequence; identical to SEQ ID NO: 4
  Synthetic polypeptide

SEQ ID NO: 6 - protein, 378 aa, Artificial Sequence
  Synthetic polypeptide
  MOD_RES (1)..(1): Any amino acid
  MOD_RES (4)..(4): Any amino acid
  MOD_RES (360)..(363): Any amino acid
  MOD_RES (377)..(377): Any amino acid
XPSXNILFTLRRRYTMSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGERQHMDSPDLGTDDDDKMATLEKLMKAFESLKSFQQQQQQQQQQQQQQQQQQQQQQQQQQQQQQQQQQQQQQQQQQQQQQPPPPPPPPPPPQLPQPPPQAQPLLPQPQPPPPPPPPPPGPAVAEEPLHRGSGHHHHHHAAALEHHHHHHDPAANKARKEAELAAATAEQLAPLGASKRVLRGFLLKGGTISGLANGTRPVAAHARRVWWLRAAPLHLPAPRPLLSLSSLPXXXXSPAFPVKLIGGSLXS

SEQ ID NO: 7 - protein, 265 aa, Artificial Sequence
  Synthetic polypeptide
KFCLTLRRRYTMSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGERQHMDSPDLGTDDDDKMATLEKLMKAFESLKSFQQQQQQQQQQQQQQQQQQQQQQQQQQQQQQQQQQQQQQQQQQQQQQPPPPPPPPPPPQLPQPPPQAQPLLPQPQPPPPPPPPPPGPAVAEEPLHRGSGHHHHHHAA

SEQ ID NO: 8 - DNA, 25 bp, Artificial Sequence
  Synthetic primer
tcatcagcttttccagggtcgccat

SEQ ID NO: 9 - DNA, 26 bp, Artificial Sequence
  Synthetic primer
cgcaggctagggctgtcaatcatgct

SEQ ID NO: 10 - DNA, 28 bp, Artificial Sequence
  Synthetic primer
actccactcacggcaaattcaacggcac

SEQ ID NO: 11 - DNA, 28 bp, Artificial Sequence
  "Synthetic primer"
ggtcatgagcccttccacaatgccaaag